(19) United States
(12) Patent Application Publication
(10) Pub. No.: US 2024/0221868 A1
(43) Pub. Date: Jul. 4, 2024

(54) METHODS FOR SINGLE CELL NANOPORE SEQUENCING TECHNOLOGY AND DATA ANALYSIS

(71) Applicant: NORTHWESTERN UNIVERSITY, Evanston, IL (US)

(72) Inventors: Ruli Gao, Evanston, IL (US); Cheng-Kai Shiau, Evanston, IL (US); Lina Lu, Evanston, IL (US)

(21) Appl. No.: 18/531,821

(22) Filed: Dec. 7, 2023

Related U.S. Application Data
(60) Provisional application No. 63/430,727, filed on Dec. 7, 2022.
(60) Provisional application No. 63/469,901, filed on May 31, 2023.

(51) Int. Cl. G16B 30/00; G16B 25/10

(52) U.S. Cl. CPC G16B 30/00; G16B 25/10

(57) ABSTRACT

An analysis and computational tool, single cell nanopore sequencing analysis of Genotype-Phenotype simultaneously (scNanoGPS), to deconvolute barcoded long reads into single cells and single molecules without short reads curation nor guidance of barcode whitelist and calculate both phenotypes (gene expression, isoform) and genotypes (mutations) of same cells.

### CROSS-REFERENCE TO RELATED PATENT APPLICATIONS

This application claims priority to and the benefit of U.S. Provisional Patent Application Ser. No. 63/430,727, filed on Dec. 7, 2022, and U.S. Provisional Patent Application Ser. No. 63/469,901, filed on May 31, 2023, all of which are incorporated herein in their entireties by reference.

### STATEMENT AS TO RIGHTS UNDER FEDERALLY-SPONSORED RESEARCH

This invention was made with government support under grant number HL 160552 awarded by the National Institutes of Health. The government has certain rights in the invention.

### STATEMENT REGARDING THE SEQUENCE LISTINGS

The Sequence Listings associated with this application are provided in xml format in lieu of a paper copy and are hereby incorporated by reference into the specification. The name of the xml file containing the Sequence Listings is 0116936.271US2.xml. The xml file is about 10 KB, was created on Mar. 19, 2024.

### FIELD OF THE INVENTION

The present disclosure relates generally to field of bioinformatics, and more particularly to a tool to deconvolute barcoded long reads into single cell and single molecules without short reads curation nor whitelists of barcodes, and calculate both phenotypes and genotypes of the same cells for thousands of cells in parallel.

### BACKGROUND OF THE INVENTION

The background description provided herein is for the purpose of generally presenting the context of the present invention. The subject matter discussed in the background of the invention section should not be assumed to be prior art merely as a result of its mention in the background of the invention section. Similarly, a problem mentioned in the background of the invention section or associated with the subject matter of the background of the invention section should not be assumed to have been previously recognized in the prior art. The subject matter in the background of the invention section merely represents different approaches, which in and of themselves may also be inventions.

Human tissues including tumors represent complex ecological systems of diverse cell types with dynamic genetic evolution and phenotypic remodeling. However, there is a lack of robust methods for tracking both genotypes (e.g., mutations) and phenotypes (e.g., gene expressions, isoforms) of individual cells to precisely trace the cellular and molecular dynamics during tissue development, particularly during tumor evolution and treatment response.

Recently, long-read single cell Nanopore sequencing of full-length RNAs (scNanoRNAseq) is emerging as a powerful technology to simultaneously profile phenotypes and genotypes of same cells, which however is challenged by lacking robust analysis and computational tools and requiring parallel short reads to curate sequencing errors.

In particular, scNanoRNAseq is transforming single cell multi-omics analysis through direct measurement of nucleotide sequences of whole gene bodies without algorithmic reconstructions. Nowadays, high throughput long-read single cell sequencing methods take advantage of droplet barcoding systems (commonly, 10× Genomics Chromium system) to barcode full-length cDNAs of single cells and sequence them on ultra-high yield third-generation sequencing (TGS) platforms. Of note, the Oxford Nanopore Technology (ONT) platform, PromethION can yield ˜100 million reads per flow cell, providing adequate coverages of thousands of single cell transcriptomes. The PacBio system, Sequel II system can yield ˜8 million high fidelity reads, which can measure hundreds of single cell transcriptomes.

However, as discussed above, the broad applications of this powerful technology are computationally challenged due to the complexity of calculating same-cell multi-omics from these long-read data. Moreover, due to higher error rates in cell barcodes (CBs) and unique molecule identifiers (UMIs) of TGS data comparing to next-generation sequencing (NGS), current methods rely on generating paralleled NGS short-read data to guide the deconvolution of CBs and UMIs, which can drastically increase experimental costs and computational complexity and often results in partial usage of data. Recently, two methods called Sockeye and BLAZE were released to detect CBs without usages of paralleled NGS data. However, both methods relied on theoretical barcode whitelist (10× Genomics states there are ˜3.6 million unique sequences for 3′ GEX with v3 chemistry). The whitelist dependence makes both methods compromised because the manufactured barcodes may deviate from theoretical random combinations, particularly, the pool size reaches to millions of molecules that are 2 edit-distance apart. Moreover, different versions of protocols may have different whitelists and misusages of wrong whitelists are not easy to tell due to their similarities. Therefore, a robust computational tool for analyzing high throughput single cell long-read data is still missing.

Therefore, there remains an imperative need for analysis and computational tools to deconvolute barcoded long reads into single cell and single molecules without short reads curation nor barcode whitelist guidance, and calculate both phenotypes and genotypes of the same cells.

### SUMMARY OF THE INVENTION

In light of the foregoing, this invention discloses an analysis and computational tool, called single cell Nanopore sequencing analysis of Genotype-Phenotype simultaneously (scNanoGPS), to deconvolute barcoded long reads into single cells and single molecules without short reads curation nor the guidance of barcode whitelist, and calculate both phenotypes (gene expression, isoform) and genotypes (mutations) of same cells.

In one aspect of the invention, a method of performing single cell nanopore sequencing of genotype-phenotype simultaneously (scNanoGPS). The method comprises obtaining barcoded full-length cDNAs sequencing information of single cells; scanning the barcoded full-length cDNAs sequencing information to acquire barcoded information; curating errors in the barcode information to produce curated barcoded information; producing at least one BAM file based on the curated barcoded information; and calculating multi-omics information of the single cells based on the at least one BAM file.

In one embodiment, the barcoded full-length cDNAs sequencing information is obtained via long-read single cell nanopore sequencing technology.

In one embodiment, the method further comprises producing a FASTQ file based on the barcoded information acquired in step (2).

In one embodiment, the method further comprises refining the barcoded information acquired in step (2) before step (3) to produce refined barcoded information to detect true CBs.

In one embodiment, the method further comprises obtaining Unique Molecular Identifiers (UMIs) from the refined barcoded information; and curating at least one error in the Unique Molecular Identifiers (UMIs) in step (3) for transcriptome analysis.

In one embodiment, the method further comprises obtaining transcripts from the refined barcoded information; and curating at least one error in the transcripts in step (3) for the transcriptome analysis.

In one embodiment, the single cells multi-omics information includes at least one of gene expression matrix, isoforms profile and mutations profile.

In one embodiment, the single cells gene expression matrix is generated via: (a) calculating a UMI counts of genes in the single cells using the at least one BAM files; wherein each of the at least one BAM files is individually mapped; (b) selecting consensus reads that mapped to mature mRNA references to detect transcriptional isoforms of the single cells; and (c) calculating single cell mutation profiles from the consensus reads of the single cells.

In one embodiment, the method obviates using short read sequencing information of the single cells nor whitelist of barcode sequences as guidance for processing the barcoded full-length cDNAs sequencing information.

In one embodiment, the single cells include approximately 3000 cells per run of the long-read single cell nanopore sequencing.

In another aspect of the invention, a non-transitory computer readable medium storing a program causing a computer to execute a process of performing single cell nanopore sequencing of genotype-phenotype simultaneously (scNanoGPS), the process comprising obtaining barcoded full-length cDNAs sequencing information of single cells; scanning the barcoded full-length cDNAs sequencing information to acquire barcoded information; curating errors in the barcode information to produce curated barcoded information; producing at least one BAM file based on the curated barcoded information; and calculating multi-omics information of the single cells based on the at least one BAM file.

These and other aspects of the present invention will become apparent from the following description of the preferred embodiment taken in conjunction with the following drawings, although variations and modifications therein may be affected without departing from the spirit and scope of the novel concepts of the disclosure.

### BRIEF DESCRIPTION OF THE DRAWINGS

Illustrative embodiments of the invention will hereafter be described with reference to the accompanying drawings, wherein like numerals denote like elements.

FIG. 1 shows a schematic diagram of experimental workflow and library structure of scNanoRNAseq.

FIG. 2 shows a schematic diagram of computational workflow of scNanoGPS.

FIG. 3 shows illustration of scNanoGPS methods, blue boxes represent execution items, while others indicate files.

FIG. 4 shows length distribution of raw reads of two cancer cell lines during scNanoRNAseq data processing by scNanoGPS.

FIG. 5 shows intersection of true cell barcodes detected by scNanoGPS and standard NGS approaches.

FIG. 6 shows pair-wised scatter plots of UMI counts per cell detected by two approaches (Pearson correlation P-value <2.2e-16) during scNanoRNAseq data processing by scNanoGPS.

FIG. 7 shows pair-wised scatter plots of gene detection versus UMI counts of single cells.

FIG. 8 shows single cell gene expression levels calculated by scNanoGPS and standard NGS approaches. (Pearson correlation P-value <2.2e-16.)

FIG. 9 shows heatmap of genes with significantly different expression levels in scNanoGPS and standard NGS approaches.

FIG. 10 shows density plots of length of genes with significantly different expression levels in scNanoGPS and standard NGS approaches.

FIG. 11 shows heatmap of single cell copy number profiles of H2030 calculated by CopyKAT from matched single cell transcriptomes of two approaches.

FIG. 12 shows size distributions of cDNAs, namely, the TapeStation traces of full-length cDNAs of A375 (upper panel) and H2030 (lower panel) before making sequencing libraries.

FIG. 13 shows identification of low-quality cells, namely, UMAPs of low-quality cells detected by NGS and scNanoGPS in A375 (upper panel), and UMAPs of low-quality cells detected by NGS and scNanoGPS in H2030 (lower panel).

FIG. 14 shows saturation analysis of scNanoRNAseq depths in A375 and H2030 cell lines. Pearson's correlation of single cell transcriptome profiles with ground truth (NGS 3′scRNAseq consensus profile) of A375 (upper left panel) and H2030 (upper right panel). Pearson's correlation of mini-bulk (averages of single cells within binned read depths) long read transcriptomes with ground truth of A375 (lower left panel) and H2030 (lower right panel).

FIG. 15 shows performance of scNanoGPS in dissecting cell types in the tumor microenvironment. The upper panel shows UMAP projection of major cell types of 4 frozen tumors using data processed by scNanoGPS; the middle panel shows UMAP projection of major cell types of 4 frozen tumors using NGS data; the lower panel shows concordance of cell typing results between scNanoGPS and NGS approaches. Only cells detected in both scNanoGPS and standard NGS approaches were used for calculation.

FIG. 16 shows classification of tumor and normal cells in tumor samples. The left panel shows heatmaps of single cell copy number profiles inferred from scNanoRNAseq data of four frozen tumors, and the right panel shows UMAPs of gene expression levels of known cancer markers, e.g., EGFR in RCCs and MET in melanomas.

FIG. 17 shows numbers of genes with DCIs in 7 cell types of a kidney tumor, when scNanoGPS is applied for profiling cell-type-specific isoforms in the tumor microenvironment.

FIG. 18 shows stratification of genes with DCIs (different combination of isoforms) based on status of gene expression levels and most dominant transcripts (MDTs). dMDT, distinct among cell types; sMDT, shared across cell types.

FIG. 19 shows heatmap of the cellular frequencies of top cell-type-specific MDTs.

FIG. 20 shows numbers of exons of tumor and normal cell preferred isoforms.

FIG. 21 shows pair-wised scatter plot of the numbers of exons of expressed genes in tumor and normal cells.

FIG. 22 shows top gene ontologies (MSigDB: GO: BP) of cell-type-specific DCI genes.

FIG. 23 shows examples of cellular frequencies of isoforms of genes expressing different MDTs in different cell types.

FIG. 24 shows examples of cellular frequencies of isoforms of genes expressing same MDTs in different cell types.

FIG. 25 shows comparison of isoforms detected in long and short-read sequencing data of a frozen kidney tumor RCC1. The upper panel shows ven diagrams of isoforms detected in master FASTQ files of scNanoRNAseq data (TGS pseudo-bulk), traditional NGS-based bulk RNAseq (NGS bulk) and aggregated list of scNanoGPS results (scTGS aggregated); the lower panel shows density plots of Pearson's correlations of isoform expression levels calculated from the above-mentioned three data types.

FIG. 26 shows cell type specific genes with DCIs, namely, violin plots of gene expression levels of 6 example genes with cell-type-specific DCIs.

FIG. 27 shows cell type specific genes with DCIs, namely, IGV visualization of reads mapped to 3 example DCIs genes expressing different MDTs in different cell types.

FIG. 28 shows cell type specific genes with DCIs, namely, IGV visualization of reads mapped to 3 example DCIs genes expressing same MDTs in different cell types.

FIG. 29 shows transcriptome-wide mutation profiling of different cell types in the tumor microenvironment by scNanoGPS, particularly, pie chart of relative fractions of all mutations in different gene regions.

FIG. 30 shows transcriptome-wide mutation profiling of different cell types in the tumor microenvironment by scNanoGPS, particularly, pie chart of mutation types of all mutations.

FIG. 31 shows transcriptome-wide mutation profiling of different cell types in the tumor microenvironment by scNanoGPS, particularly, number of mutations in different chromosomes.

FIG. 32 shows transcriptome-wide mutation profiling of different cell types in the tumor microenvironment by scNanoGPS, particularly, heatmap of cell-type-specific somatic deMuts in all cells.

FIG. 33 shows transcriptome-wide mutation profiling of different cell types in the tumor microenvironment by scNanoGPS, particularly, UMAP projections of single cells labeled with 8 examples of tumor-cell-specific deMuts after SoupX.

FIG. 34 shows mutation detection efficiency and frequencies in different cell types in a frozen kidney tumor RCC1, particularly, density plots of percentages of cells expressed mutated transcripts overall all cells. Top, all point mutations; middle, germline mutations; bottom, somatic mutations.

FIG. 35 shows density plots of cellular frequencies of mutated transcripts over cells with coverages. Top, all point mutations; middle, germline mutations; bottom, somatic mutations.

FIG. 36 shows comparison of point mutations detected in long and short-read sequencing data of a frozen kidney tumor RCC1, particularly, ven diagrams of germline (left panel) or somatic (right panel) mutations detected in master FASTQ files of scNanoRNAseq data (TGS pseudo-bulk), traditional NGS-based bulk RNAseq (NGS bulk) and aggregated list of scNanoGPS results (scTGS aggregated).

FIG. 37 shows density plots of distributions of detected mutations across relative gene body positions.

FIG. 38 shows shared mutation hotspots in all major cell types from a frozen kidney tumor, particularly, cellular frequencies of all mutations, i.e., percentages of cells expressed mutants over all cells that had coverages.

FIG. 39 shows shared mutation hotspots in all major cell types from a frozen kidney tumor, particularly, cellular frequencies of mutations located in 4 hot spots in each major cell type.

FIG. 40 shows single cell transcriptome-wide mutation profiles in a frozen kidney tumor RCC, particularly, Boxplot showed overall mutations (upper left), germline mutations (upper middle), and somatic mutations (upper right) detected in single cells of each cell type in RCC1, and heatmap of single cell mutations located in SPLICESOME genes (lower panel).

FIG. 41 shows examples of tumor-cell-specific deMuts in single cells of a frozen kidney tumor RCC1, particularly, UMAP projections of single cells labeled with 8 examples of tumor-cell-specific deMuts before SoupX.

FIG. 42 shows examples of tumor-cell-specific deMuts in single cells of a frozen kidney tumor RCC1, particularly, UMAP projections of single cells with false positive mutation calling affected by ambient RNAs highlighted in magenta.

### DETAILED DESCRIPTION OF THE INVENTION

The invention will now be described more fully hereinafter with reference to the accompanying drawings, in which exemplary embodiments of the invention are shown. This invention may, however, be embodied in many different forms and should not be construed as limited to the embodiments set forth herein. Rather, these embodiments are provided so that this invention will be thorough and complete, and will fully convey the scope of the invention to those skilled in the art. Like reference numerals refer to like elements throughout.

The terms used in this specification generally have their ordinary meanings in the art, within the context of the invention, and in the specific context where each term is used. Certain terms that are used to describe the invention are discussed below, or elsewhere in the specification, to provide additional guidance to the practitioner regarding the description of the invention. For convenience, certain terms may be highlighted, for example using italics and/or quotation marks. The use of highlighting has no influence on the scope and meaning of a term; the scope and meaning of a term is the same, in the same context, whether or not it is highlighted. It will be appreciated that same thing can be said in more than one way. Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of other synonyms. The use of examples anywhere in this specification including examples of any terms discussed herein is illustrative only, and in no way limits the scope and meaning of the invention or of any exemplified term. Likewise, the invention is not limited to various embodiments given in this specification.

One of ordinary skill in the art will appreciate that starting materials, biological materials, reagents, synthetic methods, purification methods, analytical methods, assay methods, and biological methods other than those specifically exemplified can be employed in the practice of the invention without resort to undue experimentation. All art-known functional equivalents, of any such materials and methods are intended to be included in this invention. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the invention has been specifically disclosed by preferred embodiments and optional features, modification and variation of the concepts herein disclosed may be resorted to by those skilled in the art, and that such modifications and variations are considered to be within the scope of this invention as defined by the appended claims.

Whenever a range is given in the specification, for example, a temperature range, a time range, or a composition or concentration range, all intermediate ranges and subranges, as well as all individual values included in the ranges given are intended to be included in the invention. It will be understood that any subranges or individual values in a range or subrange that are included in the description herein can be excluded from the claims herein.

It will be understood that, as used in the description herein and throughout the claims that follow, the meaning of “a”, “an”, and “the” includes plural reference unless the context clearly dictates otherwise. Thus, for example, reference to “a cell” includes a plurality of such cells and equivalents thereof known to those skilled in the art. As well, the terms “a” (or “an”), “one or more” and “at least one” can be used interchangeably herein. It is also to be noted that the terms “comprising”, “including”, and “having” can be used interchangeably.

It will be understood that when an element is referred to as being “on”, “attached” to, “connected” to, “coupled” with, “contacting”, etc., another element, it can be directly on, attached to, connected to, coupled with or contacting the other element or intervening elements may also be present. In contrast, when an element is referred to as being, for example, “directly on”, “directly attached” to, “directly connected” to, “directly coupled” with or “directly contacting” another element, there are no intervening elements present. It will also be appreciated by those of skill in the art that references to a structure or feature that is disposed “adjacent” another feature may have portions that overlap or underlie the adjacent feature.

It will be understood that, although the terms first, second, third etc. may be used herein to describe various elements, components, regions, layers and/or sections, these elements, components, regions, layers and/or sections should not be limited by these terms. These terms are only used to distinguish one element, component, region, layer or section from another element, component, region, layer or section. Thus, a first element, component, region, layer or section discussed below could be termed a second element, component, region, layer or section without departing from the teachings of the invention.

Furthermore, relative terms, such as “lower” or “bottom” and “upper” or “top,” may be used herein to describe one element's relationship to another element as illustrated in the figures. It will be understood that relative terms are intended to encompass different orientations of the device in addition to the orientation depicted in the figures. For example, if the device in one of the figures is turned over, elements described as being on the “lower” side of other elements would then be oriented on “upper” sides of the other elements. The exemplary term “lower”, can therefore, encompasses both an orientation of “lower” and “upper,” depending of the particular orientation of the figure. Similarly, if the device in one of the figures is turned over, elements described as “below” or “beneath” other elements would then be oriented “above” the other elements. The exemplary terms “below” or “beneath” can, therefore, encompass both an orientation of above and below.

It will be further understood that the terms “comprises” and/or “comprising”, or “includes” and/or “including”, or “has” and/or “having”, or “carry” and/or “carrying”, or “contain” and/or “containing”, or “involve” and/or “involving”, “characterized by”, and the like are to be open-ended, i.e., to mean including but not limited to. When used in this disclosure, they specify the presence of stated features, regions, integers, steps, operations, elements, and/or components, but do not preclude the presence or addition of one or more other features, regions, integers, steps, operations, elements, components, and/or groups thereof.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and the invention, and will not be interpreted in an idealized or overly formal sense unless expressly so defined herein.

As used in the disclosure, “around”, “about”, “approximately” or “substantially” shall generally mean within 20 percent, preferably within 10 percent, and more preferably within 5 percent of a given value or range. Numerical quantities given herein are approximate, meaning that the term “around”, “about”, “approximately” or “substantially” can be inferred if not expressly stated.

As used in the disclosure, the phrase “at least one of A, B, and C” should be construed to mean a logical (A or B or C), using a non-exclusive logical OR. As used in the disclosure, the term “and/or” includes any and all combinations of one or more of the associated listed items.

As used in the disclosure, the term “control” or “control experiment” is used in accordance with its plain and ordinary meaning and refers to an experiment in which the subjects or reagents of the experiment are treated as in a parallel experiment except for omission of a procedure, reagent, or variable of the experiment. In some instances, the control is used as a standard of comparison in evaluating experimental effects.

As used in the disclosure, the term “complement” is used in accordance with its plain and ordinary meaning and refers to a nucleotide (e.g., RNA nucleotide or DNA nucleotide) or a sequence of nucleotides capable of base pairing with a complementary nucleotide or sequence of nucleotides. As described herein and commonly known in the art the complementary (matching) nucleotide of adenosine is thymidine in DNA, or alternatively in RNA the complementary (matching) nucleotide of adenosine is uracil, and the complementary (matching) nucleotide of guanosine is cytosine. Thus, a complement may include a sequence of nucleotides that base pair with corresponding complementary nucleotides of a second nucleic acid sequence. The nucleotides of a complement may partially or completely match the nucleotides of the second nucleic acid sequence. Where the nucleotides of the complement completely match each nucleotide of the second nucleic acid sequence, the complement forms base pairs with each nucleotide of the second nucleic acid sequence. Where the nucleotides of the complement partially match the nucleotides of the second nucleic acid sequence only some of the nucleotides of the complement form base pairs with nucleotides of the second nucleic acid sequence. Examples of complementary sequences include coding and non-coding sequences, wherein the non-coding sequence contains complementary nucleotides to the coding sequence and thus forms the complement of the coding sequence. A further example of complementary sequences are sense and antisense sequences, wherein the sense sequence contains complementary nucleotides to the antisense sequence and thus forms the complement of the antisense sequence. “Duplex” means at least two oligonucleotides and/or polynucleotides that are fully or partially complementary undergo Watson-Crick type base pairing among all or most of their nucleotides so that a stable complex is formed.

As described herein, the complementarity of sequences may be partial, in which only some of the nucleic acids match according to base pairing, or complete, where all the nucleic acids match according to base pairing. Thus, two sequences that are complementary to each other, may have a specified percentage of nucleotides that complement one another (e.g., about 60%, preferably 65%, 70%, 75%, 80%, 85%, 90%, 91%, 92%, 93%, 94%, 95%, 96%, 97%, 98%, 99%, or higher complementarity over a specified region). In embodiments, two sequences are complementary when they are completely complementary, having 100% complementarity. In embodiments, sequences in a pair of complementary sequences form portions of a single polynucleotide with non-base-pairing nucleotides (e.g., as in a hairpin structure, with or without an overhang) or portions of separate polynucleotides. In embodiments, one or both sequences in a pair of complementary sequences form portions of longer polynucleotides, which may or may not include additional regions of complementarity.

As used in the disclosure, the term “contacting” is used in accordance with its plain ordinary meaning and refers to the process of allowing at least two distinct species (e.g. chemical compounds including biomolecules or cells) to become sufficiently proximal to react, interact or physically touch. However, the resulting reaction product can be produced directly from a reaction between the added reagents or from an intermediate from one or more of the added reagents that can be produced in the reaction mixture. The term “contacting” may include allowing two species to react, interact, or physically touch, wherein the two species may be a compound, nucleic acid, a protein, or enzyme (e.g., a DNA polymerase).

As used in the disclosure, the term “nucleic acid” is used in accordance with its plain and ordinary meaning and refers to nucleotides (e.g., deoxyribonucleotides or ribonucleotides) and polymers thereof in either single-, double- or multiple-stranded form, or complements thereof. The terms “polynucleotide,” “oligonucleotide,” “oligo” or the like refer, in the usual and customary sense, to a sequence of nucleotides. The term “nucleotide” refers, in the usual and customary sense, to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA, and hybrid molecules having mixtures of single and double stranded DNA and RNA with linear or circular framework. Non-limiting examples of polynucleotides include a gene, a gene fragment, an exon, an intron, intergenic DNA (including, without limitation, heterochromatic DNA), messenger RNA (mRNA), transfer RNA, ribosomal RNA, a ribozyme, cDNA, a recombinant polynucleotide, a branched polynucleotide, a plasmid, a vector, isolated DNA of a sequence, isolated RNA of a sequence, a nucleic acid probe, and a primer. Polynucleotides useful in the methods of the disclosure may comprise natural nucleic acid sequences and variants thereof, artificial nucleic acid sequences, or a combination of such sequences. A “nucleoside” is structurally similar to a nucleotide, but is missing the phosphate moieties. An example of a nucleoside analogue would be one in which the label is linked to the base and there is no phosphate group attached to the sugar molecule. As may be used in the disclosure, the terms “nucleic acid oligomer” and “oligonucleotide” are used interchangeably and are intended to include, but are not limited to, nucleic acids having a length of 200 nucleotides or less. In some embodiments, an oligonucleotide is a nucleic acid having a length of 2 to 200 nucleotides, 2 to 150 nucleotides, 5 to 150 nucleotides or 5 to 100 nucleotides.

As used in the disclosure, the term “primer” is defined to be one or more nucleic acid fragments that may specifically hybridize to a nucleic acid template, be bound by a polymerase, and be extended in a template-directed process for nucleic acid synthesis. A primer can be of any length depending on the particular technique it will be used for. For example, PCR primers are generally between 10 and 40 nucleotides in length. In some embodiments, a primer has a length of 200 nucleotides or less. In certain embodiments, a primer has a length of 10 to 150 nucleotides, 15 to 150 nucleotides, 5 to 100 nucleotides, 5 to 50 nucleotides or 10 to 50 nucleotides. The length and complexity of the nucleic acid fixed onto the nucleic acid template is not critical to the invention. One of skill can adjust these factors to provide optimum hybridization and signal production for a given hybridization procedure, and to provide the required resolution among different genes or genomic locations. The primer permits the addition of a nucleotide residue thereto, or oligonucleotide or polynucleotide synthesis therefrom, under suitable conditions well-known in the art. In an embodiment the primer is a DNA primer, i.e., a primer consisting of, or largely consisting of, deoxyribonucleotide residues. The primers are designed to have a sequence that is the complement of a region of template/target DNA to which the primer hybridizes. The addition of a nucleotide residue to the 3′ end of a primer by formation of a phosphodiester bond results in a DNA extension product. The addition of a nucleotide residue to the 3′ end of the DNA extension product by formation of a phosphodiester bond results in a further DNA extension product. In another embodiment the primer is an RNA primer. In embodiments, a primer is hybridized to a target polynucleotide. A “primer” comprises a sequence that is complementary to a polynucleotide template, and complexes by hydrogen bonding or hybridization with the template to give a primer/template complex for initiation of synthesis by a polymerase, which is extended by the addition of covalently bonded bases linked at its 3′ end complementary to the template in the process of DNA synthesis.

As used in the disclosure, the terms “solid support” and “substrate” and “solid surface” refers to discrete solid or semi-solid surfaces to which a plurality of primers may be attached. A solid support may encompass any type of solid, porous, or hollow sphere, ball, cylinder, or other similar configuration composed of plastic, ceramic, metal, or polymeric material (e.g., hydrogel) onto which a nucleic acid may be immobilized (e.g., covalently or non-covalently). A solid support may comprise a discrete particle that may be spherical (e.g., microspheres) or have a non-spherical or irregular shape, such as cubic, cuboid, pyramidal, cylindrical, conical, oblong, or disc-shaped, and the like. Solid supports in the form of discrete particles may be referred to herein as “beads,” which alone does not imply or require any particular shape. A bead can be non-spherical in shape. A solid support may further comprise a polymer or hydrogel on the surface to which the primers are attached (e.g., the splint primers are covalently attached to the polymer, wherein the polymer is in direct contact with the solid support). Exemplary solid supports include, but are not limited to, glass and modified or functionalized glass, plastics (including acrylics, polystyrene and copolymers of styrene and other materials, polypropylene, polyethylene, polybutylene, polyurethanes, Teflon™, cyclic olefin copolymers, polyimides etc.), nylon, ceramics, resins, Zeonor, silica or silica-based materials including silicon and modified silicon, carbon, metals, inorganic glasses, optical fiber bundles, photopatternable dry film resists, UV-cured adhesives and polymers. The solid supports for some embodiments have at least one surface located within a flow cell. The solid support, or regions thereof, can be substantially flat. The solid support can have surface features such as wells, pits, channels, ridges, raised regions, pegs, posts or the like. The term solid support is encompassing of a substrate (e.g., a flow cell) having a surface comprising a polymer coating covalently attached thereto. In embodiments, the solid support is a flow cell. The term “flow cell” as used herein refers to a chamber including a solid surface across which one or more fluid reagents can be flowed. Examples of flow cells and related fluidic systems and detection platforms that can be readily used in the methods of the present disclosure are described, for example, in Bentley et al., Nature 456:53-59 (2008).

In some embodiments, a nucleic acid comprises a capture nucleic acid. A capture nucleic acid refers to a nucleic acid that is attached to a substrate (e.g., covalently attached). In some embodiments, a capture nucleic acid comprises a primer. In some embodiments, a capture nucleic acid is a nucleic acid configured to specifically hybridize to a portion of one or more nucleic acid templates (e.g., a template of a library). In some embodiments a capture nucleic acid configured to specifically hybridize to a portion of one or more nucleic acid templates is substantially complementary to a suitable portion of a nucleic acid template, or an amplicon thereof. In some embodiments a capture nucleic acid is configured to specifically hybridize to a portion of an adapter, or a portion thereof. In some embodiments a capture nucleic acid, or portion thereof, is substantially complementary to a portion of an adapter, or a complement thereof. In embodiments, a capture nucleic acid is a probe oligonucleotide. Typically, a probe oligonucleotide is complementary to a target polynucleotide or portion thereof, and further comprises a label (such as a binding moiety) or is attached to a surface, such that hybridization to the probe oligonucleotide permits the selective isolation of probe-bound polynucleotides from unbound polynucleotides in a population. A probe oligonucleotide may or may not also be used as a primer.

Nucleic acids, including e.g., nucleic acids with a phosphothioate backbone, can include one or more reactive moieties. As used in the disclosure, the term reactive moiety includes any group capable of reacting with another molecule, e.g., a nucleic acid or polypeptide through covalent, non-covalent or other interactions. By way of example, the nucleic acid can include an amino acid reactive moiety that reacts with an amino acid on a protein or polypeptide through a covalent, non-covalent, or other interaction.

A polynucleotide is typically composed of a specific sequence of four nucleotide bases: adenine (A); cytosine (C); guanine (G); and thymine (T) (uracil (U) for thymine (T) when the polynucleotide is RNA). Thus, the term “polynucleotide sequence” is the alphabetical representation of a polynucleotide molecule; alternatively, the term may be applied to the polynucleotide molecule itself. This alphabetical representation can be input into databases in a computer having a central processing unit and used for bioinformatics applications such as functional genomics and homology searching. Polynucleotides may optionally include one or more non-standard nucleotide(s), nucleotide analog(s) and/or modified nucleotides.

As used in the disclosure, the term “template nucleic acid” refers to any polynucleotide molecule that may be bound by a polymerase and utilized as a template for nucleic acid synthesis. A template nucleic acid may be a target nucleic acid. In general, the term “target nucleic acid” refers to a nucleic acid molecule or polynucleotide in a starting population of nucleic acid molecules having a target sequence whose presence, amount, and/or nucleotide sequence, or changes in one or more of these, are desired to be determined. In general, the term “target sequence” refers to a nucleic acid sequence on a single strand of nucleic acid. The target sequence may be a portion of a gene, a regulatory sequence, genomic DNA, cDNA, RNA including mRNA, miRNA, rRNA, or others. The target sequence may be a target sequence from a sample or a secondary target such as a product of an amplification reaction. A target nucleic acid is not necessarily any single molecule or sequence. For example, a target nucleic acid may be any one of a plurality of target nucleic acids in a reaction, or all nucleic acids in a given reaction, depending on the reaction conditions. For example, in a nucleic acid amplification reaction with random primers, all polynucleotides in a reaction may be amplified. As a further example, a collection of targets may be simultaneously assayed using polynucleotide primers directed to a plurality of targets in a single reaction. As yet another example, all or a subset of polynucleotides in a sample may be modified by the addition of a primer-binding sequence (such as by the ligation of adapters containing the primer binding sequence), rendering each modified polynucleotide a target nucleic acid in a reaction with the corresponding primer polynucleotide(s). In the context of selective sequencing, “target nucleic acid(s)” refers to the subset of nucleic acid(s) to be sequenced from within a starting population of nucleic acids.

In embodiments, a target nucleic acid is a cell-free nucleic acid. In general, the terms “cell-free,” “circulating,” and “extracellular” as applied to nucleic acids (e.g. “cell-free DNA” (cfDNA) and “cell-free RNA” (cfRNA)) are used interchangeably to refer to nucleic acids present in a sample from a subject or portion thereof that can be isolated or otherwise manipulated without applying a lysis step to the sample as originally collected (e.g., as in extraction from cells or viruses). Cell-free nucleic acids are thus unencapsulated or “free” from the cells or viruses from which they originate, even before a sample of the subject is collected. Cell-free nucleic acids may be produced as a byproduct of cell death (e.g. apoptosis or necrosis) or cell shedding, releasing nucleic acids into surrounding body fluids or into circulation. Accordingly, cell-free nucleic acids may be isolated from a non-cellular fraction of blood (e.g. serum or plasma), from other bodily fluids (e.g. urine), or from non-cellular fractions of other types of samples.

As used in the disclosure, the terms “analogue” and “analog”, in reference to a chemical compound, refers to compound having a structure similar to that of another one, but differing from it in respect of one or more different atoms, functional groups, or substructures that are replaced with one or more other atoms, functional groups, or substructures. In the context of a nucleotide, a “nucleotide analog” and “modified nucleotide” refer to a compound that, like the nucleotide of which it is an analog, can be incorporated into a nucleic acid molecule (e.g., an extension product) by a suitable polymerase, for example, a DNA polymerase in the context of a nucleotide analogue. The terms also encompass nucleic acids containing known nucleotide analogs or modified backbone residues or linkages, which are synthetic, naturally occurring, or non-naturally occurring, which have similar binding properties as the reference nucleic acid, and which are metabolized in a manner similar to the reference nucleotides. Examples of such analogs include, include, without limitation, phosphodiester derivatives including, e.g., phosphoramidate, phosphorodiamidate, phosphorothioate (also known as phosphothioate having double bonded sulfur replacing oxygen in the phosphate), phosphorodithioate, phosphonocarboxylic acids, phosphonocarboxylates, phosphonoacetic acid, phosphonoformic acid, methyl phosphonate, boron phosphonate, or O-methylphosphoroamidite linkages (see, e.g., see Eckstein, OLIGONUCLEOTIDES AND ANALOGUES: A PRACTICAL APPROACH, Oxford University Press) as well as modifications to the nucleotide bases such as in 5-methyl cytidine or pseudouridine; and peptide nucleic acid backbones and linkages. Other analog nucleic acids include those with positive backbones; non-ionic backbones, modified sugars, and non-ribose backbones (e.g. phosphorodiamidate morpholino oligos or locked nucleic acids (LNA)), including those described in U.S. Pat. Nos. 5,235,033 and 5,034,506, and Chapters 6 and 7, ASC Symposium Series 580, CARBOHYDRATE MODIFICATIONS IN ANTISENSE RESEARCH, Sanghui & Cook, eds. Nucleic acids containing one or more carbocyclic sugars are also included within one definition of nucleic acids. Modifications of the ribose-phosphate backbone may be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made. In embodiments, the internucleotide linkages in DNA are phosphodiester, phosphodiester derivatives, or a combination of both.

As used in the disclosure, a “native” nucleotide is used in accordance with its plain and ordinary meaning and refers to a naturally occurring nucleotide that does not include an exogenous label (e.g., a fluorescent dye, or other label) or chemical modification such as those that may characterize a nucleotide analog (e.g., a reversible terminating moiety). Examples of native nucleotides useful for carrying out procedures described herein include: dATP (2′-deoxyadenosine-5′-triphosphate); dGTP (2′-deoxyguanosine-5′-triphosphate); dCTP (2′-deoxycytidine-5′-triphosphate); dTTP (2′-deoxythymidine-5′-triphosphate); and dUTP (2′-deoxyuridine-5′-triphosphate).

As used in the disclosure, the terms “hybridization” and “hybridizing” refer to a reaction in which one or more polynucleotides react to form a complex that is stabilized via hydrogen bonding between the bases of the nucleotide residues. The hydrogen bonding may occur by Watson Crick base pairing, Hoogstein binding, or in any other sequence specific manner according to base complementarity. The complex may comprise two strands forming a duplex structure, three or more strands forming a multi-stranded complex, a single self-hybridizing strand, or any combination of these. A hybridization reaction may constitute a step in a more extensive process, such as the initiation of PCR, or the enzymatic cleavage of a polynucleotide by an endonuclease. A second sequence that is perfectly complementary to a first sequence, or is polymerized by a polymerase using the first sequence as template, is referred to as “the complement” of the first sequence. The term “hybridizable” as applied to a polynucleotide refers to the ability of the polynucleotide to form a complex that is stabilized via hydrogen bonding between the bases of the nucleotide residues in a hybridization reaction. In some embodiments, a hybridizable sequence of nucleotides is at least about 50%, 60%, 70%, 75%, 80%, 85%, 90%, 95%, or 100% complementary to the sequence to which it hybridizes. In some embodiments, a hybridizable sequence is one that hybridizes to one or more target sequences as part of, and under the conditions of, a step in a multi-step process (e.g., a ligation reaction, or an amplification reaction). The propensity for hybridization between nucleic acids depends on the temperature and ionic strength of their milieu, the length of the nucleic acids and the degree of complementarity. The effect of these parameters on hybridization is described in, for example, Sambrook J., Fritsch E. F., Maniatis T., Molecular cloning: a laboratory manual, Cold Spring Harbor Laboratory Press, New York (1989). As used in the disclosure, hybridization of a primer, or of a DNA extension product, respectively, is extendable by creation of a phosphodiester bond with an available nucleotide or nucleotide analogue capable of forming a phosphodiester bond, therewith. For example, hybridization can be performed at a temperature ranging from 15° C. to 95° C. In some embodiments, the hybridization is performed at a temperature of about 20° C., about 25° C., about 30° C., about 35° C., about 40° C., about 45° C., about 50° C., about 55° C., about 60° C., about 65° C., about 70° C., about 75° C., about 80° C., about 85° C., about 90° C., or about 95° C. In other embodiments, the stringency of the hybridization can further altered by the addition or removal of components of the buffered solution. A specific hybridization discriminates over non-specific hybridization interactions (e.g., two nucleic acids that a not configured to specifically hybridize, e.g., two nucleic acids that are 80% or less, 70% or less, 60% or less or 50% or less complementary) by about 2-fold or more, often about 10-fold or more, and sometimes about 100-fold or more, 1000-fold or more, 10,000-fold or more, 100,000-fold or more, or 1,000,000-fold or more. Two nucleic acid strands that are hybridized to each other can form a duplex which comprises a double-stranded portion of nucleic acid. The terms “hybridize” and “anneal”, and grammatical variations thereof, are used interchangeably herein. In some embodiments nucleic acids, or portions thereof, that are configured to specifically hybridize are often about 80% or more, 81% or more, 82% or more, 83% or more, 84% or more, 85% or more, 86% or more, 87% or more, 88% or more, 89% or more, 90% or more, 91% or more, 92% or more, 93% or more, 94% or more, 95% or more, 96% or more, 97% or more, 98% or more, 99% or more or 100% complementary to each other over a contiguous portion of nucleic acid sequence.

As used in the disclosure, the term “label” or “labels” is used in accordance with their plain and ordinary meanings and refer to molecules that can directly or indirectly produce or result in a detectable signal either by themselves or upon interaction with another molecule. Non-limiting examples of detectable labels include fluorescent dyes, biotin, digoxin, haptens, and epitopes. In general, a dye is a molecule, compound, or substance that can provide an optically detectable signal, such as a colorimetric, luminescent, bioluminescent, chemiluminescent, phosphorescent, or fluorescent signal. In embodiments, the label is a dye. In embodiments, the dye is a fluorescent dye. Non-limiting examples of dyes, some of which are commercially available, include CF dyes (Biotium, Inc.), Alexa Fluor dyes (Thermo Fisher), DyLight dyes (Thermo Fisher), Cy dyes (GE Healthscience), IRDyes (Li-Cor Biosciences, Inc.), and HiLyte dyes (Anaspec, Inc.). In embodiments, a particular nucleotide type is associated with a particular label, such that identifying the label identifies the nucleotide with which it is associated. In embodiments, the label is luciferin that reacts with luciferase to produce a detectable signal in response to one or more bases being incorporated into an elongated complementary strand, such as in pyrosequencing. In embodiments, a nucleotide comprises a label (such as a dye). In embodiments, the label is not associated with any particular nucleotide, but detection of the label identifies whether one or more nucleotides having a known identity were added during an extension step (such as in the case of pyrosequencing).

In embodiments, the detectable label is a fluorescent dye. In embodiments, the detectable label is a fluorescent dye capable of exchanging energy with another fluorescent dye (e.g., fluorescence resonance energy transfer (FRET) chromophores).

As used in the disclosure, the term “polymerase” and “nucleic acid polymerase” are used in accordance with their plain ordinary meanings and refer to enzymes capable of synthesizing nucleic acid molecules from nucleotides (e.g., deoxyribonucleotides). Exemplary types of polymerases that may be used in the compositions and methods of the present disclosure include the nucleic acid polymerases such as DNA polymerase, DNA- or RNA-dependent RNA polymerase, and reverse transcriptase. In some cases, the DNA polymerase is 9° N polymerase or a variant thereof, E. Coli DNA polymerase I, Bacteriophage T4 DNA polymerase, Sequenase, Taq DNA polymerase, DNA polymerase from Bacillus stearothermophilus, Bst 2.0 DNA polymerase, 9° N polymerase (exo-)A485L/Y409V, Phi29 DNA Polymerase (429 DNA Polymerase), T7 DNA polymerase, DNA polymerase II, DNA polymerase III holoenzyme, DNA polymerase IV, DNA polymerase V, VentR DNA polymerase, Therminator™ II DNA Polymerase, Therminator™ III DNA Polymerase, or Therminator™ IX DNA Polymerase. In embodiments, the polymerase is a protein polymerase. Typically, a DNA polymerase adds nucleotides to the 3′ end of a DNA strand, one nucleotide at a time. In embodiments, the DNA polymerase is a Pol I DNA polymerase, Pol II DNA polymerase, Pol III DNA polymerase, Pol IV DNA polymerase, Pol V DNA polymerase, Pol β DNA polymerase, Pol μ DNA polymerase, Pol λ DNA polymerase, Pol σ DNA polymerase, Pol α DNA polymerase, Pol δ DNA polymerase, Pol ε DNA polymerase, Pol η DNA polymerase, Pol τ DNA polymerase, Pol κ DNA polymerase, Pol ζ DNA polymerase, Pol γ DNA polymerase, Pol θ DNA polymerase, Pol ν DNA polymerase, or a thermophilic nucleic acid polymerase (e.g. Therminator γ, 9° N polymerase (exo-), Therminator™ II, Therminator™ III, or Therminator™ IX). In embodiments, the DNA polymerase is a modified archaeal DNA polymerase. In embodiments, the polymerase is a reverse transcriptase. In embodiments, the polymerase is a mutant P. abyssi polymerase (e.g., such as a mutant P. abyssi polymerase described in WO 2018/148723 or WO 2020/056044). In embodiments, the polymerase is a reverse transcriptase such as HIV type M or O reverse transcriptase, avian myeloblastosis virus reverse transcriptase, or Moloney Murine Leukemia Virus (MMLV) reverse transcriptase, or telomerase.

The terms “DNA ligase” and “ligase” are used in accordance with their ordinary meaning in the art and refer to an enzyme capable catalyzing the formation of a phosphodiester bond between two nucleic acids. In embodiments, the DNA ligase covalently joins the phosphate backbone of a nucleic acid with a compatible nucleotide residue (e.g., a second blunt ended strand). In embodiments, the ligase is a ligation enzyme (e.g., CircLigase™ enzyme, Taq DNA Ligase, HiFi Taq DNA Ligase, T4 ligase, PBCV-1 DNA Ligase (also known as SplintR ligase) or Ampligase DNA Ligase). Non-limiting examples of ligases include DNA ligases such as DNA Ligase I, DNA Ligase II, DNA Ligase III, DNA Ligase IV, T4 DNA ligase, T7 DNA ligase, T3 DNA Ligase, E. coli DNA Ligase, PBCV-1 DNA Ligase (also known as SplintR ligase) or a Taq DNA Ligase. In embodiments, a ligase is provided in a buffer containing ATP and a divalent ion (e.g., Mn2+ or Mg2+). In embodiments, the ligase is provided in a buffer containing PEG, which is known to increase the ligation efficiency of nucleic acid molecules. As used in the disclosure, the term “exonuclease activity” is used in accordance with its ordinary meaning in the art, and refers to the removal of a nucleotide from a nucleic acid by a DNA polymerase. For example, during polymerization, nucleotides are added to the 3′ end of a primer or extension strand. Occasionally, a DNA polymerase incorporates an incorrect nucleotide to the 3′-OH terminus of the primer strand, wherein the incorrect nucleotide cannot form a hydrogen bond to the corresponding base in the template strand. Such a nucleotide, added in error, is removed from the primer or extension product as a result of the 3′ to 5′ exonuclease activity of the DNA polymerase. In embodiments, exonuclease activity may be referred to as “proofreading.” When referring to 3′-5′ exonuclease activity, it is understood that the DNA polymerase facilitates a hydrolyzing reaction that breaks phosphodiester bonds at either the 3′ end of a polynucleotide chain to excise the nucleotide. In embodiments, 3′-5′ exonuclease activity refers to the successive removal of nucleotides in single-stranded DNA in a 3′→5′ direction, releasing deoxyribonucleoside 5′-monophosphates one after another. Methods for quantifying exonuclease activity are known in the art, for example Southworth et al. PNAS Vol 93, 8281-8285 (1996).

As used in the disclosure, the term “selective” or “selectivity” is used in accordance with its ordinary meaning in the art, and in the context of a compound refers to a compound's ability to discriminate between molecular targets.

As used in the disclosure, the terms “specific”, “specifically”, and “specificity”, are used in accordance with their ordinary meaning in the art, and in the context of a compound refer to the compound's ability to cause a particular action, such as binding, to a particular molecular target with minimal or no action to other proteins in the cell.

As used in the disclosure, the terms “bind” and “bound” are used in accordance with their plain and ordinary meanings and refer to an association between atoms or molecules. The association can be direct or indirect. For example, bound atoms or molecules may be directly bound to one another, e.g., by a covalent bond or non-covalent bond (e.g. electrostatic interactions (e.g. ionic bond, hydrogen bond, halogen bond), van der Waals interactions (e.g. dipole-dipole, dipole-induced dipole, London dispersion), ring stacking (pi effects), hydrophobic interactions and the like). As a further example, two molecules may be bound indirectly to one another by way of direct binding to one or more intermediate molecules, thereby forming a complex.

As used in the disclosure, the term “extension” or “elongation” is used in accordance with its plain and ordinary meanings and refer to synthesis by a polymerase of a new polynucleotide strand complementary to a template strand by adding free nucleotides (e.g., dNTPs) from a reaction mixture that are complementary to the template in the 5′-to-3′ direction. Extension includes condensing the 5′-phosphate group of the dNTPs with the 3′-hydroxy group at the end of the nascent (elongating) DNA strand.

As used in the disclosure, the term “hybridization pad” refers to one or both of two regions on either end of an interposing oligonucleotide barcode that are capable of hybridizing to single-stranded template nucleic acids. In embodiments, hybridization pads are a complement to the original target nucleic acid. In embodiments, each hybridization pad is composed of about 3 to about 40 random nucleotides (e.g. NNNNN, wherein N represents A, T, C, G nucleotides). In embodiments, each hybridization pad is composed of about 3 to about 5 random nucleotides. In embodiments, the first hybridization pad includes about 3 to about 5 nucleotides (e.g., random nucleotides) and the second hybridization pad includes about 3 to 25 nucleotides (e.g., random nucleotides). In embodiments, the first hybridization pad includes about 5 to about 15 nucleotides (e.g., random nucleotides) and the second hybridization pad includes about 5 to 15 nucleotides (e.g., random nucleotides). In embodiments, the first hybridization pad includes about 10 to about 15 nucleotides (e.g., random nucleotides) and the second hybridization pad includes about 10 to 15 nucleotides (e.g., random nucleotides). In embodiments, the hybridization pad includes a targeted primer sequence, or a portion thereof. A “targeted primer sequence” refers to a nucleic acid sequence that is complementary to a known nucleic acid region (e.g., complementary to a universally conserved region, or complementary sequences to target specific genes or mutations that have relevancy to a particular cancer phenotype). The hybridization pads may include sequences designed through computational software, e.g., Primer BLAST, LaserGene (DNAStar), Oligo (National Biosciences, Inc.), Mac Vector (Kodak/IBI) or the GCG suite of programs to optimize desired properties. In embodiments, the hybridization pad includes a limited-diversity sequence. A “limited-diversity sequence” refers to a nucleic acid sequence that includes random nucleotide regions and fixed nucleotide regions (e.g., NNANN, ANNTN, TNCNA, etc., wherein N represents random nucleotides and A, T, C, G represent fixed nucleotides). In embodiments, each hybridization pad is composed of 3 random nucleotides and 1 to 2 non-random nucleotides. In embodiments, each hybridization pad is composed of 4 random nucleotides and 1 to 2 non-random nucleotides.

As used in the disclosure, the term “barcode sequence” (which may be referred to as a “tag,” a “molecular barcode,” a “molecular identifier,” an “identifier sequence,” or a “unique molecular identifier”) refers to any material (e.g., a nucleotide sequence, a nucleic acid molecule feature) that is capable of distinguishing an individual molecule in a large heterogeneous population of molecules. Generally, a barcode sequence is unique in a pool of barcode sequences that differ from one another in sequence, or is uniquely associated with a particular sample polynucleotide in a pool of sample polynucleotides. In embodiments, the barcode sequence is a nucleotide sequence that forms a portion of a larger polynucleotide, such as an “interposing oligonucleotide barcode” (also referred to herein as an “interposing barcode” or an “oligonucleotide barcode”). In embodiments, every barcode sequence in a pool of interposing oligonucleotide barcodes is unique, such that sequencing reads comprising the barcode sequence can be identified as originating from a single sample polynucleotide molecule on the basis of the barcode sequence alone. In other embodiments, individual barcode sequences may be used more than once, but interposing oligonucleotide barcodes comprising the duplicate barcode sequences hybridize to different sample polynucleotides and/or in different arrangements of neighboring interposing oligonucleotide barcodes, such that sequence reads may still be uniquely distinguished as originating from a single sample polynucleotide molecule on the basis of a barcode sequence and adjacent sequence information (e.g., sample polynucleotide sequence, and/or one or more adjacent barcode sequences). In embodiments, barcode sequences are about or at least about 5, 6, 7, 8, 9, 10, 15, 20, 25, 30, 40, 50, 75 or more nucleotides in length. In embodiments, barcode sequences are shorter than 20, 15, 10, 9, 8, 7, 6, or 5 nucleotides in length. In embodiments, barcode sequences are about 10 to about 50 nucleotides in length, such as about 15 to about 40 or about 20 to about 30 nucleotides in length. In a pool of different barcode sequences, barcode sequences may have the same or different lengths. In general, barcode sequences are of sufficient length and include sequences that are sufficiently different to allow the identification of sequencing reads that originate from the same sample polynucleotide molecule. In embodiments, each barcode sequence in a plurality of barcode sequences differs from every other barcode sequence in the plurality by at least three nucleotide positions, such as at least 3, 4, 5, 6, 7, 8, 9, 10, or more nucleotide positions. In some embodiments, substantially degenerate barcode sequences may be known as random. In some embodiments, a barcode sequence may include a nucleic acid sequence from within a pool of known sequences. In some embodiments, the barcode sequences may be pre-defined.

As used in the disclosure, the term “random” in the context of a nucleic acid sequence or barcode sequence refers to a sequence where one or more nucleotides has an equal probability of being present. In embodiments, one or more nucleotides is selected at random from a set of two or more different nucleotides at one or more positions, with each of the different nucleotides selected at one or more positions represented in a pool of oligonucleotides including the random sequence. For example, a random sequence may be represented by a sequence composed of N's, where N can be any nucleotide (e.g., A, T, C, or G). For example, a four base random sequence may have the sequence NNNN, where the Ns can independently be any nucleotide (e.g., AATC). IBCs that contain a random sequence, collectively, have sequences composed of Ns within the hybridization pads, stem region, or loop region. Further, the IBCs have barcode sequences that may contain random sequence. In embodiments, a pool of IBCs may be represented by a fully random sequence, with the caveat that certain sequences have been excluded (e.g., runs of three or more nucleotides of the same type, such as “AAA” or “GGG”). In embodiments, nucleotide positions that are allowed to vary (e.g., by two, three, or four nucleotides) may be separated by one or more fixed positions (e.g., as in “NGN”).

As used in the disclosure, the terms “sequencing”, “sequence determination”, and “determining a nucleotide sequence”, are used in accordance with their ordinary meaning in the art, and refer to determination of partial as well as full sequence information of the polynucleotide being sequenced, and particular physical processes for generating such sequence information. That is, the term includes sequence comparisons, fingerprinting, and like levels of information about a target polynucleotide, as well as the express identification and ordering of nucleotides in a target polynucleotide. The term also includes the determination of the identification, ordering, and locations of one, two, or three of the four types of nucleotides within a target polynucleotide. Sequencing methods, such as those outlined in U.S. Pat. No. 5,302,509 can be carried out using the nucleotides described herein. The sequencing methods are preferably carried out with the target polynucleotide arrayed on a solid substrate. Multiple target polynucleotides can be immobilized on the solid support through linker molecules, or can be attached to particles, e.g., microspheres, which can also be attached to a solid substrate. In embodiments, the solid substrate is in the form of a chip, a bead, a well, a capillary tube, a slide, a wafer, a filter, a fiber, a porous media, or a column. In embodiments, the solid substrate is gold, quartz, silica, plastic, glass, diamond, silver, metal, or polypropylene. In embodiments, the solid substrate is porous.

Where a range of values is provided, it is understood that each intervening value, to the tenth of the unit of the lower limit unless the context clearly indicates otherwise, between the upper and lower limit of that range, and any other stated or unstated intervening value in, or smaller range of values within, that stated range is encompassed within the invention. The upper and lower limits of any such smaller range (within a more broadly recited range) may independently be included in the smaller ranges, or as particular values themselves, and are also encompassed within the invention, subject to any specifically excluded limit in the stated range. Where the stated range includes one or both of the limits, ranges excluding either or both of those included limits are also included in the invention.

As used in the disclosure, the term “sequencing read” is used in accordance with its plain and ordinary meaning and refers to an inferred sequence of base pairs (or base pair probabilities) corresponding to all or part of a single DNA fragment. Sequencing technologies vary in the length of reads produced. Reads of length 20-40 base pairs (bp) are referred to as ultra-short. Typical sequencers produce read lengths in the range of 100-500 bp. A sequencing read may include 10, 20, 30, 40, 50, 60, 70, 80, 90, 100, 150, 200, 250, or more nucleotide bases. Read length is a factor which can affect the results of biological studies. For example, longer read lengths improve the resolution of de novo genome assembly and detection of structural variants.

As used in the disclosure, the term “sequencing cycle” is used in accordance with its plain and ordinary meaning and refers to incorporating one or more nucleotides (e.g., nucleotide analogues) to the 3′ end of a polynucleotide with a polymerase, and detecting one or more labels that identify the one or more nucleotides incorporated. The sequencing may be accomplished by, for example, sequencing by synthesis, pyrosequencing, and the like. In embodiments, a sequencing cycle includes extending a complementary polynucleotide by incorporating a first nucleotide using a polymerase, wherein the polynucleotide is hybridized to a template nucleic acid, detecting the first nucleotide, and identifying the first nucleotide. In embodiments, to begin a sequencing cycle, one or more differently labeled nucleotides and a DNA polymerase can be introduced. Following nucleotide addition, signals produced (e.g., via excitation and emission of a detectable label) can be detected to determine the identity of the incorporated nucleotide (based on the labels on the nucleotides). Reagents can then be added to remove the 3′ reversible terminator and to remove labels from each incorporated base. Reagents, enzymes and other substances can be removed between steps by washing. Cycles may include repeating these steps, and the sequence of each cluster is read over the multiple repetitions.

As used in the disclosure the term “determine” can be used to refer to the act of ascertaining, establishing or estimating. A determination can be probabilistic. For example, a determination can have an apparent likelihood of at least 50%, 75%, 90%, 95%, 98%, 99%, 99.9% or higher. In some cases, a determination can have an apparent likelihood of 100%. An exemplary determination is a maximum likelihood analysis or report. As used in the disclosure, the term “identify,” when used in reference to a thing, can be used to refer to recognition of the thing, distinction of the thing from at least one other thing or categorization of the thing with at least one other thing. The recognition, distinction or categorization can be probabilistic. For example, a thing can be identified with an apparent likelihood of at least 50%, 75%, 90%, 95%, 98%, 99%, 99.9% or higher. A thing can be identified based on a result of a maximum likelihood analysis. In some cases, a thing can be identified with an apparent likelihood of 100%.

As used in the disclosure, term “gene” refers to a polynucleotide that is capable of conferring biological function after being transcribed and/or translated.

Present invention describes herein an analysis and computational tool, single cell nanopore sequencing analysis of Genotype-Phenotype simultaneously (scNanoGPS), to deconvolute barcoded long reads into single cells and single molecules without short reads curation and calculate both phenotypes (gene expression, isoform) and genotypes (mutation, fusion) of same cells.

In one embodiment, the present invention discloses scNanoGPS achieves independent deconvolution of raw data without the guidance of short-reads nor barcode whitelist and calculates genotypes-phenotypes of thousands of individual cells, addressing the major computational challenges of an emerging scNanoRNAseq technology.

In particular, the present invention performs completely independent deconvolution of error-prone long-reads into single-cells and single-molecules and calculate both genotypes and phenotypes in individual cells from scNanoRNAseq data. In concert with this tool, the present invention has removed all NGS sequencing steps and increased throughput to 3,000-6,000 transcriptomes per flow cell (PromethION). The present invention has demonstrated the accuracy and robustness of scNanoGPS and identified cell-type-specific isoforms and mutations in addition to gene expression profiles, enabling synchronous cell-lineage (genotype) and cell-fate (phenotype) tracing in human tissues.

In one embodiment, the present invention is applied scNanoGPS onto 23,587 long-read transcriptomes from 4 tumors and 2 cell-lines. Standalone, scNanoGPS deconvoluted error-prone long-reads into single-cells and single-molecules, and simultaneously accessed both phenotypes and genotypes of individual cells. The analyses of the present invention has revealed that tumor and stroma/immune cells expressed distinct combinations of isoforms (DCIs). In a kidney tumor, the present invention has identified 924 DCI genes involved in cell-type-specific functions such as PDE10A in tumor cells and CCL3 in lymphocytes. Moreover, transcriptome-wide mutation analyses identified many cell-type-specific mutations including VEGFA mutations in tumor cells and HLA-A mutations in immune cells, highlighting critical roles of different populations in tumors. Together, scNanoGPS facilitates applications of single-cell long-read sequencing technologies.

In one embodiment, scNanoGPS of the present invention performs independent barcode learning and assignment without additional NGS data, and calculate both genotypes (mutations, fusions) and phenotypes (gene expressions, isoforms) simultaneously from same cells using single cell nanopore sequencing data. scNanoGPS enables the construction of a unified cell lineage and cell fate roadmap of tumor cells to precisely delineate tumor evolution and therapeutic response.

Computational Workflow of scNanoGPS in Analyzing scNanoRNAseq Data

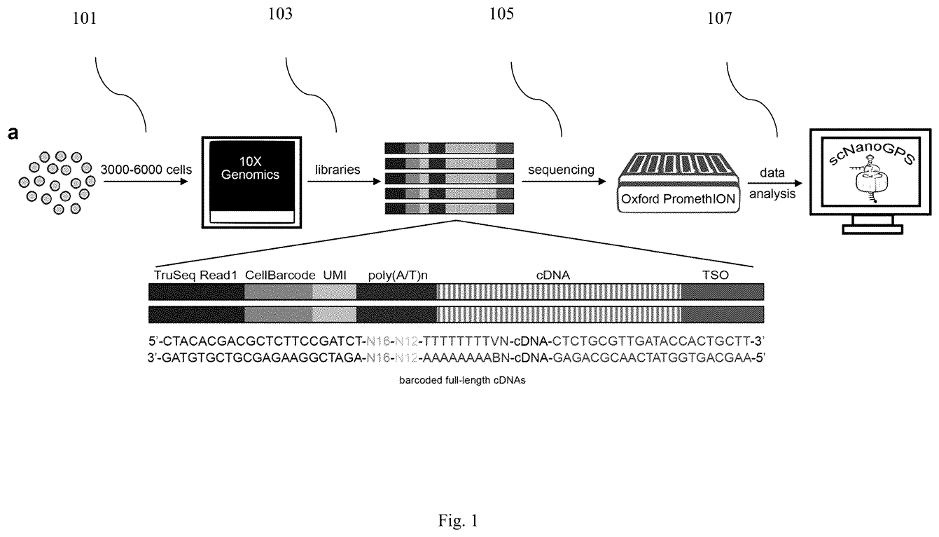

As shown in FIG. 1, after about 3,000-6,000 cells or nuclei are prepared, the high throughput scNanoRNAseq data are generated through 2 major steps: i) barcoding full-length cDNAs of single cells/nuclei using high throughput droplet barcoding system (10× Genomics), as steps 103 and 105 in FIG. 1; ii) performing high throughput long-read sequencing using PromethION (Oxford Nanopore Technologies), as steps 105 of FIG. 1. The sequencing data are then being processed by a computational workflow in step 107.

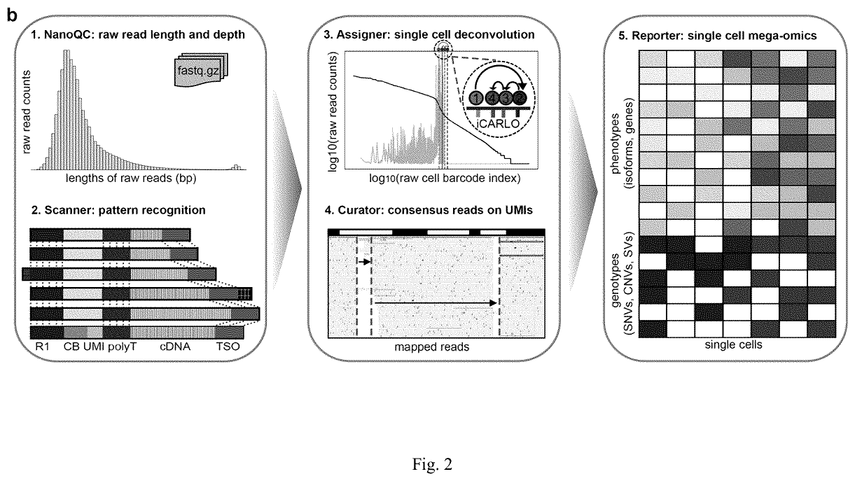

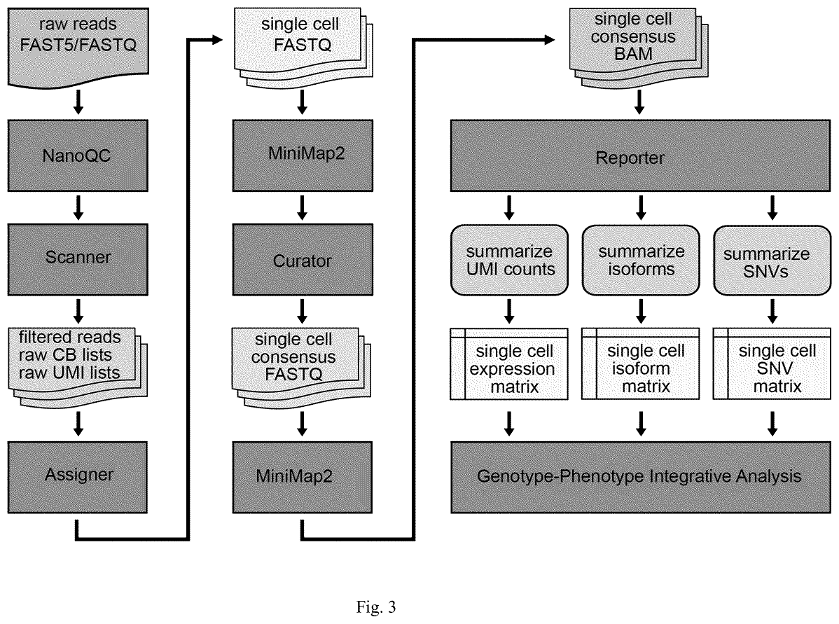

Accordingly, as shown in FIGS. 2-3, the computational workflow of scNanoGPS begins with quality control and scanning reads that have expected patterns of adaptor sequences, i.e., TruSeq RI adaptor on 5′-ends and TSO adaptor on 3′-ends of raw reads. Next, the present invention discloses an algorithm called integrated Crude Anchoring and Refinery Local Optimization (iCARLO) to detect true cell barcodes (CBs). In this algorithm, a raw list of CBs is determined by searching for a crude anchoring point through thresholding partial derivatives of the supporting reads of individual CBs. The threshold is then extended from crude anchoring point by an empirical percentage (˜10%) to rescue true CBs that have much less reads than others in the same experiment. Next, the CBs within two Levenshtein Distances (LDs) are curated and merged to rescue mis-assigned reads due to errors in CB sequences. CBs with less than 300 genes are filtered out by default. iCARLO is implemented in the “Assigner” function, which outputs a list of true CBs and then deconvolutes all qualified reads into single cell FASTQ files.

In one embodiment, to identify true UMIs to accurately measure gene expression levels, scNanoGPS first maps single cell reads against reference genome GRCh38 using MiniMap2, as shown in FIGS. 2 and 3. Reads that map to the same genomic regions (within <5 bp) are grouped into read clusters to achieve batch computing. All reads belonging to same read-clusters and having UMIs within two LDs are considered to be amplified from same RNA molecules, thus their corresponding UMIs are curated to be identical. To overcome sequencing errors in single molecules, reads with identical UMIs are collapsed into consensus sequences using SPOA for better detection of point mutations in gene bodies. scNanoGPS then re-maps single cell consensus reads to generate single cell consensus BAM files. Lastly, to better facilitate scNanoRNAseq data analysis, the present invention has compiled existing long-read specific tools into scNanoGPS to form a complete pipeline to calculates gene expression, isoform expression and point mutation profiles of individual cells from single cell consensus BAM files. Furthermore, single cell copy numbers are calculated using previously published algorithm CopyKAT. These data can then be used to detect cell-type-specific isoforms and mutations as well as gene expressions to demonstrate the applications of scNanoGPS in investigating the underlying mechanisms of human diseases including but not limited to tumors.

To summarize, scNanoGPS achieves complete independence through iCARLO algorithm to detect true CBs and constructs consensus molecules from reads with same UMIs to accurately call mutations and count RNA molecules. On the contrast, both Sockeye and BLAZE relies on barcode whitelist and arbitrary cutoff to detect possible CBs, as shown in Table 1. While BLAZE doesn't support UMI detection, Sockeye UMI function depends on high quality of reads that filters out large portion of data. Moreover, scNanoGPS assembles variant detection pipeline together to call mutations from consensus reads. Taken together, scNanoGPS of the present invention provides a full spectrum of functional modules to analyze scNanoRNAseq data from raw FASTQ data to same-cell multi-omics profiles of thousands of cells.

Performance of scNanoGPS in Processing High Throughput scNanoRNAseq Data

To test the technical feasibility, the present invention has applied scNanoGPS to process the scNanoRNAseq data of two cancer cell lines, A375 and H2030. Strikingly, PromethION yielded 67.4 million reads of the A375 library on one flow cell and 105.3 million reads of the H2030 library on another flow cell.

This resulted in averaged depths of 15,944 reads per cell in A375 and 21,710 reads per cell in H2030, which were close to NGS scRNAseq depths, as shown in Table 2.

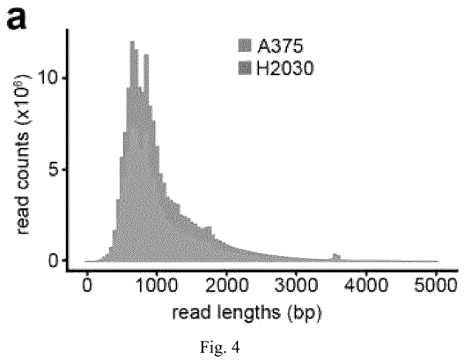

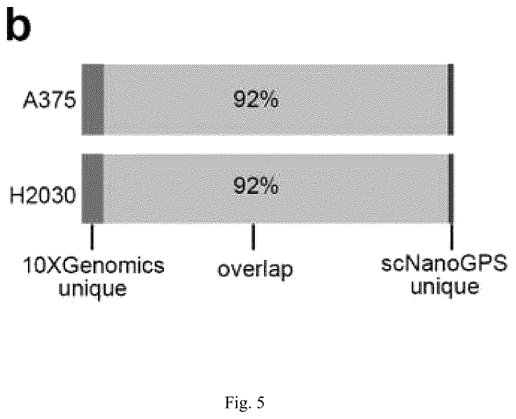

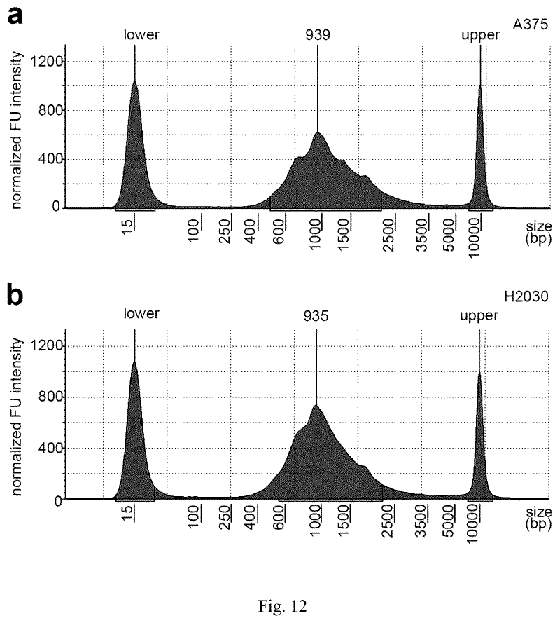

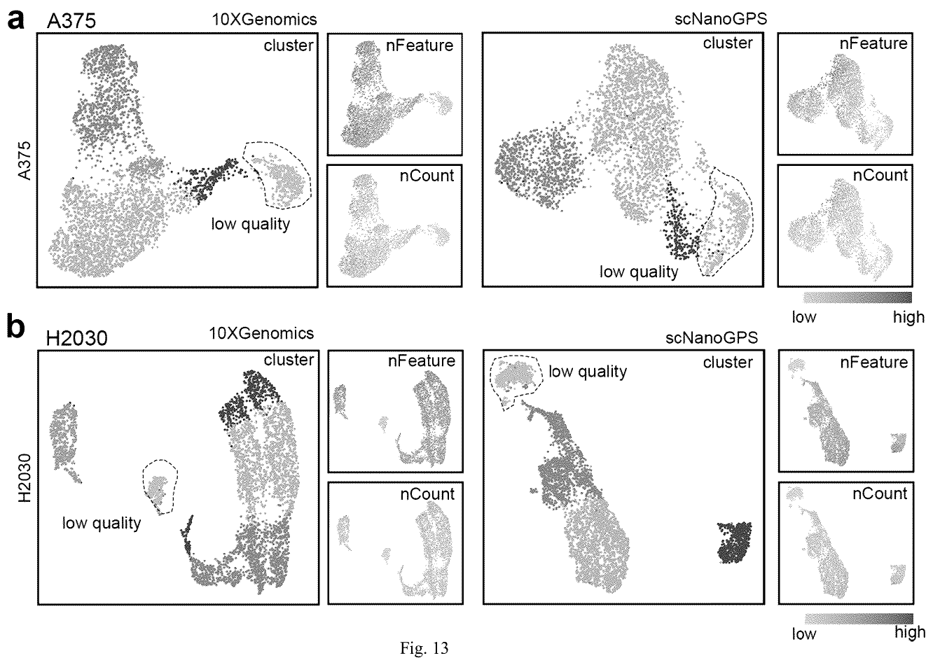

The median read lengths of both datasets were around 900 bp, consistent with the traces of pre-sequencing cDNAs, as shown in FIG. 4 and FIG. 12. The scNanoGPS results showed that most reads (A375: 86.80%, H2030: 87.31%) had the expected pattern of adaptor sequences. In total, the present invention detected 3,649 and 4,212 cells with averaged coverages of 2,688 and 3,553 genes per cell, respectively. Standard NGS 3′-scRNAseq (10× Genomics) data was generated from the same cDNA pools as previously described. Since 3′-scRNAseq (10× Genomics) was a widely used mature method and sequenced on Illumina sequencer (NovaSeq 6000) that has very low error rates in CBs and UMIs, the present invention treated these data as gold standard to benchmark scNanoGPS barcode detection accuracy. The analysis showed that scNanoGPS achieved high concordance (92%) with the standard 10× Genomics data in detecting true CBs, as shown in FIG. 5, with minor dis-concordance in thresholding low-quality cells that could be mitigated by secondary analyses, as shown in FIG. 13. scNanoGPS standalone achieved high accuracies in detecting true CBs, comparable to the performance of both whitelist-dependent approaches, i.e., BLAZE and Sockeye. While the time consumed in CB detection step were similar, scNanoGPS used less memories than BLAZE, according to Table 3. The present invention recorded time and space usages of each functional module of scNanoGPS when analyzing the example dataset, A375 in Table 4 to provide guidance of preparedness of computing resource.

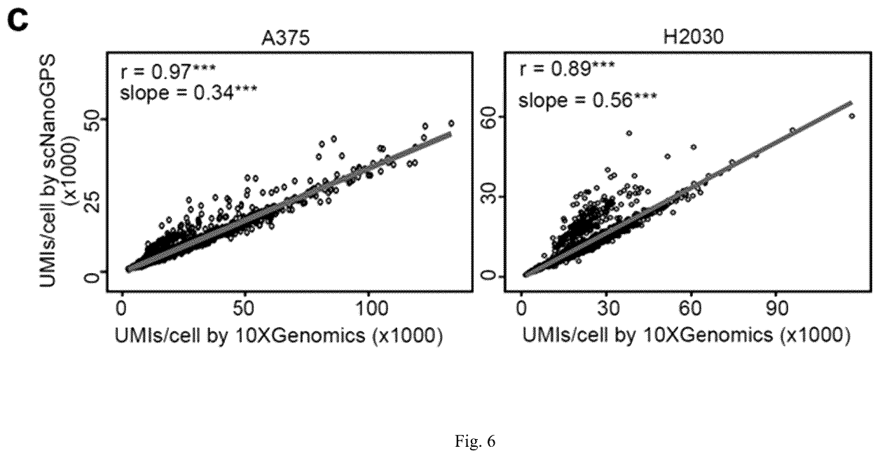

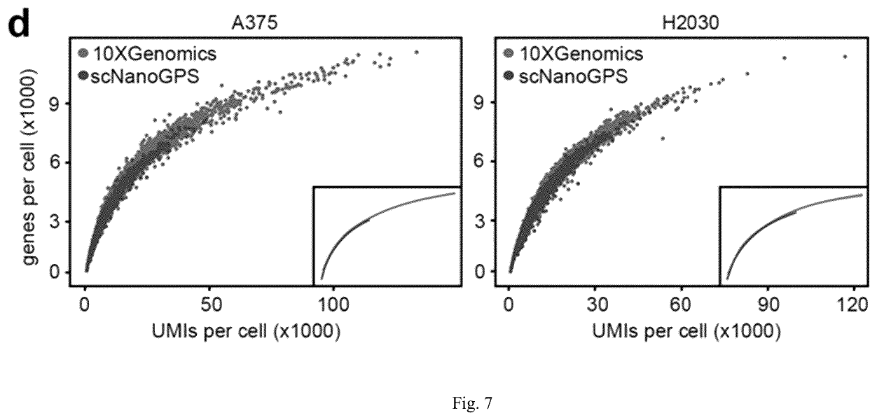

Next, the present invention compared the number of UMIs detected by scNanoGPS to standard 10× Genomics data. The results revealed significantly high correlations (A375: Pearson's r=0.97, P-value <2.2e-16; H2030: Pearson's r=0.89, P-value <2.2e-16), according to FIG. 6. The gene detection rates per UMI were similar as well, although the numbers of UMIs per cell were fewer in long-read data compared to 10× Genomics data (A375: coef-34%; H2030: coef=56%) due to lower sequencing depths, as shown in FIGS. 6-7. The combination of CBs, UMIs and genes detected by scNanoGPS reached up to 72% (SD: 2%) concordance on average with NGS approach, confirming the reliability of barcoding detection results, according to Table 5.

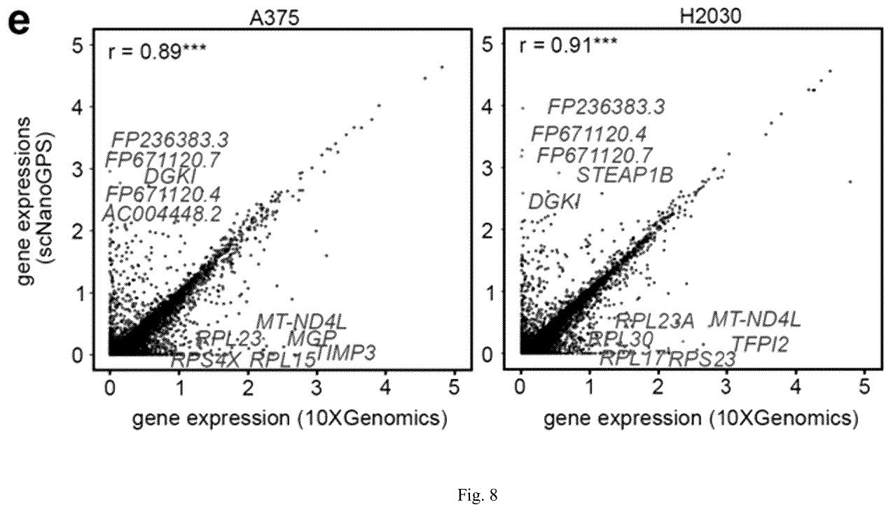

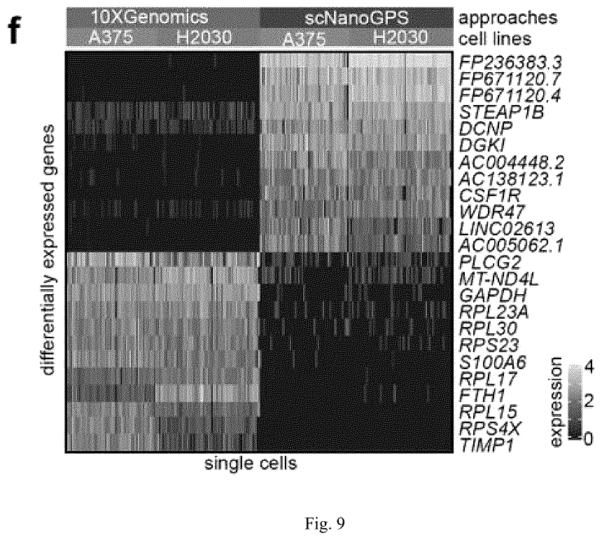

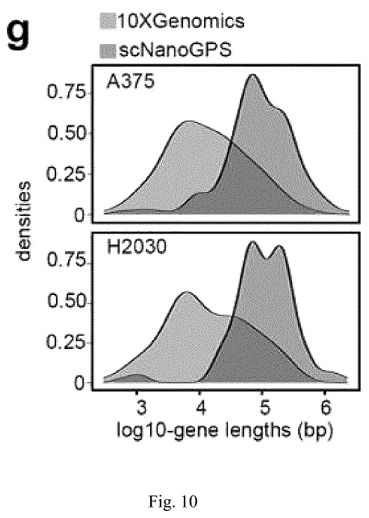

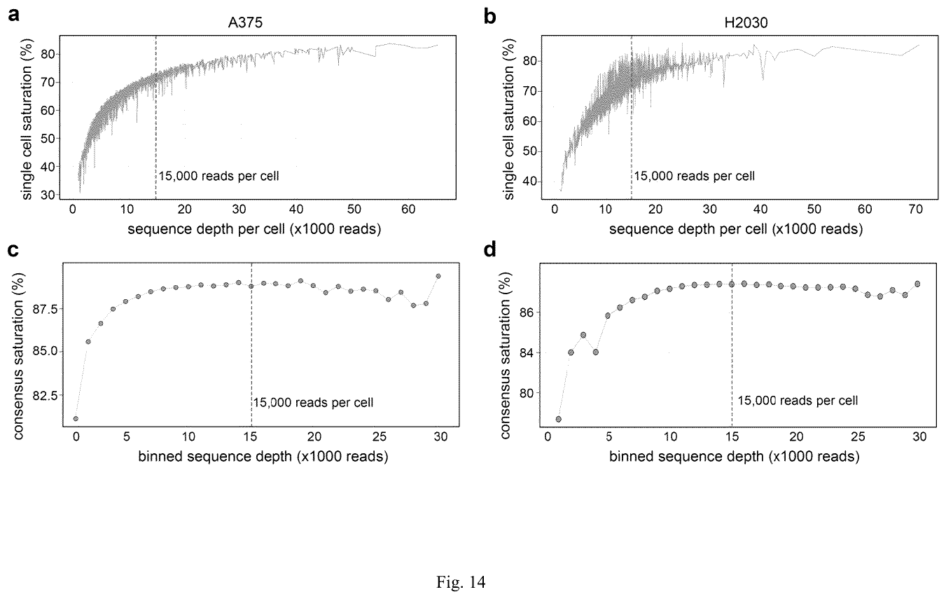

In one embodiment, the present invention further compared single cell transcriptomes computed by scNanoGPS to standard 10× Genomics 3′-scRNAseq data. The results again showed that scNanoGPS achieved high concordance (A375: Pearson's r=0.89, P-value <2.2e-16; H2030: Pearson's r=0.91, P-value <2.2e-16) in measuring gene expression levels compared to standard 10× Genomics data, as shown in FIG. 8. Down-sampling analysis suggested that ˜15,000 long-reads per cell on average are needed to robustly profile single cell full-length transcriptomes, as shown in FIG. 14. There were only small fractions (A375: 0.92%; H2030: 0.79%) of detected genes showing significantly different expression levels (FDR-adjusted P-values <0.05, |log 2(Fold Changes)|≥1) between the two approaches according to FIG. 8. Of note, 10× Genomics 3′-scRNAseq showed a higher chance of detecting ribosomal genes, whereas scNanoGPS detected more long noncoding RNA (lncRNA) genes and pseudogenes, according to FIGS. 8-9. The lengths of scNanoGPS enriched genes were significantly longer than NGS enriched genes, A375: P-value=8.16×10⁻¹⁰; H2030: P-value=1.96×10⁻⁹, as shown in FIG. 10, which is suspected as due to minor fragmentation bias against longer genes in NGS library preparation as previously reported.

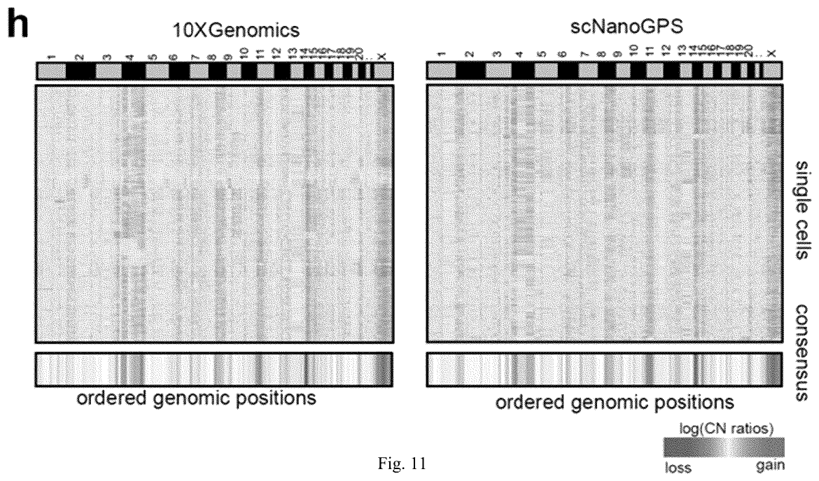

In one embodiment, to investigate whether long-read single cell transcriptome data could serve as a data source for inferring genome-wide DNA copy number alterations (CNAs), CopyKAT was run on H2030 which was known to have aneuploidy. As expected, the results showed that H2030 had genome-wide amplifications on Chr2, 3q, 5, 6q, 7, 8q, 14, 15p and deletions on Chr 4, 11p, in FIG. 11. The averaged pair-wised Pearson's correlation between the two approaches reached up to 94%, confirming the feasibility of inferring CNAs using scNanoRNAseq data.

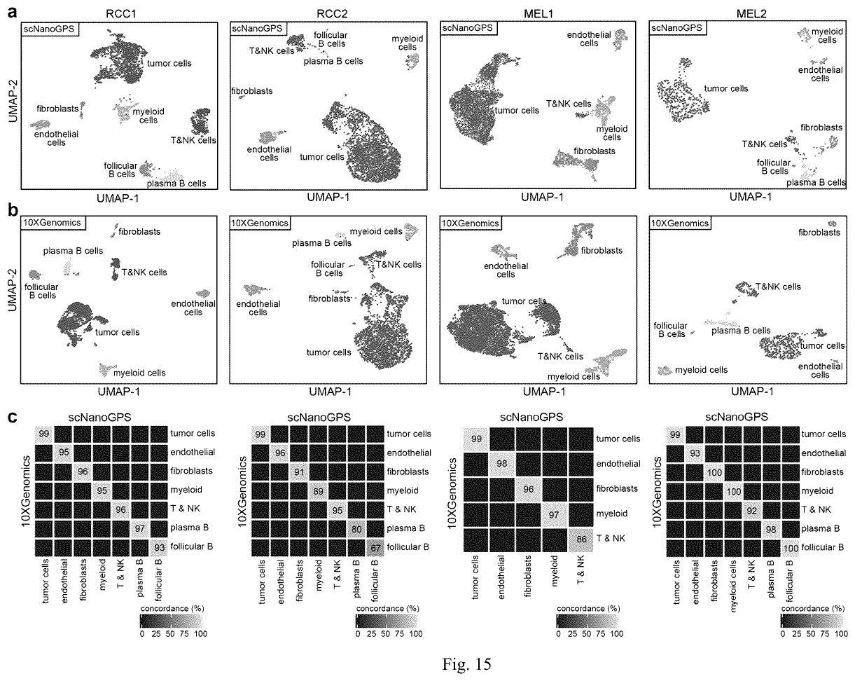

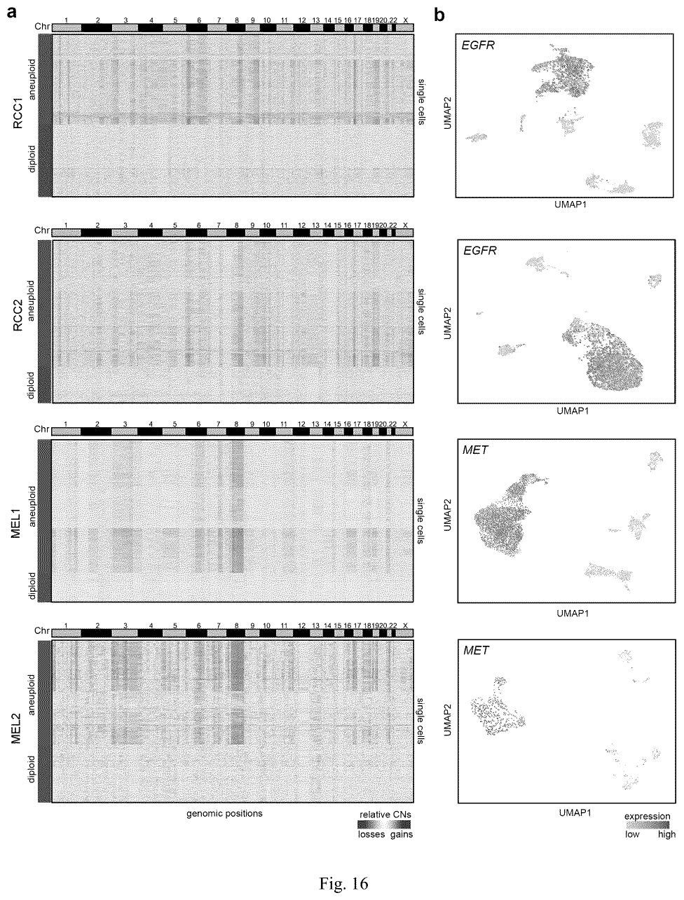

In one embodiment, to evaluate whether scNanoGPS can robustly detect major cell types in human tumors, the present invention performed scNanoRNAseq on 4 frozen tumors collected from Renal Cell Carcinoma (RCC1, RCC2) and Melanoma (MEL1, MEL2) patients according to Table 2. The present invention performed unbiased clustering of all cells within each tumor as shown in FIG. 15 upper panel, and annotated epithelial cells having both aneuploidy and high expression levels of known cancer-type specific genes as tumor cells, as shown in FIG. 16. In consistence with previous studies, the present invention's data showed that tumor cells outcompeted normal epithelial cells in all 4 tumors due to stronger fitness advantage. The non-tumor cell type clusters were annotated using known cell-type markers. For fair comparisons, the same clustering and annotations were conducted on paralleled 10× Genomics data, as shown in FIG. 15 middle panel. The results confirmed high concordance between the two approaches, as shown in FIG. 15 lower panel, except that scNanoGPS rescued more lymphocytes that expressed far fewer genes than other cells in the same experiment.

To summarize, the present invention analyses showed that scNanoGPS reliably deconvoluted long-reads into single-cells and single-molecules to detect single cell transcriptomes and dissect the tumor microenvironment (TME) from scNanoRNAseq data. scNanoGPS enables detection of cell-type-specific splicing profiles in human tumors

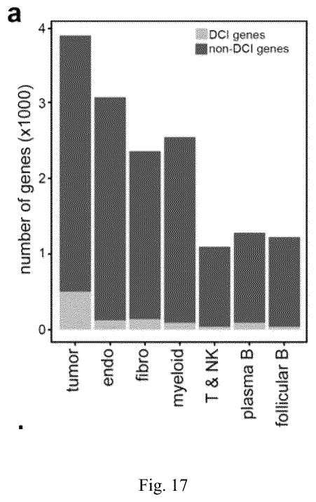

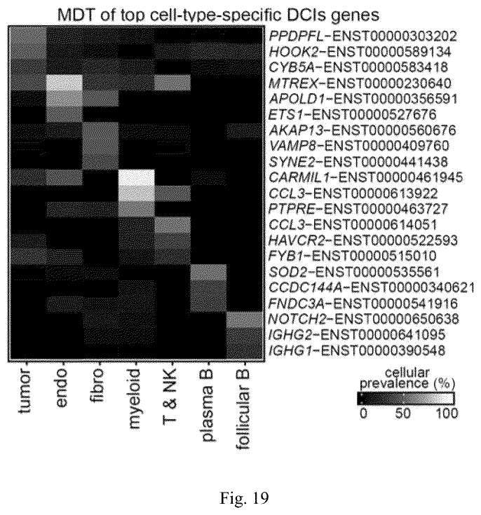

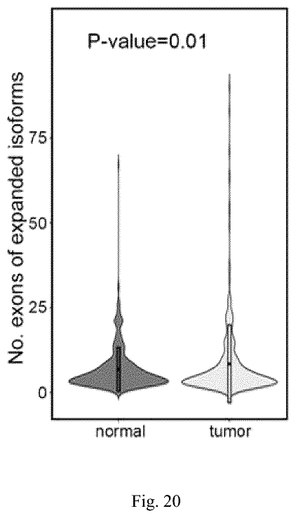

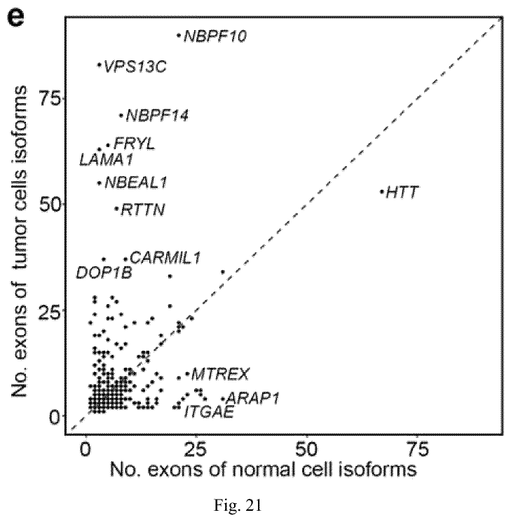

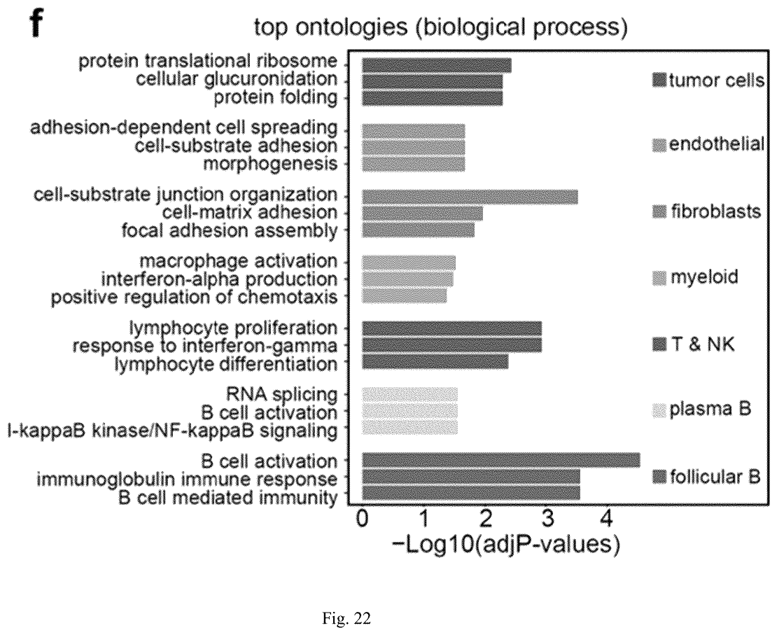

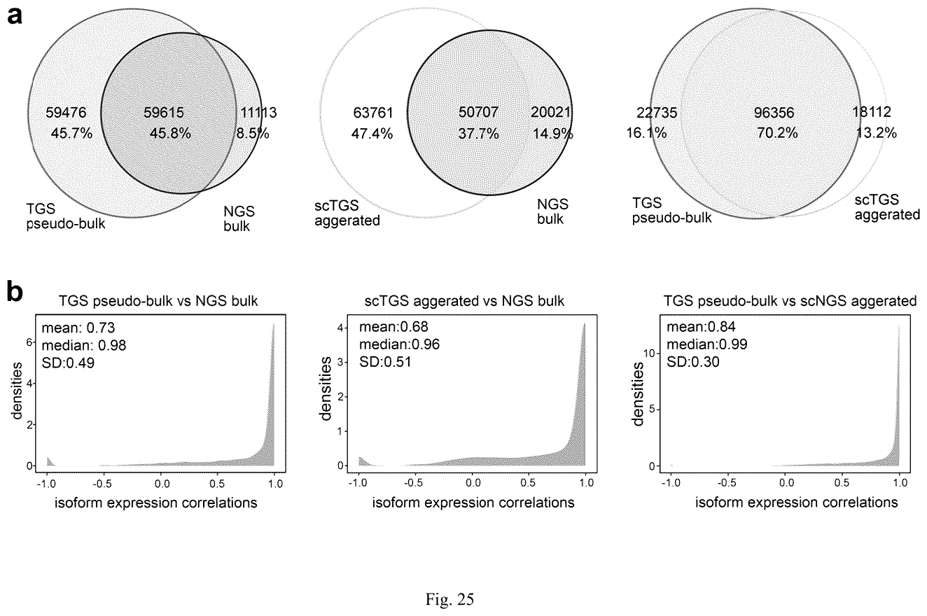

In one embodiment, to access the robustness of scNanoGPS in discovering splicing isoforms of different cell types in the TME, the present invention performed detailed isoform analysis of a frozen kidney tumor (RCC1). On average, scNanoGPS detected 6 transcripts per gene by referring to all known transcripts in GENCODE (v32). Comparisons with bulk RNAseq (NGS) data of same tumor revealed that up to 47% isoforms were only detected by TGS approaches, as shown in FIG. 25 upper panel, while isoform expression levels showed moderate to high concordance: median: 96%; mean: 68%; as shown in FIG. 25 lower panel. Further analyses showed that each cell type tended to express a combination of multiple isoforms, consistent with a recent study that reported isoform specificity in mouse cortex. To identify cell-type-specific preference of isoforms, the present invention compared the relative compositions of different isoforms of each gene among all 7 major cell types. Analysis of the present invention identified 1,014 genes that preferably expressed significantly different combinations of isoforms (DCIs, Chi-sq test P-values<0.05 and |Prevalence Differences|≥ 10%) among all cell types, including 499 DCI genes in tumor cells, 122 in endothelial cells, 137 in fibroblasts, 90 in myeloid cells, 38 in T & NK cells, 90 in plasma B cells and 38 in follicular B cells, according to FIGS. 17-19, and Table 5-11. Of note, the present invention detected 2-4 times more genes with DCIs in tumor cells compared to immune and stromal cell types. The top-ranked tumor-cell-specific DCI genes were PDE10A and NR4A2 involved in cAMP pathways. Additionally, the present invention observed that tumor-cell-preferred isoforms had slightly more exons, paired T-test P-value=0.01, as shown in FIG. 20, particularly in genes with more than 20 exons such as NBPF10, VPS13C, and NBPF14, shown in FIG. 21. According to FIG. 22, Geneset (MSigDB, GO:BP) analysis showed that cell-type-specific DCI genes were commonly enriched in pathways related to cell-type-specific functions, such as cAMP pathway and resistance associated glucuronidation pathway in tumor cells, interferon-alpha production pathways in myeloid cells, lymphocyte proliferation pathway in lymphocytes, and immunoglobin-mediated immune responses in B cells.

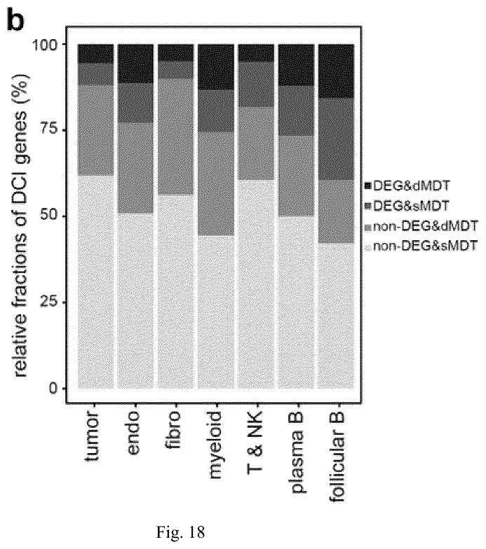

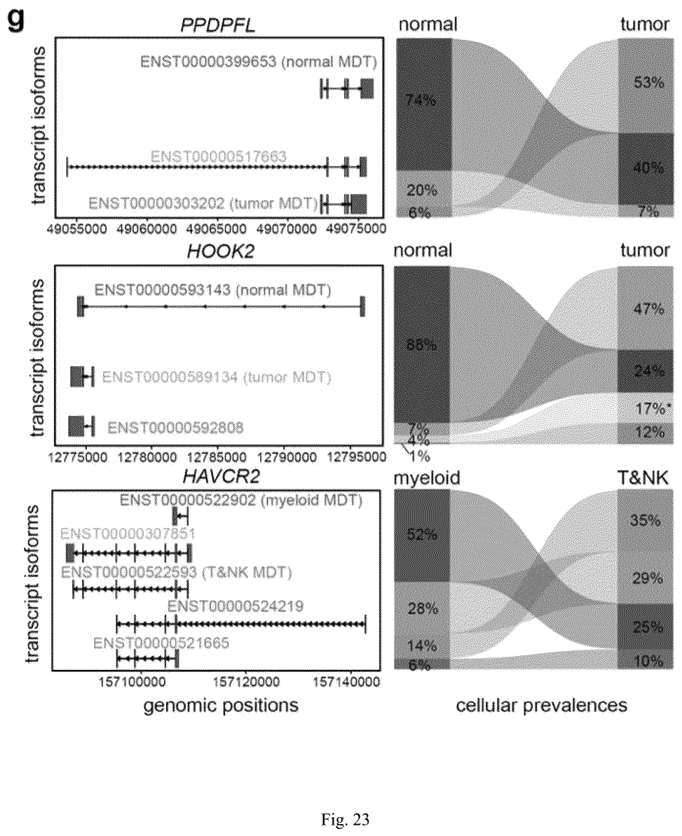

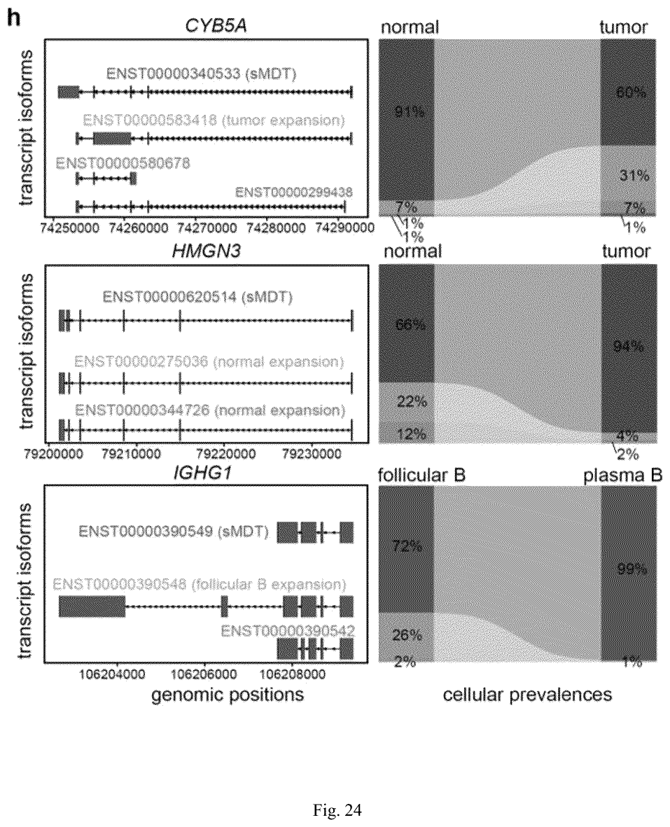

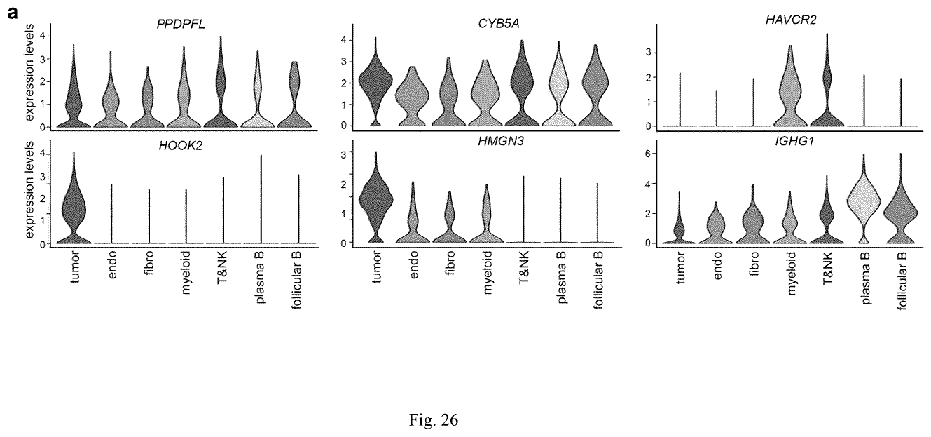

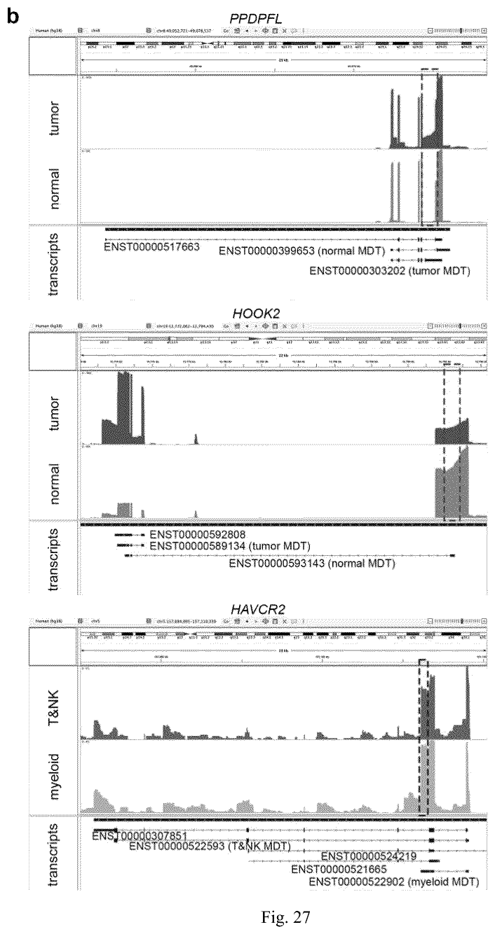

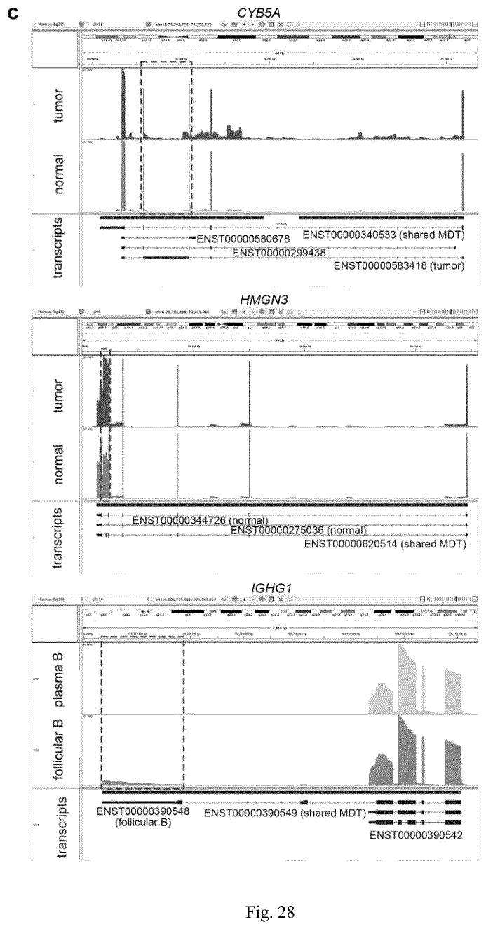

Notably, the present invention observed that a large portion (mean: 83%, SD: 9.9%) of cell-type-specific DCI genes were not detected as differentially expressed genes (DEGs) in all cell types, as shown in FIG. 18. Tumor-cell-specific DCI genes preferably expressed different most dominant transcripts (MDTs) regardless of their overall gene expression levels. For instance, the proliferation gene PPRPFL, expressed MDTs ENST00000303202 in tumor cells and ENST00000399653 in normal cells, although the overall expression levels of this gene in the two cell types were not significantly different, as shown in FIGS. 26-27 and FIG. 23. On the other hand, the organelle hook protein coding gene HOOK2 preferably expressed ENST00000589134 in tumor cells and ENST00000593143 in normal cells and had significantly higher expression levels in tumor cells compared to normal cells, as shown in FIG. 23 and FIG. 27. In addition, the present invention observed a small fraction of cell-type-specific DCI genes that expressed same MDTs, but their cellular fractions were different between tumor and normal cells. One example was CYB5A which expressed isoform ENST00000340533 in 60% of tumor cells but 91% of normal cells. Another example was HMGN3 which expressed isoform ENST00000620514 in 94% of tumor cells but 66% of normal cells, as shown in FIG. 24 and FIG. 28.

Interestingly, the present invention also observed isoform preference in immune and stromal cell types, although fewer genes were involved compared to tumor cells. For instance, myeloid and T cells both expressed HAVCR2, yet the MDTs of this gene were distinct in the two immune cell types, as shown in FIG. 23 and FIG. 27. In contrast, the B cell specific gene, IGHG1 expressed the same MDT (ENST00000390549) in both follicular and plasma B cells, but the cellular prevalence of this MDT were significantly different in the two B cell subtypes (72% in follicular B cells, 99% in plasma B cells), as shown in FIG. 24, indicating its relevance to sub-cell-type specific functions.

In summary, the present invention demonstrated the usages of scNanoGPS in studying cell-type-specific splicing isoforms in tumors. The results showed that a larger portion of genes utilized different MDTs in both tumor and immune cell types. Genes expressing same MDTs may have distinct cellular prevalence in different cell types regardless of overall gene expression levels.

### Transcriptome-Wide Mutations of Different Cell Types in the Tumor Microenvironment

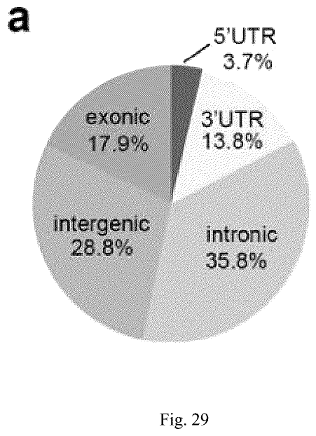

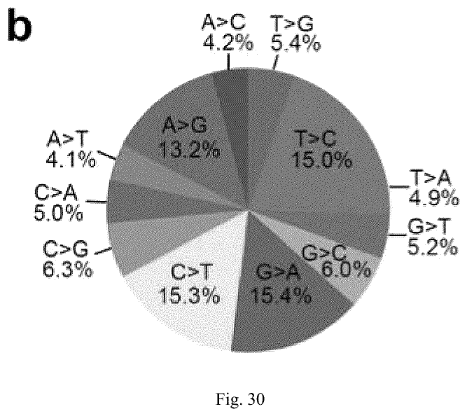

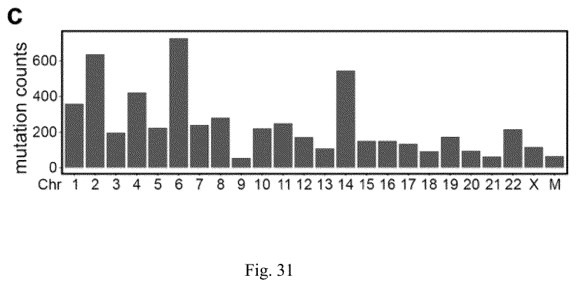

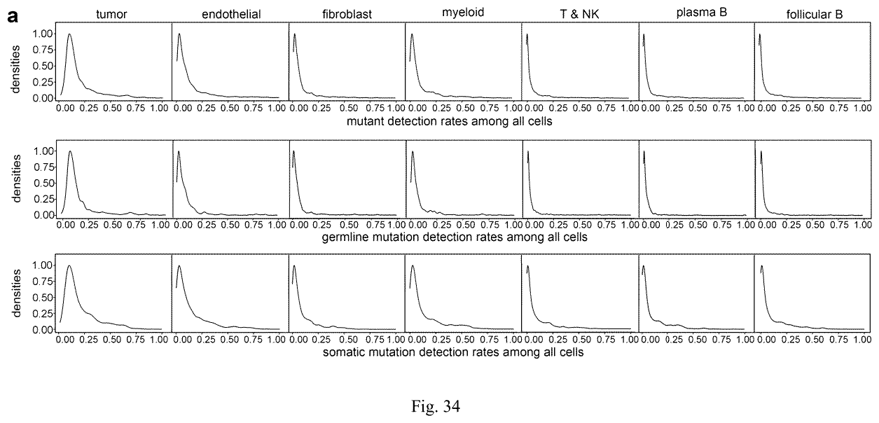

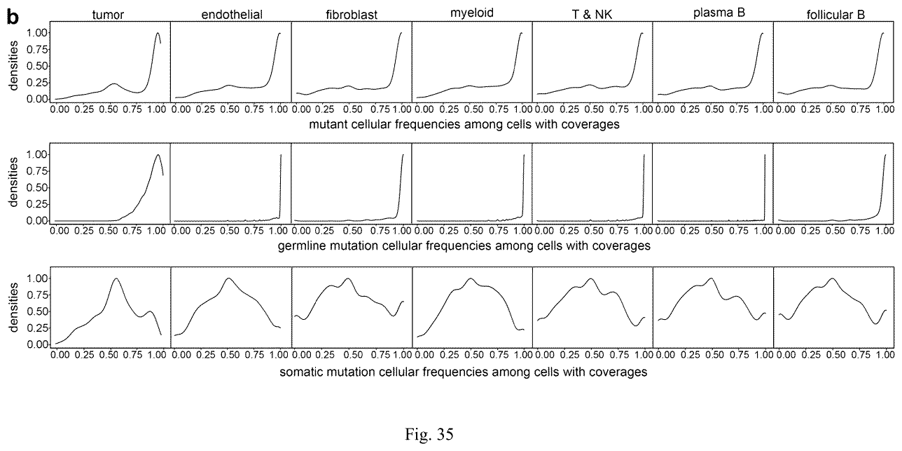

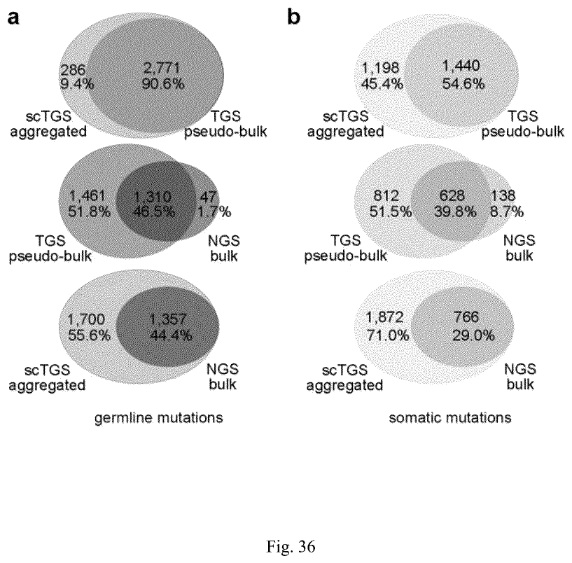

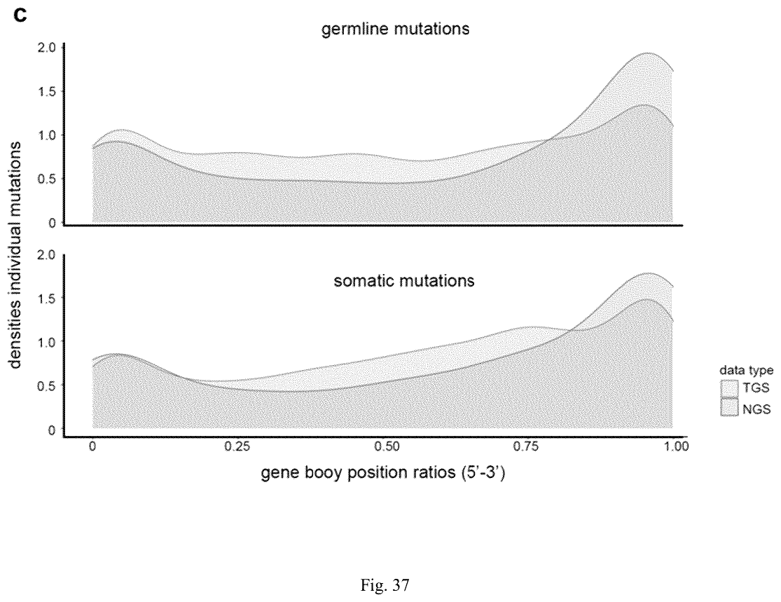

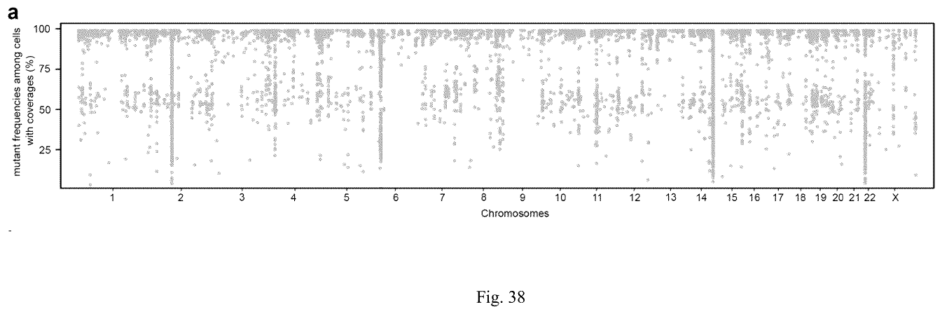

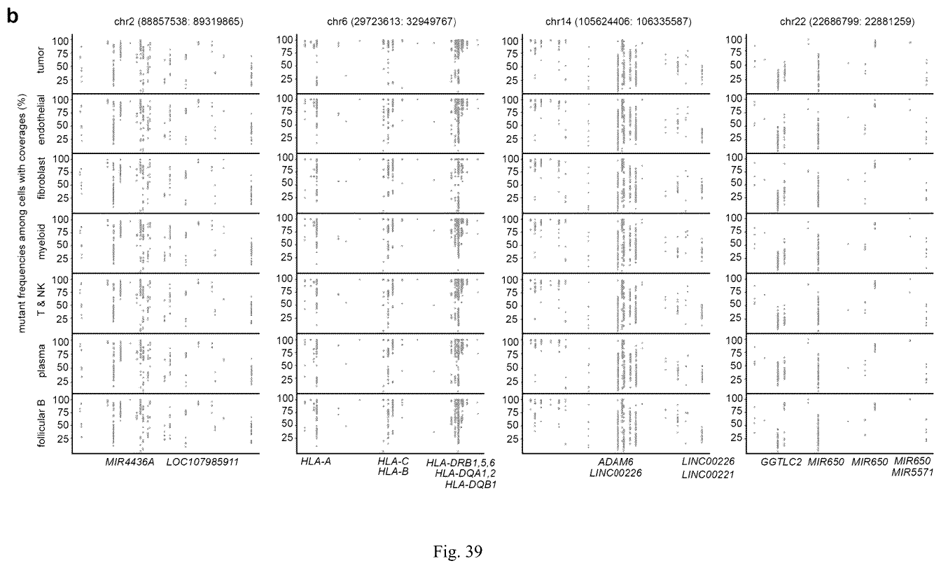

To accurately detect transcriptome-wide mutations in single cells, the present invention built consensus sequences of single molecules and required at least 2 variant supporting reads. In addition, the present invention filtered out mutations that were detected in less than 1% of cells over all cells or less than 5% within individual cell types, which removed most random errors, as shown in Table 13. In total, the present invention detected 6,390 mutations from 3,470 single nuclei transcriptomes of a frozen kidney tumor (RCC1). Further, in one embodiment, the present invention classified these mutations into germline mutations detected in >90% cells with coverages and somatic mutations detected in a wide range of cells across all cell types, as shown in FIGS. 34-35. The analysis showed that 90.6% germline mutations aggregated from scNanoGPS results were detected in pseudo-bulk long-read data, as shown in FIG. 36. However, the concordance between TGS with NGS were moderate (germline: 46.5% and somatic: 39.8%), which largely were due to differences in gene body coverages, e.g., NGS bulk RNAseq data had poorer uniform gene body coverages, as shown in FIG. 37. Among all mutations, 17.9% were in exonic regions, while others were spreading across non-coding regions (35.8% intronic, 28.8% intergenic, 3.7% 5′UTR and 13.8% 3′UTR), as shown in FIG. 29. Statistical analysis revealed that the odds of mutation detection were significantly (all chi-sq P-values <2.2e-16) increased in exonic (odds ratio: 7.8), 5′-UTR (odd ratio: 5.2) and 3′-UTR (odds ratio: 4.8) regions and decreased in intergenic (odds ratio: 0.65) and intronic (odds ratio: 0.46) regions as expected. Of note, it was observed 53.3% exon mutations were nonsynonymous. According to FIG. 30, transition mutations were more frequent (13-15%) than transversion mutation types (4-6%). The potential RNA editing (C>U) events were not distinguishable from C>T transition (15.3%). The data of the present invention showed that these transcriptome-wide mutations were distributed across all chromosomes except for Y-chromosome, as shown in FIG. 31. The data revealed 4 shared mutation hotspots on Chr2, 6, 14 and 22 in all 7 cell types, where Chr6 mutation hotspot was known to affect HLA gene clusters in both tumor and normal development26 and Chr14 hotspot harbored mutations in LncRNAs, according to FIGS. 38-39.

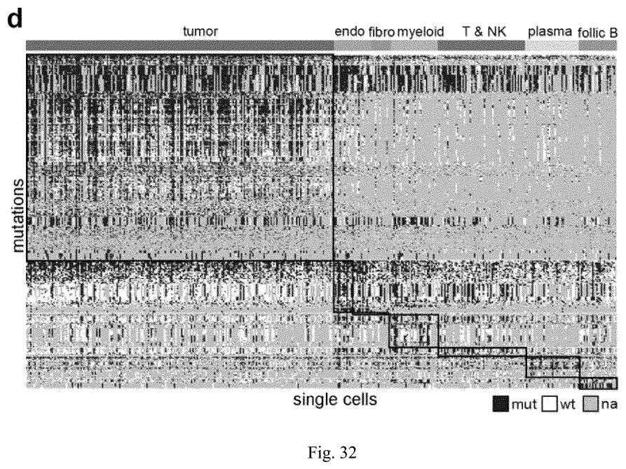

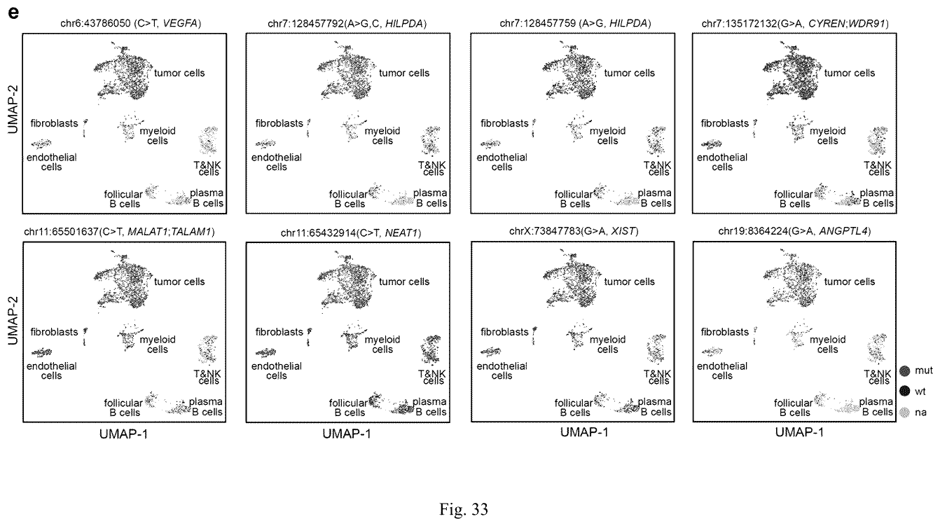

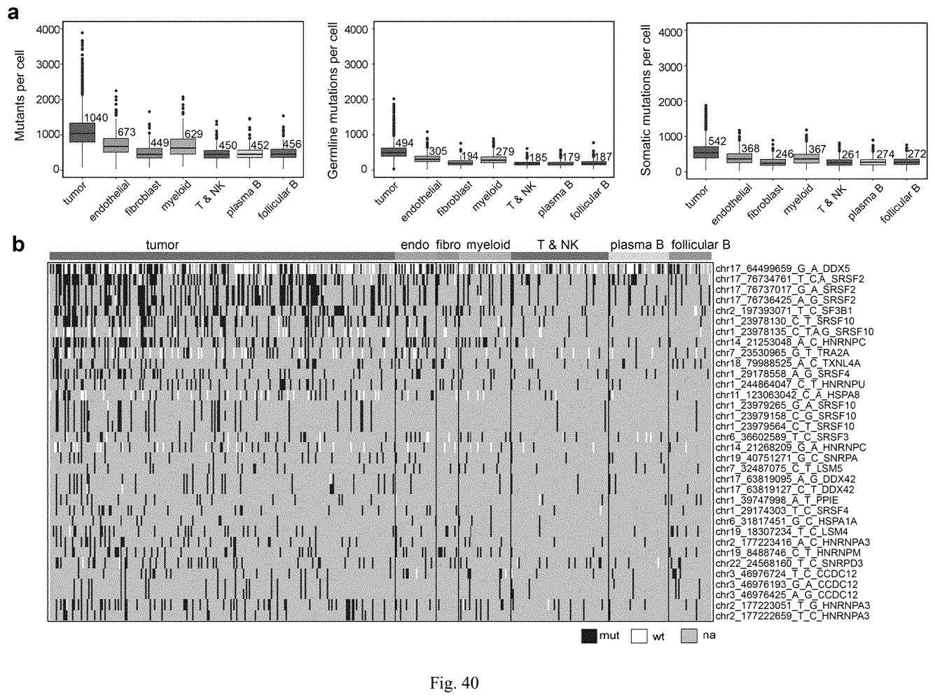

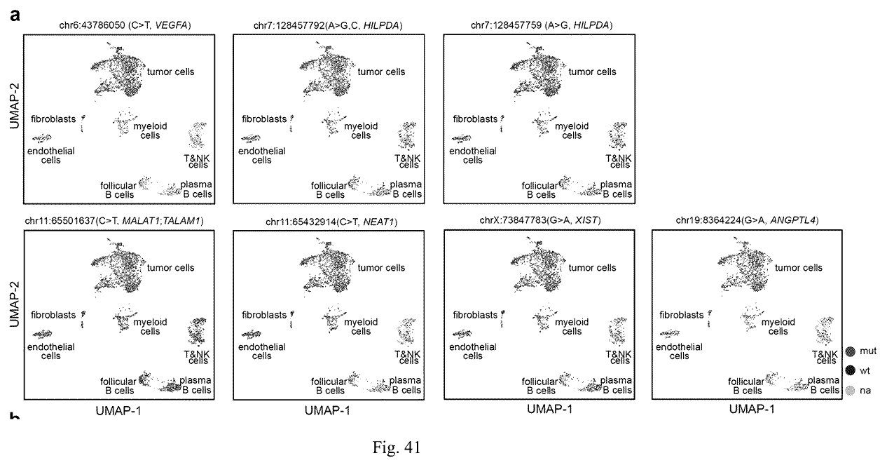

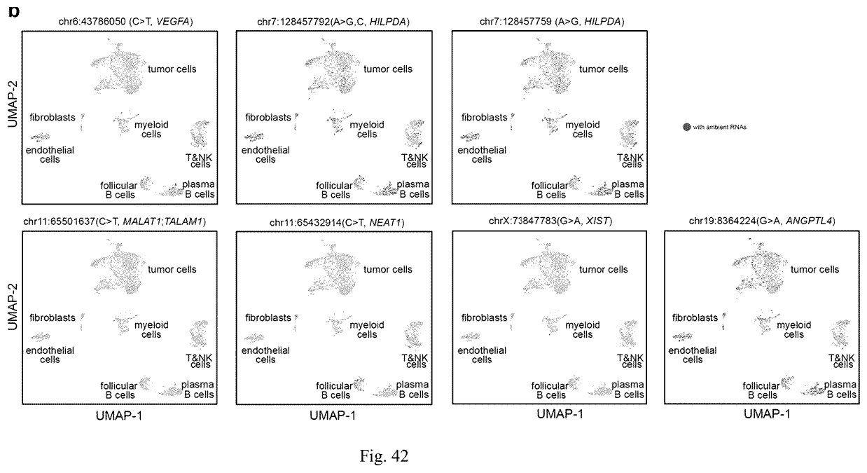

In one embodiment, the data showed that the mutated transcripts were detected in 15-25% cells of each cell type, according to FIG. 34, indicating the dependency of mutation detection rates on gene expression levels and transcript capture efficiency in single cell RNA sequencing technology. The number of total mutations in individual cells varied across cell types from 1119 SNVs in tumor cells, 758 in endothelial cells, 506 in fibroblasts, 703 in myeloid, 461 in lymphoid, 484 in plasma cells and 501 in follicular B cells, as shown in FIG. 40. To mitigate false positive detection of mutations due to ambient RNAs, SoupX30 was run to de-noise the data and removed mutations that were either landed on ambient RNAs or mapped to non-coding regions, as shown in FIGS. 41-42. Next, the present invention compared the cellular frequencies of mutations among different cell types to identify mutations that were differentially expanded (deMuts). The results showed that tumor cells had the largest number (N-609) of deMuts, followed by myeloid cells (N=99), plasma B cells (N=63), endothelial cells (N=57), follicular B (N=29), lymphocytes (N=26) and fibroblasts (N=1), according to FIG. 32 and Table 14-20. Observation of lower mutation burden in normal cell types is consistent with the prior knowledge of spontaneous mutation accumulation in normal organs throughout life time. Tumor cell specific deMuts included many COSMIC genes, such as VEGFA, NEAT1, MALAT1, HILPDA etc., as shown in FIG. 33. Further, the present invention detected several genes that were both mutated and differentially spliced in the same cell types, such as HMGN3 and UBE2G2 in tumor cells, CD74 and IFI30 in myeloid cells, and RPS2 in endothelial cells indicating their important roles in tumorigenicity. Additionally, it is observed several cell-type-specific deMuts involved in the spliceosome (GSEA: KEGG pathway) such as SRSF3 and HNRNPC mutants in tumor cells and DDX5 mutant in endothelial cells, according to FIG. 40, lower panel.

In summary, the present demonstrated that scNanoGPS can robustly detect cell-type and cell-state specific mutations. The data highlighted the importance of identifying population-specific mutations to understand their functional roles in cancer progression.

### Conclusion

In one embodiment, the present invention discloses a computational tool called scNanoGPS to facilitate high throughput single cell long-read sequencing data analysis. scNanoGPS achieves independent deconvolution of raw data without the guidance of short-reads nor barcode whitelist and calculates genotypes-phenotypes of thousands of individual cells, addressing the major computational challenges of an emerging scNanoRNAseq technology.

Two previous methods called Sicelore and scNapBar were developed to deconvolute raw reads, however, both relied on the guidance of paralleled NGS data. An experimental method called scCOLOR-seq was developed to reduce error rates in barcodes by designing bi-nucleotide repeats in barcode sequences, but it still relied on prior knowledge of true barcodes to curate errors. Moreover, scCOLOR-seq required customized synthesis of gel-beads to adapt to the droplet system. The other NGS-independent barcode deconvolution tools, Sockeye and BLAZE relied on comparison of long-reads data with barcode whitelist and large LDs. This is worrisome due to barcode manufacturing deviations and misassignment introduced by large LDs. In comparison, scNanoGPS achieves >80% accuracy of detecting true CBs directly from long-reads data using a multi-step method, iCARLO, without guidance of NGS data nor barcode whitelist. Sockeye also provided UMI detection function, which unfortunately dropped off a large portion of data due to over-filtering and/or over-merging.

In one embodiment, dysregulation of transcript isoforms plays critical roles in tumor progression. Previous studies showed that only ˜20-40% of transcriptional isoforms can be reconstructed from NGS bulk RNAseq data. Long-read single cell sequencing technologies enable in-depth annotation of splicing isoforms at single cell levels. scNanoGPS provides a robust computational tool to achieve this goal. The disclosed analysis of a frozen kidney tumor revealed that all major cell types in the tumor commonly express a combination of different isoforms instead of one canonical isoform.

In one embodiment, another important finding is that tumor cells preferably express different MDTs of tumor suppressors although their overall gene expression levels are not significantly different from normal cells. The results implied that the discovery of cancer-specific genes using gene expression levels may only be revealing the tip of iceberg of transcriptional diversities in cancer.

scNanoGPS provides a powerful approach for synchronic tracing of cell-lineage and cell-fate by measuring both plastic phenotypic markers (genes, isoforms) and stable genetic markers (mutations, copy numbers) of the same cells to study tumor evolution and therapeutic responses. scNanoGPS detects transcriptome-wide point-mutations with accuracy by building consensus sequences of single molecules and performing consensus filtering of cellular prevalence, which removes most false calls due to random sequencing errors.

In addition, scNanoGPS has broad applications in many other genomic areas, such as measuring single cell gene fusions, tandem repeats, splicing velocities, repetitive genes, or long non-coding genes to investigate diverse mechanisms of human diseases including but not limited to cancer.

In one embodiment, the present invention also discloses scNanoFUSE, which provides several methods for detecting fusion reads, removing false positives, predicting accurate fusion location, and assembling the consensus fusion transcripts.

### Data and Software Availability

Raw sequencing data and processed data are submitted to Gene Expression Omnibus (GEO): GSE212945. Software is available at GitHub: github.com/gaolabtools/scNanoGPS Codes of statistical tests are provided in the Supplementary Note.

### Methods

### Cancer Cell Line and Tumor Tissue Samples

The two cancer cell lines (A375, H2030) and the four frozen tumors (RCC1, RCC2, MEL1, MEL2) were provided by Dr. Jason Huse at UT MD Anderson Cancer Center. The cell lines are standard commercialized cancer lines. The tumor tissues were collected with consent under IRB approval at UT MD Anderson Cancer Center.

### Preparation of Single Nucleus Suspension

Single nucleus suspensions of two cancer cell lines were prepared by following the 10× Genomics protocol (CG000365 Rev C), which is hereby incorporated by reference. Single nucleus suspensions of frozen tumor tissues were prepared according to the method as previously described. Frozen tissue was cut into tiny pieces in 10-cm Petri dish with 500 ul-2 ml NST-DAPI buffer for 10-15 minutes and filtered through a 40 mm Flowmi into 1.5 ml LowBind Eppendorf tube and centrifuged at 4° C. 300 g for 5 minutes. The resulting nuclei pellet was washed three times with cold Nuclei Wash and Resuspension Buffer. After cell counting, the nuclear suspension was centrifuged again and resuspended in the appropriate volume depending on the nuclei counting results. Preparation of NST-DAPI buffer: Mix 800 ml of NST solution (146 mM NaCl, 10 mM Tris-base (pH 7.8), 1 mM CaCl2, 21 mM MgCl2, 0.05% (wt/vol) BSA and 0.2% (v/v) Nonidet P-40) with 200 ml of DAPI solution (106 mM MgCl2 and 10 mg of DAPI). The solution is filter-sterilized and is stored at 4° C. in the dark for up to 1 year. Preparation of Nuclei Wash and Resuspension Buffer: 1×PBS with 1.0% BSA and 0.2 U/ul RNase Inhibitor.

Preparation of Barcoded Full-Length cDNAs of Single Nuclei

Single nucleus suspensions were loaded onto 10× Genomics Chromium Controller (iX) with Chip J to capture 3000-6000 single nuclei. The full-length mRNAs and/or pre-mRNAs were barcoded with cell barcodes (cellBCs) and unique molecular identifiers (UMIs) through cDNA amplification using 10× Genomics protocol. In one embodiment, the present invention modified the cDNA amplification protocol by extend the elongation time to 3 minutes to enrich longer molecules as previously described.

The barcoded full-length cDNA transcripts (10 ng) were amplified for five cycles with the following two customized primers synthesized by Integrated DNA Technologies (Coralville, IA): 1) 5′-biotinylated TruSeq Read 1 forward primer 5′-/5Biosg/AA AAA CTA CAC GAC GCT CTT CCG ATC T-3′ (25 nM); 2) 3′ partial TSO reverse primer 5′-NNN AAG CAG TGG TAT CAA CGC AGA GTA CAT-3′. The amplified single-cell cDNA transcripts were subjected to 0.8×SPRIselect reagent (Beckman Coulter, CA) clean-up to remove unbound and excess biotinylated primers, where the bound cDNA was eluted off the bead matrix in 45 uL of Qiagen Buffer EB (Qiagen; Valencia, CA). The eluted cDNA was further purified through the binding of the biotinylated template to Dynabeads™ M-270 Streptavidin beads (Invitrogen; Waltham, MA). Prior to the selection of the cDNA template, 15 uL of the Dynabeads™ M-270 Streptavidin beads were washed in 1 mL of 1×SSPE solution (UltraPure™ 20×SSPE Buffer (Invitrogen) freshly prepared with nuclease-free water). A magnetic stand was used to separate the streptavidin beads from the initial wash solution. The streptavidin beads were then washed three times with 15 uL of 1×SSPE with removal from the magnet and resuspension of the beads in a fresh wash solution with each repeat wash. Following the final wash, the streptavidin beads were resuspended in 10 uL of 5×SSPE solution and the biotinylated cDNA template was added to the washed beads. This mixture was placed on a tube rotator at room temperature for 15 minutes. Post incubation on the rotator, with the biotinylated cDNA template bound to the washed streptavidin beads, the sample was placed back on the magnetic stand for separation. The cDNA-bound beads were washed twice with 100 uL 1×SSPE solution and a final wash with 100 uL Buffer EB. The use of the biotinylated forward primer and subsequent purification with the Dynabeads™ M-270 Streptavidin beads allowed for the selective depletion of cDNA missing the terminal poly(A)/poly(T) tail.

The streptavidin beads containing bound biotinylated cDNA were then resuspended in 100 uL of PCR master mix for a secondary amplification for five cycles with regular PCR primers: 1) TruSeq read 1 forward primer 5′-NNN CTA CAC GAC GCT CTT CCG ATC T-3′ and 3′ partial TSO reverse primer 5′-NNN AAG CAG TGG TAT CAA CGC AGA GTA CAT-3′. The PCR amplified product was purified with 0.8×SPRIselect reagent into a final elution of 51 uL in Buffer EB to allow for adequate template/volume for the necessary assessment of quality control (QC) metrics and PromethION library preparation.

The KAPA Biosystems HiFi HotStart PCR Kit (Roche; Basel, Switzerland) was used to prepare all PCR amplification mixtures for Nanopore library preparations. The following PCR conditions were followed for amplification: initial denaturation, 3 mins at 95° C.; five cycles of denaturation for 30 secs at 98° C., annealing for 15 secs at 64° C., and extension for 5 mins at 72° C.; followed by a final extension for 10 mins at 72° C.

Nanopore Sequencing Library Preparation for Full-Length cDNAs

Based on the sample molarity and average cDNA transcript length derived from the QC metrics, the sample input volume was calculated and used to progress into nanopore library preparation. The SQK-LSK110 ligation sequencing kit (Oxford Nanopore) was used to generate PromethION long-read cDNA libraries. The final sequencing was run on PromethION flow cell (v9.4.2) with one sample per flow cell by the DNA technology core at UC Davis using R9.4 chemistry. The output data was base-called live during the run using base-caller guppy (v5.0.12) in super-accurate base-calling model.

Generating Benchmarking Data with Paralleled NGS Sequencing of Fragmentized cDNAs

The same aliquot (25% volume) of barcoded full-length cDNAs were fragmented and subjected to next generation sequencing library preparation by following 10× Genomics Next GEM Single Cell Gene Expression protocol. The final libraries were sequenced on the Illumina Novaseq 6000 sequencer at the NUcore at Northwestern University. The sequencing data were processed using 10× Genomics software CellRanger ARC (v2.0)29.

Quality Control of scNanoRNAseq Data

The raw FASTQ files were processed by the scNanoGPS ‘NanoQC’ function to scan the distribution of raw read lengths, which generated a PNG plot named ‘read_length.png’ and a tab-separated table named ‘read_length.tsv’. The first and last 100 nucleotides of raw reads were extracted for sequencing quality analysis FastQC39.

Scanning Long-Reads with Expected Adaptor Patterns

The raw FASTQ files were processed by scNanoGPS ‘Scanner’ function to scan the expected adaptor patterns using the parameters (match: 2, mismatch: −3, gap opening: −5, gap extension: −2, sequence identity ≥70%) equivalent to NCBI Basic Local Alignment Tool (BLAST) algorithm. Raw reads with insert length less than 200 bp were excluded from scanning. After ‘Scanner’, a compressed file containing parsed raw cell barcodes (CBs) named ‘barcode_list.tsv.gz’ and a filtered read sequence file named ‘processed.fastq.gz’ were generated.

Deconvolution of Long-Reads into Single Cells

The raw reads that passed QC and pattern filtering steps were demultiplexed into single cells using scNanoGPS ‘Assigner’ function. Another input was the parsed barcode lists generated by ‘Scanner’. The true list of CBs was retrieved through an integrated algorithm called iCARLO. This algorithm included 4 steps. First, all CBs were ordered decreasingly by the number of supporting reads. The number of supporting reads and the order index were transformed into log 10 scale, as shown in FIG. 2, step 3 Assigner. The raw list of true CBs was estimated by thresholding the maximal partial derivatives of supporting reads against the rank of CBs. To buffer the changes, the present invention smoothed the partial derivatives within each 0.001 window of log 10-scaled CB ranks.

Let X be the number of supporting reads of CBs, i be the rank order of all CBs, and w be the 0.001 smoothing windows as defined in equation (1).

$$∀ X , i , w ∈ ( 1 , 2 , 3 , … , N ) \qquad ( 1 )$$

Each window w contains a set of CBs, allowing empty, according to their rank order i in log 10 scale per 0.001 tick as shown in equation (2).

$$0.001 \times w < {log}_{10} i < 0.001 \times ( w + 1 ) \qquad ( 2 )$$

The partial derivatives were calculated for each CB and then smoothed by taking median average of all values within each window w as shown in equation (3). The crude anchoring point was defined as a threshold cutoff where a smoothing window w had maximal partial derivative. The present invention defined the raw number of CBs (Cr) at this crude anchoring point as follows:

$$Cr = {10}^{0.001 \times {arg}_{w} max {med}_{i} [ \frac{{log}_{10} {X}_{i + 1} - {log}_{10} X}{{log}_{10} ( i + 1 ) - {log}_{10} i} ]} \qquad ( 3 )$$

The present invention then extended this crude anchoring point by an empirical percentage (10%) to an external boundary (N=Cr2) of true CBs to rescue as many CBs as possible. In the third step, the present invention exhaustively computed pair-wised LD distances of all Cr2 CBs. CBs within 2 LD were merged back one directionally to the CB harboring more supporting reads and obtained a collapsed list of true CBs (N=Cr3). Lastly, the present invention retrieved the final list of true CBs (N=Cr4) by removing CBs which cover less than 300 genes. According to this final list of true CBs, the master FASTQ file resulting from ‘Scanner’ was split into single cell FASTQ files stored in a temporal folder holding all the meta files for further usages.

### Curation of Sequencing Errors in Single Molecules

Detection of the true list of UMIs and curation of sequencing errors in single molecules was performed by using scNanoGPS ‘Curator’ function. To detect true UMIs, deconvolution of the single cell FASTQ files were first aligned to the reference genome (GRCh38) using MiniMap2 under the splicing mode (−ax splice). Reads mapped to the same genomic regions (coordinates within 5 bp) were grouped into batches. Batch calculation was conducted by paralleled computing cores. For the reads, that belong to the same clusters and have UMI within two LDs, were considered as reads of the same molecules. These UMI sequences were curated to be identical. To curate errors in gene bodies, the reads with the same curated UMIs were collapsed into consensus sequences of single molecules using SPOA13. Finally, the consensus reads were re-mapped against the reference genome (GRCh38) using MiniMap2 under splicing mode (−ax splice) to generate consensus BAM files for all downstream analyses.

Calculation of Same-Cell Multi-Omics from Consensus Reads

The consensus BAM files of single cells were used as input to calculate single cell multi-omics, including transcriptomes, isoforms and point mutations using scNanoGPS ‘Reporter’ functions. The single cell gene expression profiles were calculated using FeatureCount from the Subread package that support long-read gene level counting. The single cell isoforms were calculated using LIQA, a method designed to calculate isoforms from long-read data of spliced mRNAs. The present invention used default parameters (weight of bias correction: 1, maximal distance: 20 bp) when running LIQA. The single cell point mutation detection was conducted using a robust long-read variant detection tool, LongShot. The present invention benchmarked the LongShot results in terms of the number of SNVs with different cell prevalence and different number of supporting reads, as shown in Table 1. With the elbow method, the present invention required that each alternative allele should be supported by at least two consensus reads. The resulting VCF files of single cells were merged using BCFtools (v1.15). A final list of mutations of all cells was obtained by consensus filtering, where all variants detected in less than 1% of the cells were considered as random errors and removed from analysis. To differentiate between true wild-types and missing values, the present invention re-scanned the read depths of all loci in the final list by Samtools (v1.15). Loci with 0/0 genotypes with supporting reads >0 were defined as true wildtypes, otherwise, as missing values if none supporting reads were found. The final mutation loci were annotated using ANNOVAR, which included dbSNP (v150) and COSMIC (v96) as references. The single cell copy numbers were calculated using the previously published method, ‘CopyKAT’ (v1.0.6) with default parameters, which is hereby incorporated by reference. In all final output matrices, features/genes were put in rows while cell barcodes were in the columns.

### Single Cell Gene Expression Data Analysis: QC and Defining Major Cell Types

The gene expression matrices of NGS-based 3′-scRNAseq data were processed using CellRanger ARC29 (10× Genomics) and sent for downstream analysis using the ‘Seurat’ R package (v4.1.1). In 4 tumor samples, doublets were removed using R package ‘DoubletFinder’ (v2.0.3) with an assumed doublet rates of 0.8% per 1000 cells. Cells with more than 10,000 genes, or more than 100,000 UMIs, considered as doublets, were removed as outliers and suspected doublets. Cells with less than 300 genes were filtered out due to low gene coverages. Furthermore, cells with extremely higher fractions of mitochondrial genes were filtered out using arbitrary outlier cutoffs (5% in RCC1 and RCC2; 20% in MEL1 and MEL2). UMI count matrices were normalized using ‘LogNormalize’ method in ‘NormalizeData’ function and scaled across cells using ‘ScaleData’ function in ‘Seurat’. The top 2000 highly variable genes were selected with ‘FindVariableFeatures’ function based on ‘vst’ method and used for Principal Component Analysis (PCA). Next, the present invention performed PCA and uniform manifold approximation and projection (UMAP) for dimension reduction with the top 30 PCs. ‘FindNeighbors’ function based on the top 30 PCs and ‘FindClusters’ functions were applied to perform unbiased clustering of cells. In all the samples, the present invention defined a cluster of low-quality cells that did not express known cell type markers and had much fewer genes and UMIs compared to other cells in the same experiments. Final clustering analyses were reperformed without these low-quality cells. To identify the tumor cells, the present invention used the UMI count matrix as input to infer chromosomal CNA profiles using R package ‘CopyKAT’ (v1.0.6). Cells with genome-wide CNAs were labeled as tumor cells.

### Analyses of Cell-Type-Specific Splicing Isoforms

Cell-type-specific splicing isoform analyses started from single cell isoform expression matrices that summarized the expression levels of all known isoforms based on GENCODE (v32) in single cells. For pairwise two group (cell type) comparisons, the present invention filtered out sporadically expressed genes, which were detected in less than 5% cells in both comparison groups. Additionally, genes with only one isoform were excluded from this analysis. To mitigate false discovery driven by dropouts, the isoform expression levels of a given gene were aggregated across all cells within each comparison group. The aggregated counts of expressed molecules of all isoforms of a given genes in both comparison groups were sent for Chi-square tests to test whether the relative composition of different isoforms of a given gene was significantly different in the two comparison groups or not. P-values were adjusted using Benjamin-Hochberg (BH) correction for multiple testing with a 5% false discovery rate. The relative frequency of all isoforms of a given genes and the differences in two comparison groups were also calculated. Finally, the genes expressing significantly different combination of isoforms (DCIs) were defined as having FDR adjusted P-values <0.05 and at least one isoform had different cellular prevalence in two comparison groups >=10%.

### Detection of Cell-Type-Specific SNVs

To further remove random errors, the present invention filtered out the called positions that were detected in less than 5% cells in each cell type or in comparing group. Next, the present invention generated a count matrix that included the number of wild-type cells (expressed only reference alleles) and the number of mutated cells (expressed variant alleles) of all candidate SNVs in both groups that were under comparison. In one embodiment, the present invention performed a Chi-square test to measure whether a candidate SNV had significantly different cellular frequencies. P-values were adjusted using BH correction for multiple testing with a false discovery rate of 5%. This analysis was only performed using data of cells that had read coverages. Cells without read coverages were not included to calculate the cellular frequencies of mutations. It was determined the candidate SNVs with FDR adjusted P-value <0.05 and the differences in cellular frequencies >0.1 as population specific differentially expanded SNVs (deMuts).

### Summary of Statistical Methods

The present invention applied Chi-sq tests to compare the relative frequencies of isoforms or mutations in two comparison groups. P-values were adjusted using BH method to adjust for multiple test errors with a false discovery rate of 5%. Pearson's correlation and P-value were applied to measure the similarity of gene expression profiles and the total counts of UMIs per cell between scNanoGPS and NGS approaches. Paired-two-side t-test was performed to compare the number of exons of different isoforms of same genes between tumor and normal cells. All significance cutoffs used in this study were set at 0.05.

TABLE 1
Comparison of functional modules of scNanoGPS with existing tools

| Function modules | scNanoGPS | Sockeye | BLAZE |
|---|---|---|---|
| Detection of true | Scan possible CBs from raw data using 4-step | Compare to barcode whitelist to | Compare to barcode |
| CBs | iCARLO algorithm; Curate errenous CBs with | identify possible CBs, allowing 2 | whitelist to identify possible |
| | top-ranked CBs (having more supporting | Levenshtein Distance | CBs, allowing 5 Levenshtein |
| | reads); allowing 2 Levenshtein Distance | | Distance |
| Detection of UMIs | Mapped to same genomic positions (<5 bp), | Network-based clustering, | N/A, by the time of |
| | allowing 2 Levenshtein Distance | allowing 2 Levenshtein Distance | submission |
| Construction of | Collapse reads with same UMIs | N/A, by the time of submission | N/A, by the time of |
| consensus reads | | | submission |
| Expression profile | Output gene by cell matrix | Output gene by cell matrix | Output gene by cell matrix |
| calculation | | | |
| Isoform profile | Output isoform by cell matrix | N/A, by the time of submission | Output isoform by cell |
| calculation | | | matrix |
| SNV profile | Output SNV by cell matrix | N/A, by the time of submission | N/A, by the time of |
| calculation | | | submission |

TABLE 2
Sample quality metrics

| | | | | | Pass | | | Aver- | | Aver- | | | |
|---|---|---|---|---|---|---|---|---|---|---|---|---|---|
| | | | | Reads | rates | | | aged | | aged | | | |
| | | | | past | of | | | quality | Detected | number | | | Inter- |
| Sam- | | | | adaptor | adaptor | Median | Maximal | score | number | of raw | Exonic | Intronic | genic |
| ple | Cancer | Patient | Total | pattern | pattern | read | read | in first | of | reads | mapping | mapping | mapping |
| ID | type | ID | reads | scanning | scanning | length | length | 100 bp | cells | per cell | rates | rates | rates |
| RCC | renal | RCC | 98,349,656 | 76,630,302 | 77.92% | 921 bp | 190,885 bp | 20.47 | 3,470 | 22,084 | 13.91% | 70.20% | 15.89% |
| 1 | cell | 12 | | | | | | | | | | | |
| | carcinoma | | | | | | | | | | | | |
| RCC | renal | RCC | 87,800,959 | 68,137,378 | 78.83% | 830 bp | 386,890 bp | 20.91 | 3,638 | 18,729 | 15.50% | 69.95% | 14.55% |
| 2 | cell | 12 | | | | | | | | | | | |
| | carcinoma- | | | | | | | | | | | | |
| | brain | | | | | | | | | | | | |
| | metastasis | | | | | | | | | | | | |
| MEL | melano- | MEL | 72,828,041 | 55,845,250 | 78.04% | 890 bp | 183,138 bp | 21.05 | 7,426 | 7,520 | 12.65% | 65.92% | 21.43% |
| 1 | ma | 3 | | | | | | | | | | | |
| MEL | melanoma- | MEL | 98,363,542 | 75,990,450 | 78.47% | 801 bp | 637,942 bp | 20.93 | 1,192 | 63,750 | 13.67% | 59.620% | 26.71% |
| 2 | brain | 3 | | | | | | | | | | | |
| | metastasis | | | | | | | | | | | | |
| H20 | non- | NA | 105,347,205 | 91,444,891 | 87.31% | 856 bp | 411,989 bp | 21.81 | 4,212 | 21,711 | 11.84% | 59.84% | 28.32% |
| 30 | small | | | | | | | | | | | | |
| | cell | | | | | | | | | | | | |
| | carcinoma | | | | | | | | | | | | |
| A37 | melanoma | NA | 67,436,356 | 58,178,979 | 86.80% | 899 bp | 282,743 bp | 21.75 | 3,649 | 15,944 | 11.91% | 54.91% | 33.18% |
| 5 | | | | | | | | | | | | | |

TABLE 3
Comparison of CB detection results of three tools

| | A375 | | | H2030 | | |
|---|---|---|---|---|---|---|
| Items | scNanoGPS | BLAZE | Sockeye | scNanoGPS | BLAZE | Sockeye |
| True positive rate* | 97.99% | 95.84% | 95.85% | 98.32% | 97.27% | 97.23% |
| False positive rate# | 2.01% | 4.16% | 4.15% | 1.68% | 2.73% | 2.78% |
| True negative rate | 99.91% | 99.94% | 99.94% | 99.93% | 99.97% | 99.97% |
| False negative rate | 0.90% | 0.60% | 0.60% | 0.70% | 0.30% | 0.30% |
| F1 score | 0.90 | 0.92 | 0.92 | 0.93 | 0.96 | 0.96 |
| Time (H:M:S) | 18:17:17 | 5:01:37 | 15:37:25 | 28:02:05 | 9:40:45 | 23:10:01 |
| Peak memory | 1.21 GB | 7.14 G | NA | 1.30 GB | 8.31 GB | NA |

*detected CBs existed in CellRanger list/total detected CBs
#detected CBs not existed in CellRanger list/total detected CBs

TABLE 4
Usages of computing resources of analyzing
A375 data with scNanoGPS

| Function | Time spent | Memory | | Storage | |
|---|---|---|---|---|---|
| Modules* | (H:M:S) | usage | | usage | |
| Input FastQ files | — | — | | 89 | Gb |
| Scanner | 18:17:17.84 | 1.30 | Gb | 52.87 | Gb |
| Assigner | 18:29:35.18 | 576 | Gb | 86.50 | Mb |
| Curator | 53:15:38.13 | 16.82 | Gb | 74.69 | Gb∧ |
| Reporter_expression | 01:49:48.48 | 6.64 | Gb | 11.65 | Mb |
| Reporter_isoform | 11:01:22.24 | 2.76 | Gb | 23.81 | Mb |
| Reporter_SNV | 04:38:56.50 | 42.55 | Gb | 12.27 | Mb |

*with 30 cores;
∧temporary files

TABLE 5
Concordance of combinations of CBs, UMIs
and genes of scNanoGPS results with
CellRanger

| | Total | Overlapped | |
|---|---|---|---|
| | combi- | combi- | |
| | nations | nations | |
| | of UMIs | of | Con- |
| | and | UMIs and | cord- |
| | Genes | Genes | ance |
| Cells | (counts) | (counts) | (%) |
| CCTGCTCCATTAGGCC | 6307 | 4423 | 70% |
| (SEQ ID No. 1) | | | |
| GAGCTTAGTTCGCTCA | 7541 | 5288 | 70% |
| (SEQ ID No. 2) | | | |
| TTGATGTCATTGTCCT | 7995 | 5567 | 70% |
| (SEQ ID No. 3) | | | |
| AGCGGATAGCAGCTAT | 11327 | 7718 | 68% |
| (SEQ ID No. 4) | | | |
| GAGCGGTCAGGCTGTT | 14193 | 9889 | 70% |
| (SEQ ID No. 5) | | | |
| GATAACGAGCTGTAAC | 14696 | 10103 | 69% |
| (SEQ ID No. 6) | | | |
| GCCATGATCACCAATA | 19799 | 13542 | 68% |
| (SEQ ID No. 7) | | | |
| CGCTTAACAGGTCCTG | 37762 | 26986 | 71% |
| (SEQ ID No. 8) | | | |
| GCAACAGCATGTCAGC | 58376 | 41477 | 71% |
| (SEQ ID No. 9) | | | |
| GTATGTGGTATTGAGT | 101695 | 75438 | 74% |
| (SEQ ID No. 10) | | | |
| Combinations of CBs, | 279691 | 200431 | 72% |
| UMIs and Genes | | | |
| (counts) | | | |

TABLE 6
Tumor cell specific DCI genes

| | | Pre- | Pre- | Pre- | | Pre- | Pre- | Pre- | | |
|---|---|---|---|---|---|---|---|---|---|---|
| | | valance | valance | valance | | valance | valance | valance | | |
| | | of tumor | of tumor | differ- | | of | of | differ- | | |
| | | cell | cell | ence | | normal | normal | ence | | |
| | | pre- | pre- | of | | cell | cell | of | | |
| | Tumor | ferred | ferred | tumor | Normal | preferred | preferred | normal | | |
| | cell | isoform | isoform | cell | cell | isoform | isoform | cell | | |
| | preferred | in | in non- | pre- | preferred | in | in | pre- | | FDR |
| Gene | isoform | tumor | tumor | ferred | isoform | tumor | normal | ferred | | adj |
| Name | ID | cells | cells | isoform | ID | cells | cells | isoform | P-values | P-values |
| HMGN3 | ENST00000620514 | 94.40% | 66.35% | 28.05% | ENST00000275036 | 3.74% | 21.62% | 17.88% | 4.66E−285 | 1.81E−281 |
| HOOK2 | ENST00000589134 | 46.97% | 7.08% | 39.88% | ENST00000593143 | 24.43% | 87.80% | 63.36% | 1.62E−150 | 3.16E−147 |
| CYB5A | ENST00000583418 | 30.62% | 6.87% | 23.74% | ENST00000340533 | 60.49% | 90.61% | 30.12% | 1.13E−101 | 1.46E−98 |
| PPDPFL | ENST00000303202 | 52.78% | 5.68% | 47.10% | ENST00000399653 | 39.65% | 73.78% | 34.13% | 4.20E−97 | 4.08E−94 |
| CALD1 | ENST00000361675 | 65.14% | 45.60% | 19.53% | ENST00000475772 | 0.11% | 10.91% | 10.80% | 1.19E−96 | 9.28E−94 |
| HSPB1 | ENST00000447574 | 32.17% | 6.20% | 25.96% | ENST00000248553 | 67.27% | 93.73% | 26.46% | 1.15E−65 | 6.40E−63 |
| KCP | ENST00000460528 | 38.23% | 5.49% | 32.73% | ENST00000492679 | 29.52% | 84.98% | 55.46% | 6.06E−60 | 2.94E−57 |
| ARHGAP24 | ENST00000264343 | 61.08% | 28.84% | 32.24% | ENST00000509709 | 2.49% | 23.57% | 21.08% | 3.49E−54 | 1.51E−51 |
| CHCHD3 | ENST00000262570 | 19.35% | 7.36% | 11.99% | ENST00000457942 | 48.29% | 86.22% | 37.93% | 7.78E−45 | 3.02E−42 |
| MT1E | ENST00000330439 | 51.21% | 7.64% | 43.57% | ENST00000306061 | 45.54% | 91.67% | 46.13% | 2.61E−44 | 9.24E−42 |
| MT1X | ENST00000568370 | 49.67% | 8.87% | 40.80% | ENST00000394485 | 49.87% | 89.24% | 39.37% | 6.62E−42 | 2.14E−39 |
| DST | ENST00000361203 | 10.68% | 2.72% | 7.95% | ENST00000340834 | 11.44% | 31.73% | 20.30% | 1.22E−41 | 3.64E−39 |
| MSR1 | ENST00000518343 | 54.18% | 3.70% | 50.48% | ENST00000381998 | 7.77% | 56.75% | 48.98% | 1.96E−41 | 5.43E−39 |
| GLRX | ENST00000507605 | 54.25% | 13.77% | 40.48% | ENST00000237858 | 16.89% | 62.12% | 45.23% | 3.10E−39 | 8.04E−37 |
| VPS13C | ENST00000249837 | 42.96% | 22.43% | 20.53% | ENST00000559119 | 15.14% | 33.85% | 18.72% | 4.65E−36 | 1.13E−33 |
| ERN1 | ENST00000433197 | 63.83% | 39.97% | 23.86% | ENST00000606895 | 12.21% | 37.42% | 25.21% | 2.87E−34 | 5.58E−32 |
| UQCRH | ENST00000486951 | 21.14% | 2.95% | 18.19% | ENST00000311672 | 65.48% | 88.55% | 23.07% | 2.56E−34 | 5.58E−32 |
| RPS28 | ENST00000449223 | 18.02% | 4.91% | 13.11% | ENST00000600659 | 75.51% | 93.47% | 17.97% | 2.85E−34 | 5.58E−32 |
| C15orf48 | ENST00000558632 | 48.63% | 12.27% | 36.36% | ENST00000396650 | 31.58% | 56.77% | 25.19% | 4.17E−32 | 7.71E−30 |
| MRPS6 | ENST00000483977 | 43.06% | 26.69% | 16.37% | ENST00000399312 | 20.09% | 55.96% | 35.87% | 8.21E−31 | 1.45E−28 |
| NBEAL1 | ENST00000449802 | 36.60% | 13.83% | 22.78% | ENST00000460355 | 50.32% | 76.70% | 26.38% | 2.67E−30 | 4.52E−28 |
| HSPA4L | ENST00000296464 | 16.87% | 2.88% | 13.99% | ENST00000515262 | 78.52% | 96.50% | 17.98% | 1.06E−29 | 1.72E−27 |
| ENTPD1- | ENST00000427300 | 20.39% | 4.54% | 15.85% | ENST00000416301 | 27.20% | 59.48% | 32.28% | 1.22E−29 | 1.90E−27 |
| AS1 | | | | | | | | | | |
| PLCG2 | ENST00000564138 | 91.72% | 69.27% | 22.45% | ENST00000563193 | 1.27% | 8.73% | 7.46% | 8.59E−28 | 1.28E−25 |
| ACAP1 | ENST00000575415 | 31.89% | 4.98% | 26.91% | ENST00000576628 | 24.22% | 74.90% | 50.68% | 1.24E−26 | 1.78E−24 |
| PDE4B | ENST00000371048 | 28.42% | 7.98% | 20.43% | ENST00000534463 | 10.27% | 30.45% | 20.18% | 1.76E−26 | 2.45E−24 |
| ARHGEF10 | ENST00000495593 | 27.21% | 5.75% | 21.46% | ENST00000375408 | 9.69% | 84.02% | 74.33% | 7.92E−25 | 1.06E−22 |
| L | | | | | | | | | | |
| NDRG1 | ENST00000414097 | 37.40% | 22.02% | 15.38% | ENST00000519278 | 15.49% | 31.68% | 16.19% | 1.33E−23 | 1.73E−21 |
| TC2N | ENST00000435962 | 34.51% | 8.03% | 26.48% | ENST00000555302 | 9.57% | 61.36% | 51.79% | 7.84E−23 | 9.52E−21 |
| CSTB | ENST00000640406 | 17.35% | 5.90% | 11.45% | ENST00000291568 | 77.39% | 92.29% | 14.89% | 1.28E−22 | 1.51E−20 |
| UGT1A1 | ENST00000360418 | 43.08% | 16.69% | 26.39% | ENST00000305208 | 56.92% | 83.31% | 26.39% | 2.17E−22 | 2.48E−20 |
| RPS21 | ENST00000370562 | 18.32% | 5.14% | 13.18% | ENST00000343986 | 79.05% | 93.63% | 14.58% | 1.80E−21 | 1.94E−19 |
| PGF | ENST00000555253 | 58.99% | 31.64% | 27.35% | ENST00000238607 | 22.19% | 46.77% | 24.58% | 4.14E−21 | 4.35E−19 |
| TNFAIP8 | ENST00000504771 | 28.35% | 9.76% | 18.58% | ENST00000513374 | 30.88% | 69.40% | 38.52% | 4.45E−21 | 4.56E−19 |
| WSB1 | ENST00000467843 | 52.01% | 27.44% | 24.58% | ENST00000262394 | 25.88% | 33.24% | 7.36% | 8.57E−21 | 8.54E−19 |
| UGT1A6 | ENST00000406651 | 23.18% | 10.26% | 12.93% | ENST00000305139 | 61.53% | 86.71% | 25.18% | 2.07E−20 | 2.01E−18 |
| UGT1A10 | ENST00000373445 | 35.42% | 14.73% | 20.69% | ENST00000344644 | 64.58% | 85.27% | 20.69% | 4.09E−20 | 3.88E−18 |
| UQCRQ | ENST00000498309 | 20.30% | 4.38% | 15.92% | ENST00000378670 | 74.04% | 92.34% | 18.30% | 7.54E−20 | 6.98E−18 |
| ENTPD1 | ENST00000371205 | 86.08% | 61.58% | 24.50% | ENST00000371206 | 11.18% | 28.37% | 17.19% | 1.06E−19 | 9.54E−18 |
| SYNE2 | ENST00000358025 | 22.59% | 11.27% | 11.31% | ENST00000557005 | 4.18% | 12.25% | 8.07% | 1.57E−19 | 1.39E−17 |
| PDLIM3 | ENST00000514142 | 55.52% | 44.17% | 11.35% | ENST00000284771 | 6.97% | 40.35% | 33.38% | 3.44E−19 | 2.97E−17 |
| PDE10A | ENST00000648884 | 73.58% | 20.23% | 53.34% | ENST00000649761 | 6.89% | 26.67% | 19.77% | 7.65E−19 | 6.32E−17 |
| MICOS10 | ENST00000486890 | 7.80% | 1.88% | 5.92% | ENST00000322753 | 55.53% | 81.25% | 25.72% | 1.30E−18 | 1.05E−16 |
| SNX10 | ENST00000396376 | 52.54% | 19.93% | 32.62% | ENST00000338523 | 16.25% | 63.65% | 47.39% | 6.36E−18 | 5.04E−16 |
| SOX6 | ENST00000533411 | 13.96% | 5.62% | 8.34% | ENST00000525259 | 11.36% | 51.69% | 40.33% | 6.52E−18 | 5.07E−16 |
| TRIM25 | ENST00000316881 | 91.61% | 81.80% | 9.82% | ENST00000648772 | 0.96% | 11.43% | 10.47% | 8.74E−17 | 6.54E−15 |
| PIK3R1 | ENST00000320694 | 25.03% | 10.03% | 15.00% | ENST00000521381 | 48.58% | 59.66% | 11.08% | 1.42E−16 | 1.04E−14 |
| CYTOR | ENST00000645257 | 11.58% | 5.08% | 6.49% | ENST00000642451 | 18.16% | 30.79% | 12.63% | 2.84E−16 | 2.04E−14 |
| UBE2D2 | ENST00000511725 | 82.22% | 42.86% | 39.36% | ENST00000398733 | 12.67% | 38.96% | 26.29% | 7.09E−16 | 5.01E−14 |
| MXD3 | ENST00000503473 | 38.99% | 13.93% | 25.06% | ENST00000427908 | 53.99% | 81.58% | 27.59% | 1.15E−15 | 8.02E−14 |
| EPC1 | ENST00000495790 | 37.86% | 24.12% | 13.74% | ENST00000375110 | 33.38% | 61.22% | 27.84% | 1.21E−15 | 8.25E−14 |
| ATP5MC1 | ENST00000513347 | 22.23% | 4.93% | 17.31% | ENST00000355938 | 45.24% | 56.29% | 11.05% | 1.25E−15 | 8.36E−14 |
| RPS25 | ENST00000527853 | 64.66% | 27.35% | 37.31% | ENST00000527673 | 33.71% | 71.83% | 38.11% | 2.04E−15 | 1.32E−13 |
| FAM13A | ENST00000395002 | 44.13% | 18.11% | 26.02% | ENST00000503556 | 6.20% | 19.79% | 13.58% | 3.05E−15 | 1.95E−13 |
| LINC01320 | ENST00000659537 | 31.16% | 16.13% | 15.03% | ENST00000621006 | 21.81% | 40.32% | 18.51% | 5.16E−15 | 3.23E−13 |
| HAVCR2 | ENST00000524219 | 52.86% | 12.55% | 40.30% | ENST00000522902 | 20.00% | 39.96% | 19.96% | 6.49E−15 | 4.00E−13 |
| TUBB6 | ENST00000591909 | 80.66% | 46.43% | 34.24% | ENST00000587204 | 2.11% | 23.21% | 21.10% | 6.58E−15 | 4.00E−13 |
| UGT1A4 | ENST00000450233 | 31.59% | 11.44% | 20.15% | ENST00000373409 | 68.41% | 88.56% | 20.15% | 1.30E−14 | 7.80E−13 |
| PIGX | ENST00000392391 | 76.34% | 28.07% | 48.27% | ENST00000426755 | 11.83% | 66.67% | 54.84% | 1.78E−14 | 1.03E−12 |
| RTTN | ENST00000640769 | 12.64% | 1.53% | 11.11% | ENST00000640736 | 2.10% | 13.91% | 11.81% | 3.13E−14 | 1.79E−12 |
| MBNL1 | ENST00000466565 | 12.74% | 5.15% | 7.59% | ENST00000461436 | 41.22% | 51.37% | 10.15% | 1.45E−13 | 8.17E−12 |
| OBSL1 | ENST00000373873 | 56.08% | 35.01% | 21.07% | ENST00000404537 | 3.84% | 24.27% | 20.43% | 1.61E−13 | 8.93E−12 |
| MTREX | ENST00000518955 | 22.66% | 6.33% | 16.33% | ENST00000230640 | 38.32% | 69.06% | 30.74% | 1.80E−13 | 9.84E−12 |
| MID1 | ENST00000380780 | 83.27% | 62.34% | 20.93% | ENST00000380779 | 1.42% | 11.24% | 9.82% | 2.58E−13 | 1.39E−11 |
| MRPS21 | ENST00000581066 | 77.19% | 51.06% | 26.12% | ENST00000614145 | 22.81% | 48.94% | 26.12% | 2.87E−13 | 1.52E−11 |
| IFI30 | ENST00000407280 | 94.80% | 74.50% | 20.30% | ENST00000600463 | 5.20% | 25.50% | 20.30% | 2.90E−13 | 1.52E−11 |
| NHSL1 | ENST00000426841 | 32.87% | 15.65% | 17.22% | ENST00000342260 | 1.36% | 45.58% | 44.22% | 4.17E−13 | 2.16E−11 |
| NPIPA5 | ENST00000360151 | 94.16% | 75.21% | 18.96% | ENST00000534094 | 5.68% | 24.79% | 19.11% | 4.54E−13 | 2.32E−11 |
| POU2AF1 | ENST00000529065 | 27.08% | 0.00% | 27.08% | ENST00000393067 | 49.05% | 75.00% | 25.95% | 6.53E−13 | 3.27E−11 |
| TMEM258 | ENST00000543510 | 28.33% | 12.11% | 16.21% | ENST00000537328 | 54.95% | 80.48% | 25.53% | 6.56E−13 | 3.27E−11 |
| ZNF407 | ENST00000581829 | 51.35% | 23.88% | 27.47% | ENST00000582337 | 11.25% | 40.30% | 29.05% | 9.26E−13 | 4.56E−11 |
| ITGA1 | ENST00000506275 | 17.79% | 10.89% | 6.90% | ENST00000650673 | 20.53% | 40.23% | 19.70% | 1.12E−12 | 5.44E−11 |
| ST6GAL1 | ENST00000485105 | 13.80% | 8.66% | 5.14% | ENST00000448408 | 63.04% | 81.29% | 18.24% | 1.14E−12 | 5.48E−11 |
| ATP5MPL | ENST00000414262 | 34.75% | 15.42% | 19.34% | ENST00000286953 | 44.76% | 77.09% | 32.33% | 1.24E−12 | 5.89E−11 |
| TM2D3 | ENST00000561373 | 23.73% | 12.31% | 11.42% | ENST00000560390 | 1.89% | 21.54% | 19.65% | 1.42E−12 | 6.58E−11 |
| ELMO1 | ENST00000487336 | 27.01% | 16.89% | 10.13% | ENST00000463390 | 4.90% | 22.87% | 17.98% | 1.42E−12 | 6.58E−11 |
| WWP2 | ENST00000568818 | 42.95% | 13.21% | 29.74% | ENST00000569174 | 6.85% | 34.59% | 27.74% | 2.12E−12 | 9.56E−11 |
| TMEM176 | ENST00000492607 | 50.04% | 19.61% | 30.43% | ENST00000326442 | 42.15% | 61.03% | 18.88% | 3.01E−12 | 1.34E−10 |
| B | | | | | | | | | | |
| ATP5PD | ENST00000580649 | 19.35% | 5.05% | 14.30% | ENST00000301587 | 79.70% | 94.76% | 15.06% | 4.35E−12 | 1.92E−10 |
| APOLD1 | ENST00000534843 | 94.87% | 66.18% | 28.70% | ENST00000356591 | 4.10% | 31.27% | 27.17% | 4.46E−12 | 1.95E−10 |
| HOXA6 | ENST00000521478 | 85.55% | 36.59% | 48.96% | ENST00000222728 | 14.45% | 63.41% | 48.96% | 4.71E−12 | 1.97E−10 |
| NR4A2 | ENST00000409572 | 84.33% | 38.64% | 45.69% | ENST00000339562 | 10.63% | 58.09% | 47.47% | 4.72E−12 | 1.97E−10 |
| NPIPA9 | ENST00000545114 | 24.74% | 8.38% | 16.35% | ENST00000546267 | 18.57% | 36.83% | 18.26% | 6.98E−12 | 2.86E−10 |
| PLXNC1 | ENST00000549217 | 33.43% | 6.82% | 26.61% | ENST00000549810 | 2.37% | 17.72% | 15.35% | 8.76E−12 | 3.55E−10 |
| RTN4 | ENST00000405240 | 21.76% | 12.23% | 9.53% | ENST00000317610 | 23.27% | 52.89% | 29.62% | 1.07E−11 | 4.29E−10 |
| C12orf60 | ENST00000330828 | 17.22% | 1.01% | 16.22% | ENST00000543822 | 79.43% | 98.99% | 19.57% | 1.15E−11 | 4.54E−10 |
| ZBTB38 | ENST00000509842 | 28.12% | 3.58% | 24.54% | ENST00000514251 | 22.35% | 36.04% | 13.69% | 1.20E−11 | 4.72E−10 |
| RPL36 | ENST00000347512 | 73.98% | 62.14% | 11.83% | ENST00000394580 | 17.11% | 34.06% | 16.95% | 1.76E−11 | 6.82E−10 |
| BRD7 | ENST00000401491 | 23.28% | 1.76% | 21.51% | ENST00000394688 | 56.26% | 94.12% | 37.85% | 1.83E−11 | 7.04E−10 |
| IFITM2 | ENST00000527146 | 5.45% | 2.11% | 3.34% | ENST00000399815 | 72.45% | 83.25% | 10.79% | 2.21E−11 | 8.43E−10 |
| PDCD4 | ENST00000393104 | 36.36% | 9.42% | 26.94% | ENST00000489049 | 19.07% | 38.12% | 19.05% | 3.17E−11 | 1.20E−09 |
| Clorf122 | ENST00000373043 | 79.39% | 53.35% | 26.03% | ENST00000373042 | 12.25% | 27.74% | 15.50% | 3.22E−11 | 1.20E−09 |
| ATP5IF1 | ENST00000468425 | 20.65% | 9.69% | 10.96% | ENST00000335514 | 56.12% | 73.85% | 17.73% | 3.33E−11 | 1.23E−09 |
| CIB1 | ENST00000650306 | 10.03% | 2.05% | 7.97% | ENST00000328649 | 80.10% | 94.12% | 14.02% | 4.11E−11 | 1.51E−09 |
| NF1 | ENST00000358273 | 11.25% | 4.91% | 6.34% | ENST00000581113 | 6.55% | 23.05% | 16.50% | 4.53E−11 | 1.64E−09 |
| FNDC3A | ENST00000398316 | 48.47% | 25.51% | 22.96% | ENST00000378383 | 16.38% | 27.87% | 11.48% | 6.05E−11 | 2.16E−09 |
| FRMD4B | ENST00000459638 | 47.61% | 18.76% | 28.85% | ENST00000460709 | 1.47% | 10.99% | 9.51% | 6.48E−11 | 2.29E−09 |
| PSMA1 | ENST00000527632 | 28.57% | 12.66% | 15.91% | ENST00000528018 | 7.64% | 49.37% | 41.73% | 7.33E−11 | 2.54E−09 |
| HPS3 | ENST00000296051 | 77.53% | 61.32% | 16.22% | ENST00000462030 | 12.00% | 31.94% | 19.94% | 7.56E−11 | 2.60E−09 |
| EGLN3 | ENST00000547327 | 13.44% | 6.40% | 7.04% | ENST00000250457 | 67.27% | 83.45% | 16.18% | 7.96E−11 | 2.72E−09 |
| SCAND1 | ENST00000615116 | 59.26% | 17.24% | 42.02% | ENST00000373991 | 36.57% | 75.54% | 38.97% | 1.08E−10 | 3.67E−09 |
| NDUFB4 | ENST00000485064 | 16.07% | 5.09% | 10.98% | ENST00000184266 | 80.87% | 93.72% | 12.85% | 1.98E−10 | 6.64E−09 |
| MYSM1 | ENST00000493821 | 24.59% | 6.00% | 18.59% | ENST00000481973 | 2.48% | 16.13% | 13.65% | 2.32E−10 | 7.72E−09 |
| RBIS | ENST00000611854 | 22.62% | 11.41% | 11.21% | ENST00000619594 | 33.59% | 67.93% | 34.35% | 2.57E−10 | 8.45E−09 |
| DPP4 | ENST00000461836 | 19.66% | 2.84% | 16.82% | ENST00000360534 | 47.27% | 80.85% | 33.59% | 2.80E−10 | 9.14E−09 |
| CISD1 | ENST00000489785 | 14.11% | 1.63% | 12.48% | ENST00000333926 | 79.60% | 96.16% | 16.56% | 3.02E−10 | 9.78E−09 |
| TBCA | ENST00000522370 | 6.97% | 0.36% | 6.61% | ENST00000380377 | 67.00% | 90.15% | 23.15% | 3.44E−10 | 1.11E−08 |
| HIF1A | ENST00000539097 | 10.85% | 4.60% | 6.26% | ENST00000337138 | 24.22% | 38.16% | 13.94% | 4.75E−10 | 1.51E−08 |
| PHACTR1 | ENST00000332995 | 57.59% | 36.39% | 21.21% | ENST00000482982 | 1.25% | 17.78% | 16.53% | 6.96E−10 | 2.20E−08 |
| B2M | ENST00000559220 | 15.54% | 11.85% | 3.69% | ENST00000632133 | 52.61% | 63.69% | 11.08% | 8.89E−10 | 2.79E−08 |
| PACSIN2 | ENST00000407585 | 31.89% | 12.65% | 19.25% | ENST00000263246 | 21.95% | 38.16% | 16.21% | 9.37E−10 | 2.91E−08 |
| HOXA-AS3 | ENST00000524304 | 81.23% | 37.21% | 44.02% | ENST00000518947 | 18.09% | 62.79% | 44.70% | 1.04E−10 | 3.20E−08 |
| FKBP2 | ENST00000541388 | 27.50% | 8.56% | 18.94% | ENST00000309366 | 48.21% | 74.90% | 26.69% | 1.56E−09 | 4.78E−08 |
| IVNS1ABP | ENST00000480769 | 56.71% | 42.79% | 13.92% | ENST00000367498 | 11.22% | 25.10% | 13.88% | 1.81E−09 | 5.51E−08 |
| BOLA2 | ENST00000380596 | 55.53% | 40.22% | 15.32% | ENST00000330978 | 44.47% | 59.78% | 15.32% | 2.63E−09 | 7.87E−08 |
| NNMT | ENST00000535401 | 77.78% | 47.41% | 30.37% | ENST00000542647 | 4.08% | 20.52% | 16.44% | 3.16E−09 | 9.37E−08 |
| PDIA5 | ENST00000467157 | 54.53% | 33.70% | 20.83% | ENST00000469649 | 17.64% | 53.78% | 36.14% | 3.21E−09 | 9.47E−08 |
| CCL4 | ENST00000615863 | 72.64% | 47.99% | 24.65% | ENST00000613947 | 24.52% | 48.43% | 23.92% | 5.08E−09 | 1.48E−07 |
| CARMIL1 | ENST00000329474 | 69.24% | 38.69% | 30.55% | ENST00000461945 | 23.25% | 59.61% | 36.36% | 5.13E−09 | 1.49E−07 |
| PRSS23 | ENST00000532572 | 44.41% | 17.89% | 26.51% | ENST00000280258 | 30.59% | 68.41% | 37.82% | 5.35E−09 | 1.52E−07 |
| SOD2 | ENST00000538183 | 44.48% | 28.96% | 15.51% | ENST00000546260 | 13.46% | 18.83% | 5.36% | 7.52E−09 | 2.07E−07 |
| COA4 | ENST00000544575 | 32.53% | 15.15% | 17.38% | ENST00000355693 | 49.35% | 76.26% | 26.91% | 7.96E−09 | 2.18E−07 |
| SMARCB1 | ENST00000646911 | 21.77% | 4.74% | 17.03% | ENST00000644036 | 60.20% | 91.16% | 30.96% | 8.04E−09 | 2.19E−07 |
| PSMG2 | ENST00000400514 | 64.21% | 21.31% | 42.90% | ENST00000317615 | 20.15% | 62.29% | 42.14% | 9.83E−09 | 2.64E−07 |
| GOLGA8B | ENST00000569100 | 22.95% | 11.13% | 11.81% | ENST00000564575 | 18.28% | 32.88% | 14.60% | 2.22E−08 | 5.79E−07 |
| AC009133.1 | ENST00000564980 | 34.34% | 15.51% | 18.84% | ENST00000569981 | 45.77% | 68.64% | 22.87% | 2.27E−08 | 5.87E−07 |
| TNFSF10 | ENST00000472804 | 28.81% | 12.41% | 16.40% | ENST00000241261 | 38.91% | 60.26% | 21.35% | 2.52E−08 | 6.50E−07 |
| CBWD1 | ENST00000618061 | 20.28% | 12.19% | 8.08% | ENST00000382393 | 36.66% | 51.33% | 14.67% | 2.70E−08 | 6.90E−07 |
| PLPP3 | ENST00000472957 | 14.83% | 2.41% | 12.42% | ENST00000459962 | 14.66% | 26.27% | 11.60% | 3.70E−08 | 9.33E−07 |
| CSAD | ENST00000267085 | 69.61% | 41.90% | 27.70% | ENST00000424990 | 8.64% | 23.42% | 14.78% | 4.06E−08 | 1.02E−06 |
| ARHGAP22 | ENST00000460425 | 63.66% | 42.68% | 20.97% | ENST00000493012 | 12.16% | 23.17% | 11.01% | 5.07E−08 | 1.26E−06 |
| RHOF | ENST00000546227 | 7.39% | 0.00% | 7.39% | ENST00000267205 | 6.21% | 23.14% | 16.93% | 5.16E−08 | 1.28E−06 |
| FXYD2 | ENST00000317594 | 20.84% | 1.60% | 19.23% | ENST00000292079 | 71.95% | 94.65% | 22.71% | 5.31E−08 | 1.31E−06 |
| C4orf19 | ENST00000381980 | 75.95% | 29.03% | 46.92% | ENST00000508175 | 21.00% | 54.84% | 33.84% | 5.78E−08 | 1.41E−06 |
| PSMA2 | ENST00000411875 | 29.65% | 8.49% | 21.16% | ENST00000223321 | 63.60% | 87.26% | 23.66% | 5.92E−08 | 1.43E−06 |
| ARHGAP29 | ENST00000482481 | 44.94% | 33.30% | 11.64% | ENST00000260526 | 28.18% | 48.72% | 20.54% | 6.19E−08 | 1.48E−06 |
| HSBP1L1 | ENST00000587347 | 16.27% | 2.90% | 13.37% | ENST00000451882 | 31.67% | 71.01% | 39.35% | 7.11E−08 | 1.68E−06 |
| DNAAF1 | ENST00000564928 | 74.50% | 35.37% | 39.13% | ENST00000623406 | 14.34% | 49.90% | 35.56% | 7.60E−08 | 1.79E−06 |
| CBWD2 | ENST00000479583 | 14.78% | 10.87% | 3.91% | ENST00000490323 | 38.90% | 50.07% | 11.17% | 1.04E−08 | 2.42E−06 |
| HLA-DPA1 | ENST00000419277 | 71.43% | 61.35% | 10.08% | ENST00000417724 | 13.93% | 26.62% | 12.68% | 1.04E−07 | 2.43E−06 |
| SRPRA | ENST00000530680 | 42.03% | 14.81% | 27.21% | ENST00000332118 | 35.94% | 70.37% | 34.43% | 1.14E−07 | 2.63E−06 |
| NBPF10 | ENST00000583866 | 49.25% | 33.40% | 15.85% | ENST00000612520 | 45.82% | 62.90% | 17.08% | 1.19E−07 | 2.72E−06 |
| ZBTB25 | ENST00000608382 | 77.74% | 56.00% | 21.74% | ENST00000555220 | 9.59% | 29.60% | 20.01% | 1.25E−07 | 2.84E−06 |
| PCGF3 | ENST00000362003 | 85.93% | 72.99% | 12.94% | ENST00000400151 | 0.34% | 7.51% | 7.17% | 1.48E−07 | 3.35E−06 |
| DICER1 | ENST00000526495 | 61.93% | 27.52% | 34.41% | ENST00000527414 | 7.18% | 15.57% | 8.39% | 1.73E−07 | 3.86E−06 |
| DNM2 | ENST00000590806 | 10.69% | 5.34% | 5.35% | ENST00000593203 | 34.70% | 67.09% | 32.38% | 2.23E−07 | 4.92E−06 |
| RNPC3 | ENST00000531883 | 44.89% | 39.08% | 5.81% | ENST00000533834 | 10.23% | 23.30% | 13.07% | 2.43E−07 | 5.31E−06 |
| MAGI1 | ENST00000464060 | 48.76% | 34.84% | 13.92% | ENST00000330909 | 16.71% | 27.69% | 10.98% | 2.59E−07 | 5.56E−06 |
| SNX29 | ENST00000566228 | 59.28% | 42.35% | 16.93% | ENST00000564791 | 17.07% | 26.17% | 9.10% | 2.78E−07 | 5.93E−06 |
| ERCC5 | ENST00000602836 | 64.85% | 26.67% | 38.18% | ENST00000651281 | 12.42% | 26.67% | 14.24% | 3.29E−07 | 7.00E−06 |
| VDAC1 | ENST00000265333 | 22.29% | 8.96% | 13.32% | ENST00000395044 | 58.30% | 82.54% | 24.25% | 3.42E−07 | 7.22E−06 |
| RPL7 | ENST00000396467 | 47.34% | 23.81% | 23.53% | ENST00000352983 | 41.18% | 74.60% | 33.42% | 3.45E−07 | 7.24E−06 |
| TPM1 | ENST00000560131 | 18.00% | 4.26% | 13.74% | ENST00000560679 | 6.46% | 17.54% | 11.08% | 3.48E−07 | 7.27E−06 |
| COX16 | ENST00000555276 | 76.78% | 58.70% | 18.08% | ENST00000389912 | 16.10% | 29.16% | 13.06% | 3.60E−07 | 7.48E−06 |
| TFRC | ENST00000360110 | 32.59% | 23.49% | 9.10% | ENST00000392396 | 14.21% | 28.56% | 14.36% | 4.06E−07 | 8.40E−06 |
| MRTFB | ENST00000572400 | 41.28% | 18.82% | 22.47% | ENST00000572567 | 11.49% | 36.28% | 24.79% | 6.09E−07 | 1.25E−05 |
| CACNB2 | ENST00000324631 | 78.25% | 35.84% | 42.41% | ENST00000652391 | 2.91% | 43.34% | 40.42% | 7.04E−07 | 1.42E−05 |
| BOLA2B | ENST00000567436 | 22.44% | 10.19% | 12.25% | ENST00000305321 | 65.00% | 86.15% | 21.15% | 7.19E−07 | 1.44E−05 |
| POLRIC | ENST00000646700 | 47.50% | 23.29% | 24.21% | ENST00000646188 | 21.25% | 67.12% | 45.87% | 8.53E−07 | 1.70E−05 |
| AC068587.4 | ENST00000641618 | 55.72% | 13.11% | 42.61% | ENST00000635775 | 12.21% | 29.51% | 17.29% | 8.86E−07 | 1.74E−05 |
| KAT8 | ENST00000539683 | 42.98% | 29.63% | 13.35% | ENST00000543774 | 2.58% | 28.14% | 25.56% | 9.10E−07 | 1.78E−05 |
| SLC9A3R1 | ENST00000578958 | 33.83% | 15.60% | 18.23% | ENST00000262613 | 65.25% | 83.89% | 18.64% | 9.52E−07 | 1.85E−05 |
| PILRB | ENST00000452089 | 17.55% | 4.82% | 12.73% | ENST00000493091 | 66.40% | 89.32% | 22.92% | 9.72E−07 | 1.88E−05 |
| ATIC | ENST00000467388 | 46.54% | 31.17% | 15.38% | ENST00000236959 | 9.13% | 41.56% | 32.42% | 1.12E−06 | 2.15E−05 |
| FILIP1L | ENST00000398326 | 42.48% | 8.06% | 34.41% | ENST00000383694 | 18.62% | 57.51% | 38.89% | 1.21E−06 | 2.30E−05 |
| NBPF15 | ENST00000577412 | 20.83% | 8.59% | 12.24% | ENST00000584793 | 56.76% | 76.41% | 19.65% | 1.24E−06 | 2.35E−05 |
| ZNF710- | ENST00000620791 | 67.17% | 39.25% | 27.92% | ENST00000558334 | 32.83% | 60.75% | 27.92% | 1.29E−06 | 2.43E−05 |
| AS1 | | | | | | | | | | |
| SELENOS | ENST00000534014 | 55.94% | 36.48% | 19.45% | ENST00000526049 | 21.20% | 33.79% | 12.58% | 1.30E−06 | 2.44E−05 |
| AC106791.1 | ENST00000661977 | 59.61% | 16.67% | 42.94% | ENST00000670115 | 11.17% | 28.02% | 16.85% | 1.36E−06 | 2.54E−05 |
| CCNH | ENST00000505587 | 47.67% | 30.02% | 17.65% | ENST00000504878 | 7.70% | 24.12% | 16.43% | 1.40E−06 | 2.60E−05 |
| PAPPA2 | ENST00000367661 | 31.41% | 17.74% | 13.67% | ENST00000367662 | 61.88% | 77.93% | 16.05% | 1.45E−06 | 2.69E−05 |
| 7-Mar | ENST00000259050 | 40.45% | 15.34% | 25.11% | ENST00000473749 | 18.53% | 39.58% | 21.05% | 1.49E−06 | 2.75E−05 |
| MRPS34 | ENST00000569585 | 23.30% | 7.91% | 15.39% | ENST00000397375 | 76.70% | 92.09% | 15.39% | 1.59E−06 | 2.92E−05 |
| MTUS1 | ENST00000517413 | 23.84% | 12.68% | 11.17% | ENST00000297488 | 6.89% | 21.25% | 14.36% | 1.68E−06 | 3.06E−05 |
| SEC24D | ENST00000505134 | 21.95% | 10.86% | 11.09% | ENST00000511033 | 4.74% | 11.56% | 6.82% | 1.91E−06 | 3.46E−05 |
| HSPD1 | ENST00000476746 | 12.89% | 4.10% | 8.79% | ENST00000388968 | 22.69% | 34.60% | 11.91% | 1.96E−06 | 3.52E−05 |
| GNPTAB | ENST00000299314 | 75.82% | 63.28% | 12.54% | ENST00000549165 | 6.79% | 15.28% | 8.49% | 2.16E−06 | 3.86E−05 |
| SNRNP35 | ENST00000527158 | 66.06% | 39.29% | 26.77% | ENST00000526639 | 13.36% | 35.66% | 22.30% | 2.21E−06 | 3.94E−05 |
| PCSK6 | ENST00000611967 | 24.64% | 16.94% | 7.70% | ENST00000558154 | 7.14% | 23.11% | 15.98% | 2.64E−06 | 4.65E−05 |
| Z93930.2 | ENST00000458080 | 22.10% | 5.00% | 17.10% | ENST00000585003 | 65.17% | 86.00% | 20.83% | 2.95E−06 | 5.17E−05 |
| POM121C | ENST00000615331 | 42.24% | 8.10% | 34.14% | ENST00000607367 | 9.84% | 30.64% | 20.80% | 3.24E−06 | 5.65E−05 |
| ERP29 | ENST00000553161 | 15.77% | 5.58% | 10.19% | ENST00000261735 | 62.03% | 81.96% | 19.93% | 4.20E−06 | 7.15E−05 |
| ELF1 | ENST00000405737 | 36.94% | 21.20% | 15.74% | ENST00000635415 | 4.05% | 20.40% | 16.34% | 4.34E−06 | 7.36E−05 |
| PPP1R14B | ENST00000542235 | 23.64% | 6.06% | 17.58% | ENST00000309318 | 59.18% | 70.30% | 11.13% | 5.48E−06 | 9.18E−05 |
| NDUFB1 | ENST00000553666 | 27.53% | 17.41% | 10.12% | ENST00000553514 | 4.86% | 21.87% | 17.00% | 5.69E−06 | 9.50E−05 |
| AFF1 | ENST00000307808 | 31.51% | 19.61% | 11.90% | ENST00000511442 | 8.44% | 24.93% | 16.49% | 7.10E−06 | 0.00012 |
| NGLY1 | ENST00000417874 | 47.35% | 21.22% | 26.14% | ENST00000496726 | 13.36% | 32.83% | 19.48% | 7.44E−06 | 0.00012 |
| NIPSNAP2 | ENST00000497279 | 54.56% | 37.29% | 17.26% | ENST00000446692 | 20.44% | 49.46% | 29.02% | 8.02E−06 | 0.00013 |
| EIF3K | ENST00000590134 | 30.19% | 11.80% | 18.39% | ENST00000586932 | 3.79% | 17.31% | 13.52% | 8.80E−06 | 0.00014 |
| CTAG2 | ENST00000247306 | 60.17% | 30.86% | 29.30% | ENST00000369585 | 39.83% | 69.14% | 29.30% | 8.93E−06 | 0.00014 |
| AL354740.1 | ENST00000429998 | 55.25% | 35.98% | 19.27% | ENST00000593917 | 40.68% | 60.59% | 19.92% | 9.48E−06 | 0.00015 |
| AC004889.1 | ENST00000663681 | 66.12% | 47.17% | 18.95% | ENST00000498693 | 0.84% | 6.60% | 5.77% | 1.03E−05 | 0.00016 |
| EIF3B | ENST00000468611 | 41.93% | 34.28% | 7.64% | ENST00000397011 | 5.35% | 20.91% | 15.55% | 1.03E−05 | 0.00016 |
| DTYMK | ENST00000445261 | 84.34% | 63.83% | 20.51% | ENST00000420144 | 1.24% | 17.02% | 15.78% | 1.25E−05 | 0.0002 |
| PLD1 | ENST00000471075 | 23.32% | 10.28% | 13.04% | ENST00000331659 | 26.92% | 35.80% | 8.88% | 1.31E−05 | 0.0002 |
| Clorf52 | ENST00000294661 | 88.84% | 60.14% | 28.70% | ENST00000471115 | 6.12% | 27.14% | 21.02% | 1.33E−05 | 0.00021 |
| CHCHD1 | ENST00000372837 | 34.22% | 14.88% | 19.34% | ENST00000372833 | 65.78% | 85.12% | 19.34% | 1.59E−05 | 0.00025 |
| TNRC6B | ENST00000454349 | 23.84% | 11.83% | 12.01% | ENST00000301923 | 23.63% | 47.15% | 23.52% | 1.62E−05 | 0.00025 |
| CEP95 | ENST00000577960 | 12.39% | 1.41% | 10.98% | ENST00000583676 | 14.02% | 36.62% | 22.60% | 1.62E−05 | 0.00025 |
| C5AR1 | ENST00000595501 | 21.50% | 4.88% | 16.62% | ENST00000355085 | 52.47% | 70.73% | 18.27% | 1.77E−05 | 0.00027 |
| AC138969.1 | ENST00000381497 | 21.61% | 3.66% | 17.95% | ENST00000534258 | 12.36% | 18.63% | 6.27% | 1.81E−05 | 0.00027 |
| SEC11C | ENST00000585864 | 6.20% | 0.40% | 5.79% | ENST00000587834 | 69.66% | 85.22% | 15.56% | 1.91E−05 | 0.00029 |
| SNHG9 | ENST00000531523 | 91.40% | 78.62% | 12.78% | ENST00000564014 | 8.60% | 21.38% | 12.78% | 1.97E−05 | 0.0003 |
| WARS2- | ENST00000425884 | 14.67% | 1.85% | 12.81% | ENST00000670000 | 20.00% | 31.48% | 11.48% | 1.99E−05 | 0.0003 |
| AS1 | | | | | | | | | | |
| AC010618.3 | ENST00000655024 | 12.35% | 3.41% | 8.94% | ENST00000596643 | 61.27% | 79.55% | 18.28% | 2.07E−05 | 0.00031 |
| NBPF14 | ENST00000619423 | 21.84% | 8.76% | 13.08% | ENST00000606877 | 58.30% | 72.65% | 14.35% | 2.15E−05 | 0.00032 |
| UPF3A | ENST00000492270 | 28.32% | 14.78% | 13.54% | ENST00000480362 | 0.19% | 2.74% | 2.56% | 2.16E−05 | 0.00032 |
| IMMP1L | ENST00000528161 | 11.59% | 4.26% | 7.33% | ENST00000526776 | 0.58% | 14.89% | 14.32% | 2.45E−05 | 0.00036 |
| MTPAP | ENST00000263063 | 86.87% | 58.18% | 28.69% | ENST00000421701 | 12.83% | 37.74% | 24.91% | 2.47E−05 | 0.00036 |
| CDH6 | ENST00000508132 | 22.12% | 0.00% | 22.12% | ENST00000506396 | 3.81% | 21.79% | 17.98% | 2.52E−05 | 0.00037 |
| USP22 | ENST00000261497 | 17.79% | 2.95% | 14.84% | ENST00000478443 | 36.24% | 66.87% | 30.63% | 2.55E−05 | 0.00037 |
| ARID5B | ENST00000644638 | 23.95% | 10.56% | 13.39% | ENST00000309334 | 31.22% | 49.03% | 17.82% | 2.61E−05 | 0.00038 |
| TMC3-AS1 | ENST00000664001 | 31.51% | 10.00% | 21.51% | ENST00000667513 | 16.70% | 44.00% | 27.30% | 2.68E−05 | 0.00039 |
| AIG1 | ENST00000646199 | 44.88% | 29.13% | 15.75% | ENST00000367596 | 5.78% | 30.43% | 24.65% | 3.04E−05 | 0.00044 |
| FAAP20 | ENST00000497675 | 21.53% | 10.37% | 11.17% | ENST00000378546 | 31.22% | 41.41% | 10.20% | 3.09E−05 | 0.00044 |
| ZNF468 | ENST00000391781 | 90.56% | 72.41% | 18.14% | ENST00000602144 | 1.34% | 10.34% | 9.00% | 3.15E−05 | 0.00045 |
| PSMB10 | ENST00000575556 | 14.18% | 0.81% | 13.37% | ENST00000358514 | 75.30% | 87.90% | 12.60% | 3.14E−05 | 0.00045 |
| ANPEP | ENST00000559761 | 15.26% | 3.09% | 12.17% | ENST00000300060 | 67.70% | 87.71% | 20.00% | 3.16E−05 | 0.00045 |
| CKMT2- | ENST00000500148 | 31.63% | 7.41% | 24.22% | ENST00000511495 | 3.29% | 27.91% | 24.62% | 3.22E−05 | 0.00045 |
| AS1 | | | | | | | | | | |
| UGT2B7 | ENST00000502942 | 88.34% | 74.60% | 13.74% | ENST00000305231 | 7.63% | 25.40% | 17.77% | 3.26E−05 | 0.00046 |
| ZBTB1 | ENST00000554015 | 62.87% | 37.96% | 24.91% | ENST00000358738 | 34.59% | 57.41% | 22.82% | 3.58E−05 | 0.0005 |
| TMEM91 | ENST00000342187 | 51.62% | 19.51% | 32.11% | ENST00000413014 | 44.62% | 79.27% | 34.64% | 3.73E−05 | 0.00052 |
| SIPA1L3 | ENST00000601054 | 14.09% | 0.79% | 13.30% | ENST00000222345 | 59.48% | 72.22% | 12.74% | 3.86E−05 | 0.00053 |
| SNHG8 | ENST00000602520 | 68.11% | 57.65% | 10.46% | ENST00000602819 | 7.93% | 27.06% | 19.13% | 3.88E−05 | 0.00053 |
| TCEAL9 | ENST00000646896 | 36.89% | 24.18% | 12.72% | ENST00000372661 | 50.29% | 74.72% | 24.44% | 4.58E−05 | 0.00062 |
| RGPD8 | ENST00000302558 | 96.11% | 83.83% | 12.29% | ENST00000522286 | 0.83% | 8.41% | 7.58% | 4.58E−05 | 0.00062 |
| LINC-PINT | ENST00000451786 | 22.79% | 13.72% | 9.07% | ENST00000429901 | 14.94% | 25.01% | 10.07% | 4.58E−05 | 0.00062 |
| CDK6 | ENST00000265734 | 94.01% | 84.13% | 9.88% | ENST00000424848 | 4.03% | 15.87% | 11.85% | 4.90E−05 | 0.00066 |
| CNN3 | ENST00000474409 | 27.84% | 16.79% | 11.04% | ENST00000370206 | 55.14% | 78.95% | 23.81% | 5.08E−05 | 0.00068 |
| ATXN2 | ENST00000482777 | 15.16% | 5.99% | 9.17% | ENST00000642389 | 7.31% | 24.78% | 17.47% | 5.10E−05 | 0.00068 |
| MAST4 | ENST00000405643 | 30.75% | 12.99% | 17.76% | ENST00000403625 | 30.06% | 39.17% | 9.10% | 5.18E−05 | 0.00069 |
| NRP1 | ENST00000455749 | 19.53% | 8.64% | 10.89% | ENST00000374867 | 2.50% | 8.42% | 5.92% | 5.29E−05 | 0.0007 |
| GSTM4 | ENST00000326729 | 33.40% | 8.68% | 24.71% | ENST00000638994 | 56.11% | 86.05% | 29.94% | 6.15E−05 | 0.00081 |
| CREBBP | ENST00000573672 | 11.91% | 4.90% | 7.01% | ENST00000635753 | 4.84% | 18.18% | 13.34% | 6.65E−05 | 0.00087 |
| CCDC191 | ENST00000295878 | 51.27% | 33.01% | 18.25% | ENST00000481358 | 14.25% | 40.00% | 25.75% | 6.74E−05 | 0.00088 |
| SLC25A13 | ENST00000416240 | 35.90% | 21.68% | 14.22% | ENST00000472162 | 15.61% | 39.42% | 23.82% | 6.72E−05 | 0.00088 |
| TTC3 | ENST00000399017 | 63.38% | 49.51% | 13.87% | ENST00000399010 | 8.99% | 15.51% | 6.53% | 6.71E−05 | 0.00088 |
| CHFR | ENST00000536932 | 50.53% | 21.04% | 29.49% | ENST00000266880 | 23.11% | 32.76% | 9.65% | 6.83E−05 | 0.00089 |
| HSPE1 | ENST00000473395 | 19.46% | 8.19% | 11.27% | ENST00000233893 | 71.21% | 90.96% | 19.75% | 7.46E−05 | 0.00096 |
| SSU72 | ENST00000359060 | 82.26% | 60.71% | 21.55% | ENST00000291386 | 12.24% | 34.90% | 22.66% | 8.13E−05 | 0.00104 |
| CCDC58 | ENST00000479899 | 26.17% | 8.82% | 17.35% | ENST00000291458 | 19.25% | 61.76% | 42.52% | 8.12E−05 | 0.00104 |
| FAM204A | ENST00000369183 | 75.00% | 59.42% | 15.58% | ENST00000369172 | 0.38% | 11.59% | 11.21% | 8.46E−05 | 0.00107 |
| NR2F2 | ENST00000421109 | 55.86% | 30.23% | 25.63% | ENST00000394166 | 31.81% | 69.75% | 37.93% | 8.59E−05 | 0.00109 |
| AC008443.1 | ENST00000599439 | 81.79% | 65.52% | 16.27% | ENST00000511331 | 3.79% | 17.24% | 13.45% | 8.71E−05 | 0.0011 |
| TIMM17A | ENST00000484647 | 22.35% | 6.41% | 15.93% | ENST00000367287 | 49.86% | 80.77% | 30.91% | 8.72E−05 | 0.0011 |
| PRKCE | ENST00000497602 | 19.68% | 11.11% | 8.57% | ENST00000480633 | 2.02% | 14.45% | 12.43% | 8.86E−05 | 0.00111 |
| ANKRD28 | ENST00000498524 | 10.19% | 4.89% | 5.30% | ENST00000461696 | 56.17% | 73.40% | 17.22% | 9.07E−05 | 0.00113 |
| INPP4B | ENST00000513000 | 53.11% | 43.62% | 9.50% | ENST00000511838 | 3.69% | 21.26% | 17.57% | 9.27E−05 | 0.00115 |
| ZNF783 | ENST00000378052 | 38.59% | 12.20% | 26.39% | ENST00000434415 | 39.30% | 80.49% | 41.19% | 0.0001 | 0.00124 |
| SENP5 | ENST00000323460 | 87.59% | 64.58% | 23.01% | ENST00000463245 | 2.76% | 17.19% | 14.43% | 0.000101 | 0.00125 |
| VAV3 | ENST00000479977 | 20.08% | 3.64% | 16.43% | ENST00000343258 | 1.00% | 13.64% | 12.63% | 0.000107 | 0.00131 |
| AC092821.3 | ENST00000641608 | 42.05% | 17.00% | 25.05% | ENST00000642112 | 12.01% | 31.97% | 19.96% | 0.000107 | 0.00131 |
| FAM72A | ENST00000367128 | 29.91% | 3.92% | 25.99% | ENST00000468509 | 53.75% | 83.91% | 30.16% | 0.000111 | 0.00135 |
| SERF2 | ENST00000448830 | 39.03% | 28.42% | 10.61% | ENST00000381359 | 1.42% | 6.52% | 5.10% | 0.00012 | 0.00145 |
| ABCB9 | ENST00000541424 | 7.99% | 1.06% | 6.93% | ENST00000542678 | 86.98% | 97.24% | 10.26% | 0.000121 | 0.00145 |
| HSPB11 | ENST00000489675 | 16.67% | 5.10% | 11.57% | ENST00000194214 | 49.55% | 75.51% | 25.96% | 0.000122 | 0.00146 |
| SIVA1 | ENST00000556431 | 43.84% | 21.05% | 22.79% | ENST00000329967 | 52.92% | 75.44% | 22.52% | 0.000127 | 0.00152 |
| TAOK3 | ENST00000537952 | 27.89% | 16.80% | 11.09% | ENST00000542902 | 0.70% | 8.55% | 7.85% | 0.000131 | 0.00157 |
| TRIR | ENST00000589590 | 14.80% | 3.91% | 10.89% | ENST00000588213 | 32.14% | 43.02% | 10.88% | 0.000133 | 0.00158 |
| MIA3 | ENST00000470521 | 21.58% | 13.88% | 7.70% | ENST00000450260 | 1.59% | 16.85% | 15.27% | 0.00013 | 0.00161 |
| LAMA1 | ENST00000389658 | 48.97% | 14.69% | 34.27% | ENST00000490190 | 8.15% | 20.83% | 12.68% | 0.000138 | 0.00163 |
| KIFC1 | ENST00000428849 | 38.85% | 11.11% | 27.74% | ENST00000486695 | 56.45% | 88.89% | 32.44% | 0.000142 | 0.0016 |
| DUSP23 | ENST00000368107 | 55.88% | 29.48% | 26.41% | ENST00000368108 | 44.12% | 70.52% | 26.41% | 0.000148 | 0.00173 |
| GSTK1 | ENST00000479303 | 52.71% | 46.29% | 6.42% | ENST00000358406 | 30.20% | 45.12% | 14.92% | 0.00015 | 0.00175 |
| AC242426.2 | ENST00000650785 | 77.11% | 62.40% | 14.71% | ENST00000651151 | 21.42% | 32.00% | 10.58% | 0.000166 | 0.00191 |
| ARAP1 | ENST00000359373 | 23.89% | 3.18% | 20.71% | ENST00000465814 | 34.96% | 65.35% | 30.40% | 0.00017 | 0.00196 |
| AC093535.1 | ENST00000648070 | 47.17% | 17.95% | 29.22% | ENST00000512500 | 44.11% | 56.41% | 12.30% | 0.000171 | 0.00196 |
| COX20 | ENST00000411948 | 9.78% | 4.38% | 5.40% | ENST00000498262 | 33.59% | 44.64% | 11.05% | 0.000175 | 0.002 |
| RUFY2 | ENST00000473398 | 23.48% | 8.00% | 15.48% | ENST00000466187 | 49.79% | 74.48% | 24.69% | 0.000177 | 0.00201 |
| AL137186.2 | ENST00000397264 | 97.79% | 82.61% | 15.19% | ENST00000448685 | 1.47% | 17.39% | 15.92% | 0.0002 | 0.00225 |
| URI1 | ENST00000570564 | 39.45% | 25.97% | 13.47% | ENST00000573052 | 2.08% | 12.99% | 10.91% | 0.000219 | 0.00246 |
| GSAP | ENST00000491796 | 16.97% | 8.88% | 8.08% | ENST00000434084 | 8.68% | 18.71% | 10.03% | 0.000226 | 0.00253 |
| IER3 | ENST00000376377 | 45.85% | 32.74% | 13.11% | ENST00000259874 | 54.15% | 67.26% | 13.11% | 0.00023 | 0.00257 |
| MRPL16 | ENST00000525470 | 35.68% | 9.33% | 26.35% | ENST00000300151 | 47.72% | 69.33% | 21.61% | 0.00024 | 0.00268 |
| MOB1B | ENST00000309395 | 72.65% | 60.72% | 11.93% | ENST00000511449 | 10.26% | 23.68% | 13.43% | 0.000265 | 0.00293 |
| MROH1 | ENST00000423230 | 22.32% | 11.88% | 10.44% | ENST00000528664 | 4.09% | 14.85% | 10.76% | 0.000266 | 0.00293 |
| VPS9D1 | ENST00000389386 | 36.76% | 10.00% | 26.76% | ENST00000561976 | 56.10% | 88.33% | 32.23% | 0.000269 | 0.00295 |
| ITGAE | ENST00000572179 | 20.10% | 6.88% | 13.22% | ENST00000263087 | 49.25% | 68.54% | 19.29% | 0.000273 | 0.00299 |
| RBX1 | ENST00000467617 | 14.34% | 5.38% | 8.96% | ENST00000216225 | 83.61% | 94.22% | 10.61% | 0.000293 | 0.0032 |
| NDUFAB1 | ENST00000484769 | 7.49% | 0.44% | 7.05% | ENST00000007516 | 84.10% | 95.15% | 11.05% | 0.000296 | 0.00322 |
| TULP3 | ENST00000448120 | 71.41% | 48.15% | 23.26% | ENST00000540184 | 17.84% | 50.00% | 32.16% | 0.000303 | 0.00329 |
| TASOR2 | ENST00000645567 | 30.99% | 12.76% | 18.23% | ENST00000482419 | 26.64% | 44.71% | 18.07% | 0.000306 | 0.00332 |
| GNPTG | ENST00000527137 | 39.36% | 17.81% | 21.55% | ENST00000204679 | 42.58% | 72.60% | 30.02% | 0.00031 | 0.00335 |
| C12orf45 | ENST00000552951 | 87.43% | 68.37% | 19.05% | ENST00000547750 | 3.43% | 14.29% | 10.86% | 0.000343 | 0.00368 |
| SSBP1 | ENST00000489378 | 38.94% | 24.53% | 14.41% | ENST00000465582 | 10.30% | 35.85% | 25.55% | 0.000346 | 0.00369 |
| VRK2 | ENST00000432057 | 18.39% | 2.82% | 15.58% | ENST00000412104 | 0.89% | 11.27% | 10.38% | 0.00035 | 0.00372 |
| CHSY1 | ENST00000561143 | 13.47% | 3.93% | 9.54% | ENST00000254190 | 71.24% | 89.80% | 18.56% | 0.000356 | 0.00378 |
| EMP2 | ENST00000536829 | 19.62% | 1.61% | 18.00% | ENST00000359543 | 57.31% | 83.87% | 26.56% | 0.000358 | 0.0038 |
| SSH2 | ENST00000578411 | 23.88% | 7.41% | 16.48% | ENST00000649863 | 27.30% | 45.47% | 18.17% | 0.000367 | 0.00387 |
| RNMT | ENST00000592764 | 8.84% | 1.93% | 6.91% | ENST00000543302 | 26.36% | 39.17% | 12.80% | 0.000367 | 0.00387 |
| PPFIBP2 | ENST00000530181 | 42.31% | 16.56% | 25.75% | ENST00000528883 | 8.21% | 18.60% | 10.40% | 0.000374 | 0.00393 |
| CMC4 | ENST00000369479 | 71.05% | 53.27% | 17.78% | ENST00000369484 | 28.95% | 46.73% | 17.78% | 0.000397 | 0.00414 |
| ISCA1 | ENST00000326094 | 26.39% | 3.17% | 23.21% | ENST00000375991 | 72.38% | 95.24% | 22.85% | 0.000407 | 0.00423 |
| SIAE | ENST00000436137 | 23.08% | 3.95% | 19.13% | ENST00000525730 | 16.48% | 31.58% | 15.10% | 0.000408 | 0.00423 |
| PLXDC2 | ENST00000377242 | 26.11% | 9.84% | 16.27% | ENST00000377252 | 73.20% | 90.16% | 16.96% | 0.00041 | 0.00424 |
| AC018362.1 | ENST00000649053 | 45.77% | 24.00% | 21.77% | ENST00000564805 | 54.23% | 76.00% | 21.77% | 0.000416 | 0.00429 |
| MAPILC3B2 | ENST00000625301 | 16.54% | 1.89% | 14.65% | ENST00000556529 | 83.46% | 98.11% | 14.65% | 0.000418 | 0.0043 |
| AC009060.1 | ENST00000671539 | 32.29% | 8.35% | 23.94% | ENST00000502126 | 37.36% | 50.90% | 13.55% | 0.000433 | 0.00443 |
| PTPN14 | ENST00000366956 | 68.93% | 56.10% | 12.83% | ENST00000491277 | 24.91% | 41.31% | 16.40% | 0.000433 | 0.00443 |
| RMST | ENST00000667445 | 12.72% | 3.60% | 9.12% | ENST00000639542 | 28.11% | 58.31% | 30.20% | 0.000461 | 0.00469 |
| COPG1 | ENST00000504547 | 15.77% | 2.63% | 13.13% | ENST00000513965 | 21.89% | 52.63% | 30.74% | 0.00047 | 0.00476 |
| CLEC18A | ENST00000568102 | 69.69% | 58.33% | 11.36% | ENST00000449317 | 1.42% | 10.71% | 9.29% | 0.000499 | 0.00501 |
| TPT1 | ENST00000490277 | 20.47% | 10.58% | 9.88% | ENST00000530705 | 68.96% | 80.53% | 11.57% | 0.000506 | 0.00506 |
| FRYL | ENST00000358350 | 17.81% | 7.14% | 10.67% | ENST00000302806 | 21.22% | 40.72% | 19.50% | 0.000532 | 0.0053 |
| DNAJA2 | ENST00000617000 | 13.94% | 1.10% | 12.84% | ENST00000317089 | 80.52% | 98.90% | 18.38% | 0.000547 | 0.00544 |
| MRPL33 | ENST00000448427 | 82.54% | 64.46% | 18.07% | ENST00000296102 | 6.19% | 12.97% | 6.77% | 0.000569 | 0.00562 |
| SULF2 | ENST00000484875 | 14.94% | 4.67% | 10.27% | ENST00000359930 | 17.57% | 31.62% | 14.05% | 0.000572 | 0.00563 |
| SLC40A1 | ENST00000418714 | 32.51% | 8.33% | 24.18% | ENST00000261024 | 63.02% | 89.29% | 26.27% | 0.000592 | 0.0058 |
| BICD1 | ENST00000652176 | 57.86% | 35.91% | 21.95% | ENST00000552160 | 13.37% | 24.76% | 11.40% | 0.000611 | 0.00595 |
| NPIPA1 | ENST00000472413 | 47.46% | 34.40% | 13.05% | ENST00000534720 | 8.77% | 19.29% | 10.51% | 0.000612 | 0.00595 |
| TRIB3 | ENST00000615226 | 14.21% | 4.88% | 9.33% | ENST00000217233 | 82.55% | 95.12% | 12.57% | 0.000625 | 0.00605 |
| KLHL28 | ENST00000553817 | 60.98% | 44.15% | 16.83% | ENST00000396128 | 35.69% | 47.05% | 11.36% | 0.000633 | 0.00611 |
| PTPN18 | ENST00000420717 | 18.84% | 10.28% | 8.56% | ENST00000175756 | 47.79% | 71.03% | 23.23% | 0.000661 | 0.00633 |
| TRIM14 | ENST00000341469 | 72.69% | 62.39% | 10.29% | ENST00000478530 | 15.85% | 35.90% | 20.04% | 0.000685 | 0.00654 |
| PRKG1 | ENST00000643582 | 18.90% | 9.86% | 9.04% | ENST00000401604 | 11.97% | 37.61% | 25.64% | 0.000703 | 0.00668 |
| JMJDIC | ENST00000633035 | 32.36% | 21.26% | 11.10% | ENST00000399262 | 25.65% | 30.12% | 4.48% | 0.000714 | 0.00677 |
| ANKRD12 | ENST00000262126 | 45.04% | 35.26% | 9.78% | ENST00000540578 | 19.56% | 30.56% | 11.00% | 0.000727 | 0.00686 |
| ASAP1 | ENST00000357668 | 18.97% | 8.68% | 10.29% | ENST00000518957 | 20.33% | 28.99% | 8.66% | 0.000732 | 0.00687 |
| RPS6KA3 | ENST00000379565 | 69.78% | 60.91% | 8.87% | ENST00000647265 | 5.64% | 17.59% | 11.95% | 0.000732 | 0.00687 |
| ALPK1 | ENST00000509688 | 22.54% | 9.62% | 12.92% | ENST00000426472 | 9.77% | 24.74% | 14.96% | 0.000742 | 0.00695 |
| UHRF1BP1 | ENST00000192788 | 58.45% | 36.52% | 21.93% | ENST00000452449 | 41.55% | 63.48% | 21.93% | 0.000747 | 0.00697 |
| ST5 | ENST00000531237 | 84.05% | 66.67% | 17.38% | ENST00000533016 | 1.76% | 11.59% | 9.83% | 0.000748 | 0.00697 |
| HSP90AA1 | ENST00000555662 | 5.26% | 0.00% | 5.26% | ENST00000216281 | 67.89% | 81.94% | 14.05% | 0.000767 | 0.00711 |
| PI4KA | ENST00000255882 | 24.96% | 20.10% | 4.86% | ENST00000449120 | 13.72% | 30.91% | 17.19% | 0.000786 | 0.00724 |
| SLC16A1 | ENST00000478835 | 63.52% | 51.47% | 12.05% | ENST00000429288 | 14.07% | 35.29% | 21.23% | 0.000789 | 0.00725 |
| FYB1 | ENST00000515010 | 27.20% | 13.43% | 13.77% | ENST00000512982 | 3.34% | 9.35% | 6.01% | 0.000796 | 0.0073 |
| ANKRD23 | ENST00000476975 | 32.40% | 20.80% | 11.60% | ENST00000462692 | 54.62% | 75.20% | 20.58% | 0.000799 | 0.00731 |
| FAR2 | ENST00000551193 | 82.00% | 50.79% | 31.21% | ENST00000536681 | 13.40% | 22.36% | 8.96% | 0.000831 | 0.00755 |
| POGZ | ENST00000358476 | 9.40% | 3.88% | 5.52% | ENST00000485040 | 32.33% | 54.73% | 22.40% | 0.000858 | 0.00777 |
| ING5 | ENST00000313552 | 44.43% | 32.31% | 12.12% | ENST00000492488 | 3.91% | 21.54% | 17.63% | 0.00086 | 0.00778 |
| NDUFA3 | ENST00000482960 | 16.98% | 1.89% | 15.10% | ENST00000485876 | 24.28% | 62.91% | 38.63% | 0.000867 | 0.00782 |
| LINC01151 | ENST00000656927 | 11.05% | 2.59% | 8.46% | ENST00000666442 | 22.84% | 34.48% | 11.64% | 0.00087 | 0.00782 |
| PSMC1 | ENST00000555787 | 62.67% | 40.20% | 22.47% | ENST00000261303 | 33.09% | 50.19% | 17.10% | 0.000908 | 0.00811 |
| ATXN1L | ENST00000427980 | 72.98% | 48.15% | 24.84% | ENST00000569119 | 19.35% | 41.98% | 22.62% | 0.000946 | 0.00841 |
| SPCS3 | ENST00000507678 | 20.61% | 10.59% | 10.02% | ENST00000503362 | 78.93% | 87.71% | 8.78% | 0.000968 | 0.00859 |
| PPHLN1 | ENST00000549774 | 34.30% | 23.08% | 11.22% | ENST00000317560 | 0.35% | 8.65% | 8.30% | 0.000996 | 0.00878 |
| CHD2 | ENST00000394196 | 26.95% | 16.68% | 10.28% | ENST00000625990 | 1.02% | 4.15% | 3.13% | 0.000995 | 0.00878 |
| USP15 | ENST00000353364 | 9.31% | 5.23% | 4.08% | ENST00000537297 | 17.44% | 29.27% | 11.83% | 0.001056 | 0.00924 |
| RGS12 | ENST00000512266 | 51.51% | 32.10% | 19.41% | ENST00000504194 | 0.00% | 6.17% | 6.17% | 0.001059 | 0.00925 |
| MDH1 | ENST00000472098 | 36.59% | 25.64% | 10.95% | ENST00000462944 | 17.50% | 28.21% | 10.70% | 0.001065 | 0.00928 |
| MTMR10 | ENST00000568604 | 83.23% | 57.83% | 25.40% | ENST00000566338 | 4.12% | 19.28% | 15.15% | 0.001082 | 0.00936 |
| RBM25 | ENST00000261973 | 22.20% | 12.11% | 10.09% | ENST00000532683 | 64.33% | 79.21% | 14.88% | 0.001087 | 0.00937 |
| STIM2 | ENST00000478425 | 39.43% | 16.52% | 22.90% | ENST00000477474 | 31.36% | 50.87% | 19.51% | 0.001104 | 0.00947 |
| B3GALNT2 | ENST00000494378 | 24.92% | 15.04% | 9.87% | ENST00000612859 | 8.05% | 23.30% | 15.25% | 0.001103 | 0.00947 |
| MINCR | ENST00000671046 | 69.59% | 41.67% | 27.92% | ENST00000518073 | 17.05% | 35.00% | 17.95% | 0.00113 | 0.00965 |
| AC011447.3 | ENST00000585816 | 9.75% | 4.46% | 5.29% | ENST00000657925 | 77.17% | 93.58% | 16.42% | 0.001135 | 0.00968 |
| STARD13 | ENST00000487412 | 38.46% | 17.15% | 21.31% | ENST00000344312 | 15.05% | 31.97% | 16.92% | 0.001141 | 0.00971 |
| CCDC9 | ENST00000600117 | 51.74% | 28.95% | 22.79% | ENST00000221922 | 14.27% | 42.11% | 27.83% | 0.001152 | 0.00978 |
| CCP110 | ENST00000381396 | 29.11% | 9.73% | 19.38% | ENST00000566523 | 8.88% | 16.95% | 8.07% | 0.001178 | 0.00995 |
| VTI1A | ENST00000432306 | 35.06% | 27.32% | 7.74% | ENST00000489142 | 5.41% | 15.46% | 10.06% | 0.001227 | 0.01023 |
| BX005266.2 | ENST00000446626 | 94.44% | 85.00% | 9.44% | ENST00000450704 | 2.22% | 15.00% | 12.78% | 0.00123 | 0.01024 |
| SNX24 | ENST00000261369 | 43.50% | 26.19% | 17.31% | ENST00000511545 | 5.49% | 23.81% | 18.32% | 0.001267 | 0.0105 |
| SUSD6 | ENST00000342745 | 96.67% | 85.71% | 10.95% | ENST00000553497 | 2.67% | 13.84% | 11.17% | 0.00132 | 0.01087 |
| OLA1 | ENST00000497760 | 11.90% | 0.00% | 11.90% | ENST00000392560 | 4.17% | 22.22% | 18.06% | 0.001341 | 0.01102 |
| ANKRA2 | ENST00000515804 | 45.78% | 28.05% | 17.73% | ENST00000296785 | 38.23% | 51.22% | 12.99% | 0.001346 | 0.01104 |
| MYO1B | ENST00000471904 | 33.07% | 17.14% | 15.93% | ENST00000304164 | 4.05% | 12.06% | 8.01% | 0.001376 | 0.01126 |
| EPB41 | ENST00000646800 | 8.90% | 1.45% | 7.45% | ENST00000648891 | 10.98% | 26.57% | 15.59% | 0.001381 | 0.01128 |
| LINC02802 | ENST00000294715 | 23.25% | 1.92% | 21.33% | ENST00000457390 | 50.37% | 77.56% | 27.19% | 0.00143 | 0.01161 |
| NSD3 | ENST00000528828 | 11.65% | 5.22% | 6.43% | ENST00000528627 | 10.45% | 20.90% | 10.44% | 0.001433 | 0.01161 |
| FNTB | ENST00000555742 | 26.57% | 7.89% | 18.68% | ENST00000557300 | 10.55% | 36.84% | 26.29% | 0.001479 | 0.01192 |
| UXT | ENST00000376964 | 11.04% | 2.78% | 8.26% | ENST00000333119 | 76.30% | 90.74% | 14.44% | 0.001523 | 0.012265 |
| PSMB7 | ENST00000474081 | 25.59% | 14.55% | 11.05% | ENST00000259457 | 62.47% | 83.64% | 21.17% | 0.001541 | 0.0123 |
| MCMBP | ENST00000569515 | 55.00% | 29.73% | 25.27% | ENST00000360003 | 26.26% | 59.84% | 33.58% | 0.001556 | 0.01245 |
| TMEM134 | ENST00000501408 | 52.86% | 20.93% | 31.93% | ENST00000308022 | 41.40% | 76.81% | 35.40% | 0.001562 | 0.01247 |
| MED15 | ENST00000445987 | 38.99% | 19.57% | 19.43% | ENST00000489651 | 26.15% | 38.45% | 12.30% | 0.001595 | 0.01271 |
| ZC3H8 | ENST00000464305 | 11.07% | 4.11% | 6.96% | ENST00000466259 | 4.62% | 17.81% | 13.19% | 0.001632 | 0.01295 |
| PPP1R12B | ENST00000498070 | 10.95% | 2.50% | 8.45% | ENST00000634903 | 3.36% | 15.14% | 11.77% | 0.001692 | 0.01334 |
| ASXL2 | ENST00000497092 | 6.89% | 0.00% | 6.89% | ENST00000673455 | 14.23% | 27.66% | 13.43% | 0.001702 | 0.01339 |
| NUDT8 | ENST00000301490 | 38.91% | 16.67% | 22.25% | ENST00000376693 | 40.22% | 76.67% | 36.45% | 0.001723 | 0.0135 |
| MAN2C1 | ENST00000565699 | 19.35% | 6.74% | 12.61% | ENST00000564785 | 32.90% | 63.48% | 30.58% | 0.001728 | 0.01351 |
| SCLT1 | ENST00000651532 | 29.26% | 8.30% | 20.96% | ENST00000506368 | 20.49% | 35.27% | 14.77% | 0.001782 | 0.01378 |
| SH3GLB1 | ENST00000482504 | 21.79% | 12.20% | 9.59% | ENST00000370558 | 26.54% | 48.38% | 21.84% | 0.001783 | 0.01378 |
| AC007780.1 | ENST00000592030 | 29.58% | 17.67% | 11.91% | ENST00000590353 | 69.21% | 79.53% | 10.32% | 0.001838 | 0.01409 |
| PDCD6IP | ENST00000412887 | 12.82% | 4.44% | 8.38% | ENST00000459659 | 22.68% | 38.68% | 16.00% | 0.001854 | 0.01419 |
| MPP1 | ENST00000482757 | 8.74% | 0.00% | 8.74% | ENST00000491955 | 13.73% | 46.67% | 32.94% | 0.001861 | 0.01421 |
| CTPS1 | ENST00000486889 | 39.79% | 29.45% | 10.34% | ENST00000649864 | 2.43% | 18.69% | 16.25% | 0.001879 | 0.01432 |
| SLC39A1 | ENST00000356205 | 36.47% | 13.79% | 22.68% | ENST00000310483 | 38.23% | 74.14% | 35.90% | 0.001904 | 0.01446 |
| AC022364.1 | ENST00000473753 | 14.85% | 1.23% | 13.62% | ENST00000617468 | 85.15% | 98.77% | 13.62% | 0.001905 | 0.01446 |
| LPXN | ENST00000528489 | 84.31% | 57.14% | 27.17% | ENST00000395074 | 3.92% | 18.36% | 14.44% | 0.001992 | 0.01506 |
| MTHFD1 | ENST00000650853 | 17.81% | 5.48% | 12.33% | ENST00000651891 | 11.59% | 26.97% | 15.38% | 0.002006 | 0.01513 |
| NFYC | ENST00000372669 | 16.72% | 9.09% | 7.63% | ENST00000440226 | 19.10% | 31.82% | 12.71% | 0.002056 | 0.01545 |
| TRIM59 | ENST00000543469 | 78.57% | 65.28% | 13.29% | ENST00000468542 | 0.00% | 6.94% | 6.94% | 0.002099 | 0.01575 |
| ZBTB44 | ENST00000529982 | 7.41% | 1.66% | 5.75% | ENST00000525623 | 0.00% | 10.00% | 10.00% | 0.002113 | 0.01582 |
| TRAPPC3 | ENST00000462715 | 47.44% | 28.30% | 19.13% | ENST00000373166 | 37.62% | 64.15% | 26.53% | 0.00212 | 0.01584 |
| PACRGL | ENST00000503585 | 88.81% | 66.67% | 22.14% | ENST00000514663 | 1.21% | 14.28% | 13.08% | 0.00214 | 0.0159 |
| FBXO16 | ENST00000521548 | 94.66% | 86.05% | 8.62% | ENST00000520481 | 1.62% | 11.63% | 10.01% | 0.002162 | 0.01604 |
| EHMT1 | ENST00000636081 | 9.12% | 1.06% | 8.06% | ENST00000495657 | 46.82% | 63.79% | 16.97% | 0.002344 | 0.01736 |
| TRAF6 | ENST00000529150 | 25.42% | 6.90% | 18.53% | ENST00000526995 | 73.57% | 87.93% | 14.36% | 0.002357 | 0.01742 |
| AC027097.1 | ENST00000592201 | 87.93% | 66.67% | 21.26% | ENST00000591854 | 12.07% | 33.33% | 21.26% | 0.002373 | 0.0175 |
| FAM153A | ENST00000393518 | 15.85% | 9.80% | 6.05% | ENST00000360669 | 60.12% | 74.51% | 14.38% | 0.002458 | 0.01809 |
| NSA2 | ENST00000514918 | 14.38% | 1.92% | 12.46% | ENST00000610426 | 79.79% | 97.11% | 17.33% | 0.002573 | 0.01883 |
| CEMIP2 | ENST00000377055 | 7.51% | 2.92% | 4.59% | ENST00000377044 | 16.71% | 27.22% | 10.52% | 0.0026 | 0.019 |
| APBB2 | ENST00000504484 | 51.76% | 37.16% | 14.61% | ENST00000502682 | 3.46% | 11.19% | 7.73% | 0.002645 | 0.01925 |
| RPL10A | ENST00000478340 | 9.46% | 2.20% | 7.26% | ENST00000467020 | 54.90% | 67.86% | 12.95% | 0.002684 | 0.0195 |
| LINC01252 | ENST00000665563 | 65.25% | 37.78% | 27.47% | ENST00000499291 | 34.13% | 57.78% | 23.65% | 0.002857 | 0.02072 |
| AP3S1 | ENST00000515066 | 32.95% | 15.07% | 17.89% | ENST00000316788 | 54.55% | 84.93% | 30.38% | 0.002866 | 0.02074 |
| PDCD5 | ENST00000419343 | 11.64% | 2.42% | 9.21% | ENST00000221784 | 85.84% | 96.33% | 10.49% | 0.002889 | 0.02084 |
| SDHA | ENST00000264932 | 26.90% | 7.06% | 19.84% | ENST00000507522 | 31.48% | 50.49% | 19.01% | 0.002993 | 0.0214 |
| CEBPZOS | ENST00000397226 | 24.73% | 15.85% | 8.88% | ENST00000392061 | 2.19% | 12.20% | 10.00% | 0.003 | 0.0214 |
| PDXDC1 | ENST00000570001 | 9.74% | 1.56% | 8.18% | ENST00000561930 | 8.44% | 18.95% | 10.51% | 0.003027 | 0.02151 |
| HIP1R | ENST00000535831 | 44.83% | 25.69% | 19.14% | ENST00000253083 | 38.19% | 47.67% | 9.48% | 0.00304 | 0.02156 |
| UBR3 | ENST00000477461 | 34.44% | 23.24% | 11.21% | ENST00000439681 | 18.11% | 33.24% | 15.12% | 0.003056 | 0.02164 |
| HSP90B1 | ENST00000548462 | 12.49% | 3.90% | 8.59% | ENST00000299767 | 47.93% | 70.25% | 22.32% | 0.003096 | 0.02188 |
| EML4 | ENST00000318522 | 31.10% | 20.18% | 10.92% | ENST00000409040 | 27.35% | 36.92% | 9.57% | 0.003253 | 0.0229 |
| EIF4E | ENST00000450253 | 56.95% | 39.83% | 17.12% | ENST00000504472 | 17.92% | 30.51% | 12.59% | 0.003262 | 0.02293 |
| PELI1 | ENST00000358912 | 68.16% | 61.82% | 6.34% | ENST00000468869 | 4.58% | 18.12% | 13.54% | 0.003284 | 0.02304 |
| NEDD8 | ENST00000533242 | 24.18% | 9.68% | 14.50% | ENST00000250495 | 63.19% | 83.06% | 19.88% | 0.003362 | 0.0235 |
| NUTM2B- | ENST00000665716 | 6.64% | 2.86% | 3.78% | ENST00000619625 | 26.54% | 36.79% | 10.25% | 0.003379 | 0.02358 |
| AS1 | | | | | | | | | | |
| KLHDC10 | ENST00000495724 | 11.85% | 3.28% | 8.57% | ENST00000335420 | 76.94% | 91.80% | 14.86% | 0.003431 | 0.02385 |
| XPO7 | ENST00000252512 | 56.27% | 34.67% | 21.61% | ENST00000518808 | 28.85% | 44.00% | 15.15% | 0.003491 | 0.02414 |
| LINC01116 | ENST00000339037 | 20.05% | 5.45% | 14.60% | ENST00000295549 | 75.59% | 89.09% | 13.50% | 0.003509 | 0.02423 |
| SUB1 | ENST00000502453 | 47.01% | 32.95% | 14.07% | ENST00000265073 | 41.79% | 52.47% | 10.68% | 0.003539 | 0.02439 |
| SETBP1 | ENST00000649279 | 91.96% | 80.00% | 11.96% | ENST00000645568 | 3.20% | 8.42% | 5.22% | 0.003576 | 0.02451 |
| BCCIP | ENST00000278100 | 71.21% | 60.15% | 11.06% | ENST00000299130 | 11.05% | 22.93% | 11.89% | 0.003574 | 0.02451 |
| LIMS1 | ENST00000544547 | 19.98% | 14.62% | 5.36% | ENST00000393310 | 7.56% | 19.01% | 11.44% | 0.00362 | 0.02478 |
| MLLT10 | ENST00000651298 | 13.24% | 1.32% | 11.93% | ENST00000651382 | 42.33% | 67.98% | 25.65% | 0.0037 | 0.02519 |
| RBM26 | ENST00000622611 | 12.42% | 6.70% | 5.72% | ENST00000449987 | 55.81% | 68.62% | 12.80% | 0.0037 | 0.02519 |
| CDC42SE1 | ENST00000439374 | 93.58% | 78.99% | 14.59% | ENST00000483763 | 5.53% | 17.48% | 11.94% | 0.003716 | 0.02525 |
| OR2A1- | ENST00000470435 | 44.95% | 17.95% | 27.01% | ENST00000475089 | 7.54% | 25.64% | 18.10% | 0.003741 | 0.02538 |
| AS1 | | | | | | | | | | |
| CREB3L2 | ENST00000616381 | 12.78% | 8.28% | 4.50% | ENST00000420629 | 6.40% | 17.18% | 10.79% | 0.003781 | 0.0256 |
| ADH5 | ENST00000508511 | 57.74% | 35.51% | 22.23% | ENST00000296412 | 32.42% | 58.32% | 25.90% | 0.003943 | 0.02647 |
| ZSCAN31 | ENST00000396838 | 23.10% | 4.94% | 18.16% | ENST00000476001 | 66.04% | 75.00% | 8.96% | 0.003923 | 0.02647 |
| BTBD2 | ENST00000587825 | 11.59% | 1.23% | 10.35% | ENST00000611545 | 62.80% | 90.12% | 27.32% | 0.003941 | 0.02647 |
| RPS29 | ENST00000396020 | 36.05% | 26.32% | 9.73% | ENST00000539688 | 18.98% | 30.26% | 11.29% | 0.003933 | 0.02647 |
| PARVB | ENST00000495824 | 9.02% | 0.00% | 9.02% | ENST00000402876 | 3.76% | 16.00% | 12.24% | 0.003916 | 0.02647 |
| TMED5 | ENST00000370280 | 17.87% | 9.85% | 8.02% | ENST00000370282 | 28.66% | 47.65% | 19.00% | 0.004048 | 0.02708 |
| CCDC47 | ENST00000584112 | 34.51% | 18.06% | 16.46% | ENST00000225726 | 37.78% | 57.14% | 19.36% | 0.004081 | 0.02726 |
| MALSU1 | ENST00000476623 | 31.22% | 13.98% | 17.24% | ENST00000466681 | 57.56% | 76.34% | 18.78% | 0.004104 | 0.02731 |
| ZNF395 | ENST00000519730 | 15.24% | 6.03% | 9.21% | ENST00000344423 | 78.08% | 90.91% | 12.82% | 0.004171 | 0.02772 |
| GADD45B | ENST00000592937 | 62.29% | 53.80% | 8.50% | ENST00000585359 | 16.92% | 31.50% | 14.58% | 0.004201 | 0.02787 |
| AP006621.1 | ENST00000532946 | 85.71% | 74.11% | 11.61% | ENST00000528982 | 14.29% | 25.89% | 11.61% | 0.004237 | 0.02805 |
| RPL35A | ENST00000647248 | 24.63% | 10.48% | 14.15% | ENST00000329092 | 32.13% | 48.91% | 16.78% | 0.004244 | 0.02806 |
| COMMD2 | ENST00000490008 | 31.37% | 9.68% | 21.70% | ENST00000473414 | 66.34% | 90.32% | 23.98% | 0.004322 | 0.02852 |
| CHCHD5 | ENST00000409719 | 10.50% | 0.99% | 9.51% | ENST00000324913 | 64.18% | 78.22% | 14.03% | 0.004329 | 0.02852 |
| LINC00174 | ENST00000416366 | 16.73% | 3.50% | 13.23% | ENST00000638592 | 27.70% | 42.75% | 15.05% | 0.004366 | 0.02862 |
| CLIC1 | ENST00000375780 | 7.35% | 0.00% | 7.35% | ENST00000375784 | 88.23% | 100.0% | 11.76% | 0.004363 | 0.02862 |
| DOP1B | ENST00000399151 | 66.28% | 46.09% | 20.19% | ENST00000270190 | 19.77% | 37.39% | 17.62% | 0.004402 | 0.0287 |
| CBLB | ENST00000476370 | 46.30% | 32.62% | 13.69% | ENST00000646499 | 17.43% | 30.49% | 13.06% | 0.004406 | 0.0287 |
| ATP8A1 | ENST00000264449 | 23.09% | 10.88% | 12.20% | ENST00000510289 | 33.10% | 42.45% | 9.34% | 0.004408 | 0.0287 |
| PAIP2 | ENST00000510409 | 54.10% | 29.01% | 25.09% | ENST00000265192 | 37.24% | 64.57% | 27.33% | 0.004503 | 0.02908 |
| FAM13B | ENST00000033079 | 61.98% | 51.85% | 10.13% | ENST00000420893 | 2.52% | 8.20% | 5.68% | 0.004494 | 0.02908 |
| SLC25A36 | ENST00000648615 | 11.34% | 6.22% | 5.12% | ENST00000507429 | 67.84% | 79.63% | 11.79% | 0.004501 | 0.02908 |
| ARMH3 | ENST00000370033 | 79.23% | 60.28% | 18.95% | ENST00000311122 | 9.97% | 26.25% | 16.28% | 0.004518 | 0.02913 |
| CCDC88C | ENST00000554165 | 22.60% | 15.61% | 6.99% | ENST00000553437 | 10.43% | 21.39% | 10.96% | 0.004584 | 0.0295 |
| HTT | ENST00000510626 | 28.67% | 13.73% | 14.93% | ENST00000355072 | 13.21% | 27.30% | 14.09% | 0.004649 | 0.02982 |
| ENY2 | ENST00000517350 | 57.74% | 40.35% | 17.39% | ENST00000339942 | 15.42% | 26.48% | 11.06% | 0.004721 | 0.03019 |
| LCOR | ENST00000469510 | 12.54% | 1.98% | 10.56% | ENST00000421806 | 39.99% | 62.38% | 22.38% | 0.004723 | 0.03019 |
| ECPAS | ENST00000259335 | 11.38% | 3.09% | 8.29% | ENST00000602447 | 12.57% | 23.27% | 10.70% | 0.004856 | 0.03089 |
| PDK3 | ENST00000568479 | 67.29% | 59.36% | 7.93% | ENST00000493226 | 16.40% | 28.49% | 12.09% | 0.004929 | 0.0313 |
| CDC42EP1 | ENST00000430687 | 33.00% | 11.27% | 21.74% | ENST00000249014 | 65.52% | 85.92% | 20.40% | 0.004944 | 0.03135 |
| SARNP | ENST00000552207 | 34.85% | 18.18% | 16.67% | ENST00000336133 | 43.12% | 59.60% | 16.47% | 0.00513 | 0.03237 |
| NQO2 | ENST00000380472 | 12.07% | 2.70% | 9.36% | ENST00000380455 | 73.26% | 91.89% | 18.63% | 0.005283 | 0.03323 |
| PDIA3 | ENST00000434494 | 16.05% | 4.27% | 11.78% | ENST00000300289 | 75.18% | 90.15% | 14.97% | 0.005331 | 0.03347 |
| PAXX | ENST00000498095 | 84.77% | 66.05% | 18.72% | ENST00000371620 | 13.41% | 30.28% | 16.87% | 0.005596 | 0.03503 |
| MTHFS | ENST00000560919 | 29.90% | 10.91% | 18.99% | ENST00000258874 | 59.84% | 79.12% | 19.28% | 0.005625 | 0.03515 |
| LINC02422 | ENST00000662662 | 95.24% | 81.48% | 13.76% | ENST00000535163 | 4.76% | 18.52% | 13.76% | 0.005707 | 0.03549 |
| RPL31 | ENST00000264258 | 49.96% | 41.27% | 8.70% | ENST00000441435 | 18.38% | 34.92% | 16.54% | 0.00574 | 0.03564 |
| TGS1 | ENST00000523948 | 17.90% | 6.67% | 11.23% | ENST00000260129 | 78.80% | 93.33% | 14.53% | 0.00583 | 0.03603 |
| CHST11 | ENST00000547956 | 69.05% | 35.90% | 33.15% | ENST00000549016 | 22.86% | 46.15% | 23.30% | 0.005972 | 0.03669 |
| CTSC | ENST00000227266 | 44.84% | 31.72% | 13.12% | ENST00000524463 | 23.35% | 30.36% | 7.01% | 0.005975 | 0.03669 |
| IFI27L2 | ENST00000555558 | 23.86% | 14.55% | 9.31% | ENST00000238609 | 66.43% | 84.54% | 18.12% | 0.006029 | 0.03696 |
| ZNF493 | ENST00000392288 | 15.91% | 7.77% | 8.14% | ENST00000339914 | 11.28% | 25.08% | 13.80% | 0.006112 | 0.03737 |
| RAB30-DT | ENST00000669005 | 44.53% | 31.71% | 12.82% | ENST00000656330 | 34.52% | 47.56% | 13.04% | 0.006232 | 0.03803 |
| DIMT1 | ENST00000514605 | 11.83% | 4.69% | 7.14% | ENST00000199320 | 37.49% | 47.92% | 10.42% | 0.006461 | 0.0393 |
| OPTN | ENST00000378757 | 45.81% | 37.52% | 8.28% | ENST00000487935 | 7.17% | 18.94% | 11.77% | 0.006532 | 0.03967 |
| NMT2 | ENST00000466201 | 45.53% | 26.67% | 18.86% | ENST00000378165 | 40.87% | 71.81% | 30.94% | 0.006567 | 0.03982 |
| PLAGL1 | ENST00000392307 | 93.63% | 81.82% | 11.81% | ENST00000626022 | 1.98% | 9.09% | 7.11% | 0.006694 | 0.04053 |
| STRAP | ENST00000539887 | 12.31% | 3.11% | 9.19% | ENST00000419869 | 55.41% | 73.25% | 17.84% | 0.006861 | 0.04141 |
| AP002807.1 | ENST00000534517 | 90.92% | 80.29% | 10.63% | ENST00000529934 | 8.78% | 17.79% | 9.01% | 0.006888 | 0.04144 |
| PSMB4 | ENST00000474100 | 14.83% | 6.41% | 8.42% | ENST00000290541 | 35.98% | 54.32% | 18.34% | 0.006882 | 0.04144 |
| ARHGEF38 | ENST00000420470 | 22.57% | 2.17% | 20.40% | ENST00000510406 | 42.01% | 67.39% | 25.38% | 0.007111 | 0.04259 |
| ZNF771 | ENST00000564550 | 16.81% | 2.44% | 14.38% | ENST00000566625 | 71.68% | 97.56% | 25.88% | 0.007153 | 0.04271 |
| SLC25A12 | ENST00000472070 | 5.79% | 1.59% | 4.20% | ENST00000484227 | 15.03% | 36.19% | 21.15% | 0.007151 | 0.04271 |
| NEDD4L | ENST00000456986 | 7.32% | 0.74% | 6.58% | ENST00000617539 | 12.68% | 35.85% | 23.17% | 0.00718 | 0.0428 |
| GORASP2 | ENST00000234160 | 56.21% | 40.00% | 16.21% | ENST00000486498 | 16.19% | 26.67% | 10.47% | 0.007228 | 0.04302 |
| PPP4R3A | ENST00000554684 | 24.43% | 14.12% | 10.31% | ENST00000554574 | 42.65% | 62.08% | 19.43% | 0.007391 | 0.04375 |
| SNRPF | ENST00000551316 | 10.02% | 2.52% | 7.50% | ENST00000266735 | 82.31% | 92.45% | 10.14% | 0.007397 | 0.04375 |
| ATP1A1- | ENST00000608511 | 17.26% | 4.88% | 12.38% | ENST00000493908 | 78.82% | 95.12% | 16.30% | 0.007446 | 0.04385 |
| AS1 | | | | | | | | | | |
| ARHGEF12 | ENST00000530388 | 17.63% | 13.49% | 4.15% | ENST00000528225 | 19.50% | 29.65% | 10.15% | 0.007437 | 0.04385 |
| ATR | ENST00000653868 | 29.94% | 23.75% | 6.18% | ENST00000657914 | 9.04% | 20.24% | 11.20% | 0.007527 | 0.04426 |
| GPBP1L1 | ENST00000479235 | 24.63% | 8.90% | 15.73% | ENST00000496278 | 42.45% | 56.71% | 14.26% | 0.007576 | 0.04448 |
| UBE2G2 | ENST00000345496 | 20.63% | 6.46% | 14.17% | ENST00000462569 | 24.44% | 40.21% | 15.77% | 0.007738 | 0.04523 |
| RNF181 | ENST00000443647 | 17.74% | 9.43% | 8.30% | ENST00000306368 | 76.86% | 89.62% | 12.76% | 0.007728 | 0.04523 |
| UBA1 | ENST00000377269 | 63.92% | 52.79% | 11.12% | ENST00000490869 | 22.66% | 38.38% | 15.72% | 0.00778 | 0.04534 |
| SF3B6 | ENST00000478050 | 19.25% | 6.86% | 12.39% | ENST00000233468 | 80.75% | 93.14% | 12.39% | 0.007927 | 0.04606 |
| DTX2 | ENST00000467729 | 24.61% | 12.16% | 12.45% | ENST00000468546 | 35.34% | 47.30% | 11.96% | 0.007963 | 0.0462 |
| MICAL3 | ENST00000672019 | 31.03% | 19.31% | 11.71% | ENST00000495076 | 17.40% | 33.29% | 15.89% | 0.008016 | 0.04644 |
| CD320 | ENST00000598299 | 15.38% | 5.83% | 9.54% | ENST00000301458 | 79.78% | 94.17% | 14.39% | 0.008172 | 0.04727 |
| PKN2 | ENST00000370521 | 67.65% | 56.07% | 11.58% | ENST00000316005 | 26.43% | 38.62% | 12.19% | 0.008279 | 0.04782 |
| PKP4 | ENST00000389757 | 19.29% | 8.86% | 10.43% | ENST00000480171 | 11.46% | 23.21% | 11.75% | 0.008353 | 0.04811 |
| ATP6V1H | ENST00000521335 | 42.54% | 27.27% | 15.26% | ENST00000521707 | 29.05% | 59.74% | 30.69% | 0.008459 | 0.04864 |
| MRPS16 | ENST00000471251 | 13.27% | 3.91% | 9.36% | ENST00000372945 | 83.87% | 94.35% | 10.48% | 0.008478 | 0.04868 |
| UBE4B | ENST00000253251 | 12.37% | 1.14% | 11.22% | ENST00000462658 | 11.17% | 30.23% | 19.06% | 0.008499 | 0.04872 |
| ATXN2L | ENST00000568266 | 2.02% | 0.00% | 2.02% | ENST00000565845 | 1.63% | 15.00% | 13.37% | 0.008656 | 0.04949 |
| SLC25A46 | ENST00000355943 | 38.48% | 19.09% | 19.38% | ENST00000513706 | 39.82% | 58.17% | 18.35% | 0.008721 | 0.04956 |
| TFG | ENST00000481203 | 82.38% | 72.64% | 9.74% | ENST00000476228 | 7.23% | 20.24% | 13.00% | 0.008702 | 0.04956 |
| VPS39 | ENST00000318006 | 15.90% | 6.74% | 9.16% | ENST00000568029 | 41.51% | 71.15% | 29.64% | 0.008713 | 0.04956 |
| ATP13A3 | ENST00000645621 | 22.15% | 14.65% | 7.50% | ENST00000497567 | 6.03% | 16.98% | 10.95% | 0.008764 | 0.04973 |
| AL645568.1 | ENST00000659863 | 14.75% | 3.57% | 11.18% | ENST00000661267 | 23.27% | 38.10% | 14.82% | 0.008824 | 0.04998 |

TABLE 7
Endothelial cell specific DCI genes

| | | | | | | | | Prevalance | | |
|---|---|---|---|---|---|---|---|---|---|---|
| | | Prevalance | Prevalance | | | Prevalance of | | difference | | |
| | | of | of | | | non-endothelial | Prevalance of | of | | |
| | Endothelial | endothelial | endothelial | Prevalance | | cell | non-endothelial cell | non- | | |
| | cell | cell preferred | cell preferred | difference | Non-endothelial | preferred | cells | endothelial | | |
| | preferred | isoform in | isoform in | of endothelial | cell | isoform | preferred | cell | | FDR |
| Gene | isoform | endothelial | non-endothelial | cell preferred | preferred isoform | in | isoform | preferred | | adj |
| Name | ID | cells | cells | isoform | ID | endothelial cells | in non-endothelial | isoform | P-values | P-values |
| CYTOR | ENST00000642451 | 41.72% | 22.95% | 18.78% | ENST00000414030 | 7.38% | 16.36% | 8.98% | 6.15E−27 | 1.89E−23 |
| YWHAH | ENST00000397492 | 95.82% | 83.47% | 12.35% | ENST00000248975 | 0.20% | 12.87% | 12.67% | 6.48E−12 | 9.95E−09 |
| NEDD9 | ENST00000379433 | 77.72% | 33.45% | 44.28% | ENST00000504634 | 0.87% | 23.19% | 22.320% | 1.99E−11 | 2.03E−08 |
| APOLD1 | ENST00000356591 | 66.07% | 9.05% | 57.02% | ENST00000534843 | 30.19% | 89.16% | 58.97% | 4.65E−11 | 3.57E−08 |
| AL008729.1 | ENST00000606150 | 46.67% | 9.33% | 37.33% | ENST00000399446 | 20.00% | 86.67% | 66.67% | 2.92E−10 | 1.79E−07 |
| MTREX | ENST00000230640 | 86.33% | 44.57% | 41.76% | ENST00000518955 | 0.00% | 15.31% | 15.31% | 5.41E−10 | 2.77E−07 |
| MT2A | ENST00000245185 | 97.46% | 79.38% | 18.080% | ENST00000562017 | 1.880% | 11.05% | 9.17% | 6.35E−10 | 2.79E−07 |
| HERPUD1 | ENST00000569429 | 12.70% | 1.520% | 11.17% | ENST00000569569 | 12.82% | 32.11% | 19.29% | 1.22E−08 | 4.16E−06 |
| NBEAL1 | ENST00000449802 | 21.52% | 11.71% | 9.81% | ENST00000460355 | 64.84% | 79.96% | 15.12% | 1.15E−08 | 4.16E−06 |
| ARHGAP24 | ENST00000395183 | 33.95% | 7.01% | 26.94% | ENST00000509709 | 2.44% | 27.05% | 24.62% | 4.35E−08 | 1.34E−05 |
| CD74 | ENST00000353334 | 29.21% | 20.99% | 8.21% | ENST00000523836 | 12.270% | 28.76% | 16.49% | 7.99E−08 | 1.89E−05 |
| ETS1 | ENST00000527676 | 47.17% | 0.00% | 47.17% | ENST00000530924 | 3.77% | 29.55% | 25.77% | 7.81E−08 | 1.89E−05 |
| IVNS1ABP | ENST00000367498 | 27.30% | 3.90% | 23.39% | ENST00000459929 | 20.77% | 26.80% | 6.03% | 9.06E−08 | 1.99E−05 |
| MID1 | ENST00000380779 | 32.98% | 0.00% | 32.98% | ENST00000380780 | 26.45% | 80.90% | 54.45% | 1.17E−07 | 2.39E−05 |
| DLD | ENST00000417551 | 62.50% | 14.89% | 47.61% | ENST00000205402 | 30.56% | 76.60% | 46.04% | 2.45E−07 | 4.70E−05 |
| DAB2 | ENST00000515700 | 18.66% | 1.37% | 17.29% | ENST00000545653 | 3.68% | 18.00% | 14.32% | 3.25E−07 | 5.87E−05 |
| NF1 | ENST00000493220 | 16.85% | 10.66% | 6.19% | ENST00000581113 | 6.59% | 29.95% | 23.36% | 8.26E−07 | 0.000133 |
| SENP6 | ENST00000447266 | 53.37% | 8.79% | 44.57% | ENST00000436928 | 10.42% | 30.17% | 19.76% | 1.37E−06 | 0.000191 |
| PPDPFL | ENST00000303202 | 14.18% | 2.95% | 11.24% | ENST00000399653 | 61.40% | 77.76% | 16.36% | 1.53E−06 | 0.000204 |
| SH3D19 | ENST00000604440 | 17.65% | 0.00% | 17.65% | ENST00000604922 | 41.18% | 100.00% | 58.82% | 2.83E−06 | 0.000348 |
| RBP5 | ENST00000266560 | 58.06% | 21.48% | 36.59% | ENST00000542370 | 12.90% | 70.18% | 57.27% | 3.92E−06 | 0.000463 |
| CBWD5 | ENST00000468198 | 32.28% | 20.13% | 12.15% | ENST00000382404 | 5.87% | 11.69% | 5.81% | 4.75E−06 | 0.000519 |
| FNDC3A | ENST00000398316 | 47.79% | 16.82% | 30.97% | ENST00000378383 | 19.11% | 31.29% | 12.18% | 4.68E−06 | 0.000519 |
| RPS2 | ENST00000526586 | 16.22% | 1.72% | 14.49% | ENST00000529806 | 72.97% | 96.55% | 23.58% | 4.90E−06 | 0.000519 |
| LIMD1 | ENST00000474665 | 31.580% | 1.09% | 30.49% | ENST00000440097 | 0.00% | 15.22% | 15.22% | 5.42E−06 | 0.000555 |
| PLEKHG1 | ENST00000644968 | 33.67% | 4.85% | 28.81% | ENST00000644996 | 7.50% | 48.65% | 41.15% | 6.32E−06 | 0.000626 |
| AOPEP | ENST00000375315 | 34.45% | 6.90% | 27.55% | ENST00000473778 | 5.56% | 48.28% | 42.720% | 7.44E−06 | 0.000714 |
| COL4A2 | ENST00000650225 | 22.11% | 9.23% | 12.87% | ENST00000400163 | 5.60% | 12.15% | 6.55% | 9.97E−06 | 0.0009 |
| EHMT1 | ENST00000637318 | 27.78% | 9.42% | 18.36% | ENST00000495657 | 28.89% | 68.52% | 39.63% | 9.79E−06 | 0.0009 |
| ADGRF5 | ENST00000283296 | 61.19% | 12.49% | 48.70% | ENST00000265417 | 30.75% | 80.37% | 49.62% | 1.15E−05 | 0.000957 |
| CFLAR | ENST00000309955 | 64.94% | 46.09% | 18.85% | ENST00000490965 | 8.09% | 19.93% | 11.84% | 1.13E−05 | 0.000957 |
| SPTBN1 | ENST00000602898 | 26.07% | 3.85% | 22.22% | ENST00000333896 | 28.22% | 56.03% | 27.80% | 1.37E−05 | 0.001079 |
| RNF213 | ENST00000582970 | 26.52% | 13.23% | 13.30% | ENST00000319921 | 3.76% | 8.43% | 4.68% | 1.41E−05 | 0.001079 |
| LRRFIP1 | ENST00000308482 | 23.67% | 14.40% | 9.26% | ENST00000244815 | 31.99% | 44.85% | 12.86% | 1.47E−05 | 0.001098 |
| IFI44L | ENST00000370751 | 68.26% | 48.81% | 19.45% | ENST00000486882 | 5.08% | 23.55% | 18.47% | 1.64E−05 | 0.001198 |
| MATR3 | ENST00000502394 | 23.90% | 0.91% | 22.99% | ENST00000505625 | 57.23% | 89.15% | 31.920% | 1.83E−05 | 0.001303 |
| MECP2 | ENST00000303391 | 57.50% | 21.24% | 36.26% | ENST00000629277 | 0.00% | 15.93% | 15.93% | 2.02E−05 | 0.001408 |
| HPS3 | ENST00000296051 | 87.75% | 51.12% | 36.64% | ENST00000462030 | 4.51% | 42.520% | 38.01% | 2.32E−05 | 0.00155 |
| HERC4 | ENST00000515753 | 8.68% | 3.49% | 5.19% | ENST00000492996 | 15.28% | 29.48% | 14.20% | 2.43E−05 | 0.001585 |
| H2AFY | ENST00000506218 | 9.59% | 0.00% | 9.59% | ENST00000512507 | 26.94% | 42.67% | 15.73% | 3.41E−05 | 0.002182 |
| SELENOS | ENST00000526049 | 67.500% | 26.44% | 41.06% | ENST00000534014 | 20.45% | 39.98% | 19.520% | 3.48E−05 | 0.002182 |
| JMJDIC | ENST00000399262 | 34.53% | 25.61% | 8.91% | ENST00000633035 | 13.61% | 29.100% | 15.49% | 4.70E−05 | 0.002887 |
| N4BP1 | ENST00000569027 | 29.73% | 0.00% | 29.73% | ENST00000564710 | 0.00% | 17.91% | 17.91% | 4.95E−05 | 0.002979 |
| DST | ENST00000340834 | 40.73% | 23.410% | 17.32% | ENST00000487754 | 10.67% | 21.03% | 10.36% | 5.06E−05 | 0.00299 |
| COX16 | ENST00000555276 | 67.95% | 53.95% | 14.00% | ENST00000389912 | 16.67% | 35.57% | 18.90% | 5.19E−05 | 0.003008 |
| LIMS1 | ENST00000544547 | 26.32% | 8.26% | 18.06% | ENST00000422797 | 11.59% | 24.020% | 12.420% | 5.37E−05 | 0.003011 |
| POM121 | ENST00000358357 | 10.370% | 2.07% | 8.30% | ENST00000395270 | 76.40% | 95.92% | 19.52% | 5.39E−05 | 0.003011 |
| COL4A1 | ENST00000375820 | 65.28% | 47.71% | 17.57% | ENST00000647632 | 4.09% | 12.27% | 8.180% | 5.94E−05 | 0.003236 |
| KLF6 | ENST00000542957 | 25.05% | 3.31% | 21.740% | ENST00000497571 | 62.24% | 92.44% | 30.20% | 6.01E−05 | 0.003236 |
| CEMIP2 | ENST00000377044 | 34.950% | 7.38% | 27.58% | ENST00000543165 | 42.68% | 59.24% | 16.55% | 6.83E−05 | 0.003614 |
| WIPF1 | ENST00000487291 | 22.220% | 11.11% | 11.11% | ENST00000392547 | 25.54% | 32.06% | 6.51% | 7.56E−05 | 0.003937 |
| PNPT1 | ENST00000260604 | 42.57% | 2.70% | 39.86% | ENST00000447944 | 12.76% | 63.28% | 50.52% | 8.83E−05 | 0.004465 |
| USP15 | ENST00000280377 | 19.74% | 5.08% | 14.66% | ENST00000537297 | 12.85% | 33.20% | 20.35% | 9.72E−05 | 0.004815 |
| MIR29B2CHG | ENST00000657366 | 14.71% | 0.00% | 14.71% | ENST00000608023 | 85.29% | 98.20% | 12.91% | 0.0001 | 0.004907 |
| PTPRE | ENST00000455661 | 31.85% | 21.76% | 10.09% | ENST00000463727 | 16.91% | 47.84% | 30.93% | 0.0001 | 0.004907 |
| S100A4 | ENST00000354332 | 12.50% | 0.61% | 11.89% | ENST00000368716 | 81.25% | 96.95% | 15.70% | 0.00011 | 0.004945 |
| PHACTR1 | ENST00000482982 | 32.89% | 6.73% | 26.16% | ENST00000332995 | 22.95% | 46.21% | 23.26% | 0.00011 | 0.00495 |
| UPF3A | ENST00000475218 | 16.64% | 5.96% | 10.68% | ENST00000492270 | 3.01% | 25.13% | 22.12% | 0.00012 | 0.005593 |
| NPIPA1 | ENST00000472413 | 63.16% | 22.90% | 40.26% | ENST00000328085 | 11.84% | 44.10% | 32.26% | 0.00013 | 0.005621 |
| VAPA | ENST00000577901 | 36.43% | 22.73% | 13.70% | ENST00000400000 | 31.43% | 62.30% | 30.87% | 0.00015 | 0.00637 |
| DNAJB4 | ENST00000370763 | 86.640% | 25.00% | 61.64% | ENST00000484662 | 6.900% | 60.00% | 53.10% | 0.00015 | 0.006589 |
| TBCA | ENST00000651106 | 10.53% | 0.90% | 9.62% | ENST00000380377 | 80.70% | 92.63% | 11.92% | 0.00015 | 0.006589 |
| ITGA1 | ENST00000650673 | 43.530% | 32.31% | 11.22% | ENST00000504669 | 20.24% | 26.93% | 6.68% | 0.00018 | 0.007552 |
| PRKCE | ENST00000480633 | 41.580% | 1.49% | 40.09% | ENST00000497602 | 0.00% | 16.42% | 16.42% | 0.00018 | 0.007552 |
| CNP | ENST00000592861 | 32.86% | 6.45% | 26.41% | ENST00000393892 | 65.20% | 93.55% | 28.35% | 0.00019 | 0.007683 |
| DDB2 | ENST00000612309 | 8.580% | 0.00% | 8.58% | ENST00000617847 | 8.54% | 21.47% | 12.93% | 0.00023 | 0.009218 |
| CD163L1 | ENST00000545597 | 22.78% | 5.63% | 17.15% | ENST00000543276 | 19.230% | 57.14% | 37.91% | 0.00025 | 0.009905 |
| DLC1 | ENST00000358919 | 14.74% | 3.37% | 11.37% | ENST00000511869 | 0.00% | 32.45% | 32.45% | 0.00028 | 0.010624 |
| LINC-PINT | ENST00000435523 | 16.77% | 6.25% | 10.53% | ENST00000647388 | 3.23% | 23.49% | 20.27% | 0.00028 | 0.010624 |
| MRPS21 | ENST00000581066 | 68.36% | 42.31% | 26.05% | ENST00000614145 | 31.64% | 57.69% | 26.05% | 0.00028 | 0.010624 |
| PIGX | ENST00000392391 | 57.14% | 18.60% | 38.54% | ENST00000426755 | 42.86% | 74.42% | 31.56% | 0.00029 | 0.010632 |
| RGPD6 | ENST00000329516 | 56.240% | 35.51% | 20.73% | ENST00000463822 | 16.13% | 28.17% | 12.03% | 0.00032 | 0.011495 |
| SLFN12 | ENST00000452764 | 94.74% | 29.03% | 65.70% | ENST00000304905 | 0.00% | 45.16% | 45.16% | 0.00033 | 0.011727 |
| DARS | ENST00000478212 | 18.52% | 2.06% | 16.46% | ENST00000264161 | 45.70% | 66.11% | 20.41% | 0.00037 | 0.012595 |
| POMP | ENST00000460403 | 13.75% | 2.53% | 11.22% | ENST00000380842 | 86.25% | 97.47% | 11.22% | 0.00036 | 0.012595 |
| USP48 | ENST00000471752 | 22.50% | 2.82% | 19.68% | ENST00000374732 | 13.14% | 28.40% | 15.26% | 0.00038 | 0.013083 |
| ST6GAL1 | ENST00000485105 | 13.22% | 8.27% | 4.94% | ENST00000448408 | 67.02% | 82.50% | 15.48% | 0.00039 | 0.013269 |
| GOLGA8B | ENST00000569100 | 27.57% | 4.21% | 23.36% | ENST00000567956 | 9.92% | 18.94% | 9.02% | 0.0004 | 0.013503 |
| NDUFAF6 | ENST00000396111 | 12.20% | 0.96% | 11.23% | ENST00000521840 | 64.63% | 70.13% | 5.50% | 0.00041 | 0.013509 |
| H3F3B | ENST00000591893 | 34.51% | 18.33% | 16.17% | ENST00000254810 | 58.95% | 81.67% | 22.72% | 0.00045 | 0.01428 |
| KTN1 | ENST00000556631 | 13.62% | 3.92% | 9.70% | ENST00000554831 | 25.48% | 41.34% | 15.86% | 0.00044 | 0.01428 |
| OSMR | ENST00000502536 | 20.43% | 2.63% | 17.800% | ENST00000274276 | 69.99% | 90.35% | 20.35% | 0.00045 | 0.01428 |
| CBWD6 | ENST00000457288 | 17.20% | 4.56% | 12.64% | ENST00000617722 | 16.82% | 24.71% | 7.89% | 0.00046 | 0.014586 |
| PCGF3 | ENST00000430644 | 37.04% | 8.86% | 28.18% | ENST00000362003 | 53.55% | 77.68% | 24.12% | 0.00048 | 0.015194 |
| PELI1 | ENST00000358912 | 79.63% | 50.71% | 28.92% | ENST00000468869 | 1.89% | 28.24% | 26.35% | 0.00054 | 0.016651 |
| KPNA5 | ENST00000368564 | 78.98% | 55.73% | 23.240% | ENST00000413340 | 11.11% | 43.21% | 32.10% | 0.00055 | 0.01688 |
| SNHG7 | ENST00000414282 | 24.240% | 1.45% | 22.79% | ENST00000416970 | 0.00% | 17.39% | 17.39% | 0.00057 | 0.017447 |
| TFRC | ENST00000420415 | 19.07% | 10.51% | 8.56% | ENST00000392396 | 16.77% | 31.22% | 14.45% | 0.00058 | 0.017582 |
| TUBB6 | ENST00000587204 | 31.77% | 13.16% | 18.61% | ENST00000591909 | 30.70% | 64.91% | 34.21% | 0.00063 | 0.018518 |
| CHCHD1 | ENST00000372837 | 40.91% | 10.96% | 29.95% | ENST00000372833 | 59.09% | 89.04% | 29.95% | 0.00078 | 0.022789 |
| TNFRSF14 | ENST00000482602 | 20.83% | 1.49% | 19.34% | ENST00000463471 | 32.68% | 64.18% | 31.50% | 0.00079 | 0.022789 |
| NRP1 | ENST00000374867 | 10.630% | 3.41% | 7.23% | ENST00000432372 | 19.14% | 35.13% | 15.99% | 0.0008 | 0.023049 |
| TCERG1 | ENST00000509787 | 14.81% | 0.80% | 14.01% | ENST00000509810 | 5.93% | 15.28% | 9.36% | 0.00088 | 0.024878 |
| CA2 | ENST00000520996 | 17.56% | 3.74% | 13.82% | ENST00000285379 | 78.85% | 96.26% | 17.41% | 0.00093 | 0.025763 |
| PYGO2 | ENST00000483463 | 84.00% | 51.85% | 32.15% | ENST00000368457 | 14.52% | 48.15% | 33.63% | 0.00093 | 0.025763 |
| BMPR2 | ENST00000374580 | 75.820% | 18.5% | 57.31% | ENST00000479069 | 20.00% | 79.16% | 59.16% | 0.00104 | 0.028145 |
| MPC2 | ENST00000271373 | 15.00% | 0.00% | 15.00% | ENST00000367846 | 85.00% | 100.00% | 15.00% | 0.00103 | 0.028145 |
| NUTM2A-AS1 | ENST00000668225 | 7.14% | 0.93% | 6.22% | ENST00000654503 | 19.93% | 36.91% | 16.98% | 0.00106 | 0.028584 |
| CDR2 | ENST00000564542 | 30.00% | 10.23% | 19.77% | ENST00000569045 | 54.00% | 77.92% | 23.92% | 0.00114 | 0.030333 |
| FGD4 | ENST00000473513 | 9.93% | 2.59% | 7.34% | ENST00000525053 | 3.11% | 26.15% | 23.04% | 0.00115 | 0.03044 |
| RECQL | ENST00000539672 | 27.50% | 0.00% | 27.50% | ENST00000444129 | 72.50% | 100.00% | 27.50% | 0.00116 | 0.03044 |
| MICOS10 | ENST00000498067 | 16.09% | 4.38% | 11.70% | ENST00000322753 | 76.95% | 82.60% | 5.650% | 0.00117 | 0.030466 |
| SF3B6 | ENST00000478050 | 22.22% | 1.33% | 20.89% | ENST00000233468 | 77.78% | 98.67% | 20.89% | 0.00121 | 0.031114 |
| ZNF518A | ENST00000484770 | 71.09% | 59.57% | 11.52% | ENST00000563195 | 1.23% | 14.81% | 13.58% | 0.00131 | 0.033582 |
| AC027097.2 | ENST00000588925 | 40.00% | 11.71% | 28.29% | ENST00000660188 | 0.00% | 54.35% | 54.35% | 0.00137 | 0.034241 |
| COBLL1 | ENST00000439313 | 42.11% | 5.45% | 36.65% | ENST00000480873 | 5.57% | 16.42% | 10.84% | 0.00136 | 0.034241 |
| NDUFAB1 | ENST00000570319 | 5.77% | 0.00% | 5.77% | ENST00000007516 | 86.54% | 97.71% | 11.18% | 0.0014 | 0.034445 |
| PDE7B | ENST00000615259 | 57.10% | 29.82% | 27.28% | ENST00000308191 | 36.08% | 68.42% | 32.34% | 0.0014 | 0.034445 |
| DSTN | ENST00000449141 | 31.71% | 9.59% | 22.12% | ENST00000246069 | 46.34% | 78.08% | 31.74% | 0.00164 | 0.039304 |
| MAP3K8 | ENST00000375321 | 14.99% | 2.20% | 12.790% | ENST00000413724 | 26.09% | 58.59% | 32.51% | 0.00166 | 0.039403 |
| FAM13B | ENST00000513640 | 26.32% | 4.49% | 21.82% | ENST00000033079 | 36.84% | 55.06% | 18.21% | 0.00171 | 0.040306 |
| CAVI | ENST00000341049 | 72.72% | 23.40% | 49.32% | ENST00000393467 | 18.18% | 61.70% | 43.52% | 0.00176 | 0.040881 |
| DYRK2 | ENST00000542503 | 31.58% | 3.33% | 28.25% | ENST00000344096 | 68.42% | 96.67% | 28.25% | 0.00181 | 0.040881 |
| MCL1 | ENST00000620947 | 13.24% | 0.01% | 13.22% | ENST00000369026 | 76.23% | 94.73% | 18.50% | 0.00181 | 0.040881 |
| NBPF10 | ENST00000583866 | 57.50% | 30.42% | 27.07% | ENST00000612520 | 37.50% | 66.05% | 28.55% | 0.00181 | 0.040881 |
| SRGAP2 | ENST00000604423 | 22.64% | 11.28% | 11.35% | ENST00000624686 | 44.86% | 64.59% | 19.73% | 0.00179 | 0.040881 |
| TRAPPC12 | ENST00000452495 | 25.00% | 4.00% | 21.00% | ENST00000497597 | 40.00% | 66.67% | 26.67% | 0.00194 | 0.043458 |
| CCDC47 | ENST00000582331 | 29.41% | 1.82% | 27.59% | ENST00000584112 | 5.88% | 21.82% | 15.94% | 0.00199 | 0.044039 |
| ANAPC5 | ENST00000535463 | 8.57% | 0.00% | 8.57% | ENST00000538334 | 24.71% | 45.74% | 21.03% | 0.00209 | 0.045864 |
| WDR59 | ENST00000563111 | 17.97% | 3.76% | 14.21% | ENST00000569421 | 9.38% | 26.40% | 17.03% | 0.00217 | 0.047292 |
| PGM2L1 | ENST00000622957 | 57.11% | 32.14% | 24.97% | ENST00000298198 | 42.89% | 67.86% | 24.97% | 0.00227 | 0.048659 |
| SWAP70 | ENST00000534662 | 20.53% | 3.64% | 16.89% | ENST00000531814 | 2.81% | 17.73% | 14.92% | 0.00225 | 0.048659 |

TABLE 8
Myeloid cell specific DCI genes

| | | | | | | Prevalance | | | | |
|---|---|---|---|---|---|---|---|---|---|---|
| | | | | | | of | | Prevalance | | |
| | | Prevalance | Prevalance | | | non-myeloid | | difference | | |
| | | of | of | | | cell | Prevalance | of | | |
| | Myeloid | myeloid | myeloid | Prevalance | | preferred | of | non- | | |
| | cell | cell preferred | cell preferred | difference | Non-myeloid | isoform | non-myeloid cell | myeloid | | |
| | preferred | isoform in | isoform in | of | cell | in | preferred isoform | cell | | FDR |
| Gene | isoform | myeloid | non-myeloid | myeloid cell | preferred isoform | myeloid | in non-myeloid | preferred | | adj |
| Name | ID | cells | cells | preferred isoform | ID | cells | cells | isoform | P-values | P-values |
| CD74 | ENST00000523836 | 33.75% | 18.45% | 15.30% | ENST00000353334 | 14.60% | 30.19% | 15.59% | 7.59E−26 | 1.93E−22 |
| CSTB | ENST00000640406 | 16.40% | 2.49% | 13.91% | ENST00000291568 | 77.54% | 97.08% | 19.54% | 6.72E−23 | 8.55E−20 |
| CARMIL1 | ENST00000461945 | 97.53% | 18.67% | 78.86% | ENST00000329474 | 2.47% | 77.81% | 75.34% | 7.34E−21 | 6.22E−18 |
| YWHAH | ENST00000248975 | 21.04% | 0.37% | 20.66% | ENST00000397492 | 72.16% | 95.87% | 23.71% | 5.29E−19 | 3.36E−16 |
| MT2A | ENST00000561491 | 12.93% | 4.05% | 8.88% | ENST00000245185 | 70.63% | 86.86% | 16.22% | 9.13E−16 | 4.64E−13 |
| FYB1 | ENST00000512982 | 13.27% | 5.48% | 7.78% | ENST00000646444 | 10.40% | 28.81% | 18.40% | 5.20E−13 | 2.21E−10 |
| HLA-DPA1 | ENST00000417724 | 33.67% | 18.08% | 15.59% | ENST00000419277 | 52.31% | 72.29% | 19.98% | 9.79E−09 | 3.56E−06 |
| AL3547401 | ENST00000429998 | 62.64% | 26.16% | 36.47% | ENST00000593917 | 32.97% | 70.77% | 37.80% | 2.52E−08 | 8.02E−06 |
| MTREX | ENST00000518955 | 23.81% | 4.63% | 19.18% | ENST00000230640 | 22.42% | 73.60% | 51.17% | 3.58E−08 | 1.01E−05 |
| SMCHD1 | ENST00000577300 | 18.94% | 10.48% | 8.46% | ENST00000320876 | 22.52% | 40.55% | 18.03% | 4.00E−08 | 1.02E−05 |
| CCL3 | ENST00000613922 | 84.19% | 44.83% | 39.35% | ENST00000614051 | 15.12% | 54.25% | 39.12% | 4.84E−08 | 1.03E−05 |
| PRDM1 | ENST00000424894 | 9.08% | 0.17% | 8.92% | ENST00000481163 | 9.46% | 24.83% | 15.37% | 9.11E−08 | 1.78E−05 |
| NAMPT | ENST00000486949 | 23.80% | 11.86% | 11.93% | ENST00000222553 | 26.21% | 45.25% | 19.04% | 3.71E−07 | 6.74E−05 |
| PTPRE | ENST00000463727 | 57.49% | 19.79% | 37.70% | ENST00000455661 | 5.07% | 35.88% | 30.81% | 4.88E−07 | 7.76E−05 |
| ZFYVE16 | ENST00000512558 | 25.40% | 13.45% | 11.94% | ENST00000505560 | 14.30% | 41.22% | 26.93% | 4.78E−07 | 7.76E−05 |
| GLS | ENST00000320717 | 54.23% | 33.47% | 20.77% | ENST00000338435 | 22.83% | 43.73% | 20.90% | 2.68E−06 | 0.00038 |
| CBWD5 | ENST00000382404 | 17.82% | 7.85% | 9.97% | ENST00000468198 | 7.47% | 28.12% | 20.65% | 3.04E−06 | 0.00041 |
| RASGRP3 | ENST00000484909 | 38.89% | 19.83% | 19.06% | ENST00000468856 | 0.00% | 11.58% | 11.58% | 3.34E−06 | 0.00041 |
| TMEM176B | ENST00000492607 | 36.76% | 9.89% | 26.87% | ENST00000326442 | 36.77% | 74.78% | 38.01% | 3.39E−06 | 0.00041 |
| LIMS1 | ENST00000393310 | 34.70% | 8.64% | 26.06% | ENST00000544547 | 6.83% | 19.77% | 12.94% | 3.78E−06 | 0.00044 |
| GNPAT | ENST00000436239 | 19.23% | 0.00% | 19.23% | ENST00000366647 | 62.66% | 86.50% | 23.84% | 4.18E−06 | 0.00046 |
| ATP5PD | ENST00000580649 | 14.91% | 2.47% | 12.44% | ENST00000301587 | 85.09% | 97.28% | 12.19% | 5.06E−06 | 0.00054 |
| DAB2 | ENST00000545653 | 19.12% | 3.70% | 15.42% | ENST00000515700 | 1.47% | 15.43% | 13.96% | 6.17E−06 | 0.00063 |
| ERN1 | ENST00000584041 | 13.33% | 2.55% | 10.78% | ENST00000606895 | 22.22% | 39.21% | 16.99% | 8.22E−06 | 0.0008 |
| IFI30 | ENST00000600463 | 29.97% | 11.73% | 18.24% | ENST00000407280 | 70.03% | 88.27% | 18.24% | 8.82E−06 | 0.00083 |
| CCNH | ENST00000508855 | 15.48% | 3.40% | 12.08% | ENST00000607486 | 5.62% | 24.00% | 18.38% | 1.04E−05 | 0.00091 |
| TCF25 | ENST00000563636 | 11.23% | 1.83% | 9.40% | ENST00000564652 | 23.78% | 35.64% | 11.86% | 1.02E−05 | 0.00091 |
| SMAP2 | ENST00000435168 | 44.44% | 3.92% | 40.52% | ENST00000372718 | 13.78% | 68.63% | 54.850% | 1.19E−05 | 0.00101 |
| NAIP | ENST00000503719 | 10.98% | 1.52% | 9.46% | ENST00000508794 | 16.80% | 30.40% | 13.60% | 1.38E−05 | 0.0011 |
| DDX5 | ENST00000581230 | 91.87% | 80.29% | 11.57% | ENST00000225792 | 3.16% | 17.10% | 13.94% | 1.43E−05 | 0.0011 |
| TNFAIP8 | ENST00000504771 | 20.68% | 7.21% | 13.47% | ENST00000513374 | 41.71% | 75.87% | 34.16% | 2.05105 | 0.00153 |
| CYTOR | ENST00000413202 | 22.36% | 7.89% | 14.47% | ENST00000642451 | 16.25% | 31.75% | 15.50% | 3.21E−05 | 0.00233 |
| PDE4DIP | ENST00000616206 | 23.73% | 5.00% | 18.73% | ENST00000529945 | 19.37% | 38.43% | 19.06% | 3.33E−05 | 0.00234 |
| VPS13C | ENST00000249837 | 33.62% | 16.54% | 17.08% | ENST00000559119 | 17.85% | 42.28% | 24.43% | 3.41E−05 | 0.00234 |
| DSE | ENST00000606265 | 15.24% | 2.17% | 13.06% | ENST00000647046 | 6.05% | 36.96% | 30.91% | 3.72E−05 | 0.00249 |
| SND1 | ENST00000492840 | 28.37% | 1.42% | 26.95% | ENST00000489417 | 0.00% | 12.72% | 12.72% | 5.04E−05 | 0.00329 |
| MIB1 | ENST00000577749 | 46.67% | 8.33% | 38.33% | ENST00000261537 | 46.67% | 88.09% | 41.43% | 6.43E−05 | 0.00409 |
| PPDPFL | ENST00000399653 | 88.21% | 69.95% | 18.26% | ENST00000517663 | 5.46% | 24.32% | 18.86% | 7.34E−05 | 0.00455 |
| NRP1 | ENST00000432372 | 43.27% | 19.05% | 24.22% | ENST00000374816 | 7.94% | 17.38% | 9.44% | 7.87E−05 | 0.00477 |
| NBPF10 | ENST00000612520 | 71.29% | 52.38% | 18.91% | ENST00000583866 | 23.94% | 45.28% | 21.34% | 8.51E−05 | 0.00503 |
| TAOK3 | ENST00000542902 | 21.43% | 1.61% | 19.82% | ENST00000537952 | 2.38% | 24.56% | 22.180% | 9.17E−05 | 0.0053 |
| UGT1A1 | ENST00000360418 | 30.96% | 12.41% | 18.54% | ENST00000305208 | 69.040% | 87.59% | 18.54% | 9.46E−05 | 0.00535 |
| ITGA4 | ENST00000397033 | 53.74% | 40.82% | 12.93% | ENST00000476089 | 8.28% | 20.19% | 11.910% | 0.0001 | 0.00555 |
| PPA1 | ENST00000610026 | 62.96% | 30.00% | 32.96% | ENST00000373232 | 20.080% | 65.000% | 44.920% | 0.00011 | 0.00594 |
| CKLF | ENST00000526149 | 26.31% | 4.05% | 22.26% | ENST00000264001 | 7.90% | 25.590% | 17.70% | 0.00013 | 0.00617 |
| OSTC | ENST00000505745 | 30.23% | 5.44% | 24.79% | ENST00000361564 | 69.77% | 86.62% | 16.85% | 0.00012 | 0.00617 |
| SLC38A2 | ENST00000551405 | 10.53% | 1.11% | 9.42% | ENST00000256689 | 78.82% | 90.96% | 12.14% | 0.00013 | 0.00617 |
| SRP19 | ENST00000282999 | 35.71% | 5.16% | 30.56% | ENST00000503445 | 0.00% | 14.29% | 14.29% | 0.00014 | 0.0065 |
| PNKP | ENST00000629179 | 39.13% | 3.95% | 35.18% | ENST00000626274 | 30.43% | 67.85% | 37.42% | 0.00015 | 0.00719 |
| HA VCR2 | ENST00000522902 | 51.52% | 25.93% | 25.60% | ENST00000524219 | 6.11% | 20.37% | 14.26% | 0.00016 | 0.00734 |
| PIGX | ENST00000426755 | 88.64% | 52.86% | 35.78% | ENST00000392391 | 6.82% | 41.43% | 34.61% | 0.00023 | 0.01028 |
| AC243829.4 | ENST00000616926 | 98.44% | 72.79% | 25.64% | ENST00000610845 | 0.00% | 20.87% | 20.87% | 0.00023 | 0.0103 |
| HIF 1A | ENST00000337138 | 45.42% | 27.01% | 18.41% | ENST00000547430 | 13.10% | 30.56% | 17.46% | 0.00027 | 0.0117 |
| SNX29 | ENST00000564111 | 25.81% | 7.87% | 17.94% | ENST00000564791 | 18.29% | 31.98% | 13.69% | 0.00029 | 0.01246 |
| TFEC | ENST00000265440 | 27.08% | 2.29% | 24.79% | ENST00000484212 | 34.950% | 57.10% | 22.15% | 0.00029 | 0.01246 |
| RABGAP1 | ENST00000456584 | 35.48% | 7.18% | 28.30% | ENST00000373647 | 41.94% | 61.42% | 19.48% | 0.00036 | 0.01503 |
| AL0087291 | ENST00000399446 | 84.21% | 47.92% | 36.29% | ENST00000606150 | 10.53% | 31.25% | 20.72% | 0.00038 | 0.01541 |
| PGF | ENST00000555253 | 54.41% | 24.76% | 29.66% | ENST00000238607 | 32.89% | 50.97% | 18.07% | 0.00041 | 0.01644 |
| GTF2I | ENST00000491325 | 16.85% | 4.58% | 12.27% | ENST00000614048 | 8.99% | 18.07% | 9.08% | 0.00044 | 0.01733 |
| MOBIA | ENST00000463975 | 36.42% | 10.69% | 25.73% | ENST00000396049 | 58.02% | 68.70% | 10.68% | 0.00048 | 0.01829 |
| SAMHD1 | ENST00000645444 | 43.19% | 10.17% | 33.02% | ENST00000643161 | 0.00% | 15.25% | 15.25% | 0.00049 | 0.01829 |
| SYNGR2 | ENST00000592456 | 45.83% | 2.44% | 43.39% | ENST00000225777 | 33.33% | 78.05% | 44.72% | 0.00048 | 0.01829 |
| DDX60L | ENST00000504793 | 11.01% | 0.00% | 11.01% | ENST00000513103 | 0.00% | 29.46% | 29.46% | 0.00051 | 0.01873 |
| UBAC2 | ENST00000460562 | 24.44% | 3.26% | 21.18% | ENST00000376440 | 4.44% | 22.83% | 18.38% | 0.00058 | 0.02105 |
| SRGAP2 | ENST00000624686 | 67.89% | 49.23% | 18.66% | ENST00000604423 | 9.28% | 20.20% | 10.92% | 0.00066 | 0.02352 |
| UGT1A4 | ENST00000450233 | 22.78% | 8.18% | 14.60% | ENST00000373409 | 77.22% | 91.82% | 14.60% | 0.00069 | 0.0244 |
| RHOBTB3 | ENST00000504949 | 11.66% | 3.11% | 8.56% | ENST00000379982 | 65.14% | 82.35% | 17.21% | 0.00074 | 0.02564 |
| UGT1A6 | ENST00000406651 | 20.33% | 7.54% | 12.78% | ENST00000305139 | 74.18% | 90.09% | 15.91% | 0.00075 | 0.02564 |
| IFIT3 | ENST00000371818 | 63.63% | 26.52% | 37.11% | ENST00000371811 | 36.37% | 73.48% | 37.11% | 0.0008 | 0.02709 |
| OXSR1 | ENST00000483695 | 22.22% | 2.50% | 19.72% | ENST00000311806 | 55.56% | 77.50% | 21.94% | 0.00081 | 0.02709 |
| KLF6 | ENST00000497571 | 99.97% | 70.23% | 29.73% | ENST00000542957 | 0.03% | 18.30% | 18.27% | 0.00085 | 0.02823 |
| HOOK2 | ENST00000593143 | 94.61% | 83.72% | 10.89% | ENST00000589134 | 1.16% | 10.63% | 9.47% | 0.00088 | 0.02871 |
| DST | ENST00000370765 | 19.100% | 10.01% | 9.09% | ENST00000340834 | 12.82% | 38.47% | 25.65% | 0.00094 | 0.02921 |
| SMIM4 | ENST00000476842 | 53.13% | 14.58% | 38.540% | ENST00000477703 | 15.63% | 50.000% | 34.38% | 0.00091 | 0.02921 |
| STAT1 | ENST00000392323 | 27.71% | 12.250% | 15.46% | ENST00000415035 | 23.14% | 33.30% | 10.17% | 0.00094 | 0.02921 |
| IVNS1ABP | ENST00000475046 | 10.20% | 4.10% | 6.10% | ENST00000367498 | 3.67% | 25.91% | 22.24% | 0.00098 | 0.03003 |
| CSNK2A1 | ENST00000608066 | 20.59% | 0.83% | 19.75% | ENST00000645334 | 0.00% | 18.80% | 18.80% | 0.00101 | 0.03056 |
| CLK3 | ENST00000564353 | 47.06% | 17.24% | 29.82% | ENST00000563418 | 29.41% | 77.59% | 48.17% | 0.00109 | 0.03235 |
| ATG7 | ENST00000470474 | 11.83% | 1.27% | 10.56% | ENST00000354449 | 28.05% | 60.000% | 31.95% | 0.00126 | 0.03614 |
| RBBP4 | ENST00000465780 | 18.18% | 6.42% | 11.76% | ENST00000373493 | 61.36% | 78.61% | 17.25% | 0.00125 | 0.03614 |
| HSPD1 | ENST00000491249 | 25.85% | 4.30% | 21.55% | ENST00000426480 | 5.02% | 15.62% | 10.60% | 0.00134 | 0.03756 |
| GOLGA8R | ENST00000624918 | 81.48% | 39.02% | 42.46% | ENST00000327271 | 18.52% | 60.98% | 42.46% | 0.00137 | 0.03794 |
| ATP5MC1 | ENST00000393366 | 44.02% | 33.15% | 10.86% | ENST00000355938 | 45.30% | 58.84% | 13.54% | 0.00144 | 0.03909 |
| UGT1A10 | ENST00000373445 | 24.41% | 11.93% | 12.48% | ENST00000344644 | 75.59% | 88.07% | 12.48% | 0.00149 | 0.03992 |
| RGPD6 | ENST00000437167 | 18.05% | 4.85% | 13.20% | ENST00000329516 | 17.21% | 48.91% | 31.70% | 0.00153 | 0.04051 |
| SF3A3 | ENST00000461869 | 17.65% | 0.00% | 17.65% | ENST00000373019 | 58.82% | 90.54% | 31.72% | 0.00154 | 0.04051 |
| UBR1 | ENST00000546274 | 13.04% | 2.61% | 10.43% | ENST00000290650 | 56.52% | 74.78% | 18.26% | 0.00186 | 0.04738 |
| SOX6 | ENST00000530378 | 16.31% | 1.47% | 14.84% | ENST00000533870 | 0.00% | 23.53% | 23.53% | 0.0019 | 0.04779 |
| SAFB2 | ENST00000590000 | 20.00% | 10.98% | 9.02% | ENST00000592599 | 0.00% | 18.85% | 18.85% | 0.00198 | 0.04936 |
| CBLB | ENST00000476370 | 43.44% | 31.13% | 12.31% | ENST00000646499 | 24.22% | 31.35% | 7.13% | 0.00203 | 0.0496 |

TABLE 9
Follicular B cell specific DCI genes

| | | | | | | Prevalance | | | | |
|---|---|---|---|---|---|---|---|---|---|---|
| | | | | | | of | | Prevalance | | |
| | | Prevalance | Prevalance | | | non-follicular | | difference | | |
| | | of | of | | | cell | Prevalance | of | | |
| | Follicular | follicular | follicular | Prevalance | | preferred | of | non- | | |
| | cell | cell preferred | cell preferred | difference | Non-follicular | isoform | non-follicular cell | follicular | | |
| | preferred | isoform in | isoform in | of | cell | in | preferred isoform | cell | | FDR |
| Gene | isoform | follicular | non-follicular | follicular cell | preferred isoform | follicular | in non-follicular | preferred | | adj |
| Name | ID | cells | cells | preferred isoform | ID | cells | cells | isoform | P-values | P-values |
| IGHG1 | ENST00000390548 | 26.06% | 1.13% | 24.93% | ENST00000390549 | 72.20% | 98.780% | 26.58% | 6.53E−138 | 8.00E−135 |
| IGHG3 | ENST00000641136 | 27.67% | 1.14% | 26.53% | ENST00000390551 | 72.33% | 98.86% | 26.53% | 1.85E−113 | 7.56E−111 |
| IGHG2 | ENST00000641095 | 30.44% | 1.44% | 29.00% | ENST00000390545 | 69.56% | 98.56% | 29.00% | 1.66E−108 | 5.08E−106 |
| CD74 | ENST00000523836 | 36.65% | 24.36% | 12.29% | ENST00000009530 | 26.51% | 40.52% | 14.01% | 6.01E−19 | 1.47E−16 |
| NOTCH2 | ENST00000650638 | 56.76% | 4.59% | 52.17% | ENST00000256646 | 21.62% | 38.99% | 17.37% | 5.23E−15 | 1.07E−12 |
| ARHGAP24 | ENST00000509709 | 38.09% | 8.45% | 29.63% | ENST00000264343 | 18.47% | 39.64% | 21.17% | 9.81E−11 | 1.50E−08 |
| IGHA2 | ENST00000497872 | 46.67% | 1.72% | 44.94% | ENST00000390539 | 53.33% | 98.28% | 44.94% | 2.98E−09 | 4.05E−07 |
| ACTR2 | ENST00000471552 | 36.36% | 4.55% | 31.82% | ENST00000260641 | 18.18% | 79.36% | 61.18% | 5.39E−09 | 6.60E−07 |
| BCAS4 | ENST00000371608 | 25.45% | 7.44% | 18.01% | ENST00000463943 | 69.09% | 92.56% | 23.47% | 2.16E−07 | 2.41E−05 |
| GOLGA8A | ENST00000569781 | 20.51% | 3.56% | 16.96% | ENST00000473125 | 64.35% | 94.05% | 29.70% | 4.02E−07 | 4.10E−05 |
| MLLT6 | ENST00000620609 | 25.00% | 2.08% | 22.92% | ENST00000621332 | 43.75% | 78.24% | 34.49% | 7.14E−07 | 6.72E−05 |
| MT2A | ENST00000245185 | 100.00% | 80.58% | 19.42% | ENST00000562017 | 0.00% | 10.49% | 10.49% | 3.38E−06 | 0.0003 |
| MICOS10 | ENST00000498642 | 15.09% | 2.11% | 12.99% | ENST00000322753 | 67.92% | 82.90% | 14.98% | 3.96E−05 | 0.00303 |
| PAWR | ENST00000547016 | 25.00% | 0.00% | 25.00% | ENST00000551712 | 0.00% | 19.44% | 19.44% | 0.00014 | 0.00871 |
| RGPD6 | ENST00000463822 | 43.81% | 19.25% | 24.56% | ENST00000329516 | 21.59% | 48.43% | 26.84% | 0.00014 | 0.00871 |
| TMEM131 | ENST00000485245 | 26.25% | 12.96% | 13.29% | ENST00000186436 | 56.25% | 79.990% | 23.74% | 0.00013 | 0.00871 |
| INPP5D | ENST00000359570 | 19.99% | 6.34% | 13.65% | ENST00000496402 | 1.79% | 20.62% | 18.84% | 0.00017 | 0.00945 |
| PIK3AP1 | ENST00000468783 | 30.01% | 2.04% | 27.97% | ENST00000339364 | 20.07% | 57.820% | 37.75% | 0.00017 | 0.00945 |
| RGPD5 | ENST00000477523 | 35.56% | 12.530% | 23.02% | ENST00000016946 | 18.48% | 30.86% | 12.38% | 0.00021 | 0.01099 |
| LINC-PINT | ENST00000647388 | 27.91% | 16.82% | 11.09% | ENST00000451786 | 2.40% | 14.70% | 12.30% | 0.00024 | 0.01219 |
| KMT2A | ENST00000648029 | 11.11% | 0.00% | 11.11% | ENST00000534358 | 30.55% | 38.61% | 8.05% | 0.00029 | 0.01346 |
| SRSF6 | ENST00000662078 | 10.53% | 0.00% | 10.53% | ENST00000244020 | 89.47% | 98.63% | 9.16% | 0.00028 | 0.01346 |
| SP110 | ENST00000463022 | 16.77% | 0.00% | 16.77% | ENST00000489597 | 0.00% | 8.42% | 8.42% | 0.0004 | 0.01737 |
| COX16 | ENST00000557612 | 25.00% | 2.94% | 22.06% | ENST00000555276 | 50.00% | 59.35% | 9.35% | 0.00047 | 0.01964 |
| PUM3 | ENST00000382032 | 7.69% | 0.00% | 7.69% | ENST00000469168 | 0.00% | 13.07% | 13.07% | 0.00048 | 0.01964 |
| FAM214A | ENST00000561490 | 22.22% | 1.58% | 20.64% | ENST00000562351 | 29.63% | 46.51% | 16.88% | 0.00051 | 0.0202 |
| ARL17B | ENST00000570618 | 12.11% | 0.76% | 11.36% | ENST00000622877 | 2.37% | 8.41% | 6.05% | 0.00055 | 0.02088 |
| PLEKHG1 | ENST00000644996 | 59.09% | 12.67% | 46.42% | ENST00000358517 | 15.33% | 38.09% | 22.77% | 0.00063 | 0.02318 |
| DANCR | ENST00000653147 | 14.29% | 0.54% | 13.74% | ENST00000411630 | 85.71% | 95.65% | 9.93% | 0.00071 | 0.02541 |
| GTF2H2 | ENST00000518898 | 30.43% | 4.45% | 25.98% | ENST00000274400 | 13.04% | 39.25% | 26.20% | 0.0011 | 0.03539 |
| TFEC | ENST00000484212 | 57.89% | 37.71% | 20.18% | ENST00000265440 | 1.22% | 24.08% | 22.86% | 0.00109 | 0.03539 |
| CD69 | ENST00000543147 | 43.96% | 21.07% | 22.89% | ENST00000228434 | 35.15% | 56.99% | 21.84% | 0.00124 | 0.039 |
| LTB | ENST00000446745 | 30.56% | 20.59% | 9.97% | ENST00000429299 | 55.56% | 77.96% | 22.41% | 0.00138 | 0.04165 |
| SRSF4 | ENST00000605204 | 14.70% | 3.31% | 11.39% | ENST00000373795 | 9.33% | 21.09% | 11.76% | 0.0014 | 0.04165 |
| CHD9 | ENST00000564582 | 24.24% | 3.33% | 20.91% | ENST00000565442 | 26.93% | 40.32% | 13.39% | 0.00145 | 0.04218 |
| FUT8 | ENST00000556518 | 22.06% | 3.54% | 18.52% | ENST00000553924 | 7.14% | 21.83% | 14.69% | 0.00156 | 0.0444 |
| 1-Mar | ENST00000511245 | 11.51% | 0.47% | 11.04% | ENST00000274056 | 2.87% | 11.83% | 8.96% | 0.00174 | 0.04837 |
| CBWD6 | ENST00000486387 | 22.29% | 12.38% | 9.92% | ENST00000611553 | 1.72% | 14.58% | 12.85% | 0.00182 | 0.04945 |

TABLE 10
T&NK cell specific DCI genes

| | | Prevalance | Prevalance | | | Prevalance | | | | |
|---|---|---|---|---|---|---|---|---|---|---|
| | | of | of | | | of | Prevalance | Prevalance | | |
| | | T&NK | T&NK | | | non-T&NK | of | difference | | |
| | | cell | cell | | | cell | non-T&NK | of | | |
| | T&NK | preferred | preferred | Prevalance | | preferred | cell | non- | | |
| | cell | isoform | isoform | difference | Non-T&NK | isoform | preferred | T&NK | | |
| | preferred | in | in | of | cell | in | isoform | cell | | FDR |
| Gene | isoform | T&NK | non-T&NK | T&NK cell | preferred isoform | T&NK | in non-T&NK | preferred | | adj |
| Name | ID | cells | cells | preferred isoform | ID | cells | cells | isoform | P-values | P-values |
| CD74 | ENST00000353334 | 41.93% | 19.81% | 22.12% | ENST00000523836 | 7.80% | 28.53% | 20.73% | 1.52E−18 | 1.66E−15 |
| MT2A | ENST00000245185 | 98.83% | 77.95% | 20.88% | ENST00000562017 | 0.39% | 11.97% | 11.58% | 4.13E−18 | 2.26E−15 |
| CYTOR | ENST00000331944 | 22.57% | 7.87% | 14.70% | ENST00000642451 | 13.49% | 33.43% | 19.94% | 8.72E−16 | 3.19E−13 |
| PRDM1 | ENST00000481163 | 53.49% | 16.32% | 37.17% | ENST00000369096 | 25.32% | 62.87% | 37.55% | 1.67E−14 | 4.58E−12 |
| AKAP13 | ENST00000559820 | 19.67% | 4.37% | 15.30% | ENST00000394518 | 1.47% | 10.22% | 8.75% | 9.65E−11 | 2.11E−08 |
| FYB1 | ENST00000515010 | 22.940% | 7.05% | 15.90% | ENST00000512982 | 6.10% | 11.54% | 5.44% | 1.27E−08 | 1.99E−06 |
| HLA-DPA1 | ENST00000419277 | 86.40% | 58.01% | 28.39% | ENST00000417724 | 9.54% | 28.89% | 19.35% | 1.91E−08 | 2.61E−06 |
| CYB5A | ENST00000340533 | 98.48% | 87.66% | 10.82% | ENST00000583418 | 0.24% | 9.36% | 9.11% | 2.40E−08 | 2.92E−06 |
| HMGN3 | ENST00000275036 | 35.08% | 19.52% | 15.55% | ENST00000620514 | 46.61% | 69.43% | 22.820% | 2.69108 | 2.95E−06 |
| AC005670.3 | ENST00000663210 | 13.510% | 3.48% | 10.03% | ENST00000658121 | 43.240% | 76.57% | 33.32% | 5.85E−07 | 5.83E−05 |
| A2M | ENST00000472360 | 18.890% | 6.80% | 12.09% | ENST00000318602 | 60.32% | 75.140% | 14.82% | 1.06E−06 | 8.91E−05 |
| VPS13C | ENST00000559119 | 56.85% | 29.49% | 27.36% | ENST00000249837 | 4.980% | 25.74% | 20.76% | 1.69E−06 | 0.000133 |
| ITGAE | ENST00000263087 | 77.70% | 46.03% | 31.66% | ENST00000570360 | 0.00% | 15.87% | 15.87% | 2.11E−06 | 0.000154 |
| CCL3 | ENST00000614051 | 55.27% | 22.98% | 32.29% | ENST00000613922 | 43.59% | 76.49% | 32.90% | 5.20E−06 | 0.000356 |
| PDS5A | ENST00000503867 | 18.75% | 0.53% | 18.22% | ENST00000303538 | 50.00% | 59.94% | 9.94% | 8.86E−06 | 0.000571 |
| ITGA4 | ENST00000476089 | 24.09% | 10.65% | 13.44% | ENST00000397033 | 31.89% | 53.71% | 21.83% | 1.36E−05 | 0.00083 |
| NBEAL1 | ENST00000460355 | 94.15% | 71.98% | 22.17% | ENST00000449802 | 4.190% | 16.43% | 12.25% | 1.86E−05 | 0.001073 |
| BHLHE40-AS1 | ENST00000663474 | 7.58% | 0.75% | 6.82% | ENST00000668962 | 84.850% | 98.50% | 13.65% | 2.61E−05 | 0.00143 |
| ATM | ENST00000530958 | 43.10% | 28.08% | 15.03% | ENST00000531525 | 8.62% | 15.030% | 6.40% | 3.04E−05 | 0.001588 |
| SEC61B | ENST00000223641 | 100.00% | 80.02% | 19.980% | ENST00000481573 | 0.00% | 19.43% | 19.43% | 4.54E−05 | 0.002261 |
| AC243829.4 | ENST00000610845 | 22.46% | 3.45% | 19.01% | ENST00000616926 | 69.91% | 95.40% | 25.49% | 9.99E−05 | 0.004209 |
| HA VCR2 | ENST00000522593 | 33.83% | 10.52% | 23.310% | ENST00000522902 | 25.37% | 45.640% | 20.26% | 0.000112 | 0.004394 |
| ASXL2 | ENST00000673455 | 52.94% | 13.33% | 39.61% | ENST00000435504 | 31.80% | 65.00% | 33.19% | 0.000163 | 0.006162 |
| IFITM2 | ENST00000399815 | 90.30% | 80.18% | 10.12% | ENST00000602569 | 3.53% | 7.35% | 3.82% | 0.000246 | 0.008411 |
| HMGB1 | ENST00000341423 | 94.23% | 71.92% | 22.31% | ENST00000399489 | 0.00% | 13.74% | 13.74% | 0.000289 | 0.009604 |
| MZB1 | ENST00000302125 | 95.00% | 53.26% | 41.74% | ENST00000503120 | 2.50% | 41.71% | 39.21% | 0.000447 | 0.014012 |
| MED13 | ENST00000580896 | 9.43% | 0.55% | 8.88% | ENST00000397786 | 83.02% | 95.31% | 12.29% | 0.000495 | 0.014479 |
| DOCK10 | ENST00000472652 | 18.60% | 2.09% | 16.51% | ENST00000644695 | 7.01% | 14.58% | 7.57% | 0.000871 | 0.022236 |
| ANKRD36 | ENST00000421946 | 56.37% | 39.41% | 16.97% | ENST00000652721 | 13.01% | 25.20% | 12.19% | 0.001221 | 0.029081 |
| RBM5 | ENST00000494360 | 17.50% | 3.81% | 13.69% | ENST00000464087 | 27.50% | 37.73% | 10.23% | 0.001315 | 0.030676 |
| Clorf122 | ENST00000373042 | 46.87% | 23.11% | 23.77% | ENST00000373043 | 25.00% | 60.23% | 35.23% | 0.001486 | 0.033931 |
| IFI30 | ENST00000407280 | 100.00% | 73.15% | 26.85% | ENST00000600463 | 0.00% | 26.85% | 26.85% | 0.001524 | 0.034087 |
| CD69 | ENST00000228434 | 60.52% | 43.49% | 17.02% | ENST00000543147 | 18.15% | 34.11% | 15.96% | 0.001566 | 0.034335 |
| FARP1 | ENST00000319562 | 70.59% | 17.54% | 53.05% | ENST00000596580 | 20.59% | 50.35% | 29.76% | 0.002 | 0.041451 |
| CASP4 | ENST00000534356 | 25.81% | 2.33% | 23.48% | ENST00000533730 | 0.00% | 5.33% | 5.33% | 0.002151 | 0.043657 |
| HMGN4 | ENST00000477243 | 11.76% | 0.00% | 11.76% | ENST00000377575 | 88.24% | 100.00% | 11.76% | 0.002278 | 0.045349 |
| AGO3 | ENST00000324350 | 22.92% | 13.04% | 9.87% | ENST00000373191 | 48.07% | 70.42% | 22.35% | 0.002317 | 0.045349 |
| SP100 | ENST00000494901 | 21.21% | 8.41% | 12.80% | ENST00000409897 | 5.68% | 11.56% | 5.88% | 0.002633 | 0.049763 |

TABLE 11
Plasma B cell specific DCI genes

| | | | | | | Prevalance | | | | |
|---|---|---|---|---|---|---|---|---|---|---|
| | | | | | | of | | Prevalance | | |
| | | Prevalance | Prevalance | | | non-plasma | | difference | | |
| | | of | of | | | cell | Prevalance | of | | |
| | Plasma | plasma | plasma | Prevalance | | preferred | of | non- | | |
| | cell | cell preferred | cell preferred | difference | Non-plasma | isoform | non-plasma cell | plasma | | |
| | preferred | isoform in | isoform in | of | cell | in | preferred isoform | cell | | FDR |
| Gene | isoform | plasma | non-plasma | plasma cell | preferred isoform | plasma | in non-plasma | preferred | | adj |
| Name | ID | cells | cells | preferred isoform | ID | cells | cells | isoform | P-values | P-values |
| CFLAR | ENST00000490965 | 39.08% | 11.58% | 27.50% | ENST00000309955 | 24.85% | 57.48% | 32.63% | 2.86E−19 | 1.22E−16 |
| CCDC144A | ENST00000340621 | 33.58% | 2.62% | 30.95% | ENST00000399273 | 41.40% | 79.12% | 37.72% | 4.55E−14 | 9.72E−12 |
| CYTOR | ENST00000646865 | 18.40% | 2.93% | 15.47% | ENST00000642451 | 7.08% | 32.16% | 25.08% | 2.18E−13 | 4.00E−11 |
| HMGB1 | ENST00000399489 | 29.30% | 6.18% | 23.13% | ENST00000341423 | 54.51% | 82.12% | 27.61% | 2.64E−12 | 4.23E−10 |
| EHMT1 | ENST00000495657 | 81.41% | 35.90% | 45.51% | ENST00000637318 | 7.76% | 17.69% | 9.93% | 4.47E−11 | 6.36E−09 |
| CBWD5 | ENST00000441808 | 11.42% | 0.88% | 10.54% | ENST00000461932 | 12.77% | 23.07% | 10.30% | 1.66E−10 | 2.12E−08 |
| NCKAP1L | ENST00000547500 | 21.88% | 2.25% | 19.62% | ENST00000293373 | 39.72% | 77.05% | 37.33% | 2.62E−10 | 3.05E−08 |
| SOD2 | ENST00000535561 | 54.55% | 4.840% | 49.71% | ENST00000535459 | 0.00% | 16.75% | 16.75% | 4.80E−10 | 5.13E−08 |
| TMSB4X | ENST00000380636 | 20.07% | 9.04% | 11.02% | ENST00000451311 | 79.93% | 90.91% | 10.98% | 5.20E−10 | 5.13E−08 |
| ANKRD10 | ENST00000460846 | 20.97% | 2.22% | 18.75% | ENST00000267339 | 18.95% | 43.33% | 24.38% | 6.04E−10 | 5.53E−08 |
| FNDC3A | ENST00000541916 | 32.24% | 8.21% | 24.03% | ENST00000398316 | 10.79% | 33.020% | 22.23% | 2.34E−09 | 2.00E−07 |
| BAZ2B | ENST00000482503 | 31.03% | 2.62% | 28.41% | ENST00000548440 | 10.34% | 24.67% | 14.32% | 2.24E−08 | 1.80E−06 |
| NEDD9 | ENST00000504634 | 25.83% | 8.75% | 17.09% | ENST00000379433 | 22.50% | 63.34% | 40.84% | 4.19E−08 | 3.16E−06 |
| CBWD3 | ENST00000618921 | 12.29% | 1.65% | 10.64% | ENST00000614377 | 11.34% | 17.02% | 5.67% | 1.16E−07 | 8.27E−06 |
| CARMIL1 | ENST00000329474 | 95.65% | 28.84% | 66.81% | ENST00000461945 | 4.35% | 69.17% | 64.82% | 1.89E−07 | 1.28E−05 |
| STK17B | ENST00000409228 | 16.79% | 2.00% | 14.79% | ENST00000449152 | 11.29% | 25.40% | 14.11% | 3.45E−07 | 2.21E−05 |
| MED13 | ENST00000583958 | 14.29% | 0.34% | 13.95% | ENST00000397786 | 85.71% | 94.02% | 8.31% | 7.40E−07 | 4.34E−05 |
| PECAM1 | ENST00000564866 | 6.94% | 0.98% | 5.96% | ENST00000563924 | 81.94% | 96.17% | 14.22% | 7.45E−07 | 4.34E−05 |
| COX14 | ENST00000548985 | 21.74% | 0.00% | 21.74% | ENST00000550487 | 78.26% | 96.27% | 18.01% | 9.99E−07 | 5.35E−05 |
| OST4 | ENST00000429985 | 30.16% | 7.24% | 22.92% | ENST00000456793 | 69.84% | 92.76% | 22.92% | 1.00E−06 | 5.35E−05 |
| PELI1 | ENST00000468869 | 53.57% | 9.09% | 44.48% | ENST00000358912 | 32.52% | 69.280% | 36.76% | 1.35E−06 | 6.92E−05 |
| MZB1 | ENST00000503120 | 48.46% | 14.23% | 34.230% | ENST00000302125 | 44.61% | 84.640% | 40.03% | 3.14E−06 | 0.000155 |
| MT2A | ENST00000245185 | 97.71% | 80.38% | 17.33% | ENST00000562017 | 1.14% | 10.61% | 9.47% | 3.54E−06 | 0.000168 |
| ATP5ME | ENST00000505852 | 15.38% | 1.020% | 14.36% | ENST00000304312 | 84.62% | 95.90% | 11.29% | 3.92E−06 | 0.000179 |
| ST6GAL1 | ENST00000448408 | 85.18% | 69.88% | 15.30% | ENST00000470633 | 1.18% | 4.18% | 3.00% | 4.41E−06 | 0.000195 |
| NDUFB11 | ENST00000276062 | 53.33% | 19.44% | 33.89% | ENST00000377811 | 46.67% | 80.56% | 33.89% | 4.89E−06 | 0.000209 |
| AFF1 | ENST00000511442 | 59.46% | 13.63% | 45.83% | ENST00000504956 | 0.00% | 28.54% | 28.54% | 5.73E−06 | 0.000223 |
| CBWD6 | ENST00000617722 | 35.40% | 16.63% | 18.77% | ENST00000611553 | 4.67% | 17.16% | 12.50% | 5.63E−06 | 0.000223 |
| PLCG2 | ENST00000567980 | 11.22% | 1.06% | 10.17% | ENST00000564138 | 55.44% | 76.190% | 20.750% | 5.74E−06 | 0.000223 |
| CHD2 | ENST00000626782 | 17.34% | 1.62% | 15.72% | ENST00000635856 | 0.68% | 8.81% | 8.13% | 5.93E−06 | 0.000223 |
| ERN1 | ENST00000606895 | 42.92% | 26.24% | 16.67% | ENST00000433197 | 32.78% | 54.610% | 21.82% | 8.11E−06 | 0.000297 |
| LMAN1 | ENST00000587918 | 9.59% | 4.30% | 5.28% | ENST00000251047 | 83.10% | 94.57% | 11.47% | 1.05E−05 | 0.000375 |
| LINC00861 | ENST00000651667 | 25.00% | 0.00% | 25.00% | ENST00000500989 | 60.00% | 87.78% | 27.78% | 1.14E−05 | 0.000389 |
| PRDM1 | ENST00000369096 | 66.15% | 46.23% | 19.91% | ENST00000481163 | 16.36% | 29.27% | 12.91% | 1.15E−05 | 0.000389 |
| SEC61B | ENST00000481573 | 31.52% | 11.40% | 20.12% | ENST00000223641 | 67.96% | 88.18% | 20.22% | 1.45E−05 | 0.000478 |
| DENND1B | ENST00000468589 | 24.64% | 8.04% | 16.60% | ENST00000620048 | 7.83% | 43.720% | 35.89% | 1.83E−05 | 0.000585 |
| TCERG1 | ENST00000509810 | 41.11% | 6.69% | 34.42% | ENST00000506524 | 25.56% | 36.14% | 10.58% | 2.26E−05 | 0.000707 |
| AC007780.1 | ENST00000592030 | 42.86% | 13.90% | 28.95% | ENST00000590353 | 46.43% | 84.49% | 38.06% | 3.62E−05 | 0.001077 |
| AKAP13 | ENST00000560957 | 16.13% | 2.91% | 13.22% | ENST00000560676 | 0.36% | 10.42% | 10.06% | 4.08E−05 | 0.001189 |
| PSMB10 | ENST00000570304 | 26.09% | 0.99% | 25.10% | ENST00000358514 | 69.56% | 92.08% | 22.51% | 5.67E−05 | 0.001612 |
| RBM5 | ENST00000437500 | 28.57% | 2.36% | 26.20% | ENST00000492472 | 0.00% | 12.21% | 12.21% | 5.89E−05 | 0.001612 |
| GTF2H2 | ENST00000521858 | 24.14% | 4.32% | 19.81% | ENST00000274400 | 24.14% | 38.36% | 14.22% | 9.73E−05 | 0.002597 |
| PSMA3 | ENST00000554207 | 32.00% | 6.63% | 25.37% | ENST00000557290 | 32.80% | 70.55% | 37.75% | 0.0001283 | 0.003355 |
| GOLGA8N | ENST00000569536 | 31.58% | 1.09% | 30.49% | ENST00000448387 | 68.42% | 95.65% | 27.23% | 0.0001321 | 0.003383 |
| SEPTIN6 | ENST00000481072 | 41.36% | 19.05% | 22.31% | ENST00000354416 | 46.97% | 62.83% | 15.85% | 0.0001807 | 0.004538 |
| CD74 | ENST00000353334 | 34.54% | 21.5% | 13.03% | ENST00000523836 | 9.10% | 27.27% | 18.17% | 0.0002092 | 0.00509 |
| SEC62 | ENST00000460513 | 26.83% | 10.50% | 16.33% | ENST00000337002 | 19.72% | 51.50% | 31.78% | 0.0002106 | 0.00509 |
| TAPBP | ENST00000475304 | 15.56% | 1.77% | 13.79% | ENST00000434618 | 73.33% | 93.04% | 19.71% | 0.0002495 | 0.005811 |
| CCNL1 | ENST00000467849 | 18.75% | 0.00% | 18.75% | ENST00000474539 | 38.39% | 60.15% | 21.76% | 0.0003674 | 0.008116 |
| MIR4435-2HG | ENST00000409569 | 29.16% | 9.61% | 19.55% | ENST00000603310 | 4.47% | 12.450% | 7.98% | 0.0003869 | 0.008401 |
| YARS | ENST00000478828 | 31.58% | 7.14% | 24.44% | ENST00000373477 | 17.08% | 60.32% | 43.23% | 0.0004211 | 0.008991 |
| SON | ENST00000429093 | 15.91% | 1.85% | 14.06% | ENST00000381679 | 14.80% | 30.30% | 15.50% | 0.000492 | 0.010332 |
| USP48 | ENST00000527823 | 13.74% | 0.00% | 13.74% | ENST00000464577 | 2.20% | 12.38% | 10.190% | 0.0005296 | 0.010943 |
| RABGAP1L | ENST00000486220 | 21.62% | 6.22% | 15.40% | ENST00000635248 | 5.41% | 29.75% | 24.340% | 0.0005583 | 0.011351 |
| GARS-DT | ENST00000663944 | 37.340% | 10.96% | 26.38% | ENST00000656943 | 3.03% | 16.04% | 13.01% | 0.0005826 | 0.011662 |
| CWF19L2 | ENST00000462890 | 17.65% | 0.000% | 17.65% | ENST00000282251 | 82.35% | 100.00% | 17.65% | 0.0006352 | 0.012519 |
| C18orf32 | ENST00000582392 | 11.11% | 0.000% | 11.11% | ENST00000318240 | 27.78% | 47.49% | 19.71% | 0.0006872 | 0.013138 |
| TOMM5 | ENST00000540941 | 23.080% | 2.86% | 20.22% | ENST00000321301 | 76.92% | 90.48% | 13.55% | 0.0006855 | 0.013138 |
| CXCR4 | ENST00000409817 | 32.93% | 11.37% | 21.56% | ENST00000241393 | 67.07% | 88.63% | 21.56% | 0.0007137 | 0.013401 |
| RBM47 | ENST00000510871 | 33.33% | 6.77% | 26.56% | ENST00000513473 | 4.17% | 27.47% | 23.30% | 0.0007218 | 0.013401 |
| PCM1 | ENST00000523540 | 20.00% | 2.050% | 17.95% | ENST00000327578 | 4.00% | 10.93% | 6.93% | 0.0007561 | 0.013836 |
| AL354740.1 | ENST00000593917 | 85.11% | 56.63% | 28.47% | ENST00000429998 | 12.96% | 39.70% | 26.74% | 0.0008305 | 0.014983 |
| RNF149 | ENST00000424632 | 19.64% | 5.34% | 14.30% | ENST00000295317 | 38.82% | 43.20% | 4.38% | 0.0011199 | 0.019652 |
| LNPEP | ENST00000395784 | 24.24% | 5.38% | 18.87% | ENST00000231368 | 59.69% | 81.60% | 21.91% | 0.0011647 | 0.019893 |
| UBE2B | ENST00000511807 | 21.62% | 2.80% | 18.82% | ENST00000499038 | 2.70% | 21.190% | 18.49% | 0.0011612 | 0.019893 |
| AC139887.2 | ENST00000503185 | 24.000% | 2.15% | 21.85% | ENST00000660016 | 0.00% | 16.49% | 16.49% | 0.0012154 | 0.02004 |
| MRPS31 | ENST00000498078 | 19.13% | 4.19% | 14.94% | ENST00000323563 | 64.80% | 86.43% | 21.630% | 0.0011911 | 0.02004 |
| SELENOS | ENST00000398226 | 23.86% | 11.51% | 12.35% | ENST00000526049 | 17.81% | 41.20% | 23.39% | 0.0012203 | 0.02004 |
| BHLHE40 | ENST00000256495 | 84.38% | 51.36% | 33.01% | ENST00000467610 | 15.63% | 46.65% | 31.03% | 0.0013279 | 0.021531 |
| SSR1 | ENST00000462112 | 50.00% | 9.72% | 40.27% | ENST00000244763 | 20.83% | 72.22% | 51.39% | 0.0013947 | 0.022111 |
| U2AF2 | ENST00000592867 | 23.81% | 2.56% | 21.25% | ENST00000587196 | 4.76% | 26.50% | 21.73% | 0.0013981 | 0.022111 |
| RRBP1 | ENST00000468428 | 52.11% | 20.28% | 31.82% | ENST00000470422 | 43.37% | 73.58% | 30.21% | 0.0016103 | 0.025156 |
| EDEM1 | ENST00000443790 | 15.97% | 0.81% | 15.16% | ENST00000256497 | 71.60% | 88.02% | 16.430% | 0.0016507 | 0.025476 |
| SMG6 | ENST00000572205 | 37.50% | 9.17% | 28.33% | ENST00000263073 | 37.50% | 75.83% | 38.33% | 0.0017729 | 0.027036 |
| IGHA2 | ENST00000390539 | 97.89% | 80.56% | 17.34% | ENST00000497872 | 2.11% | 19.44% | 17.340% | 0.001836 | 0.02767 |
| RUNX1 | ENST00000300305 | 76.59% | 33.14% | 43.45% | ENST00000469087 | 11.90% | 27.16% | 15.260% | 0.0019933 | 0.029691 |
| SEL1L3 | ENST00000513416 | 25.05% | 12.17% | 12.88% | ENST00000509290 | 0.00% | 15.49% | 15.49% | 0.0020425 | 0.030073 |
| MRPS5 | ENST00000461916 | 29.17% | 5.26% | 23.90% | ENST00000482821 | 0.00% | 13.32% | 13.320% | 0.0022998 | 0.032734 |
| STK4 | ENST00000372806 | 8.93% | 3.67% | 5.26% | ENST00000488618 | 57.46% | 67.74% | 10.28% | 0.0022829 | 0.032734 |
| C5orf56 | ENST00000378953 | 28.05% | 13.92% | 14.12% | ENST00000612967 | 31.71% | 48.33% | 16.62% | 0.0025365 | 0.035707 |
| LAMTOR2 | ENST00000368304 | 13.51% | 1.40% | 12.12% | ENST00000368305 | 83.78% | 94.42% | 10.63% | 0.0025733 | 0.03583 |
| HDLBP | ENST00000460826 | 21.43% | 5.43% | 16.00% | ENST00000425989 | 28.57% | 51.68% | 23.11% | 0.0028858 | 0.039327 |
| PRKCB | ENST00000498058 | 15.79% | 2.65% | 13.14% | ENST00000482000 | 0.00% | 11.26% | 11.26% | 0.0028758 | 0.039327 |
| SPDYE1 | ENST00000652520 | 40.54% | 17.97% | 22.57% | ENST00000258704 | 59.46% | 82.03% | 22.57% | 0.0029923 | 0.040349 |
| PFDN2 | ENST00000468311 | 17.24% | 2.19% | 15.05% | ENST00000368010 | 82.76% | 97.81% | 15.05% | 0.0030687 | 0.040948 |
| DOCK10 | ENST00000458608 | 42.31% | 8.52% | 33.79% | ENST00000489831 | 11.54% | 23.11% | 11.57% | 0.0032831 | 0.043358 |
| PLXNC1 | ENST00000549187 | 20.00% | 2.44% | 17.56% | ENST00000258526 | 37.35% | 58.34% | 20.99% | 0.0035745 | 0.046252 |
| U2SURP | ENST00000488497 | 38.90% | 6.31% | 32.59% | ENST00000480029 | 13.73% | 32.21% | 18.48% | 0.0036316 | 0.046521 |
| FUS | ENST00000254108 | 12.82% | 2.26% | 10.56% | ENST00000487509 | 48.72% | 59.83% | 11.11% | 0.0040139 | 0.049921 |
| ZNF518A | ENST00000442635 | 31.58% | 3.53% | 28.05% | ENST00000484770 | 47.37% | 66.42% | 19.05% | 0.0039841 | 0.049921 |

TABLE 12
Fibroblast cell specific DCI genes

| | | | | | | Prevalance | | | | |
|---|---|---|---|---|---|---|---|---|---|---|
| | | | | | | of | | Prevalance | | |
| | | Prevalance | Prevalance | | | non-fibroblast | | difference | | |
| | | of | of | | | cell | Prevalance | of | | |
| | Fibroblast | fibroblast | fibroblast | Prevalance | | preferred | of | non- | | |
| | cell | cell preferred | cell preferred | difference | Non-fibroblast | isoform | non-fibroblast cell | fibroblast | | |
| | preferred | isoform in | isoform in | of fibroblast | cell | in | preferred isoform | cell | | FDR |
| Gene | isoform | fibroblast | non-fibroblast | cell preferred | preferred isoform | fibroblast | in non-fibroblast | preferred | | adj |
| Name | ID | cells | cells | isoform | ID | cells | calle | isoform | P-values | P-values |
| MT2A | ENST00000562017 | 27.18% | 5.55% | 21.63% | ENST00000245185 | 56.32% | 88.14% | 31.82% | 3.06E−42 | 7.22E−39 |
| NBEAL1 | ENST00000449802 | 38.28% | 9.49% | 28.79% | ENST00000460355 | 35.33% | 84.04% | 48.710% | 3.92E−35 | 4.62E−32 |
| VAMP8 | ENST00000409760 | 44.44% | 1.24% | 43.20% | ENST00000263864 | 55.56% | 95.36% | 39.79% | 7.74E−16 | 4.56E−13 |
| TYMP | ENST00000652237 | 25.00% | 0.00% | 25.00% | ENST00000487162 | 62.50% | 85.42% | 22.92% | 2.54E−15 | 1.20E−12 |
| SYNE2 | ENST00000441438 | 37.78% | 0.15% | 37.63% | ENST00000358025 | 4.58% | 11.61% | 7.04% | 3.69E−15 | 1.45E−12 |
| S100A10 | ENST00000368809 | 13.04% | 0.00% | 13.04% | ENST00000368811 | 86.96% | 99.36% | 12.400% | 2.90E−13 | 9.77E−11 |
| ITPR2 | ENST00000536627 | 33.33% | 0.38% | 32.96% | ENST00000381340 | 55.56% | 85.15% | 29.60% | 1.40E−11 | 4.11E−09 |
| FNDC3A | ENST00000492622 | 53.53% | 10.01% | 43.52% | ENST00000398316 | 6.04% | 26.90% | 20.86% | 1.50E−10 | 3.93E−08 |
| AKAP13 | ENST00000560676 | 44.88% | 7.85% | 37.02% | ENST00000560340 | 0.00% | 14.45% | 14.45% | 1.87E−09 | 4.01E−07 |
| NR4A2 | ENST00000339562 | 96.13% | 32.07% | 64.06% | ENST00000409572 | 3.85% | 62.44% | 58.59% | 1.78E−09 | 4.01E−07 |
| AC015712.2 | ENST00000560068 | 47.06% | 6.15% | 40.90% | ENST00000656756 | 28.32% | 93.85% | 65.53% | 2.39E−09 | 4.70E−07 |
| GOT1 | ENST00000471741 | 35.71% | 2.19% | 33.52% | ENST00000370508 | 64.29% | 97.81% | 33.52% | 7.60E−09 | 1.24E−06 |
| NAMPT | ENST00000393618 | 17.65% | 3.32% | 14.32% | ENST00000486949 | 8.82% | 20.59% | 11.77% | 7.11E−09 | 1.24E−06 |
| SERPINB6 | ENST00000644828 | 33.33% | 1.33% | 32.01% | ENST00000644178 | 33.33% | 67.07% | 33.74% | 7.91E−09 | 1.24E−06 |
| NUTM2A-AS1 | ENST00000433530 | 25.00% | 0.89% | 24.11% | ENST00000660726 | 0.00% | 8.30% | 8.30% | 4.10E−08 | 6.04E−06 |
| PHKA2 | ENST00000486231 | 33.33% | 0.00% | 33.33% | ENST00000379942 | 55.56% | 96.47% | 40.92% | 5.32E−08 | 7.38E−06 |
| ARHGAP15 | ENST00000548929 | 31.25% | 0.00% | 31.250% | ENST00000409869 | 0.00% | 19.64% | 19.640% | 6.32E−08 | 7.90E−06 |
| C15orf48 | ENST00000558632 | 46.67% | 8.83% | 37.830% | ENST00000396650 | 36.66% | 58.78% | 22.11% | 6.37E−08 | 7.90E−06 |
| PAPPA2 | ENST00000367661 | 62.76% | 13.68% | 49.080% | ENST00000367662 | 33.24% | 81.96% | 48.72% | 7.79E−08 | 9.18E−06 |
| SASH1 | ENST00000637729 | 20.69% | 0.84% | 19.85% | ENST00000470750 | 0.00% | 17.65% | 17.65% | 8.94E−08 | 1.00E−05 |
| ST3GAL6 | ENST00000495502 | 54.55% | 2.05% | 52.49% | ENST00000477899 | 18.18% | 55.46% | 37.27% | 1.11E−07 | 1.19E−05 |
| COL4A1 | ENST00000647632 | 13.66% | 3.92% | 9.74% | ENST00000375820 | 47.70% | 64.53% | 16.83% | 1.34E−07 | 1.37E−05 |
| Z93930.2 | ENST00000458080 | 50.00% | 1.09% | 48.91% | ENST00000585003 | 50.00% | 89.13% | 39.13% | 1.55E−07 | 1.53E−05 |
| RBX1 | ENST00000476110 | 12.500% | 0.00% | 12.500% | ENST00000216225 | 87.50% | 94.44% | 6.94% | 2.40E−07 | 2.26E−05 |
| UBE2L3 | ENST00000545681 | 25.00% | 0.00% | 25.00% | ENST00000342192 | 75.00% | 98.42% | 23.42% | 4.54E−07 | 4.12E−05 |
| SELENOS | ENST00000527833 | 16.67% | 0.42% | 16.25% | ENST00000534014 | 8.33% | 39.53% | 31.19% | 7.75E−07 | 6.77E−05 |
| ERO1A | ENST00000556223 | 21.35% | 1.32% | 20.03% | ENST00000395686 | 48.24% | 74.83% | 26.59% | 8.96E−07 | 7.55E−05 |
| ARHGAP26 | ENST00000469131 | 24.49% | 0.00% | 24.49% | ENST00000646213 | 10.20% | 39.66% | 29.45% | 1.30E−06 | 0.00011 |
| MYO9A | ENST00000569314 | 20.00% | 0.00% | 20.00% | ENST00000356056 | 7.49% | 30.80% | 23.31% | 1.51E−06 | 0.00012 |
| RORA | ENST00000561093 | 21.74% | 3.16% | 18.58% | ENST00000335670 | 30.43% | 52.28% | 21.85% | 1.60E−06 | 0.00012 |
| RABGAP1 | ENST00000480054 | 19.81% | 1.29% | 18.52% | ENST00000456584 | 4.16% | 13.88% | 9.72% | 1.76E−06 | 0.00013 |
| N4BP2L2 | ENST00000512755 | 54.59% | 45.19% | 9.41% | ENST00000503814 | 8.08% | 26.51% | 18.42% | 2.81E−06 | 0.0002 |
| ARL10 | ENST00000507151 | 30.00% | 0.000% | 30.00% | ENST00000310389 | 20.000% | 47.19% | 27.19% | 3.12E−06 | 0.00022 |
| FAAP20 | ENST00000420964 | 26.67% | 2.01% | 24.65% | ENST00000378546 | 0.00% | 45.58% | 45.58% | 3.69E−06 | 0.00025 |
| CD59 | ENST00000528987 | 44.44% | 2.49% | 41.96% | ENST00000642928 | 11.11% | 67.64% | 56.53% | 4.36E−06 | 0.00029 |
| LIMS1 | ENST00000434274 | 31.58% | 9.79% | 21.79% | ENST00000393310 | 0.48% | 21.00% | 20.52% | 5.42E−06 | 0.00035 |
| CD44 | ENST00000532339 | 23.08% | 0.00% | 23.08% | ENST00000525209 | 0.00% | 13.69% | 13.69% | 5.81E−06 | 0.00035 |
| PDK1 | ENST00000466437 | 10.53% | 0.00% | 10.53% | ENST00000392571 | 0.00% | 7.62% | 7.62% | 5.75E−06 | 0.00035 |
| PCNX1 | ENST00000554707 | 25.00% | 1.23% | 23.77% | ENST00000554879 | 5.00% | 18.22% | 13.22% | 6.77E−06 | 0.0004 |
| ARHGAP24 | ENST00000503917 | 30.95% | 5.44% | 25.51% | ENST00000509709 | 4.76% | 26.76% | 22.00% | 7.29E−06 | 0.00042 |
| MRPS24 | ENST00000418740 | 42.86% | 2.83% | 40.030% | ENST00000317534 | 57.14% | 94.99% | 37.85% | 7.58E−06 | 0.00043 |
| COL4A2 | ENST00000649101 | 25.47% | 15.90% | 9.57% | ENST00000650225 | 9.33% | 21.13% | 11.80% | 9.84E−06 | 0.00054 |
| ADGRF5 | ENST00000265417 | 82.01% | 31.60% | 50.41% | ENST00000283296 | 9.99% | 60.53% | 50.54% | 1.75E−05 | 0.00094 |
| FKBP2 | ENST00000449942 | 16.13% | 0.64% | 15.49% | ENST00000541388 | 0.00% | 9.73% | 9.73% | 1.94E−05 | 0.00101 |
| PHKA2-AS1 | ENST00000452900 | 44.44% | 1.43% | 43.02% | ENST00000654006 | 55.56% | 98.57% | 43.02% | 2.03E−05 | 0.00104 |
| C1orf56 | ENST00000473308 | 45.45% | 4.26% | 41.20% | ENST00000368926 | 54.55% | 91.49% | 36.94% | 2.14E−05 | 0.00107 |
| ABCA6 | ENST00000590645 | 28.03% | 0.00% | 28.03% | ENST00000284425 | 41.48% | 68.83% | 27.35% | 2.27E−05 | 0.00111 |
| CREB5 | ENST00000396298 | 30.00% | 13.12% | 16.88% | ENST00000357727 | 30.00% | 62.41% | 32.41% | 2.70E−05 | 0.00128 |
| RRBP1 | ENST00000620641 | 21.43% | 0.00% | 21.43% | ENST00000468428 | 21.43% | 35.29% | 13.86% | 2.72E−05 | 0.00128 |
| EHMT1 | ENST00000637335 | 10.71% | 0.57% | 10.14% | ENST00000495657 | 30.060% | 66.50% | 36.44% | 3.05E−05 | 0.00141 |
| PNN | ENST00000554117 | 5.26% | 0.00% | 5.26% | ENST00000216832 | 89.47% | 99.58% | 10.10% | 3.16E−05 | 0.00143 |
| RUNX1 | ENST00000358356 | 37.11% | 1.44% | 35.67% | ENST00000300305 | 14.290% | 52.30% | 38.02% | 3.31E−05 | 0.00147 |
| KCP | ENST00000616669 | 28.57% | 2.24% | 26.33% | ENST00000492679 | 57.14% | 86.49% | 29.34% | 4.12E−05 | 0.0018 |
| AFF1 | ENST00000511996 | 37.50% | 1.41% | 36.09% | ENST00000511442 | 0.00% | 26.34% | 26.34% | 4.28E−05 | 0.00183 |
| PIK3R1 | ENST00000518813 | 33.33% | 3.59% | 29.75% | ENST00000521381 | 38.20% | 61.32% | 23.12% | 4.49E−05 | 0.00189 |
| GSK3B | ENST00000474830 | 26.32% | 3.05% | 23.26% | ENST00000264235 | 47.85% | 87.79% | 39.94% | 4.64E−05 | 0.00192 |
| TTC3 | ENST00000484047 | 30.43% | 2.81% | 27.63% | ENST00000399017 | 37.04% | 50.52% | 13.48% | 5.31E−05 | 0.00216 |
| SMARCB1 | ENST00000643421 | 11.11% | 0.45% | 10.66% | ENST00000644036 | 66.67% | 92.15% | 25.49% | 6.33E−05 | 0.00253 |
| YIF1A | ENST00000359461 | 33.33% | 12.24% | 21.09% | ENST00000376901 | 50.00% | 87.75% | 37.75% | 6.99E−05 | 0.00275 |
| CYB5A | ENST00000583418 | 19.36% | 5.98% | 13.38% | ENST00000340533 | 78.66% | 91.47% | 12.80% | 7.14E−05 | 0.00276 |
| SRP19 | ENST00000505459 | 46.67% | 22.66% | 24.01% | ENST00000445150 | 0.00% | 12.95% | 12.95% | 7.70E−05 | 0.00293 |
| TMEM147 | ENST00000392204 | 45.45% | 3.31% | 42.15% | ENST00000222284 | 54.55% | 91.73% | 37.19% | 7.93E−05 | 0.00297 |
| SMAD3 | ENST00000439724 | 22.22% | 0.00% | 22.22% | ENST00000327367 | 55.56% | 91.21% | 35.65% | 8.72E−05 | 0.00321 |
| NCOA2 | ENST00000520416 | 25.00% | 0.00% | 25.00% | ENST00000452400 | 53.46% | 83.68% | 30.22% | 8.95E−05 | 0.00325 |
| SRSF3 | ENST00000339436 | 37.50% | 7.03% | 30.47% | ENST00000373715 | 20.12% | 64.62% | 44.50% | 9.99E−05 | 0.00357 |
| GBP1 | ENST00000479889 | 11.48% | 0.00% | 11.48% | ENST00000370473 | 78.69% | 91.91% | 13.22% | 0.000102 | 0.0036 |
| TMED10 | ENST00000555085 | 17.65% | 3.36% | 14.29% | ENST00000303575 | 58.82% | 85.90% | 27.08% | 0.000112 | 0.00387 |
| CALCOCO2 | ENST00000258947 | 70.59% | 20.99% | 49.60% | ENST00000510356 | 0.00% | 45.51% | 45.510% | 0.000119 | 0.00394 |
| MTREX | ENST00000508716 | 36.36% | 12.09% | 24.27% | ENST00000230640 | 27.13% | 73.35% | 46.22% | 0.000117 | 0.00394 |
| TNPO1 | ENST00000509030 | 18.75% | 1.20% | 17.55% | ENST00000337273 | 56.24% | 80.84% | 24.59% | 0.000118 | 0.00394 |
| RHOA | ENST00000265538 | 33.33% | 18.88% | 14.45% | ENST00000418115 | 53.33% | 71.33% | 18.00% | 0.000155 | 0.00499 |
| NOC3L | ENST00000461562 | 45.45% | 6.10% | 39.36% | ENST00000371361 | 30.68% | 78.05% | 47.37% | 0.000171 | 0.00545 |
| XRN1 | ENST00000467077 | 33.33% | 2.08% | 31.250% | ENST00000264951 | 40.00% | 53.28% | 13.28% | 0.000173 | 0.00545 |
| GTF2H2 | ENST00000523003 | 20.00% | 3.17% | 16.83% | ENST00000521942 | 12.00% | 26.19% | 14.19% | 0.000204 | 0.00634 |
| SEC61B | ENST00000481573 | 38.46% | 13.42% | 25.04% | ENST00000223641 | 61.54% | 86.10% | 24.56% | 0.000209 | 0.00639 |
| OAS3 | ENST00000549918 | 20.00% | 0.00% | 20.00% | ENST00000548514 | 0.00% | 34.69% | 34.69% | 0.000225 | 0.0068 |
| SEC61G | ENST00000450622 | 18.18% | 6.25% | 11.93% | ENST00000395535 | 77.27% | 93.20% | 15.940% | 0.000231 | 0.0069 |
| VPS8 | ENST00000460158 | 25.00% | 0.00% | 25.00% | ENST00000485024 | 0.00% | 29.66% | 29.66% | 0.000235 | 0.00692 |
| MIR29B2CHG | ENST00000637970 | 12.50% | 0.00% | 12.50% | ENST00000608023 | 87.50% | 96.76% | 9.26% | 0.00024 | 0.00699 |
| PPIA | ENST00000494484 | 18.90% | 5.05% | 13.85% | ENST00000468812 | 78.02% | 94.35% | 16.33% | 0.000253 | 0.00727 |
| DLC1 | ENST00000511869 | 34.13% | 0.00% | 34.13% | ENST00000276297 | 42.72% | 60.44% | 17.72% | 0.00026 | 0.00738 |
| PPA1 | ENST00000373230 | 30.00% | 0.54% | 29.460% | ENST00000610026 | 20.00% | 44.960% | 24.96% | 0.000272 | 0.00763 |
| TOMM70 | ENST00000483945 | 44.44% | 5.21% | 39.24% | ENST00000284320 | 55.56% | 88.54% | 32.99% | 0.000277 | 0.00767 |
| BOLA2B | ENST00000565525 | 15.38% | 0.49% | 14.89% | ENST00000567436 | 0.00% | 10.84% | 10.84% | 0.000284 | 0.0078 |
| AC011447.3 | ENST00000592022 | 18.18% | 0.57% | 17.61% | ENST00000657925 | 72.73% | 94.89% | 22.160% | 0.000293 | 0.00786 |
| STX12 | ENST00000472285 | 31.58% | 12.500% | 19.08% | ENST00000373943 | 34.94% | 59.17% | 24.22% | 0.00029 | 0.00786 |
| IGFBP7 | ENST00000514062 | 15.97% | 5.42% | 10.55% | ENST00000295666 | 72.44% | 90.25% | 17.81% | 0.000302 | 0.00791 |
| KLHL28 | ENST00000556239 | 18.90% | 0.00% | 18.90% | ENST00000396128 | 37.50% | 47.64% | 10.14% | 0.000314 | 0.00813 |
| SPATA1 | ENST00000490879 | 57.14% | 37.21% | 19.93% | ENST00000460286 | 28.57% | 49.61% | 21.04% | 0.000372 | 0.00943 |
| RGS12 | ENST00000502947 | 25.00% | 0.00% | 25.00% | ENST00000382788 | 0.00% | 44.52% | 44.52% | 0.000387 | 0.0097 |
| RHOT1 | ENST00000652713 | 37.50% | 0.00% | 37.50% | ENST00000581031 | 0.00% | 21.13% | 21.13% | 0.000391 | 0.00972 |
| FAM91A1 | ENST00000519721 | 19.85% | 0.79% | 19.05% | ENST00000520246 | 0.00% | 12.32% | 12.32% | 0.000497 | 0.01221 |
| NUP58 | ENST00000495460 | 16.67% | 2.65% | 14.02% | ENST00000463407 | 4.17% | 22.93% | 18.77% | 0.000504 | 0.01225 |
| TAGLN | ENST00000533863 | 42.86% | 1.45% | 41.41% | ENST00000392951 | 57.14% | 95.65% | 38.51% | 0.00054 | 0.01299 |
| ANPEP | ENST00000558740 | 40.00% | 1.91% | 38.09% | ENST00000300060 | 60.00% | 88.59% | 28.59% | 0.000592 | 0.0141 |
| AOPEP | ENST00000473778 | 54.55% | 13.33% | 41.21% | ENST00000375315 | 9.09% | 28.67% | 19.58% | 0.000629 | 0.01484 |
| SNRPF | ENST00000549580 | 27.27% | 2.70% | 24.57% | ENST00000266735 | 63.64% | 94.59% | 30.96% | 0.000641 | 0.01496 |
| SMPD4 | ENST00000433118 | 9.52% | 1.05% | 8.48% | ENST00000409031 | 88.09% | 98.95% | 10.86% | 0.000715 | 0.0162 |
| PSME4 | ENST00000488687 | 69.23% | 41.33% | 27.90% | ENST00000404125 | 0.00% | 44.78% | 44.78% | 0.00074 | 0.01662 |
| PNKD | ENST00000483797 | 11.11% | 0.00% | 11.11% | ENST00000258362 | 0.00% | 5.24% | 5.24% | 0.000806 | 0.01776 |
| TNFAIP8 | ENST00000504771 | 45.45% | 8.11% | 37.35% | ENST00000513374 | 36.360% | 70.93% | 34.57% | 0.000832 | 0.01817 |
| IFIT3 | ENST00000371811 | 90.38% | 50.68% | 39.70% | ENST00000371818 | 9.62% | 49.32% | 39.70% | 0.000865 | 0.01871 |
| MICOS13 | ENST00000587950 | 17.650% | 2.170% | 15.48% | ENST00000309324 | 82.35% | 95.04% | 12.69% | 0.000962 | 0.02062 |
| MIS18BP1 | ENST00000451174 | 28.57% | 12.57% | 16.01% | ENST00000469020 | 0.00% | 14.20% | 14.20% | 0.000971 | 0.02063 |
| C5orf56 | ENST00000378947 | 31.82% | 13.56% | 18.25% | ENST00000612967 | 31.82% | 45.67% | 13.85% | 0.001042 | 0.02175 |
| LAMTOR2 | ENST00000368302 | 15.79% | 1.290% | 14.500% | ENST00000368305 | 78.95% | 93.99% | 15.040% | 0.001056 | 0.02184 |
| AC245297.3 | ENST00000668239 | 12.00% | 1.17% | 10.83% | ENST00000619653 | 65.33% | 74.14% | 8.81% | 0.001118 | 0.02279 |
| LSM1 | ENST00000523511 | 28.57% | 0.000% | 28.57% | ENST00000311351 | 28.57% | 74.42% | 45.85% | 0.001121 | 0.02279 |
| RNF216 | ENST00000476345 | 33.33% | 2.27% | 31.06% | ENST00000425013 | 33.33% | 65.74% | 32.410% | 0.001154 | 0.02315 |
| STAT6 | ENST00000556155 | 46.67% | 1.46% | 45.20% | ENST00000557781 | 23.33% | 67.02% | 43.69% | 0.001169 | 0.02315 |
| CHCHD3 | ENST00000262570 | 24.03% | 6.45% | 17.58% | ENST00000457942 | 69.23% | 87.14% | 17.91% | 0.001202 | 0.02361 |
| DKK3 | ENST00000528188 | 25.00% | 0.00% | 25.00% | ENST00000396505 | 75.00% | 96.08% | 21.08% | 0.001235 | 0.02407 |
| ABCE1 | ENST00000510321 | 64.29% | 30.85% | 33.44% | ENST00000296577 | 2.06% | 64.89% | 62.83% | 0.001372 | 0.0263 |
| DST | ENST00000487754 | 30.43% | 13.93% | 16.51% | ENST00000340834 | 18.48% | 33.69% | 15.21% | 0.001437 | 0.02732 |
| SUPT5H | ENST00000598520 | 72.73% | 12.630% | 60.10% | ENST00000599335 | 18.18% | 46.32% | 28.130% | 0.001452 | 0.02739 |
| HECTD1 | ENST00000611816 | 37.50% | 4.80% | 32.70% | ENST00000399332 | 18.75% | 28.57% | 9.82% | 0.00152 | 0.02845 |
| DENR | ENST00000539463 | 52.22% | 14.84% | 37.38% | ENST00000537955 | 0.000% | 30.84% | 30.84% | 0.001598 | 0.02943 |
| TENM4 | ENST00000529798 | 22.22% | 0.00% | 22.22% | ENST00000278550 | 66.77% | 96.49% | 29.73% | 0.001597 | 0.02943 |
| 7-Mar | ENST00000421037 | 17.62% | 0.00% | 17.62% | ENST00000259050 | 0.58% | 17.85% | 17.27% | 0.001761 | 0.03195 |
| QSOX1 | ENST00000367600 | 43.10% | 20.41% | 22.69% | ENST00000367602 | 50.00% | 78.50% | 28.51% | 0.001898 | 0.03391 |
| HNRNPA2B1 | ENST00000356674 | 23.28% | 7.28% | 16.00% | ENST00000463181 | 18.45% | 36.49% | 18.05% | 0.001963 | 0.03455 |
| DNAJC10 | ENST00000491074 | 50.00% | 3.42% | 46.58% | ENST00000650903 | 0.00% | 19.44% | 19.44% | 0.002048 | 0.03543 |
| H2AFJ | ENST00000501744 | 22.22% | 3.35% | 18.870% | ENST00000544848 | 77.78% | 96.65% | 18.87% | 0.002063 | 0.03543 |
| KMT2E | ENST00000257745 | 30.30% | 6.02% | 24.27% | ENST00000476671 | 26.92% | 38.73% | 11.80% | 0.002073 | 0.03543 |
| SENP6 | ENST00000493959 | 31.25% | 4.05% | 27.20% | ENST00000424947 | 6.25% | 16.89% | 10.64% | 0.002051 | 0.03543 |
| SLC20A1 | ENST00000492076 | 41.43% | 16.190% | 25.24% | ENST00000272542 | 0.03% | 33.870% | 33.840% | 0.002187 | 0.0371 |
| HMCN1 | ENST00000485744 | 28.57% | 2.29% | 26.280% | ENST00000271588 | 71.43% | 97.71% | 26.28% | 0.002213 | 0.03727 |
| NBPF15 | ENST00000577412 | 20.41% | 5.47% | 14.95% | ENST00000584793 | 56.86% | 81.58% | 24.71% | 0.002257 | 0.03775 |
| CBWD5 | ENST00000485088 | 22.58% | 7.38% | 15.21% | ENST00000468198 | 14.84% | 24.38% | 9.54% | 0.002403 | 0.03971 |
| UVRAG | ENST00000528264 | 25.00% | 0.75% | 24.25% | ENST00000356136 | 62.50% | 81.09% | 18.59% | 0.002408 | 0.03971 |
| SNAPC3 | ENST00000461041 | 25.00% | 3.57% | 21.43% | ENST00000380821 | 28.66% | 76.79% | 48.13% | 0.002556 | 0.04185 |
| ZNF431 | ENST00000598331 | 28.57% | 6.32% | 22.26% | ENST00000311048 | 57.39% | 90.52% | 33.13% | 0.002607 | 0.04239 |
| DLD | ENST00000489184 | 14.29% | 0.00% | 14.29% | ENST00000205402 | 35.71% | 58.55% | 22.84% | 0.002766 | 0.04438 |
| ATAD2B | ENST00000474583 | 42.86% | 4.900% | 37.96% | ENST00000238789 | 16.06% | 54.19% | 38.13% | 0.002983 | 0.04739 |
| SYNE1 | ENST00000409694 | 28.29% | 2.50% | 25.80% | ENST00000478916 | 27.35% | 60.49% | 33.14% | 0.002994 | 0.04739 |
| IRF2 | ENST00000509274 | 37.50% | 1.65% | 35.85% | ENST00000512020 | 0.00% | 27.95% | 27.95% | 0.003143 | 0.0494 |
| RIC1 | ENST00000251879 | 32.50% | 9.23% | 23.27% | ENST00000276898 | 38.75% | 85.51% | 46.75% | 0.003194 | 0.04987 |

TABLE 13
Consensus filtering of RCC1 variants

| | | Minimal cellular prevalence across all cells | | | | | | |
|---|---|---|---|---|---|---|---|---|
| SNV counts past | | 0% (1 | 0.1% (3 | 0.2% (6 | 0.4% (13 | 0.8% (27 | 1% (34 | 5% (173 |
| filtering | | cell) | cells) | cells) | cells) | cells) | cells) | cells) |
| Minimal | 1 | 3,001,137 | 249,149 | 86,212 | 26,895 | 9,146 | 6,632 | 1,004 |
| consensus | 2 | 2,801,998 | 240,527 | 83,188 | 25,939 | 8,805 | 6,390* | 980 |
| reads | 3 | 556,867 | 43,562 | 15,399 | 5,960 | 2,951 | 2,399 | 568 |
| supporting | 4 | 121,169 | 11,979 | 5,548 | 2,855 | 1,678 | 1,382 | 383 |
| | 5 | 35,991 | 6,464 | 3,424 | 1,948 | 1,124 | 954 | 296 |
| | 6 | 17,311 | 4,639 | 2,634 | 1,472 | 870 | 739 | 245 |

*default

TABLE 14
Expanded mutations in tumor cells

| | | | | REF_tumor | REF_normal | ALT_tumor | ALT_normal | FDR | diff_ALT_cellular |
|---|---|---|---|---|---|---|---|---|---|
| | | | | cell | cell | cell | cell | adj | prevalence_tumor vs |
| CHR | POS | REF | ALT | numbers | numbers | numbers | numbers | P-values | normal |
| chr2 | 8.8E+07 | T | G | 21 | 41 | 142 | 17 | 3.33E−15 | 0.578062196 |
| chr6 | 3.1E+07 | A | T, C | 47 | 171 | 80 | 30 | 2.09E−17 | 0.480667528 |
| chr6 | 3.1E+07 | C | T | 31 | 156 | 303 | 117 | 3.77E−34 | 0.4786142 |
| chr6 | 3.1E+07 | T | C | 29 | 155 | 311 | 120 | 4.25E−35 | 0.478342246 |
| chr6 | 3.1E+07 | C | G | 40 | 155 | 295 | 110 | 4.90E−31 | 0.465502675 |
| chr3 | 1.5E+07 | G | A | 13 | 40 | 275 | 39 | 1.54E−22 | 0.461190225 |
| chr17 | 8376238 | A | T | 3 | 14 | 88 | 15 | 9.78E−08 | 0.449791588 |
| chr7 | 1.4E+08 | G | A | 355 | 240 | 1014 | 101 | 2.75E−50 | 0.444498949 |
| chr2 | 1.4E+07 | G | A | 410 | 158 | 885 | 50 | 8.46E−32 | 0.443013068 |
| chr6 | 3.1E+07 | C | T | 41 | 152 | 286 | 117 | 8.29E−28 | 0.439673499 |
| chr2 | 1.4E+07 | G | A, T | 496 | 176 | 817 | 40 | 1.28E−30 | 0.437053962 |
| chr6 | 3.1E+07 | T | C | 69 | 172 | 241 | 92 | 3.90E−23 | 0.428934506 |
| chr6 | 3.1E+07 | C | T | 36 | 145 | 309 | 129 | 2.17E−28 | 0.424849254 |
| KI270467.1 | 3371 | C | T, A | 464 | 148 | 708 | 33 | 3.55E−24 | 0.421775121 |
| KI270467.1 | 2940 | A | G, C | 286 | 90 | 792 | 41 | 7.83E−21 | 0.421716778 |
| chr8 | 4.2E+07 | C | T | 259 | 68 | 487 | 21 | 9.56E−13 | 0.416859957 |
| KI270467.1 | 3002 | T | C | 420 | 106 | 684 | 27 | 2.97E−18 | 0.416557699 |
| KI270467.1 | 3210 | T | A, G | 384 | 124 | 787 | 43 | 6.40E−23 | 0.41459012 |
| KI270467.1 | 2905 | A | T, G | 396 | 112 | 715 | 34 | 1.61E−19 | 0.410687644 |
| KI270467.1 | 2420 | T | A | 303 | 155 | 937 | 83 | 8.53E−33 | 0.406905665 |
| chr7 | 1.3E+08 | A | G, C | 308 | 275 | 973 | 151 | 1.98E−49 | 0.405102748 |
| KI270467.1 | 2835 | T | G, A | 317 | 108 | 801 | 49 | 8.88E−22 | 0.40435605 |
| chr3 | 3.8E+07 | C | T | 13 | 54 | 254 | 66 | 7.09E−20 | 0.401310861 |
| KI270467.1 | 2382 | G | T, A | 302 | 154 | 938 | 85 | 4.86E−32 | 0.400803077 |
| chr2 | 1.4E+07 | C | T | 336 | 131 | 966 | 68 | 1.42E−27 | 0.400226941 |
| KI270467.1 | 2279 | A | G | 323 | 167 | 918 | 88 | 8.46E−32 | 0.394627988 |
| KI270467.1 | 2820 | G | A, T, C | 298 | 102 | 814 | 52 | 6.12E−21 | 0.394352051 |
| KI270467.1 | 2074 | A | G, T | 380 | 181 | 854 | 77 | 4.86E−30 | 0.393608734 |
| chr2 | 1.4E+07 | G | A | 506 | 153 | 792 | 43 | 1.03E−22 | 0.390781736 |
| KI270467.1 | 2217 | T | A, G | 396 | 177 | 840 | 72 | 1.71E−28 | 0.390455024 |
| KI270467.1 | 2472 | A | G | 433 | 174 | 799 | 61 | 2.32E−26 | 0.388964493 |
| chr17 | 1.8E+07 | G | A | 6 | 20 | 85 | 24 | 2.70E−06 | 0.388611389 |
| KI270467.1 | 3221 | C | A | 410 | 121 | 762 | 43 | 1.52E−19 | 0.387975527 |
| KI270467.1 | 2253 | C | A, G | 324 | 161 | 901 | 86 | 9.50E−30 | 0.387332066 |
| KI270467.1 | 2898 | G | C, T, A | 415 | 119 | 698 | 38 | 3.64E−18 | 0.385095656 |
| KI270467.1 | 3082 | G | A | 389 | 98 | 734 | 36 | 2.72E−16 | 0.384949695 |
| chr6 | 3E+07 | C | G | 14 | 55 | 158 | 63 | 1.76E−12 | 0.384706346 |
| KI270467.1 | 3743 | G | A, T | 451 | 91 | 568 | 19 | 5.70E−13 | 0.384681952 |
| chr17 | 7626829 | G | C | 8 | 44 | 198 | 60 | 1.04E−15 | 0.384241972 |
| KI270467.1 | 2576 | A | T | 421 | 163 | 804 | 61 | 6.17E−25 | 0.384005102 |
| KI270467.1 | 2525 | A | C, T | 469 | 182 | 755 | 56 | 3.21E−25 | 0.381535948 |
| chr2 | 1.4E+07 | G | A | 502 | 127 | 772 | 37 | 1.60E−18 | 0.380355707 |
| KI270467.1 | 3368 | T | A | 519 | 151 | 659 | 33 | 6.77E−20 | 0.380074924 |
| KI270467.1 | 2841 | A | G | 431 | 117 | 688 | 36 | 3.36E−17 | 0.379540556 |
| chr6 | 3.1E+07 | G | C | 40 | 112 | 257 | 107 | 1.31E−18 | 0.37673539 |
| KI270467.1 | 2594 | T | A | 446 | 160 | 771 | 56 | 1.09E−22 | 0.374265802 |
| KI270467.1 | 3306 | T | C, G, A | 424 | 132 | 743 | 47 | 2.16E−19 | 0.374105403 |
| KI270467.1 | 2245 | T | C, A | 424 | 180 | 812 | 71 | 5.80E−26 | 0.374089403 |
| KI270467.1 | 1964 | T | G, A | 341 | 120 | 823 | 60 | 7.86E−21 | 0.37371134 |
| chr6 | 3E+07 | T | C | 13 | 53 | 117 | 59 | 2.47E−09 | 0.373214286 |
| KI270467.1 | 3252 | G | A | 339 | 114 | 847 | 59 | 2.09E−20 | 0.373124799 |
| KI270467.1 | 2927 | T | C, A | 462 | 104 | 617 | 26 | 3.62E−14 | 0.371825765 |
| chr3 | 3.4E+07 | C | T | 8 | 21 | 85 | 25 | 1.16E−05 | 0.370500234 |
| KI270467.1 | 3125 | C | T, A | 404 | 99 | 709 | 36 | 5.04E−15 | 0.370350404 |
| KI270467.1 | 3161 | G | A, C | 349 | 98 | 776 | 46 | 1.05E−16 | 0.370333333 |
| KI270467.1 | 2868 | G | A | 210 | 81 | 882 | 63 | 8.32E−21 | 0.370192308 |
| KI270467.1 | 3404 | G | T | 466 | 144 | 709 | 44 | 2.27E−19 | 0.369361702 |
| chr7 | 1.3E+08 | A | G | 531 | 334 | 748 | 92 | 5.94E−37 | 0.368869459 |
| KI270467.1 | 2292 | T | C | 334 | 165 | 910 | 95 | 8.12E−28 | 0.366126639 |
| KI270467.1 | 2338 | C | T | 162 | 122 | 1079 | 124 | 1.76E−37 | 0.365395072 |
| KI270467.1 | 2979 | T | C | 481 | 111 | 644 | 29 | 1.19E−14 | 0.365301587 |
| KI270467.1 | 2191 | A | C | 573 | 213 | 671 | 45 | 1.53E−24 | 0.364970463 |
| KI270467.1 | 3098 | C | T | 362 | 84 | 755 | 38 | 6.27E−14 | 0.364442227 |
| KI270467.1 | 3344 | C | G, T | 503 | 133 | 661 | 34 | 6.29E−17 | 0.364276601 |
| KI270467.1 | 3183 | G | T, C | 420 | 104 | 703 | 37 | 6.02E−15 | 0.363590433 |
| chr3 | 7E+07 | C | T | 5 | 14 | 190 | 22 | 4.65E−11 | 0.363247863 |
| KI270467.1 | 2442 | G | A | 553 | 194 | 679 | 45 | 9.92E−23 | 0.362851845 |
| KI270467.1 | 2165 | A | G | 276 | 148 | 966 | 106 | 8.46E−29 | 0.360454943 |
| KI270467.1 | 2296 | T | C, G | 342 | 162 | 913 | 94 | 1.39E−26 | 0.36030254 |
| chr6 | 4.4E+07 | C | T | 403 | 300 | 621 | 98 | 9.97E−32 | 0.360214157 |
| KI270467.1 | 2920 | C | T, G | 337 | 81 | 728 | 39 | 2.30E−13 | 0.358568075 |
| KI270467.1 | 2080 | T | C | 400 | 177 | 841 | 83 | 4.33E−25 | 0.358448522 |
| KI270467.1 | 2012 | T | C | 265 | 118 | 930 | 86 | 6.12E−24 | 0.35667405 |
| KI270467.1 | 2624 | C | A, T, G | 223 | 105 | 977 | 89 | 4.30E−25 | 0.35540378 |
| KI270467.1 | 3158 | T | A, C | 584 | 130 | 545 | 19 | 8.80E−15 | 0.355211299 |
| chr6 | 1.6E+08 | G | A | 5 | 15 | 237 | 25 | 1.43E−13 | 0.354338843 |
| chr7 | 1.4E+08 | C | T | 225 | 175 | 1148 | 163 | 4.37E−40 | 0.353876752 |
| chr7 | 1.4E+08 | C | T | 485 | 226 | 874 | 92 | 2.71E−28 | 0.353811765 |
| KI270467.1 | 2284 | A | G | 341 | 162 | 903 | 96 | 9.16E−26 | 0.353791221 |
| KI270467.1 | 3109 | C | T, A | 314 | 82 | 795 | 47 | 1.81E−14 | 0.352520953 |
| chrX | 7.4E+07 | T | C | 10 | 50 | 327 | 81 | 3.35E−22 | 0.352005799 |
| KI270467.1 | 2629 | G | T, A | 239 | 110 | 966 | 90 | 2.35E−24 | 0.351659751 |
| chr7 | 1.4E+08 | T | C | 237 | 183 | 1139 | 167 | 2.03E−39 | 0.350618771 |
| KI270467.1 | 3049 | T | G, A | 407 | 95 | 679 | 36 | 4.52E−13 | 0.350421042 |
| KI270467.1 | 1950 | T | C | 434 | 127 | 731 | 49 | 1.05E−16 | 0.34905872 |
| chr1 | 2.4E+08 | A | G | 174 | 44 | 224 | 12 | 1.78E−05 | 0.348528356 |
| KI270467.1 | 3184 | G | C, A, T | 588 | 124 | 531 | 18 | 1.15E−13 | 0.347770268 |
| chr6 | 3.1E+07 | G | C | 86 | 287 | 109 | 77 | 4.25E−15 | 0.347435897 |
| chr6 | 3.1E+07 | T | C | 85 | 281 | 110 | 78 | 7.58E−15 | 0.346832369 |
| KI270467.1 | 2955 | A | C | 444 | 97 | 634 | 31 | 2.97E−12 | 0.34593866 |
| chr4 | 1.9E+08 | C | T | 446 | 82 | 526 | 20 | 8.22E−10 | 0.345073832 |
| KI270467.1 | 1961 | T | C | 375 | 116 | 776 | 57 | 1.05E−16 | 0.344716582 |
| KI270467.1 | 2512 | A | T, G, C | 654 | 213 | 584 | 31 | 1.54E−21 | 0.344679414 |
| KI270467.1 | 2921 | A | G, C | 287 | 82 | 800 | 53 | 1.04E−14 | 0.343377969 |
| KI270467.1 | 3301 | G | A, T | 499 | 132 | 677 | 40 | 1.53E−15 | 0.343122133 |
| chr2 | 1.4E+07 | G | A, C | 397 | 79 | 776 | 37 | 9.08E−12 | 0.34258606 |
| KI270467.1 | 2339 | G | A, T | 503 | 172 | 717 | 56 | 1.42E−19 | 0.342090883 |
| KI270467.1 | 3300 | G | T, C | 476 | 123 | 677 | 40 | 8.91E−15 | 0.341765147 |
| KI270467.1 | 3180 | T | C, A | 523 | 115 | 601 | 28 | 6.85E−13 | 0.338893313 |
| KI270467.1 | 3436 | G | A, T | 549 | 139 | 609 | 32 | 4.99E−15 | 0.338772233 |
| KI270467.1 | 2171 | T | A | 270 | 141 | 971 | 113 | 8.00E−26 | 0.337551632 |
| KI270467.1 | 2622 | C | T | 154 | 94 | 1050 | 108 | 8.12E−29 | 0.337439558 |
| KI270467.1 | 3329 | C | T, G, A | 475 | 124 | 684 | 42 | 1.19E−14 | 0.337151886 |
| KI270467.1 | 2996 | G | A, T, C | 243 | 74 | 864 | 59 | 1.99E−15 | 0.336878782 |
| KI270467.1 | 2048 | G | T, C | 260 | 125 | 951 | 102 | 1.19E−23 | 0.335962197 |
| KI270467.1 | 2449 | T | G, A | 617 | 187 | 610 | 36 | 1.05E−18 | 0.335712537 |
| KI270467.1 | 2175 | G | T | 329 | 153 | 912 | 102 | 2.19E−23 | 0.334891217 |
| KI270467.1 | 3134 | G | T, A | 570 | 123 | 551 | 23 | 7.26E−13 | 0.333991177 |
| KI270467.1 | 2034 | A | G | 256 | 133 | 969 | 112 | 4.30E−25 | 0.333877551 |
| KI270467.1 | 3257 | A | T | 545 | 134 | 618 | 33 | 2.43E−14 | 0.33377956 |
| KI270467.1 | 3309 | T | C, G | 481 | 125 | 676 | 42 | 2.43E−14 | 0.332772657 |
| KI270467.1 | 2287 | C | T, G | 227 | 131 | 1008 | 123 | 3.55E−27 | 0.331942363 |
| chr3 | 4984576 | A | G | 8 | 26 | 167 | 43 | 8.60E−10 | 0.331097308 |
| KI270467.1 | 3464 | G | T | 613 | 143 | 520 | 21 | 3.25E−14 | 0.330909737 |
| KI270467.1 | 2610 | G | T, A | 503 | 153 | 705 | 52 | 9.40E−17 | 0.329950735 |
| KI270467.1 | 1954 | A | G, T | 388 | 116 | 776 | 59 | 2.04E−15 | 0.32952381 |
| KI270467.1 | 2294 | T | C, A | 266 | 134 | 967 | 112 | 3.61E−24 | 0.328981465 |
| chr11 | 6.6E+07 | C | T | 337 | 640 | 1107 | 499 | 1.39E−61 | 0.328516899 |
| KI270467.1 | 2121 | A | C, G | 190 | 122 | 1045 | 131 | 4.35E−29 | 0.328367285 |
| KI270467.1 | 1966 | T | G, A | 512 | 134 | 647 | 40 | 2.19E−14 | 0.328354804 |
| chr2 | 1.4E+07 | C | T | 195 | 97 | 1120 | 107 | 5.53E−26 | 0.327201223 |
| KI270467.1 | 2152 | T | C, G | 703 | 227 | 545 | 28 | 9.47E−21 | 0.326894796 |
| KI270467.1 | 3243 | A | G, C | 635 | 149 | 553 | 24 | 1.58E−14 | 0.326759892 |
| KI270467.1 | 2133 | T | C, G, A | 674 | 209 | 564 | 32 | 5.65E−19 | 0.322793423 |
| KI270467.1 | 2025 | G | T, A | 262 | 116 | 945 | 99 | 5.95E−21 | 0.322467775 |
| chr11 | 6.5E+07 | C | T | 507 | 355 | 838 | 153 | 1.02E−32 | 0.321867225 |
| chr11 | 6.5E+07 | G | A | 446 | 396 | 935 | 219 | 3.51E−38 | 0.320948058 |
| KI270467.1 | 2565 | G | A | 157 | 97 | 1064 | 119 | 1.31E−27 | 0.320490945 |
| KI270467.1 | 2015 | T | C, G | 262 | 111 | 937 | 95 | 5.63E−20 | 0.320319522 |
| KI270467.1 | 1945 | C | G | 505 | 116 | 622 | 35 | 4.03E−12 | 0.32011964 |
| KI270467.1 | 3078 | T | C | 602 | 120 | 527 | 21 | 1.80E−11 | 0.317848595 |
| chr11 | 6.5E+07 | A | G | 601 | 437 | 795 | 147 | 1.82E−35 | 0.317771912 |
| chr11 | 6.5E+07 | A | G | 658 | 460 | 736 | 123 | 9.58E−36 | 0.316999343 |
| chr6 | 1.6E+08 | T | C | 8 | 14 | 234 | 26 | 4.89E−10 | 0.316942149 |
| KI270467.1 | 3061 | A | T | 457 | 94 | 636 | 34 | 2.47E−10 | 0.316259721 |
| chr17 | 7626953 | C | T | 20 | 55 | 239 | 85 | 5.87E−13 | 0.315637066 |
| chr3 | 4979718 | C | T | 29 | 63 | 290 | 92 | 1.81E−14 | 0.315542522 |
| KI270467.1 | 3418 | C | T | 591 | 144 | 573 | 31 | 1.74E−13 | 0.315125184 |
| KI270467.1 | 3448 | G | C | 661 | 148 | 496 | 19 | 1.89E−13 | 0.314922446 |
| KI270467.1 | 2626 | T | A, C | 595 | 166 | 617 | 40 | 2.06E−15 | 0.31490115 |
| KI270467.1 | 3074 | T | A, G | 587 | 117 | 540 | 23 | 3.97E−11 | 0.314862467 |
| chr9 | 1.4E+08 | T | C | 9 | 20 | 66 | 26 | 0.001296 | 0.314782609 |
| KI270467.1 | 3176 | A | G, T, C | 444 | 99 | 670 | 40 | 5.04E−11 | 0.313666482 |
| KI270467.1 | 2656 | A | G, T | 224 | 92 | 958 | 91 | 6.85E−19 | 0.313222934 |
| KI270467.1 | 3302 | G | T, A | 579 | 140 | 596 | 34 | 4.17E−13 | 0.311831744 |
| KI270467.1 | 3132 | T | G, C | 604 | 122 | 524 | 22 | 2.88E−11 | 0.311761229 |
| KI270467.1 | 3122 | A | G, T, C | 618 | 123 | 493 | 19 | 3.89E−11 | 0.309941558 |
| KI270467.1 | 3512 | A | T | 646 | 142 | 492 | 20 | 1.47E−12 | 0.308880644 |
| KI270467.1 | 3293 | T | C, G | 478 | 126 | 707 | 51 | 3.84E−13 | 0.308488879 |
| chr8 | 1.3E+08 | A | G | 20 | 48 | 157 | 66 | 3.48E−08 | 0.308058281 |
| KI270467.1 | 3761 | C | G, T | 502 | 79 | 473 | 17 | 1.44E−07 | 0.308044872 |
| KI270467.1 | 2375 | C | T, A, G | 621 | 201 | 621 | 48 | 2.81E−17 | 0.307228916 |
| KI270467.1 | 2791 | A | T, C | 505 | 97 | 516 | 24 | 3.46E−09 | 0.307039768 |
| chr7 | 1.3E+08 | G | A | 319 | 234 | 955 | 186 | 6.42E−29 | 0.306750392 |
| chr3 | 7E+07 | G | A | 2 | 8 | 137 | 17 | 5.72E−07 | 0.305611511 |
| KI270467.1 | 2908 | C | T, A, G | 545 | 112 | 557 | 28 | 2.16E−10 | 0.305444646 |
| chr12 | 9.7E+07 | T | C | 233 | 37 | 267 | 11 | 0.000699 | 0.304833333 |
| chr1 | 1.7E+08 | C | T | 215 | 52 | 241 | 15 | 4.80E−05 | 0.304628175 |
| KI270467.1 | 2170 | T | C | 635 | 204 | 606 | 46 | 4.14E−17 | 0.304315874 |
| chr11 | 6.5E+07 | C | T | 581 | 419 | 779 | 154 | 7.89E−32 | 0.30403321 |
| KI270467.1 | 2168 | T | C | 684 | 216 | 560 | 37 | 1.16E−17 | 0.303915712 |
| KI270467.1 | 1927 | C | A | 226 | 67 | 860 | 64 | 6.05E−13 | 0.303347251 |
| KI270467.1 | 3073 | G | C, A | 648 | 120 | 469 | 16 | 2.05E−10 | 0.302227605 |
| KI270467.1 | 2137 | C | T, A | 688 | 223 | 556 | 38 | 7.10E−18 | 0.301351468 |
| chr11 | 6.5E+07 | T | C | 177 | 297 | 1251 | 403 | 9.16E−52 | 0.300336134 |
| KI270467.1 | 3129 | A | C, T, G | 565 | 108 | 537 | 25 | 1.30E−09 | 0.299325901 |
| KI270467.1 | 3682 | A | T, G, C | 569 | 102 | 477 | 19 | 6.41E−09 | 0.298998151 |
| KI270467.1 | 2033 | T | G, C | 617 | 193 | 604 | 47 | 6.57E−16 | 0.298843161 |
| KI270467.1 | 2354 | T | G | 661 | 211 | 581 | 43 | 6.78E−17 | 0.298502542 |
| KI270467.1 | 2161 | T | A, G | 555 | 163 | 643 | 51 | 2.77E−14 | 0.298410123 |
| KI270467.1 | 3181 | T | C | 690 | 134 | 445 | 14 | 3.27E−11 | 0.29747589 |
| KI270467.1 | 2762 | A | G | 541 | 108 | 520 | 26 | 1.98E−09 | 0.296073825 |
| KI270467.1 | 3776 | T | A, G | 406 | 68 | 578 | 28 | 4.62E−07 | 0.295731707 |
| KI270467.1 | 2007 | G | A, T | 254 | 107 | 947 | 104 | 6.72E−18 | 0.29561858 |
| KI270467.1 | 2177 | A | G | 234 | 118 | 994 | 125 | 6.43E−21 | 0.295042962 |
| KI270467.1 | 3111 | T | C, A, G | 698 | 123 | 421 | 11 | 3.12E−10 | 0.294139223 |
| KI270467.1 | 2436 | T | C, A | 328 | 130 | 903 | 102 | 4.19E−17 | 0.293894787 |
| KI270467.1 | 3388 | A | C, G | 531 | 133 | 649 | 46 | 7.06E−12 | 0.29301676 |
| KI270467.1 | 3731 | A | T | 492 | 87 | 539 | 26 | 6.73E−08 | 0.292704909 |
| KI270467.1 | 2504 | G | T, A | 785 | 218 | 457 | 18 | 6.83E−17 | 0.291683725 |
| KI270467.1 | 2305 | C | A, G, T | 739 | 224 | 512 | 30 | 5.73E−17 | 0.291162346 |
| chr11 | 6.5E+07 | T | A | 531 | 394 | 840 | 187 | 1.23E−29 | 0.290832602 |
| KI270467.1 | 2544 | A | C, G, T | 158 | 89 | 1047 | 122 | 1.32E−22 | 0.290680616 |
| KI270467.1 | 3100 | G | A | 598 | 104 | 506 | 21 | 1.05E−08 | 0.290333333 |
| KI270467.1 | 3194 | G | T, A | 684 | 128 | 445 | 15 | 2.80E−10 | 0.289259014 |
| KI270467.1 | 3113 | C | G | 651 | 113 | 456 | 16 | 4.09E−09 | 0.287893111 |
| KI270467.1 | 2774 | A | G | 534 | 105 | 518 | 27 | 8.18E−09 | 0.287849983 |
| KI270466.1 | 997 | C | T | 282 | 49 | 470 | 25 | 2.33E−05 | 0.287162162 |
| KI270467.1 | 3096 | C | A | 558 | 98 | 562 | 27 | 2.52E−08 | 0.285785714 |
| KI270467.1 | 3469 | G | T, A, C | 697 | 150 | 450 | 18 | 1.47E−11 | 0.285184955 |
| KI270467.1 | 2793 | A | T | 510 | 98 | 512 | 27 | 3.27E−08 | 0.284978474 |
| KI270467.1 | 1957 | C | T, A | 173 | 71 | 966 | 92 | 2.28E−16 | 0.283695201 |
| chr6 | 3.1E+07 | G | A | 618 | 98 | 497 | 19 | 6.66E−08 | 0.283346748 |
| chr11 | 6.5E+07 | A | G | 453 | 376 | 930 | 240 | 4.83E−30 | 0.282840803 |
| KI270467.1 | 3543 | C | T, A | 632 | 125 | 478 | 22 | 1.35E−09 | 0.280970767 |
| KI270467.1 | 3848 | T | C, G | 333 | 65 | 651 | 40 | 2.60E−07 | 0.280632985 |
| KI270467.1 | 2767 | A | T | 514 | 104 | 546 | 32 | 1.56E−08 | 0.279800222 |
| KI270467.1 | 3890 | C | A | 298 | 41 | 545 | 24 | 0.000119 | 0.277269824 |
| KI270467.1 | 2780 | C | T, A | 428 | 83 | 603 | 37 | 1.44E−07 | 0.276535726 |
| KI270467.1 | 2471 | T | C | 749 | 211 | 485 | 28 | 8.80E−15 | 0.275875982 |
| chr9 | 3.6E+07 | A | G | 9 | 23 | 85 | 39 | 0.000515 | 0.275223061 |
| chr6 | 3E+07 | C | T | 17 | 57 | 392 | 123 | 1.81E−18 | 0.275101874 |
| KI270467.1 | 1873 | T | C, A | 362 | 46 | 581 | 24 | 9.45E−05 | 0.273261627 |
| KI270467.1 | 1175 | C | T | 258 | 27 | 411 | 14 | 0.005654 | 0.272886361 |
| KI270467.1 | 2138 | C | G, T | 625 | 188 | 602 | 53 | 2.99E−13 | 0.270710534 |
| chr11 | 6.5E+07 | A | G | 668 | 429 | 712 | 140 | 1.06E−25 | 0.269896335 |
| chr6 | 4031764 | A | G | 13 | 49 | 230 | 103 | 3.37E−11 | 0.268870479 |
| chr11 | 6.5E+07 | T | C, A, G | 680 | 413 | 654 | 119 | 4.64E−24 | 0.266570662 |
| chr11 | 6.5E+07 | G | A | 622 | 359 | 666 | 120 | 9.23E−22 | 0.266558825 |
| KI2704671 | 2450 | T | C, G, A | 757 | 205 | 477 | 28 | 1.24E−13 | 0.266376138 |
| KI2704671 | 3460 | A | T, C | 691 | 150 | 457 | 23 | 3.11E−10 | 0.265135647 |
| chr12 | 9.7E+07 | A | G | 244 | 35 | 264 | 12 | 0.005312 | 0.26436589 |
| chr11 | 6.6E+07 | C | T | 156 | 421 | 1294 | 711 | 3.95E−54 | 0.26432192 |
| KI270467.1 | 3537 | A | G | 706 | 141 | 418 | 17 | 1.12E−09 | 0.264291184 |
| KI270467.1 | 2760 | T | A | 585 | 110 | 477 | 25 | 8.82E−08 | 0.263967357 |
| KI270467.1 | 2315 | T | G, C | 712 | 219 | 546 | 45 | 4.04E−14 | 0.263567712 |
| chr6 | 3.3E+07 | C | A | 19 | 56 | 111 | 81 | 2.94E−05 | 0.262605278 |
| KI270467.1 | 3519 | C | T, A | 611 | 116 | 479 | 25 | 4.81E−08 | 0.262144577 |
| chr6 | 3.1E+07 | C | A | 76 | 39 | 1032 | 79 | 2.41E−18 | 0.261916417 |
| KI270466.1 | 407 | A | G, T | 228 | 73 | 801 | 78 | 1.41E−10 | 0.261869365 |
| KI270467.1 | 3259 | G | C, T | 592 | 126 | 566 | 37 | 6.53E−09 | 0.261779883 |
| KI270467.1 | 1933 | G | A, T | 207 | 61 | 897 | 75 | 8.54E−11 | 0.261029412 |
| KI270467.1 | 1932 | G | A, C | 461 | 91 | 628 | 42 | 2.01E−07 | 0.260886376 |
| chr6 | 3E+07 | A | T | 254 | 267 | 345 | 123 | 3.07E−14 | 0.260575318 |
| KI270467.1 | 2298 | T | C, A | 723 | 219 | 538 | 44 | 9.85E−14 | 0.259345139 |
| chr7 | 1.3E+08 | A | G | 832 | 390 | 448 | 39 | 7.45E−23 | 0.259090909 |
| chr7 | 1.3E+08 | A | G, T | 784 | 374 | 495 | 55 | 2.45E−21 | 0.258815982 |
| KI270467.1 | 1928 | C | A | 485 | 92 | 606 | 39 | 4.02E−07 | 0.257743789 |
| chr11 | 6.5E+07 | C | T | 561 | 338 | 739 | 153 | 1.99E−20 | 0.256852577 |
| KI270467.1 | 2863 | A | G | 696 | 130 | 413 | 17 | 1.41E−08 | 0.256761316 |
| KI270467.1 | 3351 | G | A, C, T | 770 | 159 | 410 | 16 | 2.30E−10 | 0.256029056 |
| chrX | 7.4E+07 | A | C | 10 | 26 | 364 | 66 | 1.93E−14 | 0.255870728 |
| chrX | 7.4E+07 | A | T | 5 | 18 | 162 | 45 | 3.42E−07 | 0.255774166 |
| KI270466.1 | 424 | C | T | 239 | 77 | 799 | 82 | 3.18E−10 | 0.254026248 |
| KI270467.1 | 3364 | C | G, A | 162 | 71 | 1008 | 110 | 2.04E−15 | 0.253803655 |
| KI270467.1 | 3811 | G | A, C | 305 | 53 | 657 | 40 | 1.36E−05 | 0.252844656 |
| KI270467.1 | 2351 | A | T, G | 799 | 229 | 452 | 28 | 7.65E−14 | 0.252361535 |
| chr4 | 2.1E+07 | A | G | 372 | 64 | 495 | 30 | 4.37E−05 | 0.25178532 |
| chrX | 7.4E+07 | T | C | 103 | 139 | 960 | 260 | 2.31E−28 | 0.251475349 |
| chr6 | 3.1E+07 | G | T, C | 163 | 446 | 142 | 122 | 4.15E−13 | 0.250785038 |
| KI270467.1 | 3203 | A | G | 739 | 134 | 410 | 16 | 1.66E−08 | 0.250165361 |
| chr11 | 6.5E+07 | C | T, A | 399 | 177 | 649 | 104 | 1.76E−12 | 0.249168048 |
| KI270467.1 | 1943 | T | C | 642 | 124 | 491 | 28 | 7.47E−08 | 0.249152227 |
| chr7 | 1.3E+08 | A | G | 667 | 320 | 588 | 90 | 2.26E−17 | 0.249013701 |
| chr19 | 5.1E+07 | G | A | 9 | 11 | 107 | 23 | 0.003693 | 0.245943205 |
| KI270466.1 | 691 | T | A | 244 | 51 | 610 | 45 | 1.30E−05 | 0.245535714 |
| chr3 | 2.8E+07 | A | T, C | 17 | 25 | 122 | 43 | 0.000567 | 0.245344901 |
| chr6 | 2.6E+07 | T | C | 143 | 22 | 230 | 13 | 0.040157 | 0.245193412 |
| KI270467.1 | 2392 | C | T | 89 | 76 | 1156 | 164 | 4.76E−26 | 0.245180723 |
| chr6 | 3.1E+07 | G | A | 1 | 3 | 198 | 9 | 6.58E−06 | 0.244974874 |
| chr6 | 4.4E+07 | C | T | 630 | 165 | 712 | 66 | 1.32E−10 | 0.24483713 |
| chr6 | 3E+07 | G | A | 12 | 52 | 443 | 140 | 2.41E−19 | 0.244459707 |
| chr3 | 4984323 | A | G | 9 | 19 | 107 | 40 | 0.000539 | 0.244447691 |
| KI2704671 | 3719 | G | T | 644 | 99 | 399 | 16 | 3.80E−06 | 0.243419901 |
| chr8 | 4.2E+07 | C | T | 157 | 32 | 321 | 24 | 0.003383 | 0.242976689 |
| KI270467.1 | 2757 | G | A, C | 472 | 89 | 582 | 40 | 3.12E−06 | 0.242104644 |
| chr19 | 8364724 | G | A | 343 | 55 | 354 | 20 | 0.000792 | 0.241224295 |
| chr6 | 3E+07 | G | A | 168 | 183 | 391 | 155 | 1.82E−11 | 0.240883446 |
| chr4 | 2.2E+07 | A | T | 243 | 23 | 340 | 12 | 0.043586 | 0.240333252 |
| KI270466.1 | 1096 | C | T | 323 | 44 | 365 | 18 | 0.002926 | 0.240200675 |
| KI270467.1 | 2821 | A | G | 737 | 141 | 382 | 16 | 2.55E−08 | 0.239465401 |
| KI270467.1 | 3224 | T | C, G | 814 | 155 | 366 | 12 | 3.00E−09 | 0.238313204 |
| KI270467.1 | 1366 | C | T | 154 | 19 | 601 | 24 | 0.00292 | 0.237886955 |
| chr8 | 1.5E+08 | A | G | 147 | 18 | 476 | 20 | 0.010845 | 0.237729154 |
| KI270467.1 | 3330 | A | T, C | 756 | 153 | 413 | 20 | 8.56E−09 | 0.237686477 |
| chr6 | 3E+07 | C | A | 7 | 21 | 144 | 53 | 1.06E−05 | 0.237426168 |
| KI270467.1 | 3361 | G | C, A | 772 | 161 | 403 | 19 | 3.08E−09 | 0.237423168 |
| chr6 | 3E+07 | A | G | 18 | 54 | 434 | 141 | 1.08E−16 | 0.237100068 |
| chr4 | 2.1E+07 | A | G | 415 | 62 | 413 | 22 | 0.000397 | 0.236887509 |
| KI270467.1 | 2910 | A | G, C | 529 | 103 | 576 | 41 | 1.48E−06 | 0.236544746 |
| KI270467.1 | 3491 | G | A, C | 677 | 139 | 476 | 30 | 7.37E−08 | 0.235321287 |
| KI270467.1 | 1905 | G | C, T | 466 | 66 | 548 | 29 | 0.000141 | 0.235170767 |
| KI270467.1 | 1910 | T | C | 476 | 77 | 562 | 34 | 3.28E−05 | 0.235119513 |
| chrX | 7.4E+07 | T | C | 108 | 53 | 134 | 25 | 0.00336 | 0.233206188 |
| chr6 | 3.7E+07 | T | C | 21 | 43 | 139 | 75 | 7.66E−05 | 0.23315678 |
| KI270466.1 | 649 | T | G, C | 222 | 51 | 639 | 53 | 1.04E−05 | 0.232544894 |
| chr6 | 3.3E+07 | C | G | 21 | 54 | 108 | 83 | 0.000356 | 0.231369886 |
| chr11 | 6.5E+07 | A | G | 610 | 232 | 513 | 68 | 1.33E−11 | 0.230145444 |
| KI270467.1 | 3631 | T | C, G | 423 | 75 | 620 | 43 | 2.32E−05 | 0.230032338 |
| KI270467.1 | 3369 | T | G | 787 | 167 | 391 | 19 | 4.31E−09 | 0.229767968 |
| chr7 | 1.3E+08 | A | G | 834 | 378 | 445 | 51 | 7.31E−18 | 0.22904695 |
| KI270467.1 | 1148 | G | A | 205 | 23 | 519 | 22 | 0.011248 | 0.22796194 |
| chr15 | 3.3E+07 | C | G | 363 | 151 | 551 | 91 | 5.02E−09 | 0.226811581 |
| chr4 | 2.1E+07 | T | C | 371 | 62 | 542 | 36 | 0.000202 | 0.226300378 |
| chr11 | 6.5E+07 | G | A | 431 | 496 | 964 | 433 | 2.11E−25 | 0.224946854 |
| chrX | 7.4E+07 | G | A | 547 | 317 | 733 | 169 | 1.02E−15 | 0.224919624 |
| chrX | 7.4E+07 | T | C | 11 | 33 | 300 | 94 | 6.20E−11 | 0.224472745 |
| chr5 | 1.4E+08 | A | G | 445 | 69 | 644 | 40 | 7.93E−05 | 0.224395751 |
| chr6 | 3.3E+07 | A | G | 28 | 40 | 173 | 70 | 7.25E−05 | 0.224332881 |
| KI270467.1 | 2470 | A | C, T | 853 | 209 | 377 | 19 | 8.01E−11 | 0.223170732 |
| chrX | 7.4E+07 | T | C | 118 | 63 | 163 | 35 | 0.001455 | 0.222928317 |
| KI270467.1 | 3406 | C | G, A, T | 787 | 165 | 380 | 19 | 1.54E−08 | 0.222360382 |
| KI2704671 | 3913 | C | T | 187 | 23 | 513 | 24 | 0.010221 | 0.222218845 |
| KI270466.1 | 976 | C | T, A | 353 | 49 | 340 | 18 | 0.004801 | 0.221963774 |
| chr18 | 5.9E+07 | T | C | 366 | 216 | 300 | 64 | 2.91E−09 | 0.221879022 |
| KI270467.1 | 3657 | C | A, T, G | 654 | 83 | 334 | 11 | 0.000142 | 0.221035404 |
| KI270467.1 | 1853 | T | A, C | 439 | 43 | 425 | 16 | 0.009145 | 0.220711707 |
| KI270467.1 | 2221 | C | G, A | 742 | 201 | 481 | 42 | 1.12E−09 | 0.22045567 |
| KI270466.1 | 461 | C | G, T | 659 | 139 | 349 | 20 | 4.65E−07 | 0.220443995 |
| chr4 | 2.1E+07 | A | T | 374 | 69 | 557 | 42 | 0.000115 | 0.219903039 |
| chr6 | 3.3E+07 | G | C | 5 | 16 | 100 | 44 | 0.000893 | 0.219047619 |
| KI270467.1 | 1915 | T | C, A | 471 | 74 | 566 | 36 | 0.000151 | 0.21853248 |
| KI270467.1 | 3323 | A | G | 756 | 156 | 419 | 25 | 9.68E−08 | 0.218474198 |
| KI270467.1 | 2804 | G | C, A | 737 | 136 | 371 | 18 | 6.75E−07 | 0.217954428 |
| chr7 | 1.3E+08 | T | A, C, G | 816 | 343 | 421 | 48 | 4.18E−15 | 0.217577383 |
| KI270467.1 | 2620 | T | G, C, A | 765 | 174 | 439 | 30 | 1.97E−08 | 0.217559117 |
| KI270467.1 | 1906 | T | A | 535 | 79 | 489 | 28 | 0.000229 | 0.21585682 |
| KI270467.1 | 3695 | G | T, A | 536 | 83 | 509 | 31 | 0.000144 | 0.215151515 |
| chr11 | 6.5E+07 | G | A, C | 696 | 356 | 635 | 127 | 9.09E−15 | 0.21414494 |
| chr11 | 6.5E+07 | C | T | 406 | 167 | 652 | 113 | 2.94E−09 | 0.21268566 |
| chr3 | 8.2E+07 | G | A | 7 | 4 | 175 | 12 | 0.015726 | 0.211538462 |
| chrX | 7.4E+07 | G | A | 575 | 358 | 644 | 166 | 1.33E−14 | 0.211507994 |
| chr2 | 3.5E+07 | A | C | 276 | 35 | 356 | 19 | 0.022456 | 0.211439287 |
| chr8 | 4.2E+07 | G | A | 356 | 64 | 413 | 31 | 0.001079 | 0.210745329 |
| KI270467.1 | 3335 | A | G, T | 149 | 61 | 1023 | 120 | 1.25E−11 | 0.209883469 |
| KI270466.1 | 294 | G | A | 510 | 103 | 489 | 40 | 3.24E−05 | 0.20976921 |
| chr19 | 8364224 | G | A | 423 | 186 | 567 | 106 | 5.05E−09 | 0.209713574 |
| KI270467.1 | 3619 | T | C | 579 | 97 | 477 | 31 | 7.39E−05 | 0.209517045 |
| chr8 | 1.3E+08 | G | A, C | 469 | 68 | 525 | 32 | 0.000752 | 0.208169014 |
| chr9 | 7.2E+07 | G | A | 21 | 47 | 80 | 66 | 0.010146 | 0.208008411 |
| KI270467.1 | 1981 | T | C | 831 | 182 | 359 | 19 | 1.93E−08 | 0.207153309 |
| chr11 | 295527 | T | C | 104 | 47 | 403 | 67 | 4.58E−05 | 0.207152497 |
| KI270466.1 | 298 | T | C | 545 | 108 | 467 | 37 | 3.53E−05 | 0.206290037 |
| KI270466.1 | 595 | A | C, G | 532 | 99 | 426 | 31 | 9.29E−05 | 0.206214871 |
| KI270467.1 | 3284 | T | A, C | 713 | 137 | 466 | 32 | 3.16E−06 | 0.2059011 |
| chrX | 7.8E+07 | C | G | 5 | 16 | 106 | 48 | 0.001051 | 0.204954955 |
| KI270466.1 | 326 | T | A, C | 626 | 112 | 375 | 23 | 3.91E−05 | 0.204255004 |
| chr11 | 6.6E+07 | T | C, G, A | 589 | 343 | 456 | 104 | 2.22E−12 | 0.203701444 |
| chrX | 7.4E+07 | A | G | 6 | 17 | 199 | 56 | 2.19E−06 | 0.20360842 |
| chr11 | 295445 | A | C | 102 | 43 | 340 | 56 | 0.00043 | 0.203574204 |
| chr9 | 1.4E+08 | A | G | 9 | 20 | 125 | 54 | 0.000827 | 0.203106091 |
| KI270467.1 | 2795 | G | A | 729 | 127 | 374 | 20 | 9.29E−06 | 0.203020828 |
| chr8 | 1.3E+08 | G | A | 240 | 43 | 758 | 54 | 0.00017 | 0.202818007 |
| chr4 | 2.1E+07 | A | G | 355 | 63 | 587 | 46 | 0.000525 | 0.201123902 |
| chr6 | 3E+07 | C | T | 59 | 67 | 401 | 137 | 2.72E−08 | 0.200170503 |
| KI270466.1 | 554 | T | C | 364 | 89 | 648 | 70 | 1.92E−05 | 0.200064633 |
| KI270466.1 | 247 | T | A, C | 579 | 98 | 339 | 20 | 0.000205 | 0.19978952 |
| chr11 | 6.6E+07 | C | T | 102 | 283 | 1332 | 764 | 7.64E−39 | 0.199166377 |
| KI270466.1 | 1129 | T | C | 324 | 42 | 293 | 16 | 0.028336 | 0.199016375 |
| KI270467.1 | 1987 | C | T, G, A | 780 | 165 | 392 | 26 | 5.66E−07 | 0.198345335 |
| KI270467.1 | 1956 | A | G | 759 | 144 | 402 | 25 | 3.66E−06 | 0.198324236 |
| KI270467.1 | 1119 | C | T, A | 173 | 21 | 578 | 28 | 0.016112 | 0.198211908 |
| KI270467.1 | 3022 | T | C, G, A | 854 | 128 | 251 | 4 | 2.24E−06 | 0.196846291 |
| KI270467.1 | 3782 | G | A, C | 311 | 47 | 660 | 44 | 0.001544 | 0.196195154 |
| chr11 | 6.5E+07 | A | G | 892 | 473 | 488 | 89 | 4.80E−16 | 0.195260199 |
| chr4 | 1.9E+08 | G | A | 315 | 54 | 671 | 51 | 0.00065 | 0.194813098 |
| KI270466.1 | 290 | G | A | 613 | 116 | 381 | 27 | 7.03E−05 | 0.19448861 |
| chr6 | 3.3E+07 | A | G | 2 | 14 | 109 | 52 | 0.000318 | 0.194103194 |
| chr8 | 1.3E+08 | T | C | 511 | 63 | 438 | 23 | 0.004687 | 0.194096601 |
| chr6 | 3.1E+07 | T | C | 56 | 207 | 762 | 582 | 9.08E−24 | 0.193897757 |
| chrX | 7.4E+07 | A | G, C | 513 | 278 | 633 | 156 | 1.45E−10 | 0.192909016 |
| chr6 | 3E+07 | T | G | 12 | 20 | 136 | 53 | 0.00178 | 0.192891522 |
| chr11 | 295670 | G | A | 118 | 41 | 289 | 44 | 0.0052 | 0.192426651 |
| chr6 | 3.1E+07 | C | T | 61 | 199 | 653 | 521 | 3.95E−19 | 0.190954715 |
| KI270467.1 | 3735 | T | A, C | 686 | 99 | 338 | 16 | 0.000312 | 0.19094769 |
| chr8 | 1.3E+08 | T | C, A | 571 | 60 | 333 | 13 | 0.009298 | 0.19028064 |
| chr2 | 2.3E+08 | T | G | 147 | 45 | 171 | 24 | 0.03201 | 0.189909762 |
| chr11 | 295343 | G | A | 95 | 37 | 299 | 49 | 0.003673 | 0.189115807 |
| KI270467.1 | 1875 | T | A, C | 567 | 56 | 378 | 15 | 0.013429 | 0.188732394 |
| KI270467.1 | 1459 | T | G, C | 409 | 45 | 508 | 26 | 0.01763 | 0.187783188 |
| chr7 | 1.3E+08 | A | G | 986 | 411 | 293 | 18 | 1.59E−16 | 0.187127181 |
| KI270467.1 | 1883 | G | T, C | 493 | 52 | 410 | 19 | 0.018304 | 0.186436448 |
| chr6 | 3.3E+07 | G | A | 21 | 63 | 218 | 167 | 2.71E−06 | 0.186046935 |
| chr14 | 2.1E+07 | G | A | 120 | 51 | 160 | 32 | 0.022404 | 0.185886403 |
| chr1 | 1.8E+08 | G | A | 301 | 95 | 291 | 42 | 0.000882 | 0.184984711 |
| KI270467.1 | 2502 | T | G, A | 94 | 59 | 1138 | 167 | 4.28E−15 | 0.184763246 |
| chr8 | 4.2E+07 | C | G, T, A | 160 | 32 | 583 | 48 | 0.002214 | 0.184656797 |
| chr6 | 3.1E+07 | T | G, A | 59 | 199 | 748 | 576 | 4.06E−21 | 0.183663909 |
| chrX | 7.4E+07 | C | T | 54 | 37 | 224 | 61 | 0.002785 | 0.183306416 |
| chr6 | 7.9E+07 | C | T | 7 | 6 | 391 | 24 | 3.82E−06 | 0.18241206 |
| KI270467.1 | 1990 | T | C, A, G | 106 | 38 | 961 | 97 | 2.18E−08 | 0.182137526 |
| chr11 | 6.5E+07 | A | G | 527 | 191 | 588 | 101 | 4.85E−07 | 0.181463849 |
| chr16 | 8.9E+07 | T | C | 91 | 43 | 141 | 32 | 0.043473 | 0.181091954 |
| chr6 | 3.1E+07 | G | A | 104 | 246 | 728 | 560 | 2.61E−17 | 0.180210918 |
| chr7 | 1.4E+08 | G | T | 453 | 105 | 677 | 76 | 6.86E−05 | 0.179225541 |
| chr11 | 6.8E+07 | T | C | 241 | 79 | 488 | 76 | 0.000273 | 0.17908757 |
| chr6 | 3.3E+07 | C | G | 2 | 13 | 109 | 53 | 0.000779 | 0.178951679 |
| chr6 | 2888837 | A | C | 3 | 11 | 210 | 46 | 3.62E−06 | 0.178897949 |
| chr6 | 3E+07 | C | T | 19 | 23 | 138 | 54 | 0.009197 | 0.17768219 |
| chr6 | 2889003 | T | C | 5 | 10 | 156 | 38 | 0.00077 | 0.177277433 |
| chr6 | 3E+07 | C | G | 17 | 35 | 378 | 124 | 3.38E−09 | 0.177087811 |
| chrX | 5.6E+07 | C | G | 4 | 8 | 216 | 33 | 4.14E−05 | 0.176940133 |
| chr11 | 6.8E+07 | T | G | 232 | 78 | 500 | 80 | 0.000256 | 0.176730995 |
| chr4 | 2.1E+07 | T | C, A | 506 | 65 | 215 | 9 | 0.01125 | 0.176575327 |
| chr2 | 3.5E+07 | A | G | 538 | 59 | 182 | 5 | 0.014926 | 0.174652778 |
| chr6 | 3.1E+07 | C | G | 74 | 199 | 666 | 527 | 4.40E−16 | 0.174104683 |
| chr6 | 3.1E+07 | C | G | 50 | 174 | 687 | 546 | 2.16E−18 | 0.173824062 |
| chr6 | 3.3E+07 | T | C | 5 | 13 | 98 | 46 | 0.01102 | 0.171795294 |
| chr6 | 3.1E+07 | G | T, C | 51 | 176 | 712 | 562 | 1.05E−18 | 0.171640969 |
| chr6 | 3.3E+07 | C | T | 2 | 11 | 107 | 47 | 0.001774 | 0.171306549 |
| chr6 | 3E+07 | A | G | 26 | 40 | 363 | 128 | 2.22E−07 | 0.171257192 |
| KI270467.1 | 3786 | T | C | 645 | 78 | 347 | 17 | 0.006406 | 0.170851019 |
| chr8 | 1.3E+08 | C | G | 160 | 42 | 959 | 92 | 6.80E−06 | 0.170448028 |
| chr6 | 3.1E+07 | A | C, T | 57 | 181 | 691 | 553 | 2.09E−17 | 0.170390797 |
| chr19 | 1.8E+07 | G | T | 142 | 77 | 272 | 73 | 0.002223 | 0.170338164 |
| chr6 | 3.1E+07 | A | T | 53 | 186 | 774 | 608 | 3.28E−20 | 0.170169865 |
| KI270467.1 | 1936 | T | A, C | 690 | 113 | 430 | 31 | 0.000748 | 0.168650794 |
| chr6 | 3E+07 | C | A | 19 | 32 | 357 | 114 | 1.55E−07 | 0.168646167 |
| chr6 | 3.1E+07 | T | C | 61 | 195 | 770 | 611 | 3.92E−19 | 0.168529948 |
| KI270467.1 | 1890 | G | C, T, A | 537 | 55 | 397 | 19 | 0.033804 | 0.168296776 |
| chrX | 4.9E+07 | G | A | 20 | 35 | 292 | 116 | 3.61E−06 | 0.167685515 |
| chr1 | 1.7E+08 | T | C | 36 | 12 | 399 | 36 | 0.003711 | 0.167241379 |
| chr6 | 3.3E+07 | C | T | 16 | 36 | 169 | 106 | 0.000562 | 0.16703464 |
| KI270466.1 | 651 | G | A | 371 | 60 | 474 | 39 | 0.012383 | 0.167007352 |
| chr17 | 7014384 | C | T | 27 | 75 | 396 | 250 | 1.07E−09 | 0.166939444 |
| chr6 | 3.1E+07 | C | A | 44 | 167 | 727 | 579 | 4.09E−19 | 0.166791848 |
| chr6 | 3.1E+07 | G | C | 51 | 182 | 796 | 622 | 2.43E−20 | 0.166155644 |
| chr9 | 7.3E+07 | A | C | 7 | 19 | 134 | 70 | 0.001929 | 0.163837756 |
| chrM | 2001 | C | T | 296 | 472 | 1170 | 819 | 6.00E−20 | 0.163698097 |
| chr19 | 1.8E+07 | G | C | 135 | 79 | 254 | 76 | 0.003889 | 0.162633718 |
| chr6 | 3.1E+07 | C | A | 57 | 200 | 882 | 697 | 1.11E−21 | 0.162262565 |
| chr11 | 6.5E+07 | A | G | 590 | 204 | 514 | 89 | 8.31E−06 | 0.161825444 |
| KI270467.1 | 2796 | T | C, A, G | 846 | 132 | 246 | 9 | 0.000107 | 0.161444938 |
| chr14 | 2.7E+07 | A | G | 493 | 121 | 451 | 56 | 0.000721 | 0.161370056 |
| chr6 | 3.1E+07 | A | G | 77 | 213 | 831 | 653 | 2.41E−18 | 0.161156667 |
| chr6 | 3.1E+07 | T | C | 62 | 188 | 776 | 613 | 7.56E−18 | 0.160720937 |
| chr21 | 4.5E+07 | T | C | 174 | 56 | 203 | 34 | 0.042662 | 0.160683761 |
| chr15 | 3.5E+07 | C | T | 437 | 163 | 569 | 111 | 2.73E−05 | 0.160496873 |
| chr6 | 3.1E+07 | A | G | 53 | 182 | 795 | 636 | 4.16E−19 | 0.159993888 |
| chr6 | 3E+07 | T | G, A | 19 | 29 | 289 | 102 | 1.83E−05 | 0.159685734 |
| chr6 | 3.1E+07 | G | C | 42 | 168 | 790 | 632 | 4.93E−20 | 0.159519231 |
| KI270467.1 | 1836 | A | T, C, G | 71 | 10 | 568 | 27 | 0.041011 | 0.159159159 |
| chrX | 1.5E+08 | G | A | 414 | 110 | 421 | 58 | 0.001499 | 0.158953522 |
| KI270466.1 | 207 | G | T, A | 460 | 75 | 440 | 37 | 0.011614 | 0.158531746 |
| chr6 | 3.1E+07 | G | A | 54 | 176 | 771 | 611 | 6.03E−18 | 0.158179508 |
| chr6 | 3.1E+07 | T | C | 69 | 205 | 830 | 668 | 1.96E−18 | 0.158070505 |
| chr6 | 4.4E+07 | G | A | 5 | 12 | 149 | 51 | 0.00163 | 0.158008658 |
| chr6 | 3.1E+07 | A | T | 54 | 195 | 909 | 716 | 6.78E−22 | 0.157975728 |
| chr6 | 3E+07 | C | T | 71 | 171 | 748 | 529 | 2.20E−15 | 0.157594628 |
| chr6 | 3.1E+07 | C | T | 56 | 171 | 711 | 571 | 4.89E−16 | 0.157446487 |
| chr15 | 3.5E+07 | G | A | 477 | 155 | 467 | 79 | 0.000166 | 0.157096552 |
| chr6 | 3.3E+07 | G | A | 3 | 10 | 90 | 43 | 0.020259 | 0.156421181 |
| chrX | 1.5E+08 | C | T | 490 | 112 | 326 | 36 | 0.002656 | 0.156266561 |
| chr6 | 3.3E+07 | C | T | 40 | 77 | 206 | 165 | 0.000611 | 0.155580192 |
| chr6 | 3.3E+07 | C | G | 20 | 46 | 169 | 130 | 0.001282 | 0.155543531 |
| chr12 | 1.1E+07 | T | C | 119 | 66 | 186 | 55 | 0.025556 | 0.155290611 |
| chr6 | 3.1E+07 | T | G | 56 | 193 | 907 | 717 | 8.22E−21 | 0.153936303 |
| chr17 | 7312217 | T | C, G | 16 | 35 | 106 | 88 | 0.026097 | 0.153405305 |
| chr11 | 6.5E+07 | C | G | 265 | 138 | 444 | 124 | 0.000181 | 0.15295169 |
| chr10 | 7806641 | A | G | 9 | 8 | 204 | 33 | 0.006648 | 0.15286843 |
| chr11 | 6.6E+07 | C | T | 90 | 243 | 1357 | 890 | 4.21E−28 | 0.152277195 |
| chr6 | 3.1E+07 | A | C | 49 | 163 | 745 | 599 | 1.00E−16 | 0.152197915 |
| chr6 | 4.4E+07 | T | A | 29 | 47 | 204 | 123 | 0.001276 | 0.152007069 |
| chr6 | 3.3E+07 | C | G | 95 | 167 | 460 | 350 | 1.18E−07 | 0.151846237 |
| KI270466.1 | 379 | G | C, T | 692 | 127 | 328 | 26 | 0.001306 | 0.151633987 |
| KI270466.1 | 131 | T | C | 508 | 79 | 399 | 32 | 0.01685 | 0.151623509 |
| chr6 | 3.1E+07 | T | G | 51 | 163 | 747 | 594 | 2.03E−16 | 0.151413872 |
| chr11 | 1.1E+08 | G | A | 646 | 556 | 473 | 207 | 3.31E−10 | 0.151401328 |
| chr11 | 6.5E+07 | C | T | 529 | 158 | 453 | 71 | 0.000315 | 0.151259794 |
| chr6 | 3E+07 | A | G | 16 | 29 | 375 | 122 | 3.00E−07 | 0.151132264 |
| chr7 | 1.4E+08 | C | T | 236 | 36 | 818 | 60 | 0.007479 | 0.151091082 |
| KI270466.1 | 430 | A | G, T | 719 | 135 | 318 | 25 | 0.000882 | 0.150403809 |
| chr6 | 3.1E+07 | G | A | 62 | 195 | 894 | 711 | 3.32E−19 | 0.150378232 |
| chr10 | 4.5E+07 | G | A | 382 | 118 | 284 | 45 | 0.003649 | 0.150352807 |
| chr6 | 2.6E+07 | G | A | 4 | 5 | 340 | 26 | 3.43E−05 | 0.149662416 |
| chr6 | 3.1E+07 | G | A | 54 | 187 | 924 | 726 | 1.43E−20 | 0.149604553 |
| chr10 | 7.2E+07 | T | A | 295 | 104 | 629 | 118 | 0.000284 | 0.149204399 |
| chr6 | 3.1E+07 | C | G, T | 51 | 168 | 793 | 635 | 3.05E−17 | 0.148788902 |
| chr6 | 3.1E+07 | G | A | 88 | 205 | 771 | 611 | 3.26E−14 | 0.148780787 |
| KI270467.1 | 1465 | G | T, C | 670 | 68 | 242 | 9 | 0.030975 | 0.14846776 |
| chr6 | 3.1E+07 | G | A | 55 | 170 | 781 | 625 | 1.95E−16 | 0.148047004 |
| chr6 | 3.1E+07 | G | A | 70 | 197 | 877 | 691 | 1.31E−17 | 0.147929212 |
| chr6 | 3.1E+07 | C | T | 50 | 167 | 792 | 639 | 3.55E−17 | 0.147813607 |
| chr6 | 3.1E+07 | A | T, C, G | 67 | 177 | 712 | 580 | 6.75E−14 | 0.147809999 |
| KI270467.1 | 3778 | G | A, C | 659 | 77 | 322 | 17 | 0.024534 | 0.14738543 |
| chr6 | 3E+07 | T | C | 58 | 168 | 947 | 653 | 1.68E−19 | 0.146917059 |
| chrX | 1.5E+08 | C | T | 588 | 153 | 465 | 64 | 0.000619 | 0.146664566 |
| KI270467.1 | 2631 | T | G | 896 | 180 | 313 | 23 | 7.71E−05 | 0.145591153 |
| chr11 | 6.5E+07 | G | C | 198 | 54 | 339 | 51 | 0.036146 | 0.14557063 |
| chr7 | 1.3E+08 | G | A | 276 | 93 | 556 | 102 | 0.001287 | 0.145192308 |
| chr6 | 3.1E+07 | A | G | 79 | 204 | 877 | 692 | 1.79E−16 | 0.145042588 |
| chr11 | 6.5E+07 | T | A | 167 | 48 | 398 | 61 | 0.022263 | 0.144791751 |
| chr6 | 3.1E+07 | C | T | 52 | 160 | 762 | 607 | 1.25E−15 | 0.14472289 |
| chr6 | 3.1E+07 | T | C | 108 | 214 | 689 | 550 | 3.61E−11 | 0.144596556 |
| chrX | 7.4E+07 | A | C, G, T | 707 | 400 | 470 | 137 | 1.02E−07 | 0.144199263 |
| chr6 | 3.3E+07 | C | G, A | 103 | 172 | 468 | 358 | 4.99E−07 | 0.144143013 |
| KI270466.1 | 673 | T | C, G, A | 93 | 27 | 780 | 81 | 0.000256 | 0.14347079 |
| chr6 | 3.1E+07 | G | C | 101 | 216 | 630 | 551 | 2.15E−10 | 0.143449794 |
| chr6 | 3.1E+07 | A | G | 87 | 212 | 879 | 697 | 9.57E−16 | 0.143161211 |
| chr6 | 3.1E+07 | C | A | 101 | 213 | 758 | 604 | 1.46E−12 | 0.143131335 |
| chr6 | 3.3E+07 | C | G | 19 | 43 | 190 | 141 | 0.001218 | 0.142786561 |
| chr6 | 3E+07 | A | T, C | 67 | 170 | 916 | 637 | 4.11E−17 | 0.142498056 |
| chr6 | 3E+07 | T | C | 23 | 30 | 359 | 118 | 1.78E−05 | 0.142493279 |
| chr6 | 3.1E+07 | T | C | 56 | 168 | 802 | 641 | 6.40E−16 | 0.142395717 |
| chr11 | 6.5E+07 | G | A, C | 778 | 340 | 501 | 113 | 7.24E−07 | 0.142264152 |
| chr6 | 3.1E+07 | T | G | 54 | 178 | 916 | 723 | 8.16E−19 | 0.141888165 |
| chr6 | 3.3E+07 | C | T | 19 | 44 | 195 | 147 | 0.001011 | 0.141581445 |
| chr6 | 3.1E+07 | G | C | 52 | 158 | 749 | 609 | 8.50E−15 | 0.141078541 |
| chr6 | 3.1E+07 | C | T | 48 | 173 | 922 | 737 | 2.07E−19 | 0.140625354 |
| chr6 | 3.1E+07 | T | A | 50 | 158 | 759 | 623 | 3.72E−15 | 0.14050004 |
| chrX | 7.4E+07 | G | A | 7 | 9 | 130 | 38 | 0.040018 | 0.140394471 |
| chr6 | 3E+07 | A | T | 12 | 27 | 394 | 132 | 1.05E−07 | 0.140254671 |
| KI270467.1 | 2782 | C | T, G | 625 | 87 | 367 | 26 | 0.023395 | 0.139871182 |
| chr6 | 3.3E+07 | A | C | 23 | 49 | 163 | 137 | 0.005936 | 0.139784946 |
| chrM | 1948 | C | T | 335 | 474 | 1132 | 814 | 2.39E−14 | 0.139655231 |
| KI270466.1 | 690 | G | C | 601 | 81 | 252 | 15 | 0.029549 | 0.139177902 |
| chr6 | 3.1E+07 | G | A | 58 | 167 | 799 | 643 | 4.78E−15 | 0.138494893 |
| chr6 | 3.1E+07 | G | C, T | 49 | 150 | 761 | 605 | 8.60E−15 | 0.13818167 |
| chr4 | 2.2E+07 | T | C | 26 | 7 | 435 | 29 | 0.022296 | 0.138045312 |
| KI270467.1 | 3652 | T | A, C | 769 | 98 | 274 | 14 | 0.011087 | 0.137703739 |
| chr6 | 3E+07 | T | A | 63 | 157 | 810 | 595 | 3.13E−14 | 0.136611647 |
| chr6 | 3.1E+07 | C | T | 103 | 219 | 857 | 680 | 2.18E−13 | 0.136312338 |
| chr15 | 3.5E+07 | G | A | 266 | 103 | 664 | 141 | 0.000445 | 0.136109642 |
| chr6 | 3.3E+07 | C | A | 23 | 45 | 180 | 136 | 0.005035 | 0.135318292 |
| chr6 | 3.1E+07 | T | C | 79 | 189 | 823 | 661 | 1.32E−13 | 0.134769793 |
| chr6 | 3.3E+07 | T | C | 25 | 47 | 179 | 136 | 0.006384 | 0.134281581 |
| chr14 | 3.9E+07 | A | G | 207 | 90 | 288 | 73 | 0.020202 | 0.133965421 |
| chr6 | 3.3E+07 | C | G, T | 108 | 168 | 458 | 350 | 6.04E−06 | 0.133511603 |
| chr6 | 3E+07 | C | T | 63 | 156 | 873 | 622 | 6.95E−15 | 0.133206447 |
| KI270467.1 | 3620 | A | G | 747 | 108 | 314 | 21 | 0.011535 | 0.133156522 |
| chr3 | 1.9E+08 | C | T | 312 | 276 | 712 | 355 | 5.70E−07 | 0.132713451 |
| chr6 | 3.3E+07 | A | G | 116 | 179 | 497 | 377 | 2.50E−06 | 0.132709167 |
| chr14 | 1.1E+08 | T | G, A, C | 118 | 192 | 50 | 38 | 0.013479 | 0.132401656 |
| chr6 | 3.3E+07 | A | C | 25 | 51 | 198 | 159 | 0.003396 | 0.13074952 |
| chr19 | 1.8E+07 | T | A | 143 | 73 | 237 | 71 | 0.043645 | 0.130628655 |
| chr6 | 3.3E+07 | T | G, A | 108 | 172 | 486 | 379 | 3.90E−06 | 0.130341528 |
| chr6 | 3.3E+07 | G | T | 102 | 165 | 517 | 396 | 1.63E−06 | 0.129335741 |
| chr3 | 1.9E+08 | T | C | 338 | 284 | 679 | 332 | 2.65E−06 | 0.128688912 |
| chr6 | 3E+07 | T | G | 45 | 146 | 975 | 701 | 5.00E−18 | 0.128255434 |
| chr6 | 3E+07 | A | T, G | 47 | 149 | 976 | 707 | 7.83E−18 | 0.128122117 |
| KI270466.1 | 410 | T | A | 736 | 136 | 306 | 27 | 0.005508 | 0.128021855 |
| chr6 | 3.3E+07 | C | G | 17 | 42 | 203 | 163 | 0.001591 | 0.127605322 |
| chr6 | 3E+07 | A | G | 47 | 149 | 998 | 716 | 4.15E−18 | 0.127278259 |
| chr6 | 3E+07 | A | G | 42 | 133 | 887 | 642 | 4.96E−16 | 0.126403 |
| KI270467.1 | 2322 | C | A, G | 1040 | 225 | 199 | 8 | 5.63E−06 | 0.126278634 |
| chr6 | 3.3E+07 | C | G, A | 96 | 154 | 478 | 371 | 8.20E−06 | 0.126085947 |
| chr3 | 1.9E+08 | C | T | 307 | 269 | 720 | 364 | 2.06E−06 | 0.126031586 |
| KI270467.1 | 1904 | G | A | 60 | 19 | 963 | 84 | 4.10E−05 | 0.125814993 |
| chr6 | 3E+07 | A | G | 52 | 145 | 941 | 669 | 5.86E−16 | 0.125766112 |
| chr6 | 3E+07 | A | G | 20 | 30 | 403 | 144 | 1.07E−05 | 0.125132469 |
| chr6 | 3E+07 | G | C | 60 | 147 | 868 | 628 | 1.12E−13 | 0.125022247 |
| chr6 | 3E+07 | A | G | 49 | 139 | 863 | 639 | 1.34E−14 | 0.124935169 |
| KI270467.1 | 1898 | T | A | 73 | 20 | 952 | 82 | 0.000212 | 0.124858919 |
| chr6 | 3E+07 | C | G | 48 | 143 | 950 | 685 | 2.09E−16 | 0.124609122 |
| chrM | 1905 | C | T, G, A | 398 | 507 | 1065 | 774 | 1.05E−10 | 0.123740798 |
| chr6 | 3E+07 | C | T | 60 | 152 | 941 | 676 | 7.13E−15 | 0.123634819 |
| chr14 | 6.2E+07 | T | C | 5 | 3 | 254 | 18 | 0.046604 | 0.123552124 |
| chr6 | 3.3E+07 | G | T | 108 | 162 | 485 | 369 | 1.76E−05 | 0.122959957 |
| chr6 | 3.3E+07 | A | G | 19 | 44 | 151 | 144 | 0.019928 | 0.122277847 |
| chr6 | 1.7E+08 | T | G | 3 | 14 | 192 | 88 | 0.000393 | 0.121870287 |
| chr6 | 3E+07 | G | A | 29 | 35 | 433 | 155 | 3.60E−05 | 0.121439964 |
| KI270467.1 | 3208 | A | G | 118 | 33 | 1024 | 114 | 0.000229 | 0.1211623 |
| chr6 | 3E+07 | C | T | 19 | 29 | 416 | 147 | 9.76E−06 | 0.121094566 |
| KI270467.1 | 3839 | T | G, C, A | 779 | 86 | 178 | 6 | 0.028682 | 0.120780519 |
| chr6 | 3.3E+07 | T | G | 111 | 167 | 502 | 387 | 1.74E−05 | 0.120367371 |
| chr6 | 3E+07 | A | C | 53 | 137 | 851 | 629 | 3.63E−13 | 0.120222856 |
| chr10 | 1.3E+08 | C | T | 223 | 129 | 337 | 120 | 0.010766 | 0.119858003 |
| chr6 | 3E+07 | G | C, T | 44 | 136 | 986 | 709 | 2.39E−16 | 0.118228299 |
| KI270467.1 | 3823 | G | C | 65 | 16 | 889 | 70 | 0.001395 | 0.11791234 |
| chr6 | 3E+07 | A | G | 71 | 156 | 918 | 667 | 1.22E−12 | 0.117760739 |
| chrM | 1792 | G | A | 537 | 620 | 929 | 661 | 6.70E−09 | 0.117694013 |
| chr6 | 3.3E+07 | C | T, G | 115 | 167 | 493 | 378 | 4.21E−05 | 0.117277282 |
| chr11 | 6.5E+07 | T | G, C, A | 1103 | 540 | 276 | 49 | 2.86E−09 | 0.116953182 |
| chr6 | 3E+07 | A | T, C | 51 | 130 | 861 | 622 | 6.82E−13 | 0.116951288 |
| chr6 | 3.3E+07 | C | T | 119 | 171 | 419 | 337 | 0.000299 | 0.115424582 |
| chr1 | 6.7E+07 | C | T | 0 | 3 | 303 | 23 | 1.01E−05 | 0.115384615 |
| chr6 | 3E+07 | cl | T | 67 | 151 | 940 | 682 | 9.05E−13 | 0.114738249 |
| chr6 | 3.3E+07 | | A, T | 19 | 41 | 209 | 166 | 0.005134 | 0.1147343 |
| chr2 | 2.2E+08 | C | T | 394 | 190 | 272 | 79 | 0.00758 | 0.114728111 |
| chr6 | 3E+07 | C | T, G | 64 | 141 | 815 | 612 | 7.94E−11 | 0.114440985 |
| chr6 | 3.3E+07 | C | T, G | 109 | 163 | 496 | 391 | 5.22E−05 | 0.114058537 |
| chr6 | 3.3E+07 | G | C, T | 111 | 168 | 500 | 401 | 4.94E−05 | 0.113585439 |
| chr6 | 1.6E+08 | A | T | 20 | 15 | 241 | 64 | 0.035557 | 0.113245065 |
| chr6 | 3E+07 | G | T | 46 | 133 | 968 | 706 | 8.31E−15 | 0.113157159 |
| chr6 | 3E+07 | C | T | 72 | 152 | 911 | 668 | 1.56E−11 | 0.112120686 |
| chr10 | 3136689 | C | T | 341 | 106 | 454 | 90 | 0.030965 | 0.111885509 |
| chr6 | 3.2E+07 | A | G | 73 | 119 | 297 | 266 | 0.003454 | 0.111793612 |
| chr6 | 3.3E+07 | T | G, A | 111 | 160 | 483 | 376 | 0.000119 | 0.111638776 |
| chr6 | 3E+07 | G | A | 57 | 134 | 872 | 641 | 9.15E−12 | 0.111546929 |
| chrX | 1.5E+08 | G | A | 590 | 135 | 401 | 56 | 0.025018 | 0.111448059 |
| chrM | 8019 | C | T | 254 | 367 | 1199 | 916 | 7.59E−11 | 0.111237588 |
| chr12 | 1.2E+08 | G | A | 286 | 201 | 490 | 218 | 0.001544 | 0.111156903 |
| chr16 | 3069430 | C | T | 536 | 444 | 343 | 172 | 8.73E−05 | 0.110995376 |
| chr6 | 3.2E+07 | G | A | 64 | 107 | 330 | 285 | 0.001549 | 0.110522635 |
| chr10 | 3136723 | C | T | 294 | 91 | 455 | 90 | 0.043123 | 0.110239066 |
| chr6 | 3E+07 | G | A | 48 | 131 | 971 | 702 | 4.61E−14 | 0.1101579 |
| chr17 | 2.7E+07 | A | G | 544 | 139 | 299 | 45 | 0.027538 | 0.110120429 |
| chr6 | 3E+07 | T | C | 54 | 129 | 821 | 622 | 5.95E−11 | 0.110056686 |
| chr6 | 3E+07 | G | A | 64 | 148 | 988 | 721 | 6.74E−13 | 0.1094742 |
| chr6 | 3.3E+07 | A | C | 109 | 159 | 516 | 402 | 7.90E−05 | 0.10902246 |
| chr6 | 3E+07 | C | T | 24 | 27 | 357 | 130 | 0.00111 | 0.108982396 |
| chr1 | 2563650 | G | T, A | 451 | 159 | 334 | 74 | 0.021152 | 0.10788114 |
| chr17 | 4.9E+07 | G | C | 266 | 179 | 360 | 157 | 0.009694 | 0.107817967 |
| chr6 | 3.3E+07 | T | C | 28 | 48 | 195 | 158 | 0.027194 | 0.107449171 |
| chr6 | 3.2E+07 | T | G | 64 | 103 | 277 | 246 | 0.007575 | 0.107445655 |
| chr6 | 3.3E+07 | G | A | 160 | 205 | 475 | 366 | 0.000486 | 0.107050761 |
| chrM | 9777 | G | A | 553 | 618 | 894 | 646 | 2.75E−07 | 0.106754044 |
| KI270466.1 | 650 | A | C, T | 59 | 18 | 787 | 84 | 0.002514 | 0.106730636 |
| chr6 | 3.3E+07 | C | T | 15 | 35 | 200 | 165 | 0.00938 | 0.105232558 |
| chr6 | 3.3E+07 | T | C | 24 | 44 | 208 | 167 | 0.017764 | 0.10508253 |
| chr19 | 8371280 | C | T | 270 | 125 | 365 | 111 | 0.037004 | 0.104464167 |
| chrM | 3954 | C | T | 674 | 658 | 680 | 435 | 3.11E−06 | 0.104228466 |
| chr6 | 3.3E+07 | G | A | 16 | 36 | 203 | 168 | 0.01102 | 0.103411228 |
| chr6 | 3E+07 | C | T | 56 | 134 | 973 | 715 | 3.62E−12 | 0.103410976 |
| chr6 | 3.3E+07 | A | G | 22 | 42 | 210 | 170 | 0.016346 | 0.103285621 |
| chr6 | 3.3E+07 | T | G | 131 | 178 | 467 | 375 | 0.000748 | 0.102817106 |
| chr6 | 3.3E+07 | A | G | 117 | 163 | 464 | 373 | 0.000687 | 0.102727541 |
| chr6 | 3.3E+07 | C | A | 149 | 191 | 455 | 356 | 0.001195 | 0.102488589 |
| chr6 | 3.3E+07 | G | A, C | 103 | 148 | 479 | 382 | 0.000439 | 0.102269338 |
| chr6 | 3.3E+07 | T | C | 136 | 180 | 494 | 386 | 0.000575 | 0.102148186 |
| chr3 | 1.9E+08 | C | G | 338 | 229 | 438 | 197 | 0.005125 | 0.101991675 |
| chr6 | 3.3E+07 | T | G, A | 86 | 132 | 478 | 387 | 0.000298 | 0.101852991 |
| chr6 | 3E+07 | G | T | 28 | 119 | 1144 | 831 | 3.80E−18 | 0.101372373 |
| chr6 | 3E+07 | G | A | 47 | 130 | 1056 | 776 | 6.50E−14 | 0.100876798 |
| chr1 | 6.7E+07 | A | T | 2 | 3 | 317 | 25 | 0.003177 | 0.100873265 |
| chr11 | 6.5E+07 | A | G | 1001 | 1057 | 479 | 303 | 2.62E−08 | 0.100854531 |
| chr20 | 4.9E+07 | T | G, A, C | 288 | 141 | 68 | 14 | 0.032722 | 0.100688655 |
| chrM | 7792 | C | T | 594 | 650 | 855 | 623 | 1.72E−06 | 0.100666982 |
| chr14 | 1.1E+08 | C | T, G | 39 | 91 | 238 | 286 | 0.011087 | 0.100585087 |

| | Gene | | | | | |
|---|---|---|---|---|---|---|
| CHR | Name | RefSeq_ID | Coding | Func_type | Mutation_type | Cosmic |
| chr2 | MIR4435-1; | . | | intergenic | . | haematopoietic_and_lymphoid_tissue; |
| | ANAPC1P4 | | | | | cervix; lung |
| chr6 | HLA-C | . | | intronic | . | lung; large_intestine |
| chr6 | HLA-C | . | | intronic | . | large_intestine |
| chr6 | HLA-C | . | | intronic | . | large_intestine |
| chr6 | HLA-C | . | | intronic | . | lung; large_intestine |
| chr3 | MRPS25 | . | | intronic | . | large_intestine |
| chr17 | KRBA2 | . | | UTR5 | . | salivary_gland; |
| | | | | | | large_intestine; |
| | | | | | | urinary_tract; |
| | | | | | | liver; |
| | | | | | | stomach; |
| | | | | | | haematopoietic_and_lymphoid_tissue |
| chr7 | CYREN; | . | | intergenic | . | large_intestine; |
| | WDR91 | | | | | haematopoietic_and_lymphoid_tissue |
| chr2 | LOC100506474; | . | | intergenic | . | pancreas |
| | LINC00276 | | | | | |
| chr6 | HLA-C | . | | intronic | . | large_intestine |
| chr2 | LOC100506474; | . | | intergenic | . | pancreas |
| | LINC00276 | | | | | |
| chr6 | HLA-C | . | | intronic | . | large_intestine |
| chr6 | HLA-C | . | | intronic | . | lung; |
| | | | | | | large_intestine |
| KI270467.1 | NONE; NONE | . | | intergenic | . | liver |
| KI270467.1 | NONE; NONE | . | | intergenic | . | liver |
| chr8 | GPAT4 | . | | intronic | . | oesophagus |
| KI270467.1 | NONE; NONE | . | | intergenic | . | liver |
| KI270467.1 | NONE; NONE | . | | intergenic | . | liver |
| KI270467.1 | NONE; NONE | . | | intergenic | . | liver |
| KI270467.1 | NONE; NONE | . | | intergenic | . | liver |
| chr7 | HILPDA | . | | UTR3 | . | lung |
| KI270467.1 | NONE; NONE | . | | intergenic | . | liver |
| chr3 | EXOG | . | | intronic | . | oesophagus |
| KI270467.1 | NONE; NONE | . | | intergenic | . | liver |
| chr2 | LOC100506474; | . | | intergenic | . | pancreas |
| | LINC00276 | | | | | |
| KI270467.1 | NONE; NONE | . | | intergenic | . | liver |
| KI270467.1 | NONE; NONE | . | | intergenic | . | liver |
| KI270467.1 | NONE; NONE | . | | intergenic | . | liver |
| chr2 | LOC100506474; | . | | intergenic | . | pancreas |
| | LINC00276 | | | | | |
| KI270467.1 | NONE; NONE | . | | intergenic | . | liver |
| KI270467.1 | NONE; NONE | . | | intergenic | . | liver |
| chr17 | FLII | NM_001256265 | c.C3612T | exonic | synonymous_SNV | haematopoietic_and_lymphoid_tissue; |
| | | | | | | large_intestine; |
| | | | | | | thyroid; |
| | | | | | | urinary_tract; |
| | | | | | | lung |
| KI270467.1 | NONE; NONE | . | | intergenic | . | liver |
| KI270467.1 | NONE; NONE | . | | intergenic | . | liver |
| KI270467.1 | NONE; NONE | . | | intergenic | . | liver |
| KI270467.1 | NONE; NONE | . | | intergenic | . | liver |
| chr6 | HLA-F | . | | intronic | . | thyroid |
| KI270467.1 | NONE; NONE | . | | intergenic | . | liver |
| chr17 | SAT2; | . | | intronic | . | liver; |
| | SHBG | | | | | lung; |
| | | | | | | large_intestine; |
| | | | | | | haematopoietic_and_lymphoid_tissue |
| KI270467.1 | NONE; NONE | . | | intergenic | . | liver |
| KI270467.1 | NONE; NONE | . | | intergenic | . | liver |
| chr2 | LOC100506474; | . | | intergenic | . | pancreas |
| | LINC00276 | | | | | |
| KI270467.1 | NONE; NONE | . | | intergenic | . | liver |
| KI270467.1 | NONE; NONE | . | | intergenic | . | liver |
| chr6 | HLA-C | . | | intronic | . | liver |
| KI270467.1 | NONE; NONE | . | | intergenic | . | liver |
| KI270467.1 | NONE; NONE | . | | intergenic | . | liver |
| KI270467.1 | NONE; NONE | . | | intergenic | . | liver |
| KI270467.1 | NONE; NONE | . | | intergenic | . | liver |
| chr6 | HLA-F | . | | intronic | . | haematopoietic_and_lymphoid_tissue; |
| | | | | | | lung |
| KI270467.1 | NONE; NONE | . | | intergenic | . | liver |
| KI270467.1 | NONE; NONE | . | | intergenic | . | liver |
| chr3 | PDCD6IP | . | | intronic | . | oesophagus |
| KI270467.1 | NONE; NONE | . | | intergenic | . | liver |
| KI270467.1 | NONE; NONE | . | | intergenic | . | liver |
| KI270467.1 | NONE; NONE | . | | intergenic | . | liver |
| KI270467.1 | NONE; NONE | . | | intergenic | . | liver |
| chr7 | HILPDA | . | | UTR3 | . | lung |
| KI270467.1 | NONE; NONE | . | | intergenic | . | liver |
| KI270467.1 | NONE; NONE | . | | intergenic | . | liver |
| KI270467.1 | NONE; NONE | . | | intergenic | . | liver |
| KI270467.1 | NONE; NONE | . | | intergenic | . | liver |
| KI270467.1 | NONE; NONE | . | | intergenic | . | liver |
| KI270467.1 | NONE; NONE | . | | intergenic | . | liver |
| KI270467.1 | NONE; NONE | . | | intergenic | . | liver |
| chr3 | MITF | . | | intronic | . | oesophagus |
| KI270467.1 | NONE; NONE | . | | intergenic | . | liver |
| KI270467.1 | NONE; NONE | . | | intergenic | . | liver |
| KI270467.1 | NONE; NONE | . | | intergenic | . | liver |
| chr6 | LOC101929705 | . | | ncRNA_intronic | . | lung |
| KI270467.1 | NONE; NONE | . | | intergenic | . | liver |
| KI270467.1 | NONE; NONE | . | | intergenic | . | liver |
| KI270467.1 | NONE; NONE | . | | intergenic | . | liver |
| KI270467.1 | NONE; NONE | . | | intergenic | . | liver |
| KI270467.1 | NONE; NONE | . | | intergenic | . | liver |
| chr6 | EZR | . | | intronic | . | large_intestine |
| chr7 | CYREN; | . | | intergenic | . | large_intestine; |
| | WDR91 | | | | | haematopoietic_and_lymphoid_tissue |
| chr7 | CYREN; | . | | intergenic | . | large_intestine; |
| | WDR91 | | | | | haematopoietic_and_lymphoid_tissue |
| KI270467.1 | NONE; NONE | . | | intergenic | . | liver |
| KI270467.1 | NONE; NONE | . | | intergenic | . | liver |
| chrX | FTX | . | | ncRNA_intronic | . | large_intestine; |
| | | | | | | lung; |
| | | | | | | eye |
| KI270467.1 | NONE; NONE | . | | intergenic | . | liver |
| chr7 | CYREN; | . | | intergenic | . | large_intestine; |
| | WDR91 | | | | | haematopoietic_and_lymphoid_tissue |
| KI270467.1 | NONE; NONE | . | | intergenic | . | liver |
| KI270467.1 | NONE; NONE | . | | intergenic | . | liver |
| chr1 | MTR | . | | intronic | . | haematopoietic_and_lymphoid_tissue; |
| | | | | | | prostate |
| KI270467.1 | NONE; NONE | . | | intergenic | . | liver |
| chr6 | HLA-C | . | | UTR5 | . | oesophagus; |
| | | | | | | thyroid; |
| | | | | | | biliary_tract |
| chr6 | HLA-C | . | | UTR5 | . | upper_aerodigestive_tract; |
| | | | | | | urinary_tract; |
| | | | | | | haematopoietic_and_lymphoid_tissue |
| KI270467.1 | NONE; NONE | . | | intergenic | . | liver |
| chr4 | UFSP2 | . | | upstream | . | skin |
| KI270467.1 | NONE; NONE | . | | intergenic | . | liver |
| KI270467.1 | NONE; NONE | . | | intergenic | . | liver |
| KI270467.1 | NONE; NONE | . | | intergenic | . | liver |
| KI270467.1 | NONE; NONE | . | | intergenic | . | liver |
| chr2 | LOC100506474; | . | | intergenic | . | pancreas |
| | LINC00276 | | | | | |
| KI270467.1 | NONE; NONE | . | | intergenic | . | liver |
| KI270467.1 | NONE; NONE | . | | intergenic | . | liver |
| KI270467.1 | NONE; NONE | . | | intergenic | . | liver |
| KI270467.1 | NONE; NONE | . | | intergenic | . | liver |
| KI270467.1 | NONE; NONE | . | | intergenic | . | liver |
| KI270467.1 | NONE; NONE | . | | intergenic | . | liver |
| KI270467.1 | NONE; NONE | . | | intergenic | . | liver |
| KI270467.1 | NONE; NONE | . | | intergenic | . | liver |
| KI270467.1 | NONE; NONE | . | | intergenic | . | liver |
| KI270467.1 | NONE; NONE | . | | intergenic | . | liver |
| KI270467.1 | NONE; NONE | . | | intergenic | . | liver |
| KI270467.1 | NONE; NONE | . | | intergenic | . | liver |
| KI270467.1 | NONE; NONE | . | | intergenic | . | liver |
| KI270467.1 | NONE; NONE | . | | intergenic | . | liver |
| KI270467.1 | NONE; NONE | . | | intergenic | . | liver |
| KI270467.1 | NONE; NONE | . | | intergenic | . | liver |
| chr3 | BHLHE40 | . | | UTR3 | . | lung |
| KI270467.1 | NONE; NONE | . | | intergenic | . | liver |
| KI270467.1 | NONE; NONE | . | | intergenic | . | liver |
| KI270467.1 | NONE; NONE | . | | intergenic | . | liver |
| KI270467.1 | NONE; NONE | . | | intergenic | . | liver |
| chr11 | MALAT1; | . | | ncRNA_exonic | . | lung |
| | TALAM1 | | | | | |
| KI270467.1 | NONE; NONE | . | | intergenic | . | liver |
| KI270467.1 | NONE; NONE | . | | intergenic | . | liver |
| chr2 | LOC100506474; | . | | intergenic | . | pancreas |
| | LINC00276 | | | | | |
| KI270467.1 | NONE; NONE | . | | intergenic | . | liver |
| KI270467.1 | NONE; NONE | . | | intergenic | . | liver |
| KI270467.1 | NONE; NONE | . | | intergenic | . | liver |
| KI270467.1 | NONE; NONE | . | | intergenic | . | liver |
| chr11 | NEAT1 | . | | ncRNA_exonic | . | thyroid; |
| | | | | | | large_intestine |
| chr11 | NEAT1 | . | | ncRNA_exonic | . | thyroid; |
| | | | | | | large_intestine |
| KI270467.1 | NONE; NONE | . | | intergenic | . | liver |
| KI270467.1 | NONE; NONE | . | | intergenic | . | liver |
| KI270467.1 | NONE; NONE | . | | intergenic | . | liver |
| KI270467.1 | NONE; NONE | . | | intergenic | . | liver |
| chr11 | NEAT1 | . | | ncRNA_exonic | . | thyroid; |
| | | | | | | large_intestine |
| chr11 | NEAT1 | . | | ncRNA_exonic | . | thyroid; |
| | | | | | | large_intestine |
| chr6 | EZR | . | | intronic | . | large_intestine |
| KI270467.1 | NONE; NONE | . | | intergenic | . | liver |
| chr17 | SAT2 | NM_001320845 | c.G531A | exonic | synonymous_SNV | salivary_gland; |
| | | | | | | large_intestine; |
| | | | | | | urinary_tract; |
| | | | | | | liver; |
| | | | | | | stomach; |
| | | | | | | haematopoietic_and_lymphoid_tissue |
| chr3 | BHLHE40-AS1 | . | | ncRNA_intronic | . | lung |
| KI270467.1 | NONE; NONE | . | | intergenic | . | liver |
| KI270467.1 | NONE; NONE | . | | intergenic | . | liver |
| KI270467.1 | NONE; NONE | . | | intergenic | . | liver |
| KI270467.1 | NONE; NONE | . | | intergenic | . | liver |
| chr9 | EHMT1 | NM_001354259 | c.T351C | exonic | synonymous_SNV | large_intestine; |
| | | | | | | breast; |
| | | | | | | haematopoietic_and_lymphoid_tissue; |
| | | | | | | stomach |
| KI270467.1 | NONE; NONE | . | | intergenic | . | liver |
| KI270467.1 | NONE; NONE | . | | intergenic | . | liver |
| KI270467.1 | NONE; NONE | . | | intergenic | . | liver |
| KI270467.1 | NONE; NONE | . | | intergenic | . | liver |
| KI270467.1 | NONE; NONE | . | | intergenic | . | liver |
| KI270467.1 | NONE; NONE | . | | intergenic | . | liver |
| KI270467.1 | NONE; NONE | . | | intergenic | . | liver |
| chr8 | CYRIB | . | | intronic | . | haematopoietic_and_lymphoid_tissue; |
| | | | | | | large_intestine; |
| | | | | | | lung |
| KI270467.1 | NONE; NONE | . | | intergenic | . | liver |
| KI270467.1 | NONE; NONE | . | | intergenic | . | liver |
| KI270467.1 | NONE; NONE | . | | intergenic | . | liver |
| chr7 | HILPDA | . | | UTR3 | . | lung |
| chr3 | MITF | . | | intronic | . | oesophagus |
| KI270467.1 | NONE; NONE | . | | intergenic | . | liver |
| chr12 | RMST | . | | ncRNA_intronic | . | large_intestine; |
| | | | | | | pancreas; |
| | | | | | | haematopoietic_and_lymphoid_tissue |
| chr1 | PRRC2C; | . | | intergenic | . | liver; |
| | MYOCOS | | | | | breast |
| KI270467.1 | NONE; NONE | . | | intergenic | . | liver |
| chr11 | NEAT1 | . | | ncRNA_exonic | . | thyroid; |
| | | | | | | large_intestine |
| KI270467.1 | NONE; NONE | . | | intergenic | . | liver |
| KI270467.1 | NONE; NONE | . | | intergenic | . | liver |
| KI270467.1 | NONE; NONE | . | | intergenic | . | liver |
| KI270467.1 | NONE; NONE | . | | intergenic | . | liver |
| chr11 | NEAT1 | . | | ncRNA_exonic | . | thyroid; |
| | | | | | | large_intestine |
| KI270467.1 | NONE; NONE | . | | intergenic | . | liver |
| KI270467.1 | NONE; NONE | . | | intergenic | . | liver |
| KI270467.1 | NONE; NONE | . | | intergenic | . | liver |
| KI270467.1 | NONE; NONE | . | | intergenic | . | liver |
| KI270467.1 | NONE; NONE | . | | intergenic | . | liver |
| KI270467.1 | NONE; NONE | . | | intergenic | . | liver |
| KI270467.1 | NONE; NONE | . | | intergenic | . | liver |
| KI270467.1 | NONE; NONE | . | | intergenic | . | liver |
| KI270467.1 | NONE; NONE | . | | intergenic | . | liver |
| KI270467.1 | NONE; NONE | . | | intergenic | . | liver |
| KI270467.1 | NONE; NONE | . | | intergenic | . | liver |
| KI270467.1 | NONE; NONE | . | | intergenic | . | liver |
| KI270467.1 | NONE; NONE | . | | intergenic | . | liver |
| KI270467.1 | NONE; NONE | . | | intergenic | . | liver |
| KI270467.1 | NONE; NONE | . | | intergenic | . | liver |
| KI270467.1 | NONE; NONE | . | | intergenic | . | liver |
| chr11 | NEAT1 | . | | ncRNA_exonic | . | thyroid; |
| | | | | | | large_intestine |
| KI270467.1 | NONE; NONE | . | | intergenic | liver | |
| KI270467.1 | NONE; NONE | . | | intergenic | . | liver |
| KI270467.1 | NONE; NONE | . | | intergenic | liver | |
| KI270467.1 | NONE; NONE | . | | intergenic | . | liver |
| KI270467.1 | NONE; NONE | . | | intergenic | . | liver |
| KI270466.1 | NONE; NONE | . | | intergenic | . | liver |
| KI270467.1 | NONE; NONE | . | | intergenic | liver | |
| KI270467.1 | NONE; NONE | . | | intergenic | liver | |
| KI270467.1 | NONE; NONE | . | | intergenic | liver | |
| KI270467.1 | NONE; NONE | . | | intergenic | . | liver |
| chr6 | PSORS1C3 | . | | ncRNA_intronic | . | central_nervous_system; |
| | | | | | | urinary_tract; |
| | | | | | | lung |
| chr11 | NEAT1 | . | | ncRNA_exonic | . | thyroid; |
| | | | | | | large_intestine |
| KI270467.1 | NONE; NONE | . | | intergenic | liver | |
| KI270467.1 | NONE; NONE | . | | intergenic | liver | |
| KI270467.1 | NONE; NONE | . | | intergenic | liver | |
| KI270467.1 | NONE; NONE | . | | intergenic | liver | |
| KI270467.1 | NONE; NONE | . | | intergenic | liver | |
| KI270467.1 | NONE; NONE | . | | intergenic | liver | |
| chr9 | CCDC107 | NM_174923 | c.A658G | exonic | nonsynonymous_SNV | salivary_gland |
| chr6 | HLA-A | . | | intronic | . | upper_aerodigestive_tract; |
| | | | | | | soft_tissue |
| KI270467.1 | NONE; NONE | . | | intergenic | . | liver |
| KI270467.1 | NONE; NONE | . | | intergenic | . | liver |
| KI270467.1 | NONE; NONE | . | | intergenic | . | liver |
| chr11 | NEAT1 | . | | ncRNA_exonic | . | thyroid; |
| | | | | | | large_intestine |
| chr6 | PRPF4B | NM_003913 | c.A247G | exonic | nonsynonymous_SNV | haematopoietic_and_lymphoid_tissue; |
| | | | | | | lung |
| chr11 | NEAT1 | . | | ncRNA_exonic | . | thyroid; |
| | | | | | | large_intestine |
| chr11 | NEAT1 | . | | ncRNA_exonic | . | thyroid; |
| | | | | | | large_intestine |
| KI2704671 | NONE; NONE | . | | intergenic | . | liver |
| KI2704671 | NONE; NONE | . | | intergenic | . | liver |
| chr12 | RMST | . | | ncRNA_intronic | . | large_intestine; |
| | | | | | | pancreas; |
| | | | | | | haematopoietic_and_lymphoid_tissue |
| chr11 | MALAT1; | . | | ncRNA_exonic | . | central_nervous_system |
| | TALAM1 | | | | | |
| KI270467.1 | NONE; NONE | . | | intergenic | . | liver |
| KI270467.1 | NONE; NONE | . | | intergenic | . | liver |
| KI270467.1 | NONE; NONE | . | | intergenic | . | liver |
| chr6 | HLA-DQA1 | . | | UTR3 | . | liver |
| KI270467.1 | NONE; NONE | . | | intergenic | . | liver |
| chr6 | PSORS1C3 | . | | ncRNA_intronic | . | central_nervous_system; |
| | | | | | | urinary_tract; |
| | | | | | | lung |
| KI270466.1 | NONE; NONE | . | | intergenic | . | liver |
| KI270467.1 | NONE; NONE | . | | intergenic | . | liver |
| KI270467.1 | NONE; NONE | . | | intergenic | . | liver |
| KI270467.1 | NONE; NONE | . | | intergenic | . | liver |
| chr6 | HLA-A | . | | intronic | . | liver; |
| | | | | | | eye; |
| | | | | | | upper_aerodigestive_tract; |
| | | | | | | lung; |
| | | | | | | large_intestine; |
| | | | | | | thyroid; |
| | | | | | | NS |
| KI270467.1 | NONE; NONE | . | | intergenic | . | liver |
| chr7 | HILPDA | . | | UTR3 | . | lung |
| chr7 | HILPDA | . | | UTR3 | . | lung |
| KI270467.1 | NONE; NONE | . | | intergenic | . | liver |
| chr11 | NEAT1 | . | | ncRNA_exonic | . | thyroid; |
| | | | | | | large_intestine |
| KI270467.1 | NONE; NONE | . | | intergenic | . | liver |
| KI270467.1 | NONE; NONE | . | | intergenic | . | liver |
| chrX | TSIX; | . | | ncRNA_exonic | . | large_intestine; |
| | XIST | | | | | lung; eye |
| chrX | FTX | . | | ncRNA_intronic | . | large_intestine; |
| | | | | | | lung; eye |
| KI270466.1 | NONE; NONE | . | | intergenic | . | liver |
| KI270467.1 | NONE; NONE | . | | intergenic | . | liver |
| KI270467.1 | NONE; NONE | . | | intergenic | . | liver |
| KI270467.1 | NONE; NONE | . | | intergenic | . | liver |
| chr4 | KCNIP4 | . | | intronic | . | lung |
| chrX | TSIX; | . | | neRNA_exonic | . | large_intestine; |
| | XIST | | | | | lung; eye |
| chr6 | HLA-C | . | | UTR3 | . | central_nervous_system; |
| | | | | | | urinary_tract; lung |
| KI270467.1 | NONE; NONE | . | | intergenic | . | liver |
| chr11 | NEAT1 | . | | ncRNA_exonic | . | haematopoietic_and_lymphoid_tissue |
| KI270467.1 | NONE; NONE | . | | intergenic | . | liver |
| chr7 | HILPDA | . | | UTR3 | . | lung |
| chr19 | CLEC11A | NM_002975 | c.G882A | exonic | synonymous_SNV | haematopoietic_and_lymphoid_tissue; |
| | | | | | | salivary_gland; lung |
| KI270466.1 | NONE; NONE | . | | intergenic | . | liver |
| chr3 | AZI2 | . | | intronic | . | large_intestine |
| chr6 | H4C8; | . | | intergenic | . | haematopoietic_and_lymphoid_tissue; |
| | BTN3A2 | | | | | lung |
| KI270467.1 | NONE; NONE | . | | intergenic | . | liver |
| chr6 | PSORS1C3 | . | | ncRNA_intronic | . | central_nervous_system; |
| | | | | | | urinary_tract; lung |
| chr6 | VEGFA | . | | UTR3 | . | lung |
| chr6 | HLA-A | . | | intronic | . | haematopoietic_and_lymphoid_tissue |
| chr3 | BHLHE40 | . | | UTR3 | . | lung |
| KI2704671 | NONE; NONE | . | | intergenic | . | liver |
| chr8 | GPAT4 | . | | intronic | . | liver |
| KI270467.1 | NONE; NONE | . | | intergenic | . | liver |
| chr19 | ANGPTL4 | . | | intronic | . | haematopoietic_and_lymphoid_tissue |
| chr6 | HLA-A | . | | intronic | . | thyroid |
| chr4 | KCNIP4 | . | | intronic | . | lung |
| KI270466.1 | NONE; NONE | . | | intergenic | . | liver |
| KI270467.1 | NONE; NONE | . | | intergenic | . | liver |
| KI270467.1 | NONE; NONE | . | | intergenic | . | liver |
| KI270467.1 | NONE; NONE | . | | intergenic | . | liver |
| chr8 | ZNF252P-AS1; | . | | intergenic | . | bone |
| | C80rf33 | | | | | |
| KI270467.1 | NONE; NONE | . | | intergenic | . | liver |
| chr6 | RPP21; | NM_001199120 | c.C469A | exonic | nonsynonymous_SNV | thyroid |
| | TRIM39-RPP21 | | | | | |
| KI270467.1 | NONE; NONE | . | | intergenic | . | liver |
| chr6 | HLA-A | . | | intronic | . | thyroid |
| chr4 | KCNIP4 | . | | intronic | . | lung |
| KI270467.1 | NONE; NONE | . | | intergenic | . | liver |
| KI270467.1 | NONE; NONE | . | | intergenic | . | liver |
| KI270467.1 | NONE; NONE | . | | intergenic | . | liver |
| KI270467.1 | NONE; NONE | . | | intergenic | . | liver |
| chrX | FTX | . | | ncRNA_intronic | . | large_intestine; lung; eye |
| chr6 | SRSF3 | . | | UTR3 | . | lung |
| KI270466.1 | NONE; NONE | . | | intergenic | . | liver |
| chr6 | HLA-DQA1 | . | | UTR3 | . | liver |
| chr11 | NEAT1 | . | | ncRNA_exonic | . | haematopoietic_and_lymphoid_tissue |
| KI270467.1 | NONE; NONE | . | | intergenic | . | liver |
| KI270467.1 | NONE; NONE | . | | intergenic | . | liver |
| chr7 | HILPDA | . | | UTR3 | . | lung |
| KI270467.1 | NONE; NONE | . | | intergenic | . | liver |
| chr15 | LINC02256 | . | | ncRNA_exonic | . | haematopoietic_and_lymphoid_tissue |
| chr4 | KCNIP4 | . | | intronic | . | lung |
| chr11 | NEAT1 | . | | ncRNA_exonic | . | thyroid; large_intestine |
| chrX | TSIX; | . | | ncRNA_exonic | . | large_intestine; lung; eye |
| | XIST | | | | | |
| chrX | FTX | . | | neRNA_intronic | . | large_intestine; lung; eye |
| chr5 | STING1; | . | | intergenic | . | pancreas |
| | UBE2D2 | | | | | |
| chr6 | TAPBP | . | | UTR3 | . | central_nervous_system |
| KI270467.1 | NONE; NONE | . | | intergenic | . | liver |
| chrX | FTX | . | | ncRNA_intronic | . | large_intestine; lung; eye |
| KI270467.1 | NONE; NONE | . | | intergenic | . | liver |
| KI2704671 | NONE; NONE | . | | intergenic | . | liver |
| KI270466.1 | NONE; NONE | . | | intergenic | . | liver |
| chr18 | ZNF532; | . | | intergenic | . | large_intestine; kidney; |
| | OACYLP | | | | | haematopoietic_and_lymphoid_tissue |
| KI270467.1 | NONE; NONE | . | | intergenic | . | liver |
| KI270467.1 | NONE; NONE | . | | intergenic | . | liver |
| KI270467.1 | NONE; NONE | . | | intergenic | . | liver |
| KI270466.1 | NONE; NONE | . | | intergenic | . | liver |
| chr4 | KCNIP4 | . | | intronic | . | lung |
| chr6 | HLA-DRB1 | . | | intronic | . | haematopoietic_and_lymphoid_tissue; |
| | | | | | | liver; large_intestine |
| KI270467.1 | NONE; NONE | . | | intergenic | . | liver |
| KI270467.1 | NONE; NONE | . | | intergenic | . | liver |
| KI270467.1 | NONE; NONE | . | | intergenic | . | liver |
| chr7 | HILPDA | . | | UTR3 | . | lung |
| KI270467.1 | NONE; NONE | . | | intergenic | . | liver |
| KI270467.1 | NONE; NONE | . | | intergenic | . | liver |
| KI270467.1 | NONE; NONE | . | | intergenic | . | liver |
| chr11 | NEAT1 | . | | ncRNA_exonic | . | thyroid; large_intestine |
| chr11 | NEAT1 | . | | ncRNA_exonic | . | haematopoietic_and_lymphoid_tissue |
| chr3 | GBE1 | . | | UTR5 | . | oesophagus |
| chrX | XIST | . | | ncRNA_exonic | . | large_intestine; |
| | | | | | | lung; eye |
| chr2 | LINC01320 | . | | ncRNA_exonic | . | thyroid |
| chr8 | GPAT4 | . | | intronic | . | liver |
| KI270467.1 | NONE; NONE | . | | intergenic | . | liver |
| KI270466.1 | NONE; NONE | . | | intergenic | . | liver |
| chr19 | ANGPTL4 | . | | UTR5 | . | haematopoietic_and_lymphoid_tissue |
| KI270467.1 | NONE; NONE | . | | intergenic | . | liver |
| chr8 | NDRG1 | . | | intronic | . | prostate |
| chr9 | CEMIP2 | . | | intronic | . | liver; large_intestine |
| KI270467.1 | NONE; NONE | . | | intergenic | . | liver |
| chr11 | PGGHG | . | | UTR3 | . | lung |
| KI270466.1 | NONE; NONE | . | | intergenic | . | liver |
| KI270466.1 | NONE; NONE | . | | intergenic | . | liver |
| KI270467.1 | NONE; NONE | . | | intergenic | . | liver |
| chrX | ATRX | NM_138270 | c.G2671C | exonic | nonsynonymous_SNV | large_intestine; |
| | | | | | | lung; eye |
| KI270466.1 | NONE; NONE | . | | intergenic | . | liver |
| chr11 | MALAT1; | . | | ncRNA_exonic | . | central_nervous_system |
| | TALAM1 | | | | | |
| chrX | FTX | . | | ncRNA_intronic | . | large_intestine; lung; |
| | | | | | | eye |
| chr11 | PGGHG | . | | UTR3 | . | lung |
| chr9 | DPP7 | NM_013379 | c.T162C | exonic | synonymous_SNV | thyroid; large_intestine; |
| | | | | | | skin; prostate |
| KI270467.1 | NONE; NONE | . | | intergenic | . | liver |
| chr8 | NDRG1 | . | | intronic | . | prostate |
| chr4 | KCNIP4 | . | | intronic | . | lung |
| chr6 | HLA-A | . | | intronic | . | thyroid |
| KI270466.1 | NONE; NONE | . | | intergenic | . | liver |
| KI270466.1 | NONE; NONE | . | | intergenic | . | liver |
| chr11 | MALAT1; | . | | ncRNA_exonic | . | lung |
| | TALAM1 | | | | | |
| KI270466.1 | NONE; NONE | . | | intergenic | . | liver |
| KI270467.1 | NONE; NONE | . | | intergenic | . | liver |
| KI270467.1 | NONE; NONE | . | | intergenic | . | liver |
| KI270467.1 | NONE; NONE | . | | intergenic | . | liver |
| KI270467.1 | NONE; NONE | . | | intergenic | . | liver |
| KI270467.1 | NONE; NONE | . | | intergenic | . | liver |
| chr11 | NEAT1 | . | | ncRNA_exonic | . | thyroid; large_intestine |
| chr4 | UFSP2 | . | | upstream | . | skin |
| KI270466.1 | NONE; NONE | . | | intergenic | . | liver |
| chr6 | HLA-DRB1 | . | | intronic | . | haematopoietic_and_lymphoid_tissue; |
| | | | | | | liver; large_intestine |
| chr8 | NDRG1 | . | | intronic | . | lung; |
| | | | | | | haematopoietic_and_lymphoid_tissue |
| chr6 | HLA-C | NM_001243042 | c.A985G | exonic | nonsynonymous_SNV | thyroid; |
| | | | | | | large_intestine; |
| | | | | | | haematopoietic_and_lymphoid_tissue |
| chrX | XIST | . | | ncRNA_exonic | . | large_intestine; lung; |
| | | | | | | eye |
| chr6 | RPP21; | NM_001199120 | c.T468G | exonic | synonymous_SNV | upper_aerodigestive_tract |
| | TRIM39-RPP21 | | | | | |
| chr11 | PGGHG | . | | UTR3 | . | lung |
| chr6 | HLA-C | NM_001243042 | c.G201A | exonic | synonymous_SNV | oesophagus; lung; |
| | | | | | | haematopoietic_and_lymphoid_tissue |
| KI270467.1 | NONE; NONE | . | | intergenic | . | liver |
| chr8 | NDRG1 | . | | intronic | . | lung; |
| | | | | | | haematopoietic_and_lymphoid_tissue |
| chr2 | UGT1A6 | NM_001072 | c.T19G | exonic | nonsynonymous_SNV | haematopoietic_and_lymphoid_tissue; |
| | | | | | | skin; large_intestine; |
| | | | | | | thyroid; lung |
| chr11 | PGGHG | . | | UTR3 | . | lung |
| KI270467.1 | NONE; NONE | . | | intergenic | . | liver |
| KI270467.1 | NONE; NONE | . | | intergenic | . | liver |
| chr7 | HILPDA | . | | UTR3 | . | lung |
| KI270467.1 | NONE; NONE | . | | intergenic | . | liver |
| chr6 | HLA-DQA1 | . | | UTR5 | . | haematopoietic_and_lymphoid_tissue; |
| | | | | | | liver; large_intestine |
| chr14 | HNRNPC | . | | intronic | . | prostate |
| chr1 | PAPPA2 | . | | UTR5 | . | pancreas |
| KI270467.1 | NONE; NONE | . | | intergenic | . | liver |
| chr8 | GPAT4 | . | | intronic | . | liver |
| chr6 | HLA-C | NM_001243042 | c.A872T | exonic | nonsynonymous_SNV | upper_aerodigestive_tract; |
| | | | | | | large_intestine; oesophagus |
| chrX | FTX | . | | ncRNA_intronic | . | large_intestine; lung; eye |
| chr6 | HMGN3 | . | | intronic | . | lung |
| KI270467.1 | NONE; NONE | . | | intergenic | . | liver |
| chr11 | NEAT1 | . | | neRNA_exonic | . | haematopoietic_and_lymphoid_tissue |
| chr16 | GALNS | . | | UTR3 | . | thyroid |
| chr6 | HLA-C | NM_001243042 | c.C989T | exonic | nonsynonymous_SNV | haematopoietic_and_lymphoid_tissue; |
| | | | | | | thyroid; large_intestine; |
| | | | | | | upper_aerodigestive_tract |
| chr7 | CYREN; | . | | intergenic | . | large_intestine; |
| | WDR91 | | | | | haematopoietic_and_lymphoid_tissue |
| chr11 | NDUFV1 | . | | intronic | . | haematopoietic_and_lymphoid_tissue |
| chr6 | HLA-DRB1 | . | | intronic | . | haematopoietic_and_lymphoid_tissue; |
| | | | | | | liver; large_intestine |
| chr6 | LOC101927730 | . | | ncRNA_intronic | . | biliary_tract |
| chr6 | RPP21; | . | | UTR3 | . | thyroid |
| | TRIM39-RPP21 | | | | | |
| chr6 | LOC101927730 | . | | ncRNA_intronic | . | biliary_tract |
| chr6 | HLA-A | . | | intronic | . | large_intestine; liver |
| chrX | RRAGB | . | | UTR5 | . | pancreas |
| chr11 | NDUFV1 | . | | intronic | . | haematopoietic_and_lymphoid_tissue |
| chr4 | KCNIP4 | . | | intronic | . | lung |
| chr2 | LINC01320 | . | | neRNA_exonic | . | thyroid |
| chr6 | HLA-C | NM_001243042 | c.G47C | exonic | nonsynonymous_SNV | thyroid; oesophagus; |
| | | | | | | biliarytract |
| chr6 | HLA-C | . | | UTR5 | . | oesophagus; |
| | | | | | | thyroid; biliary_tract |
| chr6 | HLA-DRB1 | . | | intronic | . | haematopoietic_and_lymphoid_tissue; |
| | | | | | | liver; large_intestine |
| chr6 | HLA-C | NM_001243042 | c.C28G | exonic | nonsynonymous_SNV | oesophagus; thyroid |
| chr6 | HLA-DRB1 | . | | intronic | . | haematopoietic_and_lymphoid_tissue; |
| | | | | | | liver; large_intestine |
| chr6 | HLA-A | . | | intronic | . | large_intestine; liver |
| KI270467.1 | NONE; NONE | . | | intergenic | . | liver |
| chr8 | NDRG1 | . | | intronic | . | prostate |
| chr6 | HLA-C | NM_001243042 | c.T512A | exonic | stopgain | upper_aerodigestive_tract; |
| | | | | | | biliary_tract; thyroid |
| chr19 | GDF15 | NM_004864 | c.G420T | exonic | synonymous_SNV | stomach; large_intestine |
| chr6 | HLA-C | NM_001243042 | c.T972A | exonic | synonymous_SNV | bone; kidney |
| KI270467.1 | NONE; NONE | . | | intergenic | . | liver |
| chr6 | HLA-A | . | | intronic | . | soft_tissue; liver; |
| | | | | | | large_intestine |
| chr6 | HLA-C | NM_001243042 | c.A991G | exonic | nonsynonymous_SNV | haematopoietic_and_lymphoid_tissue; |
| | | | | | | largeintestine; thyroid |
| KI270467.1 | NONE; NONE | . | | intergenic | . | liver |
| chrX | RBM3 | . | | UTR3 | . | lung |
| chr1 | PRRC2C; | . | | intergenic | . | liver; breast |
| | MYOCOS | | | | | |
| chr6 | HLA-DMA | NM_006120 | c.G496A | exonic | nonsynonymous_SNV | central_nervous_system |
| KI270466.1 | NONE; NONE | . | | intergenic | . | liver |
| chr17 | RNASEK-C17orf49 | . | | ncRNA_exonic | . | thyroid; |
| | | | | | | haematopoietic_and_lymphoid_tissue |
| chr6 | HLA-C | NM_001243042 | c.G97T | exonic | nonsynonymous_SNV | large_intestine; oesophagus; |
| | | | | | | autonomic_ganglia |
| chr6 | HLA-C | NM_001243042 | c.C984G | exonic | synonymous_SNV | haematopoietic_and_lymphoid_tissue; |
| | | | | | | large_intestine; thyroid |
| chr9 | ANXA1 | NM_000700 | c.A931C | exonic | nonsynonymous_SNV | liver; large_intestine |
| chrM | NONE; MIR12136 | . | | intergenic | . | large_intestine; breast; |
| | | | | | | haematopoietic_and_lymphoid_tissue; |
| | | | | | | stomach |
| chr19 | GDF15 | NM_004864 | c.G25C | exonic | nonsynonymous_SNV | lung; large_intestine; |
| | | | | | | stomach; skin |
| chr6 | HLA-C | . | | UTR3 | . | central_nervous_system; |
| | | | | | | urinary_tract; lung |
| chr11 | NEAT1 | . | | ncRNA_exonic | . | haematopoietic_and_lymphoid_tissue |
| KI270467.1 | NONE; NONE | . | | intergenic | . | liver |
| chr14 | NOVA1 | . | | UTR5 | . | large_intestine |
| chr6 | HLA-C | . | | UTR3 | . | central_nervous_system; |
| | | | | | | urinary_tract; lung |
| chr6 | HLA-C | NM_001243042 | c.A925G | exonic | nonsynonymous_SNV | lung; pancreas |
| chr21 | UBE2G2 | . | | UTR3 | . | soft_tissue |
| chr15 | GOLGA8B | . | | UTR5 | . | haematopoietic_and_lymphoid_tissue |
| chr6 | HLA-C | NM_001243042 | c.T956C | exonic | nonsynonymous_SNV | pancreas; |
| | | | | | | haematopoietic_and_lymphoid_tissue; |
| | | | | | | kidney; thyroid; large_intestine |
| chr6 | HLA-A | . | | intronic | . | large_intestine |
| chr6 | HLA-C | NM_001243042 | c.C829G | exonic | nonsynonymous_SNV | upper_aerodigestive_tract; |
| | | | | | | large_intestine; oesophagus |
| KI270467.1 | NONE; NONE | . | | intergenic | . | liver |
| chrX | SLC6A8 | . | | UTR3 | . | lung |
| KI270466.1 | NONE; NONE | . | | intergenic | . | liver |
| chr6 | HLA-C | NM_001243042 | c.C987T | exonic | synonymous_SNV | upper_aerodigestive_tract; |
| | | | | | | thyroid; large_intestine; |
| | | | | | | haematopoietic_and_lymphoid_tissue |
| chr6 | HLA-C | . | | UTR3 | . | central_nervous_system; |
| | | | | | | urinary_tract; lung |
| chr6 | MRPL14 | NM_001318771 | c.C198T | exonic | synonymous_SNV | lung |
| chr6 | HLA-C | . | | UTR3 | . | central_nervous_system; |
| | | | | | | urinary_tract; lung |
| chr6 | HLA-A | NM_001242758 | c.C41T | exonic | nonsynonymous_SNV | thyroid |
| chr6 | HLA-C | NM_001243042 | c.G22A | exonic | nonsynonymous_SNV | oesophagus; thyroid; biliary_tract |
| chr15 | GOLGA8B | . | | UTR5 | . | haematopoietic_and_lymphoid_tissue |
| chr6 | HLA-DRB1 | . | | intronic | . | haematopoietic_and_lymphoid_tissue; |
| | | | | | | liver; large_intestine |
| chrX | PNCK | NM_001039582 | c.G817A | exonic | nonsynonymous_SNV | endometrium |
| chr6 | HLA-DQA1 | NM_002122 | c.C60T | exonic | synonymous_SNV | haematopoietic_and_lymphoid_tissue; |
| | | | | | | liver; large_intestine |
| chr6 | HLA-DQA1 | NM_002122 | c.C705G | exonic | synonymous_SNV | upper_aerodigestive_tract; |
| | | | | | | kidney; large_intestine; |
| | | | | | | oesophagus; thyroid; |
| | | | | | | haematopoietic_and_lymphoid_tissue |
| chr12 | PRH1-PRR4 | . | | ncRNA_exonic | . | haematopoietic_and_lymphoid_tissue; |
| | | | | | | biliary_tract |
| chr6 | HLA-C | . | | UTR3 | . | large_intestine |
| chr17 | EIF5A | . | | UTR3 | . | thyroid; |
| | | | | | | haematopoietic_and_lymphoid_tissue |
| chr11 | NEAT1 | . | | neRNA_exonic | . | thyroid; large_intestine |
| chr10 | ATP5F1C | . | | intronic | . | haematopoietic_and_lymphoid_tissue; |
| | | | | | | lung |
| chr11 | MALAT1; | . | | ncRNA_exonic | . | lung |
| | TALAM1 | | | | | |
| chr6 | HLA-C | NM_001243042 | c.T142G | exonic | nonsynonymous_SNV | haematopoietic_and_lymphoid_tissue; |
| | | | | | | breast; thyroid; oesophagus; |
| | | | | | | large_intestine; liver; |
| | | | | | | biliary_tract; |
| | | | | | | upper_aerodigestive_tract |
| chr6 | HSP90AB1 | . | | UTR5 | . | lung |
| chr6 | HLA-DRB1 | . | | UTR3 | . | haematopoietic_and_lymphoid_tissue; |
| | | | | | | liver; large_intestine |
| KI270466.1 | NONE; NONE | . | | intergenic | . | liver |
| KI270466.1 | NONE; NONE | . | | intergenic | . | liver |
| chr6 | HLA-C | NM_001243042 | c.A341C | exonic | nonsynonymous_SNV | upper_aerodigestive_tract; |
| | | | | | | thyroid; lung |
| chr11 | CRYAB | NM_001368246 | c.C286T | exonic | nonsynonymous_SNV | biliary_tract; liver |
| chr11 | NEAT1 | . | | neRNA_exonic | . | thyroid; large_intestine |
| chr6 | HLA-A | . | | intronic | . | biliary_tract |
| chr7 | CYREN; | . | | intergenic | . | large_intestine; |
| | WDR91 | | | | | haematopoietic_and_lymphoid_tissue |
| KI270466.1 | NONE; NONE | . | | intergenic | . | liver |
| chr6 | HLA-C | . | | UTR3 | . | large_intestine |
| chr10 | DEPP1 | NM_007021 | c.C611T | exonic | nonsynonymous_SNV | liver |
| chr6 | H4C8; | . | | intergenic | . | haematopoietic_and_lymphoid_tissue; |
| | BTN3A2 | | | | | lung |
| chr6 | HLA-C | . | | UTR3 | . | central_nervous_system; |
| | | | | | | urinary_tract; lung |
| chr10 | DDIT4 | . | | UTR3 | . | lung |
| chr6 | HLA-C | NM_001243042 | c.G735A | exonic | synonymous_SNV | thyroid; large_intestine; lung; |
| | | | | | | upper_aerodigestive_tract; |
| | | | | | | eye; |
| | | | | | | haematopoietic_and_lymphoid_tissue; |
| | | | | | | NS |
| chr6 | HLA-C | NM_001243042 | c.C747T | exonic | synonymous_SNV | large_intestine; thyroid; |
| | | | | | | upper_aerodigestive_tract |
| KI270467.1 | NONE; NONE | . | | intergenic | . | liver |
| chr6 | HLA-C | NM_001243042 | c.C650T | exonic | nonsynonymous_SNV | thyroid |
| chr6 | HLA-C | . | | UTR3 | . | large_intestine |
| chr6 | HLA-C | NM_001243042 | c.G744A | exonic | synonymous_SNV | large_intestine; |
| | | | | | | oesophagus; thyroid; |
| | | | | | | upper_aerodigestive_tract |
| chr6 | HLA-C | NM_001243042 | c.T539C | exonic | nonsynonymous_SNV | haematopoietic_and_lymphoid_tissue; |
| | | | | | | skin; thyroid; |
| | | | | | | upper_aerodigestive_tract; |
| | | | | | | central_nervous_system; |
| | | | | | | biliary_tract |
| KI270467.1 | NONE; NONE | . | | intergenic | . | liver |
| chr6 | HLA-A | NM_001242758 | c.T1029C | exonic | synonymous_SNV | soft_tissue; |
| | | | | | | central_nervous_system; |
| | | | | | | upper_aerodigestive_tract |
| chrX | PNCK | . | | UTR3 | . | biliary_tract; thyroid |
| KI270467.1 | NONE; NONE | . | | intergenic | . | liver |
| chr11 | NEAT1 | . | | ncRNA_exonic | . | thyroid; large_intestine |
| chr7 | HILPDA | . | | UTR3 | . | lung |
| chr6 | HLA-C | . | | UTR3 | . | large_intestine |
| chr11 | NEAT1 | . | | ncRNA_exonic | . | thyroid; large_intestine |
| chr6 | HLA-C | NM_001243042 | c.G289A | exonic | nonsynonymous_SNV | haematopoietic_and_lymphoid_tissue; |
| | | | | | | upper_aerodigestive_tract; |
| | | | | | | central_nervous_system |
| chr6 | HLA-C | NM_001243042 | c.A873G | exonic | synonymous_SNV | large_intestine; |
| | | | | | | oesophagus; central_nervous_system |
| chrX | TSIX; | . | | ncRNA_exonic | . | large_intestine; lung; eye |
| | XIST | | | | | |
| chr6 | HLA-DRB1 | . | | UTR3 | . | haematopoietic_and_lymphoid_tissue; |
| | | | | | | liver; large_intestine |
| KI270466.1 | NONE; NONE | . | | intergenic | . | liver |
| chr6 | HLA-B | NM_005514 | c.C44A | exonic | nonsynonymous_SNV | lung; |
| | | | | | | haematopoietic_and_lymphoid_tissue; |
| | | | | | | soft_tissue |
| chr6 | HLA-C | . | | UTR3 | . | large_intestine |
| chr6 | HLA-C | NM_001243042 | c.G993T | exonic | nonsynonymous_SNV | upper_aerodigestive_tract; |
| | | | | | | thyroid |
| chr6 | HLA-DQA1 | NM_002122 | c.C714G | exonic | nonsynonymous_SNV | upper_aerodigestive_tract; |
| | | | | | | kidney; large_intestine; |
| | | | | | | oesophagus; thyroid; |
| | | | | | | haematopoietic_and_lymphoid_tissue |
| chr6 | HLA-A | NM_001242758 | c.A1033C | exonic | nonsynonymous_SNV | soft_tissue; |
| | | | | | | central_nervous_system; |
| | | | | | | liver; |
| | | | | | | upper_aerodigestive_tract |
| chr6 | HLA-A | . | | intronic | . | biliary_tract |
| chr6 | HLA-C | NM_001243042 | c.A853G | exonic | nonsynonymous_SNV | oesophagus; |
| | | | | | | upper_aerodigestive_tract |
| chr11 | NEAT1 | . | | ncRNA_exonic | . | thyroid; large_intestine |
| chr6 | HLA-C | . | | UTR3 | . | large_intestine |
| chr6 | HLA-DQA1 | NM_002122 | c.C591T | exonic | synonymous_SNV | haematopoietic_and_lymphoid_tissue; |
| | | | | | | liver; large_intestine |
| chr6 | HLA-C | NM_001243042 | c.C486G | exonic | synonymous_SNV | haematopoietic_and_lymphoid_tissue; |
| | | | | | | thyroid; liver; |
| | | | | | | central_nervous_system; |
| | | | | | | biliary_tract; |
| | | | | | | upper_aerodigestive_tract |
| chr6 | HLA-C | . | | UTR3 | . | central_nervous_system; |
| | | | | | | urinary_tract; lung |
| chr6 | HLA-C | NM_001243042 | c.A361T | exonic | nonsynonymous_SNV | haematopoietic_and_lymphoid_tissue; |
| | | | | | | skin; thyroid; large_intestine; |
| | | | | | | lung |
| chrX | FTX | . | | ncRNA_intronic | . | large_intestine; |
| | | | | | | lung; |
| | | | | | | eye |
| chr6 | HLA-A | . | | intronic | . | large_intestine |
| KI270467.1 | NONE; NONE | . | | intergenic | . | liver |
| chr6 | HLA-DQA1 | NM_002122 | c.A438C | exonic | synonymous_SNV | haematopoietic_and_lymphoid_tissue; |
| | | | | | | upper_aerodigestive_tract; |
| | | | | | | thyroid; |
| | | | | | | large_intestine |
| chrM | NONE; MIR12136 | . | | intergenic | . | large_intestine; |
| | | | | | | breast; |
| | | | | | | haematopoietic_and_lymphoid_tissue; |
| | | | | | | stomach |
| KI270466.1 | NONE; NONE | . | | intergenic | . | liver |
| chr6 | HLA-C | NM_001243042 | c.C648T | exonic | synonymous_SNV | thyroid |
| chr6 | HLA-C | NM_001243042 | c.C652A | exonic | nonsynonymous_SNV | thyroid |
| chr4 | KCNIP4 | . | | intronic | . | lung |
| KI270467.1 | NONE; NONE | . | | intergenic | . | liver |
| chr6 | HLA-A | NM_001242758 | c.T97A | exonic | nonsynonymous_SNV | thyroid |
| chr6 | HLA-C | . | | UTR3 | . | large_intestine |
| chr15 | GOLGA8B | . | | UTR5 | . | haematopoietic_and_lymphoid_tissue |
| chr6 | HLA-DQA1 | NM_002122 | c.C630A | exonic | synonymous_SNV | upper_aerodigestive_tract; |
| | | | | | | kidney; |
| | | | | | | large_intestine; |
| | | | | | | oesophagus; |
| | | | | | | thyroid; |
| | | | | | | haematopoietic_and_lymphoid_tissue |
| chr6 | HLA-C | NM_001243042 | c.A1087G | exonic | nonsynonymous_SNV | thyroid |
| chr6 | HLA-DQA1 | NM_002122 | c.T708C | exonic | synonymous_SNV | upper_aerodigestive_tract; |
| | | | | | | kidney; |
| | | | | | | large_intestine; |
| | | | | | | oesophagus; |
| | | | | | | thyroid; |
| | | | | | | haematopoietic_and_lymphoid_tissue |
| chr14 | PNN | . | | UTR3 | . | breast; |
| | | | | | | stomach; |
| | | | | | | large_intestine |
| chr6 | HLA-DRB1 | . | | UTR3 | . | haematopoietic_and_lymphoid_tissue; |
| | | | | | | liver; |
| | | | | | | large_intestine |
| chr6 | HLA-A | NM_001242758 | c.C448T | exonic | synonymous_SNV | soft_tissue; |
| | | | | | | urinary_tract; |
| | | | | | | lung |
| KI270467.1 | NONE; NONE | . | | intergenic | . | liver |
| chr3 | LINC02041; | . | | intergenic | . | liver |
| | SST | | | | | |
| chr6 | HLA-DRB1 | . | | UTR3 | . | haematopoietic_and_lymphoid_tissue; |
| | | | | | | liver; |
| | | | | | | large_intestine |
| chr14 | ADAM6; | . | | intergenic | . | ovary |
| | LINC00226 | | | | | |
| chr6 | HLA-DQA1 | NM_002122 | c.A223C | exonic | nonsynonymous_SNV | haematopoietic_and_lymphoid_tissue; |
| | | | | | | thyroid |
| chr19 | GDF15 | NM_004864 | c.T142A | exonic | nonsynonymous_SNV | lung; |
| | | | | | | stomach; |
| | | | | | | haematopoietic_and_lymphoid_tissue; |
| | | | | | | soft_tissue; |
| | | | | | | skin |
| chr6 | HLA-DRB1 | . | | UTR3 | . | haematopoietic_and_lymphoid_tissue; |
| | | | | | | liver; |
| | | | | | | large_intestine |
| chr6 | HLA-DRB1 | . | | UTR3 | . | haematopoietic_and_lymphoid_tissue; |
| | | | | | | liver; |
| | | | | | | large_intestine |
| chr3 | LINC02041; | . | | intergenic | . | liver |
| | SST | | | | | |
| chr6 | HLA-A | NM_001242758 | c.T992G | exonic | nonsynonymous_SNV | liver; |
| | | | | | | eye; |
| | | | | | | upper_aerodigestive_tract; |
| | | | | | | lung; |
| | | | | | | large_intestine; |
| | | | | | | thyroid; |
| | | | | | | NS |
| chr6 | HLA-A | NM_001242758 | c.A964G | exonic | nonsynonymous_SNV | large_intestine |
| KI270466.1 | NONE; NONE | . | | intergenic | . | liver |
| chr6 | HLA-DQA1 | NM_002122 | c.C169G | exonic | nonsynonymous_SNV | lung; |
| | | | | | | thyroid; |
| | | | | | | biliary_tract; |
| | | | | | | stomach; |
| | | | | | | breast |
| chr6 | HLA-A | NM_001242758 | c.A967G | exonic | nonsynonymous_SNV | soft_tissue; |
| | | | | | | thyroid; |
| | | | | | | upper_aerodigestive_tract; |
| | | | | | | central_nervous_system |
| chr6 | HLA-A | NM_001242758 | c.A363G | exonic | nonsynonymous_SNV | thyroid; |
| | | | | | | soft_tissue |
| KI270467.1 | NONE; NONE | . | | intergenic | . | liver |
| chr6 | HLA-DRB1 | . | | UTR3 | . | haematopoietic_and_lymphoid_tissue; |
| | | | | | | liver; |
| | | | | | | large_intestine |
| chr3 | LINC02041; | . | | intergenic | . | liver |
| | SST | | | | | |
| KI270467.1 | NONE; NONE | . | | intergenic | . | liver |
| chr6 | HLA-A | NM_001242758 | c.A652G | exonic | nonsynonymous_SNV | upper_aerodigestive_tract; |
| | | | | | | thyroid; |
| | | | | | | large_intestine; |
| | | | | | | skin |
| chr6 | HLA-A | . | | intronic | . | haematopoietic_and_lymphoid_tissue; |
| | | | | | | large_intestine |
| chr6 | HLA-A | NM_001242758 | c.G259C | exonic | nonsynonymous_SNV | thyroid |
| chr6 | HLA-A | NM_001242758 | c.A524G | exonic | nonsynonymous_SNV | upper_aerodigestive_tract; |
| | | | | | | stomach |
| KI270467.1 | NONE; NONE | . | | intergenic | . | liver |
| chr6 | HLA-A | NM_001242758 | c.C649G | exonic | nonsynonymous_SNV | lung; |
| | | | | | | large_intestine; |
| | | | | | | thyroid; |
| | | | | | | upper_aerodigestive_tract |
| chrM | NONE; MIR12136 | . | | intergenic | . | large_intestine; |
| | | | | | | breast; |
| | | | | | | haematopoietic_and_lymphoid_tissue; |
| | | | | | | stomach |
| chr6 | HLA-A | NM_001242758 | c.C651T | exonic | synonymous_SNV | lung; |
| | | | | | | thyroid; |
| | | | | | | large_intestine; |
| | | | | | | skin |
| chr14 | HIF1A-AS3 | . | | ncRNA_intronic | . | thyroid; |
| | | | | | | haematopoietic_and_lymphoid_tissue |
| chr6 | HLA-DRB1 | . | | UTR3 | . | haematopoietic_and_lymphoid_tissue; |
| | | | | | | liver; |
| | | | | | | large_intestine |
| chr6 | HLA-DQB1-AS1 | . | | neRNA_exonic | . | liver |
| chr6 | MPC1 | . | | UTR3 | . | liver; |
| | | | | | | breast |
| chr6 | HLA-A | . | | intronic | . | large_intestine |
| KI270467.1 | NONE; NONE | . | | intergenic | . | liver |
| chr6 | HLA-A | . | | intronic | . | haematopoietic_and_lymphoid_tissue; |
| | | | | | | large_intestine |
| KI270467.1 | NONE; NONE | . | | intergenic | . | liver |
| chr6 | HLA-DRB1 | . | | UTR3 | . | haematopoietic_and_lymphoid_tissue; |
| | | | | | | liver; |
| | | | | | | large_intestine |
| chr6 | HLA-A | NM_001242758 | c.A502C | exonic | nonsynonymous_SNV | upper_aerodigestive_tract; |
| | | | | | | lung |
| chr10 | BNIP3 | . | | UTR3 | . | stomach; |
| | | | | | | large_intestine |
| chr6 | HLA-A | NM_001242758 | c.G829T | exonic | stopgain | breast; |
| | | | | | | soft_tissue; |
| | | | | | | skin; |
| | | | | | | lung; |
| | | | | | | large_intestine; |
| | | | | | | cervix; |
| | | | | | | central_nervous_system; |
| | | | | | | upper_aerodigestive_tract |
| KI270467.1 | NONE; NONE | . | | intergenic | . | liver |
| chr6 | HLA-A | NM_001242758 | c.A633G | exonic | synonymous_SNV | thyroid; |
| | | | | | | large_intestine; |
| | | | | | | skin |
| chrM | NONE; MIR12136 | . | | intergenic | . | large_intestine; |
| | | | | | | breast; |
| | | | | | | haematopoietic_and_lymphoid_tissue; |
| | | | | | | stomach |
| chr6 | HLA-DRB1 | . | | UTR3 | . | haematopoietic_and_lymphoid_tissue; |
| | | | | | | liver; |
| | | | | | | large_intestine |
| chr11 | NEAT1 | . | | ncRNA_exonic | . | thyroid; |
| | | | | | | large_intestine |
| chr6 | HLA-A | NM_001242758 | c.A257C | exonic | nonsynonymous_SNV | thyroid |
| chr6 | HLA-DRB1 | . | | UTR3 | . | haematopoietic_and_lymphoid_tissue; |
| | | | | | | liver; |
| | | | | | | large_intestine |
| chr1 | SGIP1 | . | | intronic | . | stomach |
| chr6 | HLA-A | NM_001242758 | c.C642T | exonic | synonymous_SNV | thyroid; |
| | | | | | | skin |
| chr6 | HLA-DQA1 | NM_002122 | c.A143T | exonic | nonsynonymous_SNV | thyroid; |
| | | | | | | urinary_tract; |
| | | | | | | lung; |
| | | | | | | pancreas; |
| | | | | | | haematopoietic_and_lymphoid_tissue |
| chr2 | SERPINE2 | . | | UTR3 | . | lung; |
| | | | | | | large_intestine |
| chr6 | HLA-A | NM_001242758 | c.C180G | exonic | nonsynonymous_SNV | thyroid |
| chr6 | HLA-DRB1 | . | | UTR3 | . | haematopoietic_and_lymphoid_tissue; |
| | | | | | | liver; |
| | | | | | | large_intestine |
| chr6 | HLA-DRB1 | . | | UTR3 | . | haematopoietic_and_lymphoid_tissue; |
| | | | | | | liver; |
| | | | | | | large_intestine |
| chr6 | EZR | . | | UTR3 | . | large_intestine |
| chr6 | HLA-A | NM_001242758 | c.G808T | exonic | nonsynonymous_SNV | soft_tissue; |
| | | | | | | NS; |
| | | | | | | lung; |
| | | | | | | upper_aerodigestive_tract; |
| | | | | | | eye; |
| | | | | | | central_nervous_system |
| chr6 | HLA-A | NM_001242758 | c.C762T | exonic | synonymous_SNV | haematopoietic_and_lymphoid_tissue; |
| | | | | | | soft_tissue; |
| | | | | | | skin; |
| | | | | | | thyroid |
| chr10 | PFKP | . | | UTR3 | . | lung |
| chr6 | HLA-DRA | . | | UTR3 | . | upper_aerodigestive_tract |
| chr6 | HLA-DRB1 | . | | UTR3 | . | haematopoietic_and_lymphoid_tissue; |
| | | | | | | liver; |
| | | | | | | large_intestine |
| chr6 | HLA-A | NM_001242758 | c.G219A | exonic | synonymous_SNV | thyroid |
| chrX | PNCK | . | | UTR3 | . | biliary_tract; |
| | | | | | | thyroid |
| chrM | MIR12136 | . | | upstream | . | large_intestine; |
| | | | | | | breast; |
| | | | | | | haematopoietic_and_lymphoid_tissue; |
| | | | | | | stomach |
| chr12 | UBC | NM_021009 | c.C1257T | exonic | synonymous_SNV | breast; |
| | | | | | | thyroid; |
| | | | | | | lung; |
| | | | | | | upper_aerodigestive_tract; |
| | | | | | | cervix |
| chr16 | IL32 | . | | UTR3 | . | lung; |
| | | | | | | liver |
| chr6 | HLA-DRA | . | | UTR3 | . | upper_aerodigestive_tract |
| chr10 | PFKP | . | | UTR3 | . | large_intestine |
| chr6 | HLA-A | NM_001242758 | c.G691A | exonic | nonsynonymous_SNV | thyroid; |
| | | | | | | large_intestine; |
| | | | | | | upper_aerodigestive_tract; |
| | | | | | | central_nervous_system; |
| | | | | | | eye; |
| | | | | | | soft_tissue; |
| | | | | | | haematopoietic_and_lymphoid_tissue; |
| | | | | | | skin |
| chr17 | WSB1 | . | | UTR3 | . | haematopoietic_and_lymphoid_tissue; |
| | | | | | | large_intestine; |
| | | | | | | thyroid; |
| | | | | | | urinary_tract; |
| | | | | | | lung |
| chr6 | HLA-A | NM_001242758 | c.T98C | exonic | nonsynonymous_SNV | thyroid |
| chr6 | HLA-A | NM_001242758 | c.G945A | exonic | synonymous_SNV | eye; large_intestine; NS; |
| | | | | | | soft_tissue; |
| | | | | | | haematopoietic_and_lymphoid_tissue |
| chr6 | HLA-DRB1 | . | | UTR3 | . | haematopoietic_and_lymphoid_tissue; |
| | | | | | | liver; large_intestine |
| chr6 | HLA-A | . | | intronic | . | large_intestine |
| chr1 | TNFRSF14 | . | | UTR3 | . | large_intestine; thyroid; |
| | | | | | | pancreas |
| chr17 | ATP5MC1 | NM_001002027 | c.G351C | exonic | synonymous_SNV | stomach |
| chr6 | HLA-DQA1 | NM_002122 | c.T102C | exonic | synonymous_SNV | kidney; thyroid; pancreas |
| chr6 | HLA-DRA | NM_019111 | c.T724G | exonic | nonsynonymous_SNV | upper_aerodigestive_tract |
| chr6 | HLA-DRB1 | . | | UTR3 | . | haematopoietic_and_lymphoid_tissue; |
| | | | | | | liver; large_intestine |
| chrM | MIR12136; | . | | intergenic | . | large_intestine; breast; |
| | NONE | | | | | haematopoietic_and_lymphoid_tissue; |
| | | | | | | stomach |
| KI270466.1 | NONE; NONE | . | | intergenic | . | liver |
| chr6 | HLA-DQA1 | NM_002122 | c.C177T | exonic | synonymous_SNV | upper_aerodigestive_tract; |
| | | | | | | thyroid |
| chr6 | HLA-DQA1 | NM_002122 | c.T229C | exonic | nonsynonymous_SNV | thyroid |
| chr19 | ANGPTL4 | NM_001039667 | c.C683T | exonic | nonsynonymous_SNV | soft_tissue; large_intestine; |
| | | | | | | salivary_gland; urinary_tract |
| chrM | NONE; MIR12136 | . | | intergenic | . | large_intestine; breast; |
| | | | | | | haematopoietic_and_lymphoid_tissue; |
| | | | | | | stomach |
| chr6 | HLA-DQA1 | NM_002122 | c.G224A | exonic | nonsynonymous_SNV | thyroid; urinary_tract |
| chr6 | HLA-A | NM_001242758 | cC987T | exonic | synonymous_SNV | thyroid; skin; soft_tissue |
| chr6 | HLA-DQA1 | NM_002122 | c.A227G | exonic | nonsynonymous_SNV | thyroid; |
| | | | | | | haematopoietic_and_lymphoid_tissue |
| chr6 | HLA-DRB1 | . | | UTR3 | . | haematopoietic_and_lymphoid_tissue; |
| | | | | | | liver; large_intestine |
| chr6 | HLA-DRB1 | . | | UTR3 | . | haematopoietic_and_lymphoid_tissue; |
| | | | | | | liver; large_intestine |
| chr6 | HLA-DRB1 | . | | UTR3 | . | haematopoietic_and_lymphoid_tissue; |
| | | | | | | liver; large_intestine |
| chr6 | HLA-DRB1 | NM_002124 | c.C736G | exonic | nonsynonymous_SNV | haematopoietic_and_lymphoid_tissue; |
| | | | | | | liver; large_intestine |
| chr6 | HLA-DRB1 | . | | UTR3 | . | haematopoietic_and_lymphoid_tissue; |
| | | | | | | liver; |
| | | | | | | large_intestine |
| chr3 | LINC02041; | . | | intergenic | . | liver |
| | SST | | | | | |
| chr6 | HLA-DRB1 | . | | UTR3 | . | haematopoietic_and_lymphoid_tissue; |
| | | | | | | liver; large_intestine |
| chr6 | HLA-A | . | | UTR3 | . | lung; |
| | | | | | | soft_tissue |
| chr6 | HLA-A | NM_001242758 | c.G1072A | exonic | nonsynonymous_SNV | biliary_tract; |
| | | | | | | urinary_tract; |
| | | | | | | soft_tissue; |
| | | | | | | haematopoietic_and_lymphoid_tissue |
| chr1 | SGIP1 | . | | intronic | . | stomach |
| chr11 | MALAT1; | . | | ncRNA_exonic | . | lung |
| | TALAM1 | | | | | |
| chr20 | ZFAS1 | . | | neRNA_intronic | . | skin |
| chrM | MIR12136 | . | | upstream | . | large_intestine; |
| | | | | | | breast; |
| | | | | | | haematopoietic_and_lymphoid_tissue; |
| | | | | | | stomach |
| chr14 | ADAM6; | . | | intergenic | . | haematopoietic_and_lymphoid_tissue |
| | LINC00226 | | | | | |

TABLE 15
Expanded mutations in myeloid cells

| | | | | | | | | | prevalence_ | | | | | | |
|---|---|---|---|---|---|---|---|---|---|---|---|---|---|---|---|
| | | | | | | | | | myeloid | | | | | | |
| | | | | | | | | FDR | vs | | | | | | |
| | | | | REF_myeloid | REF_other | ALT_myeloid | ALT_other | adj | others | | | | | | |
| | | | | cell | cell | cell | cell | P- | diff_ALT_ | Gene | | | | | |
| CHR | POS | REF | ALT | numbers | numbers | numbers | numbers | values | cellular | Name | RefSeq_ID | Coding | Func_type | Mutation_type | Cosmic |
| chr6 | 32444789 | T | A | 30 | 447 | 102 | 212 | 7.68E−19 | 0.451027728 | HLA- | — | | UTR3 | — | upper_aerodigestive_ |
| | | | | | | | | | | DRA | | | | | tract |
| chrX | 23783269 | G | T | 56 | 822 | 159 | 397 | 1.38E−26 | 0.413858099 | SAT1 | — | | UTR5 | — | haematopoietic_and_ |
| | | | | | | | | | | | | | | | lymphoid_tissue; |
| | | | | | | | | | | | | | | | lung |
| chr6 | 32578838 | G | A | 37 | 647 | 124 | 362 | 1.63E−19 | 0.411415275 | HLA- | — | | UTR3 | — | haematopoietic_and_ |
| | | | | | | | | | | DRB1 | | | | | lymphoid_tissue; |
| | | | | | | | | | | | | | | | liver;large_intestine |
| chr6 | 32578907 | A | G | 52 | 753 | 109 | 288 | 2.67E−20 | 0.400361573 | HLA- | — | | UTR3 | — | haematopoietic_and_ |
| | | | | | | | | | | DRB1 | | | | | lymphoid_tissue; |
| | | | | | | | | | | | | | | | liver;large_intestine |
| chr6 | 32444815 | G | A | 31 | 416 | 106 | 248 | 5.50E−15 | 0.400228652 | HLA- | — | | UTR3 | — | upper_aerodigestive_ |
| | | | | | | | | | | DRA | | | | | tract |
| chr6 | 32445032 | T | A | 45 | 438 | 82 | 151 | 1.26E−14 | 0.389302568 | HLA- | — | | UTR3 | — | upper_aerodigestive_ |
| | | | | | | | | | | DRA | | | | | tract |
| chr6 | 32579023 | A | G | 53 | 734 | 100 | 267 | 1.57E−18 | 0.386861505 | HLA- | — | | UTR3 | — | haematopoietic_and_ |
| | | | | | | | | | | DRB1 | | | | | lymphoid_tissue; |
| | | | | | | | | | | | | | | | liver;large_intestine |
| chrX | 23783194 | C | T | 69 | 920 | 147 | 387 | 2.24E−24 | 0.384457621 | SAT1 | — | | UTR5 | — | haematopoietic_and_ |
| | | | | | | | | | | | | | | | lymphoid_tissue; |
| | | | | | | | | | | | | | | | lung |
| chr6 | 32584298 | A | G | 48 | 596 | 98 | 243 | 3.47E−16 | 0.381602364 | HLA- | NM_002124 | c.T181C | exonic | non- | haematopoietic_and_ |
| | | | | | | | | | | DRB1 | | | | synonymous_ | lymphoid_tissue; |
| | | | | | | | | | | | | | | SNV | liver;large_intestine |
| chr6 | 32578889 | G | A | 54 | 745 | 106 | 299 | 8.17E−18 | 0.376101533 | HLA- | — | | UTR3 | — | haematopoietic_and_ |
| | | | | | | | | | | DRB1 | | | | | lymphoid_tissue; |
| | | | | | | | | | | | | | | | liver;large_intestine |
| chr6 | 32584367 | A | C | 30 | 479 | 110 | 334 | 1.10E−13 | 0.374890177 | HLA- | NM_002124 | c.T112G | exonic | non- | haematopoietic_and_ |
| | | | | | | | | | | DRB1 | | | | synonymous_ | lymphoid_tissue; |
| | | | | | | | | | | | | | | SNV | liver;large_intestine |
| chr6 | 32578921 | G | A | 54 | 732 | 107 | 310 | 8.41E−17 | 0.367091475 | HLA- | — | | UTR3 | — | haematopoietic_and_ |
| | | | | | | | | | | DRB1 | | | | | lymphoid_tissue; |
| | | | | | | | | | | | | | | | liver;large_intestine |
| chr6 | 32578920 | G | A | 51 | 702 | 110 | 329 | 4.08E−16 | 0.364122151 | HLA- | — | | UTR3 | — | haematopoietic_and_ |
| | | | | | | | | | | DRB1 | | | | | _lymphoid_tissue; |
| | | | | | | | | | | | | | | | liver;large_intestine |
| chr6 | 32584162 | G | T, | 54 | 649 | 94 | 242 | 1.52E−15 | 0.363530197 | HLA- | NM_002124 | c.C317G | exonic | non- | haematopoietic_and_ |
| | | | C | | | | | | | DRB1 | | | | synonymous_ | lymphoid_tissue; |
| | | | | | | | | | | | | | | SNV | liver;large_intestine |
| chr6 | 32584315 | A | T, | 32 | 494 | 112 | 358 | 7.82E−13 | 0.357589984 | HLA- | NM_002124 | c.T164C | exonic | non- | haematopoietic_and_ |
| | | | G | | | | | | | DRB1 | | | | synonymous_ | lymphoid_tissue; |
| | | | | | | | | | | | | | | SNV | liver;large_intestine |
| chr6 | 32584366 | C | T | 36 | 511 | 106 | 330 | 1.47E−12 | 0.354088861 | HLA- | NM_002124 | c.G113A | exonic | stopgain | haematopoietic_and_ |
| | | | | | | | | | | DRB1 | | | | | lymphoid_tissue; |
| | | | | | | | | | | | | | | | liver;large_intestine |
| chr6 | 32584193 | T | G | 30 | 494 | 120 | 404 | 7.82E−13 | 0.350111359 | HLA- | NM_002124 | c.A286C | exonic | non- | haematopoietic_and_ |
| | | | | | | | | | | DRB1 | | | | synonymous_ | lymphoid_tissue; |
| | | | | | | | | | | | | | | SNV | liver;large_intestine |
| chr6 | 32578900 | T | A | 60 | 744 | 102 | 293 | 1.52E−15 | 0.347083824 | HLA- | — | | UTR3 | — | haematopoietic_and_ |
| | | | | | | | | | | DRB1 | | | | | lymphoid_tissue; |
| | | | | | | | | | | | | | | | liver;large_intestine |
| chr6 | 32579069 | C | T | 49 | 652 | 106 | 339 | 2.17E−13 | 0.341792259 | HLA- | — | | UTR3 | — | haematopoietic_and_ |
| | | | | | | | | | | DRB1 | | | | | lymphoid_tissue; |
| | | | | | | | | | | | | | | | liver;large_intestine |
| chr6 | 32584370 | G | A | 34 | 465 | 105 | 352 | 3.08E−10 | 0.32455113 | HLA- | NM_002124 | c.C109T | exonic | synonymous_ | haematopoietic_and_ |
| | | | | | | | | | | DRB1 | | | | SNV | lymphoid_tissue; |
| | | | | | | | | | | | | | | | liver;large_intestine |
| chr6 | 32581704 | C | T | 71 | 726 | 90 | 243 | 7.82E−13 | 0.308232217 | HLA- | NM_002124 | c.G505A | exonic | non- | haematopoietic_and_ |
| | | | | | | | | | | DRB1 | | | | synonymous_ | lymphoid_tissue; |
| | | | | | | | | | | | | | | SNV | liver;large_intestine |
| chr6 | 32589702 | G | A | 68 | 675 | 77 | 196 | 5.00E−12 | 0.30600578 | HLA- | NM_002124 | c.C41T | exonic | non- | haematopoietic_and_ |
| | | | | | | | | | | DRB1 | | | | synonymous_ | lymphoid_tissue; |
| | | | | | | | | | | | | | | SNV | liver;large_intestine |
| chr6 | 32579025 | C | G, | 61 | 635 | 81 | 231 | 1.12E−10 | 0.303678886 | HLA- | — | | UTR3 | — | haematopoietic_and_ |
| | | | A, | | | | | | | DRB1 | | | | | lymphoid_tissue; |
| | | | T | | | | | | | | | | | | liver;large_intestine |
| chr6 | 32584181 | C | T | 46 | 545 | 105 | 351 | 6.65E−10 | 0.303623167 | HLA- | NM_002124 | c.G298A | exonic | non- | haematopoietic_and_ |
| | | | | | | | | | | DRB1 | | | | synonymous_ | lymphoid_tissue; |
| | | | | | | | | | | | | | | SNV | liver;large_intestine |
| chr6 | 32589729 | T | C | 67 | 646 | 79 | 211 | 1.10E−10 | 0.294888189 | HLA- | NM_002124 | c.A14G | exonic | non- | haematopoietic_and_ |
| | | | | | | | | | | DRB1 | | | | synonymous_ | lymphoid_tissue; |
| | | | | | | | | | | | | | | SNV | liver;large_intestine |
| chr7 | 23274960 | A | T | 16 | 217 | 36 | 145 | 0.002973 | 0.291755206 | GPNMB | — | | UTR3 | — | lung;haematopoietic |
| | | | | | | | | | | | | | | | and_lymphoid_ |
| | | | | | | | | | | | | | | | tissue |
| chr6 | 32581675 | C | G, | 79 | 772 | 81 | 211 | 1.62E−12 | 0.291600966 | HLA- | NM_002124 | c.G534A | exonic | synonymous_ | haematopoietic_and_ |
| | | | T | | | | | | | DRB1 | | | | SNV | lymphoid_tissue; |
| | | | | | | | | | | | | | | | liver;large_intestine |
| chr6 | 32584180 | G | T, | 75 | 676 | 67 | 149 | 5.10E−12 | 0.291224925 | HLA- | NM_002124 | c.C299G | exonic | non- | haematopoietic_and_ |
| | | | C | | | | | | | DRB1 | | | | synonymous_ | lymphoid_tissue; |
| | | | | | | | | | | | | | | SNV | liver;large_intestine |
| chr6 | 32584221 | G | A | 68 | 663 | 81 | 231 | 4.16E−10 | 0.285234899 | HLA- | NM_002124 | c.C258T | exonic | synonymous_ | haematopoietic_and_ |
| | | | | | | | | | | DRB1 | | | | SNV | lymphoid_tissue; |
| | | | | | | | | | | | | | | | liver;large_intestine |
| chr6 | 32581720 | G | A | 79 | 738 | 81 | 222 | 1.12E−10 | 0.275 | HLA- | NM_002124 | c.C489T | exonic | synonymous_ | haematopoietic_and_ |
| | | | | | | | | | | DRB1 | | | | SNV | lymphoid_tissue; |
| | | | | | | | | | | | | | | | liver;large_intestine |
| chr6 | 32581771 | G | A | 77 | 714 | 82 | 228 | 2.91E−10 | 0.273685054 | HLA- | NM_002124 | c.C438T | exonic | synonymous_ | haematopoietic_and_ |
| | | | | | | | | | | DRB1 | | | | SNV | lymphoid_tissue; |
| | | | | | | | | | | | | | | | liver;large_intestine |
| chr6 | 32581786 | G | A, | 80 | 723 | 78 | 206 | 1.61E−10 | 0.271927076 | HLA- | NM_002124 | c.C423G | exonic | non- | haematopoietic_and_ |
| | | | | | | | | | | DRB1 | | | | synonymous_ | lymphoid_tissue; |
| | | | | | | | | | | | | | | SNV | liver;large_intestine |
| chr6 | 32584354 | C | G, | 27 | 228 | 91 | 228 | 1.20E−05 | 0.271186441 | HLA- | NM_002124 | c.G125A | exonic\ | non- | haematopoietic_and_ |
| | | | T | | | | | | | DRB1; | | | x3bsplicing | synonymous_ | lymphoid_tissue; |
| | | | | | | | | | | HLA- | | | | SNV | liver;large_intestine |
| | | | | | | | | | | DRB1 | | | | | |
| chr6 | 32584283 | A | T | 67 | 628 | 80 | 238 | 1.33E−08 | 0.269390897 | HLA- | NM_002124 | c.T196A | exonic | non- | haematopoietic_and_ |
| | | | | | | | | | | DRB1 | | | | synonymous_ | lymphoid_tissue; |
| | | | | | | | | | | | | | | SNV | liver;large_intestine |
| chr6 | 32581763 | C | T, | 76 | 704 | 84 | 242 | 9.77E−10 | 0.269186047 | HLA- | NM_002124 | c.G446T | exonic | non- | haematopoietic_and_ |
| | | | A | | | | | | | DRB1 | | | | synonymous_ | lymphoid_tissue; |
| | | | | | | | | | | | | | | SNV | liver;large_intestine |
| chr6 | 29945597 | C | G | 83 | 1426 | 99 | 546 | 1.47E−11 | 0.267079776 | HLA-A | — | | UTR3 | — | upper_aerodigestive |
| | | | | | | | | | | | | | | | tract;haematopoietic_ |
| | | | | | | | | | | | | | | | and_lymphoid_ |
| | | | | | | | | | | | | | | | tissue |
| chr6 | 32517546 | T | A | 40 | 318 | 58 | 156 | 7.12E−05 | 0.262722811 | HLA- | — | | UTR3 | — | upper_aerodigestive |
| | | | | | | | | | | DRB5 | | | | | tract |
| chr6 | 32581719 | C | T | 62 | 620 | 97 | 335 | 7.93E−08 | 0.259277553 | HLA- | NM_002124 | c.G490A | exonic | non- | haematopoietic_and_ |
| | | | | | | | | | | DRB1 | | | | synonymous_ | lymphoid_tissue; |
| | | | | | | | | | | | | | | SNV | liver;large_intestine |
| chr6 | 29945653 | C | T | 86 | 1456 | 99 | 575 | 2.14E−10 | 0.252023368 | HLA-A | — | | UTR3 | — | upper_aerodigestive_ |
| | | | | | | | | | | | | | | | tract; lung |
| chr2 | 60881694 | G | A | 17 | 120 | 52 | 122 | 0.00676 | 0.249490957 | REL | — | | UTR5 | — | lung |
| chr6 | 32589752 | C | G | 73 | 649 | 71 | 209 | 1.05E−07 | 0.249465812 | HLA- | — | | UTR5 | — | haematopoietic_and_ |
| | | | | | | | | | | DRB1 | | | | | lymphoid_tissue; |
| | | | | | | | | | | | | | | | liver;large_intestine |
| chr6 | 29943309 | T | C | 64 | 1086 | 80 | 481 | 1.72E−07 | 0.248599589 | HLA-A | NM_001242758 | c.T385C | exonic | non- | thyroid;urinary_ |
| | | | | | | | | | | | | | | synonymous_ | tract |
| | | | | | | | | | | | | | | SNV | |
| chr6 | 31533435 | A | T | 26 | 420 | 24 | 127 | 0.004195 | 0.247824497 | ATP6V1G2- | — | | ncRNA | — | upper_aerodigestive |
| | | | | | | | | | | DDX39B | | | intronic | | tract; thyroid;skin;h |
| | | | | | | | | | | | | | | | aematopoietic_and |
| | | | | | | | | | | | | | | | lymphoid_tissue |
| chr6 | 32584151 | G | A | 83 | 723 | 67 | 185 | 1.73E−08 | 0.242922173 | HLA- | NM_002124 | c.C328T | exonic | non- | haematopoietic_and_ |
| | | | | | | | | | | DRB1 | | | | synonymous_ | lymphoid_tissue; |
| | | | | | | | | | | | | | | SNV | liver;large_intestine |
| chr6 | 32517630 | A | T | 47 | 327 | 54 | 135 | 0.000187 | 0.242445673 | HLA- | — | | UTR3 | — | upper_aerodigestive |
| | | | | | | | | | | DRB5 | | | | | tract |
| chr6 | 32584249 | C | T | 75 | 661 | 73 | 222 | 2.47E−07 | 0.241827615 | HLA- | NM_002124 | c.G230A | exonic | non- | haematopoietic_and_ |
| | | | | | | | | | | DRB1 | | | | synonymous_ | lymphoid_tissue; |
| | | | | | | | | | | | | | | SNV | liver;large_intestine |
| chrX | 23783776 | C | T | 61 | 294 | 127 | 226 | 1.62E−06 | 0.24091653 | SAT1 | — | | intronic | — | lung |
| chr6 | 31269077 | T | C | 67 | 1134 | 88 | 566 | 5.93E−07 | 0.234800759 | HLA-C | — | | UTR3 | — | upper_aerodigestive_ |
| | | | | | | | | | | | | | | | tract |
| chr6 | 29943494 | G | C | 79 | 1204 | 68 | 361 | 8.81E−08 | 0.231914107 | HLA-A | NM_001242758 | c.G570C | exonic | non- | thyroid;large_intestine; |
| | | | | | | | | | | | | | | synonymous_ | kidney;biliary_ |
| | | | | | | | | | | | | | | SNV | tract;haematopoietic |
| | | | | | | | | | | | | | | | and_lymphoid_ |
| | | | | | | | | | | | | | | | tissue;skin |
| chr6 | 32517567 | G | A | 42 | 310 | 57 | 163 | 0.000836 | 0.231148696 | HLA- | — | | UTR3 | — | upper_aerodigestive_ |
| | | | | | | | | | | DRB5 | | | | | tract |
| chr6 | 29943462 | T | C | 71 | 1118 | 76 | 453 | 1.13E−06 | 0.228655434 | HLA-A | NM_001242758 | c.T538C | exonic | synony | oesophagus; large_ |
| | | | | | | | | | | | | | | mous | intestine; |
| | | | | | | | | | | | | | | SNV | haematopoietic_and_ |
| | | | | | | | | | | | | | | | lymphoid_tissue |
| chr6 | 29943495 | T | G, | 72 | 1125 | 71 | 416 | 1.32E−06 | 0.226548922 | HLA-A | NM_001242758 | c.T571C | exonic | non- | upper_aerodigestive_ |
| | | | A, | | | | | | | | | | | synonymous_ | tract |
| | | | C | | | | | | | | | | | SNV | |
| chr6 | 29942954 | G | A | 70 | 1126 | 71 | 432 | 1.94E−06 | 0.226267537 | HLA-A | NM_001242758 | c.G271A | exonic | non- | thyroid |
| | | | | | | | | | | | | | | synonymous_ | |
| | | | | | | | | | | | | | | SNV | |
| chr6 | 29943445 | C | T | 69 | 1117 | 74 | 461 | 2.62E−06 | 0.225340566 | HLA-A | NM_001242758 | c.C521T | exonic | non- | upper_aerodigestive_ |
| | | | | | | | | | | | | | | synonymous_ | tract; skin |
| | | | | | | | | | | | | | | SNV | |
| chr6 | 29942965 | G | C | 70 | 1115 | 70 | 436 | 5.98E−06 | 0.218891038 | HLA-A | NM_001242758 | c.G282C | exonic | non- | thyroid |
| | | | | | | | | | | | | | | synonymous_ | |
| | | | | | | | | | | | | | | SNV | |
| chr6 | 32517639 | G | C | 49 | 330 | 51 | 137 | 0.001259 | 0.216638116 | HLA- | — | | UTR3 | — | upper_aerodigestive |
| | | | | | | | | | | DRB5 | | | | | tract |
| chr6 | 32517455 | T | G | 46 | 305 | 50 | 136 | 0.002738 | 0.212443311 | HLA- | — | | UTR3 | — | upper_aerodigestive |
| | | | | | | | | | | DRB5 | | | | | tract |
| chr19 | 48966392 | C | T | 85 | 1188 | 107 | 627 | 7.77E−07 | 0.211837121 | FTL | NM_000146 | c.C361T | exonic | non- | lung;large_intestine; |
| | | | | | | | | | | | | | | synonymous_ | thyroid;haematopoietic_ |
| | | | | | | | | | | | | | | SNV | and_lymphoid_ |
| | | | | | | | | | | | | | | | tissue |
| chr6 | 32517551 | G | T, | 53 | 342 | 48 | 123 | 0.001259 | 0.210731396 | HLA- | — | | UTR3 | — | upper_aerodigestive |
| | | | A | | | | | | | DRB5 | | | | | tract |
| chr6 | 32584271 | C | T | 87 | 698 | 60 | 175 | 3.37E−06 | 0.207705075 | HLA- | NM_002124 | c.G208A | exonic | non- | haematopoietic_and_ |
| | | | | | | | | | | DRB1 | | | | synonymous_ | lymphoid_tissue; |
| | | | | | | | | | | | | | | SNV | liver;large_intestine |
| chr6 | 32517566 | G | A | 41 | 285 | 60 | 182 | 0.004683 | 0.204337779 | HLA- | — | | UTR3 | — | upper_aerodigestive |
| | | | | | | | | | | DRB5 | | | | | tract |
| chr6 | 32517511 | G | A | 46 | 316 | 57 | 171 | 0.003813 | 0.202268695 | HLA- | — | | UTR3 | — | upper_aerodigestive |
| | | | | | | | | | | DRB5 | | | | | tract |
| chr6 | 29943483 | A | C | 72 | 1094 | 74 | 482 | 4.71E−05 | 0.201011752 | HLA-A | NM_001242758 | c.A559C | exonic | non- | thyroid;upper_ |
| | | | | | | | | | | | | | | synonymous_ | aerodigestive_tract |
| | | | | | | | | | | | | | | SNV | |
| chr6 | 32584266 | G | A | 92 | 714 | 56 | 160 | 7.50E−06 | 0.195312017 | HLA- | NM_002124 | c.C213T | exonic | non- | haematopoietic_and_ |
| | | | | | | | | | | DRB1 | | | | synonymous_ | lymphoid_tissue; |
| | | | | | | | | | | | | | | SNV | liver;large_intestine |
| chr6 | 32584262 | C | G | 92 | 703 | 55 | 158 | 1.56E−05 | 0.190642111 | HLA- | NM_002124 | c.G217C | exonic | non- | haematopoietic_and_ |
| | | | | | | | | | | DRB1 | | | | synonymous_ | lymphoid_tissue; |
| | | | | | | | | | | | | | | SNV | liver;large_intestine |
| chr6 | 32580812 | C | T | 57 | 558 | 90 | 407 | 0.000684 | 0.19048324 | HLA- | NM_002124 | c.G697A | exonic | non- | haematopoietic_and_ |
| | | | | | | | | | | DRB1 | | | | synonymous_ | lymphoid_tissue; |
| | | | | | | | | | | | | | | SNV | liver;large_intestine |
| chr6 | 29942886 | G | A | 77 | 1158 | 61 | 397 | 0.000134 | 0.186723519 | HLA-A | NM_001242758 | c.G203A | exonic | non- | thyroid |
| | | | | | | | | | | | | | | synonymous_ | |
| | | | | | | | | | | | | | | SNV | |
| chr6 | 32578849 | C | G, | 5 | 109 | 124 | 377 | 0.000105 | 0.185520145 | HLA- | — | | UTR3 | — | haematopoietic_and_ |
| | | | A, | | | | | | | DRB1 | | | | | lymphoid_tissue; |
| | | | T | | | | | | | | | | | | liver;large_intestine |
| chr6 | 32584273 | A | T | 93 | 701 | 53 | 154 | 3.83E−05 | 0.18289674 | HLA- | NM_002124 | c.T206A | exonic | non- | haematopoietic_and_ |
| | | | | | | | | | | DRB1 | | | | synonymous_ | lymphoid_tissue; |
| | | | | | | | | | | | | | | SNV | liver;large_intestine |
| chr6 | 32517624 | G | A | 49 | 315 | 53 | 160 | 0.011719 | 0.182765738 | HLA- | — | | UTR3 | — | upper_aerodigestive |
| | | | | | | | | | | DRB5 | | | | | tract |
| chr6 | 29943413 | G | A | 68 | 1015 | 78 | 552 | 0.000599 | 0.1819811 | HLA-A | NM_001242758 | c.G489A | exonic | synonymous_ | thyroid |
| | | | | | | | | | | | | | | SNV | |
| chr6 | 31356374 | C | T | 93 | 1339 | 84 | 554 | 3.88E−05 | 0.181919113 | HLA-B | NM_005514 | c.G412A | exonic | non- | soft tissue;urinary_ |
| | | | | | | | | | | | | | | synonymous_ | tract;thyroid; biliary_ |
| | | | | | | | | | | | | | | SNV | tract |
| chr6 | 29943469 | C | T | 76 | 1091 | 72 | 487 | 0.000473 | 0.177867982 | HLA-A | NM_001242758 | c.C545T | exonic | non- | breast;upper_ |
| | | | | | | | | | | | | | | synonymous_ | aerodigestive_tract |
| | | | | | | | | | | | | | | SNV | |
| chr6 | 31356248 | G | C | 97 | 1374 | 77 | 500 | 5.38E−05 | 0.175719771 | HLA-B | NM_005514 | c.C538G | exonic | non- | upper_aerodigestive_ |
| | | | | | | | | | | | | | | synonymous_ | tract |
| | | | | | | | | | | | | | | SNV | |
| chr6 | 31356246 | C | G | 98 | 1371 | 80 | 519 | 5.59E−05 | 0.174835028 | HLA-B | NM_005514 | c.G540C | exonic | synonymous_ | upper_aerodigestive_ |
| | | | | | | | | | | | | | | SNV | tract;haematopoietic_ |
| | | | | | | | | | | | | | | | and_lymphoid_ |
| | | | | | | | | | | | | | | | tissue |
| chr6 | 32584319 | G | A | 85 | 654 | 56 | 187 | 0.000447 | 0.17480878 | HLA- | NM_002124 | c.C160T | exonic | non- | haematopoietic_and_ |
| | | | | | | | | | | DRB1 | | | | synonymous_ | lymphoid_tissue; |
| | | | | | | | | | | | | | | SNV | liver;large intestine |
| chr6 | 32584310 | C | T | 84 | 650 | 61 | 212 | 0.000585 | 0.17474998 | HLA- | NM_002124 | c.G169A | exonic | non- | haematopoietic_and_ |
| | | | | | | | | | | DRB1 | | | | synonymous_ | lymphoid_tissue; |
| | | | | | | | | | | | | | | SNV | liver;large_intestine |
| chr19 | 18177259 | A | G | 34 | 139 | 76 | 150 | 0.034642 | 0.171877949 | IFI30 | NM_006332 | c.A603G | exonic | synonymous_ | skin;stomach;lung |
| | | | | | | | | | | | | | | SNV | |
| chr2 | 89040248 | A | G | 13 | 325 | 82 | 728 | 0.010396 | 0.17179987 | MIR4436A; | | | intergenic | | haematopoietic_and_ |
| | | | | | | | | | | LOC107985911 | | | | | _lymphoid_tissue |
| chr6 | 32584301 | A | T | 95 | 711 | 51 | 154 | 0.000124 | 0.171280386 | HLA- | NM_002124 | c.T178A | exonic | non- | haematopoietic_and_ |
| | | | | | | | | | | DRB1 | | | | synonymous_ | lymphoid_tissue; |
| | | | | | | | | | | | | | | SNV | liver;large_intestine |
| chr6 | 29943463 | T | G | 69 | 996 | 80 | 576 | 0.001527 | 0.170500538 | HLA-A | NM_001242758 | c.T539G | exonic | non- | soft_tissue;pancreas; |
| | | | | | | | | | | | | | | synonymous_ | large_intestine |
| | | | | | | | | | | | | | | SNV | |
| chr5 | 1.5E+08 | C | T | 76 | 931 | 117 | 720 | 0.000338 | 0.170118283 | CD74 | NM_001025158 | c.G29A | exonic | non- | kidney |
| | | | | | | | | | | | | | | synonymous_ | |
| | | | | | | | | | | | | | | SNV | |
| chr6 | 31356934 | A | C | 94 | 1313 | 77 | 513 | 0.000189 | 0.169350448 | HLA-B | NM_005514 | c.T97G | exonic | non- | upper_aerodigestive_ |
| | | | | | | | | | | | | | | synonymous_ | tract; central_ |
| | | | | | | | | | | | | | | SNV | nervous_system |
| chr6 | 31271672 | C | G | 68 | 1017 | 60 | 437 | 0.002875 | 0.168199794 | HLA-C | NM_001243042 | c.G270C | exonic | non- | kidney;lung |
| | | | | | | | | | | | | | | synonymous_ | |
| | | | | | | | | | | | | | | SNV | |
| chr6 | 32584290 | C | T | 94 | 697 | 54 | 171 | 0.000309 | 0.167860257 | HLA- | NM_002124 | c.G189A | exonic | synonymous_ | haematopoietic_and_ |
| | | | | | | | | | | DRB1 | | | | SNV | lymphoid_tissue; |
| | | | | | | | | | | | | | | | liver;large_intestine |
| chr6 | 32584323 | C | T | 94 | 699 | 51 | 162 | 0.000442 | 0.163570828 | HLA- | NM_002124 | c.G156A | exonic | synonymous_ | haematopoietic_and_ |
| | | | | | | | | | | DRB1 | | | | SNV | lymphoid_tissue; |
| | | | | | | | | | | | | | | | liver;large_intestine |
| chr6 | 32584316 | A | T | 95 | 694 | 50 | 161 | 0.000921 | 0.156523493 | HLA- | NM_002124 | c.T163A | exonic | non- | haematopoietic_and_ |
| | | | | | | | | | | DRB1 | | | | synonymous_ | lymphoid_tissue; |
| | | | | | | | | | | | | | | SNV | liver;large_intestine |
| chr6 | 32584309 | T | A | 97 | 717 | 46 | 147 | 0.000953 | 0.151539433 | HLA- | NM_002124 | c.A170T | exonic | non- | haematopoietic_and_ |
| | | | | | | | | | | DRB1 | | | | synonymous_ | lymphoid_tissue; |
| | | | | | | | | | | | | | | SNV | liver;large_intestine |
| chrX | 1.54E+08 | T | G | 5 | 167 | 77 | 641 | 0.029694 | 0.145707559 | RPL10 | — | | UTR5 | — | lung |
| chr5 | 1.5E+08 | G | A | 93 | 1025 | 92 | 557 | 0.003152 | 0.14521133 | CD74 | NM_001364083 | c.C404T | exonic | non- | kidney |
| | | | | | | | | | | | | | | synonymous_ | |
| | | | | | | | | | | | | | | SNV | |
| chr6 | 32589710 | G | A | 98 | 706 | 48 | 163 | 0.003304 | 0.141195202 | HLA- | NM_002124 | c.C33T | exonic | synonymous_ | haematopoietic_and_ |
| | | | | | | | | | | DRB1 | | | | SNV | lymphoid_tissue; |
| | | | | | | | | | | | | | | | liver;large intestine |
| chr6 | 32584279 | A | G | 97 | 696 | 45 | 152 | 0.004195 | 0.137656125 | HLA- | NM_002124 | c.T200A | exonic | non- | haematopoietic_and_ |
| | | | | | | | | | | DRB1 | | | | synonymous_ | lymphoid_tissue; |
| | | | | | | | | | | | | | | SNV | liver;large_intestine |
| chr5 | 1.5E+08 | G | A, | 83 | 922 | 107 | 692 | 0.008898 | 0.134409444 | CD74 | NM_001025158 | c.C463A | exonic | non- | haematopoietic_and_ |
| | | | T | | | | | | | | | | | synonymous_ | lymphoid_tissue; |
| | | | | | | | | | | | | | | SNV | breast; lung |
| chr5 | 1.5E+08 | G | A | 102 | 1116 | 100 | 632 | 0.005066 | 0.133493441 | CD74 | — | | UTR3 | — | haematopoietic_and_ |
| | | | | | | | | | | | | | | | _lymphoid_tissue; |
| | | | | | | | | | | | | | | | breast; lung |
| chr6 | 32584340 | A | G | 96 | 678 | 47 | 167 | 0.01018 | 0.131038193 | HLA- | NM_002124 | c.T139C | exonic | non- | haematopoietic_and_ |
| | | | | | | | | | | DRB1 | | | | synonymous_ | lymphoid_tissue; |
| | | | | | | | | | | | | | | SNV | liver;large_intestine |
| chr6 | 32589688 | G | A | 99 | 703 | 47 | 167 | 0.008826 | 0.129963785 | HLA- | NM_002124 | c.C55T | exonic | synonymous_ | haematopoietic_and_ |
| | | | | | | | | | | DRB1 | | | | SNV | lymphoid_tissue; |
| | | | | | | | | | | | | | | | liver;large_intestine |
| chr19 | 48965830 | T | C, | 34 | 541 | 156 | 1224 | 0.006011 | 0.127568212 | FTL | NM_000146 | c.T163G | exonic | non- | lung;large_intestine; |
| | | | G | | | | | | | | | | | synonymous_ | thyroid; |
| | | | | | | | | | | | | | | SNV | haematopoietic_and_ |
| | | | | | | | | | | | | | | | lymphoidtissue |
| chrM | 3021 | C | T | 82 | 1148 | 131 | 1163 | 0.028825 | 0.111778126 | NONE; | — | | intergenic | — | large_intestine; |
| | | | | | | | | | | MIR12136 | | | | | breast;haematopoietic_ |
| | | | | | | | | | | | | | | | and_lymphoid_tissue; |
| | | | | | | | | | | | | | | | stomach |
| chr6 | 32589759 | G | A | 101 | 682 | 41 | 149 | 0.040776 | 0.109430349 | HLA- | — | | UTR5 | — | haematopoietic_and_ |
| | | | | | | | | | | DRB1 | | | | | lymphoid_tissue; |
| | | | | | | | | | | | | | | | liver;large_intestine |

TABLE 16
Expanded mutations in plasma B cell

| | | | | | | | ALT_ | | | | | | | | |
|---|---|---|---|---|---|---|---|---|---|---|---|---|---|---|---|
| | | | | REF_ | REF_ | | other | | | | | | | | |
| | | | | plasma | other cell | ALT_plasma | cell | FDR adj | diff_ALT_ | Gene | | | | | |
| CHR | POS | REF | ALT | B cell | numbers | B cell | numbers | P-values | cellular | Name | RefSeq_ID | Coding | Func_type | Mutation_type | Cosmic |
| chr14 | 1.06E+08 | C | G, | 16 | 397 | 235 | 1687 | 4.41E−05 | 0.12675402 | MIR8071- | — | | intergenic | — | haematopoietic_ |
| | | | A | | | | | | | 1; | | | | | and_lymphoid |
| | | | | | | | | | | MIR8071-1 | | | | | tissue |
| chr14 | 1.06E+08 | T | C | 25 | 249 | 171 | 725 | 0.002818 | 0.128095797 | MIR8071- | — | | intergenic | — | haematopoietic_ |
| | | | | | | | | | | 1; | | | | | and_lymphoid |
| | | | | | | | | | | ELK2AP | | | | | tissue |
| chr14 | 1.06E+08 | C | G | 16 | 212 | 181 | 782 | 0.000653 | 0.132061404 | MIR8071- | — | | intergenic | — | skin |
| | | | | | | | | | | 1; | | | | | |
| | | | | | | | | | | ELK2AP | | | | | |
| chr14 | 1.06E+08 | T | C | 26 | 217 | 158 | 635 | 0.015042 | 0.113390488 | MIR8071- | — | | intergenic | — | skin |
| | | | | | | | | | | 1; | | | | | |
| | | | | | | | | | | ELK2AP | | | | | |
| chr14 | 1.06E+08 | T | C | 16 | 204 | 163 | 629 | 0.00023 | 0.155512484 | MIR8071- | — | | intergenic | — | thyroid |
| | | | | | | | | | | 1; | | | | | |
| | | | | | | | | | | ELK2AP | | | | | |
| chr14 | 1.06E+08 | C | G | 45 | 507 | 180 | 807 | 7.52E−06 | 0.185844749 | MIR8071- | — | | intergenic | — | large_intestine |
| | | | | | | | | | | 1; | | | | | |
| | | | | | | | | | | ELK2AP | | | | | |
| chr14 | 1.06E+08 | C | G | 49 | 528 | 186 | 795 | 2.90E−06 | 0.190582332 | ELK2AP; | — | | intergenic | — | large_intestine |
| | | | | | | | | | | MIR4539 | | | | | |
| chr14 | 1.06E+08 | T | C, | 14 | 246 | 224 | 1140 | 0.000201 | 0.118665648 | ELK2AP; | — | | intergenic | — | large_intestine |
| | | | A | | | | | | | MIR4539 | | | | | |
| chr14 | 1.06E+08 | G | A | 54 | 1184 | 193 | 997 | 1.08E−18 | 0.324246761 | ELK2AP; | — | | intergenic | — | large_intestine |
| | | | | | | | | | | MIR4539 | | | | | |
| chr14 | 1.06E+08 | G | A | 73 | 608 | 76 | 363 | 0.02053 | 0.136225713 | ADAM6; | — | | intergenic | — | haematopoietic_ |
| | | | | | | | | | | LINC00226 | | | | | and_lymphoid |
| | | | | | | | | | | | | | | | tissue |
| chr14 | 1.06E+08 | G | T, | 58 | 432 | 37 | 140 | 0.036763 | 0.144718439 | ADAM6; | — | | intergenic | — | haematopoietic_ |
| | | | C | | | | | | | LINC00226 | | | | | and_lymphoid |
| | | | | | | | | | | | | | | | tissue |
| chr14 | 1.06E+08 | C | T, | 102 | 717 | 33 | 117 | 0.026166 | 0.104156675 | ADAM6; | — | | intergenic | — | haematopoietic_ |
| | | | G, | | | | | | | LINC00226 | | | | | and_lymphoid |
| | | | A | | | | | | | | | | | | tissue |
| chr14 | 1.06E+08 | C | T, | 96 | 742 | 56 | 234 | 0.012183 | 0.128666954 | ADAM6; | — | | intergenic | — | haematopoietic_ |
| | | | G | | | | | | | LINC00226 | | | | | and_lymphoid |
| | | | | | | | | | | | | | | | tissue |
| chr14 | 1.06E+08 | C | G, | 107 | 787 | 41 | 165 | 0.031477 | 0.103707699 | ADAM6; | — | | intergenic | — | haematopoietic_ |
| | | | A, | | | | | | | LINC00226 | | | | | and_lymphoid |
| | | | T | | | | | | | | | | | | tissue |
| chr14 | 1.06E+08 | T | G, | 101 | 660 | 28 | 73 | 0.003794 | 0.117463541 | ADAM6; | — | | intergenic | — | haematopoietic_ |
| | | | C, | | | | | | | LINC00226 | | | | | and_lymphoid |
| | | | A | | | | | | | | | | | | tissue |
| chr14 | 1.06E+08 | T | C, | 59 | 434 | 43 | 111 | 0.000131 | 0.217898903 | ADAM6; | — | | intergenic | — | haematopoietic_ |
| | | | A, | | | | | | | LINC00226 | | | | | and_lymphoid |
| | | | G | | | | | | | | | | | | tissue |
| chr14 | 1.06E+08 | C | T | 109 | 835 | 44 | 162 | 0.004123 | 0.125094237 | ADAM6; | — | | intergenic | — | haematopoietic_ |
| | | | | | | | | | | LINC00226 | | | | | and_lymphoid |
| | | | | | | | | | | | | | | | tissue |
| chr14 | 1.06E+08 | C | T, | 45 | 315 | 52 | 185 | 0.028068 | 0.166082474 | ADAM6; | — | | intergenic | — | haematopoietic_ |
| | | | G, | | | | | | | LINC00226 | | | | | and lymphoid |
| | | | A | | | | | | | | | | | | tissue |
| chr2 | 88857538 | C | T, | 28 | 680 | 230 | 1863 | 2.73E−06 | 0.158873576 | MIR4436A; | — | | intergenic | — | haematopoietic_ |
| | | | G | | | | | | | LOC107985911 | | | | | and_lymphoid_ |
| | | | | | | | | | | | | | | | tissue; cervix; |
| | | | | | | | | | | | | | | | lung |
| chr2 | 88857650 | G | A | 17 | 771 | 241 | 1761 | 5.84E−13 | 0.238610897 | MIR4436A; | — | | intergenic | — | haematopoietic_ |
| | | | | | | | | | | LOC107985911 | | | | | _and_lymphoid |
| | | | | | | | | | | | | | | | tissue; cervix; |
| | | | | | | | | | | | | | | | lung |
| chr2 | 88861908 | T | C, | 123 | 1237 | 67 | 397 | 0.015042 | 0.109669523 | MIR4436A; | — | | intergenic | — | haematopoietic_ |
| | | | G, | | | | | | | LOC107985911 | | | | | and_lymphoid |
| | | | A | | | | | | | | | | | | tissue |
| chr2 | 88947319 | T | C, | 74 | 930 | 130 | 720 | 5.65E−06 | 0.200891266 | MIR4436A; | — | | intergenic | — | haematopoietic_ |
| | | | G | | | | | | | LOC107985911 | | | | | and_lymphoid |
| | | | | | | | | | | | | | | | tissue |
| chr2 | 88947322 | G | A | 53 | 633 | 153 | 1038 | 0.010237 | 0.121533527 | MIR4436A; | — | | intergenic | — | haematopoietic_ |
| | | | | | | | | | | LOC107985911 | | | | | and_lymphoid |
| | | | | | | | | | | | | | | | tissue |
| chr2 | 88947331 | A | C, | 110 | 1041 | 91 | 561 | 0.041735 | 0.102549052 | MIR4436A; | — | | intergenic | — | haematopoietic_ |
| | | | G, | | | | | | | LOC107985911 | | | | | and_lymphoid |
| | | | T | | | | | | | | | | | | tissue |
| chr2 | 88947332 | G | A, | 100 | 1039 | 103 | 620 | 0.004423 | 0.133670055 | MIR4436A; | — | | intergenic | — | haematopoietic_ |
| | | | C, | | | | | | | LOC107985911 | | | | | _and_lymphoid |
| | | | T | | | | | | | | | | | | tissue |
| chr2 | 88947333 | T | C, | 93 | 969 | 111 | 691 | 0.008419 | 0.127852587 | MIR4436A; | — | | intergenic | — | haematopoietic_ |
| | | | A | | | | | | | LOC107985911 | | | | | and_lymphoid |
| | | | | | | | | | | | | | | | tissue |
| chr2 | 88947343 | A | T, | 47 | 625 | 139 | 837 | 0.000219 | 0.174808408 | MIR4436A; | — | | intergenic | — | haematopoietic_ |
| | | | G | | | | | | | LOC107985911 | | | | | and_lymphoid |
| | | | | | | | | | | | | | | | tissue |
| chr2 | 88947349 | C | T | 107 | 1094 | 99 | 576 | 0.002974 | 0.135672345 | MIR4436A; | — | | intergenic | — | haematopoietic_ |
| | | | | | | | | | | LOC107985911 | | | | | and_lymphoid |
| | | | | | | | | | | | | | | | tissue |
| chr2 | 88947351 | G | C | 96 | 1023 | 110 | 642 | 0.001244 | 0.148394997 | MIR4436A; | — | | intergenic | — | haematopoietic_ |
| | | | | | | | | | | LOC107985911 | | | | | and_lymphoid |
| | | | | | | | | | | | | | | | tissue |
| chr2 | 88947353 | A | G | 125 | 1238 | 81 | 428 | 0.001076 | 0.136301122 | MIR4436A; | — | | intergenic | — | haematopoietic_ |
| | | | | | | | | | | LOC107985911 | | | | | and_lymphoid |
| | | | | | | | | | | | | | | | tissue |
| chr2 | 88947397 | G | A, | 86 | 882 | 95 | 590 | 0.018497 | 0.124046661 | MIR4436A; | — | | intergenic | — | haematopoietic_ |
| | | | T | | | | | | | LOC107985911 | | | | | and_lymphoid |
| | | | | | | | | | | | | | | | tissue |
| chr2 | 88947404 | C | T, | 95 | 964 | 87 | 510 | 0.008089 | 0.132024692 | MIR4436A; | — | | intergenic | — | haematopoietic_ |
| | | | G | | | | | | | LOC107985911 | | | | | and_lymphoid |
| | | | | | | | | | | | | | | | tissue |
| chr2 | 88947493 | G | A, | 107 | 1024 | 63 | 349 | 0.01724 | 0.116400326 | MIR4436A; | — | | intergenic | — | haematopoietic_ |
| | | | C, | | | | | | | LOC107985911 | | | | | and_lymphoid |
| | | | T | | | | | | | | | | | | tissue |
| chr2 | 88947501 | T | A, | 124 | 1151 | 51 | 252 | 0.007712 | 0.111813461 | MIR4436A; | — | | intergenic | — | haematopoietic_ |
| | | | C, | | | | | | | LOC107985911 | | | | | and_lymphoid |
| | | | G | | | | | | | | | | | | tissue |
| chr2 | 88947504 | T | C | 69 | 737 | 105 | 690 | 0.030668 | 0.119916391 | MIR4436A; | — | | intergenic | — | haematopoietic_ |
| | | | | | | | | | | LOC107985911 | | | | | and_lymphoid |
| | | | | | | | | | | | | | | | tissue |
| chr2 | 88947558 | T | A | 87 | 923 | 89 | 488 | 0.001056 | 0.159827814 | MIR4436A; | — | | intergenic | — | haematopoietic_ |
| | | | | | | | | | | LOC107985911 | | | | | and_lymphoid |
| | | | | | | | | | | | | | | | tissue |
| chr2 | 88947562 | A | G, | 74 | 788 | 79 | 476 | 0.012872 | 0.139757591 | MIR4436A; | — | | intergenic | — | haematopoietic_ |
| | | | T, | | | | | | | LOC107985911 | | | | | and_lymphoid |
| | | | C | | | | | | | | | | | | tissue |
| chr2 | 89040248 | A | G | 28 | 310 | 123 | 687 | 0.021248 | 0.125502335 | MIR4436A; | — | | intergenic | — | haematopoietic_ |
| | | | | | | | | | | LOC107985911 | | | | | and_lymphoid |
| | | | | | | | | | | | | | | | tissue |
| chr2 | 89142767 | C | T, | 131 | 1426 | 78 | 430 | 0.000287 | 0.141524707 | MIR4436A; | — | | intergenic | — | haematopoietic_ |
| | | | G, | | | | | | | LOC107985911 | | | | | and_lymphoid |
| | | | A | | | | | | | | | | | | tissue |
| chr2 | 89142777 | C | T, | 131 | 1409 | 80 | 461 | 0.001032 | 0.132622855 | MIR4436A; | — | | intergenic | — | haematopoietic_ |
| | | | A, | | | | | | | LOC107985911 | | | | | and_lymphoid |
| | | | G | | | | | | | | | | | | tissue |
| chr2 | 89319775 | C | T, | 83 | 862 | 72 | 447 | 0.028373 | 0.123034082 | MIR4436A; | — | | intergenic | — | haematopoietic_ |
| | | | A, | | | | | | | LOC107985911 | | | | | and_lymphoid |
| | | | G | | | | | | | | | | | | tissue |
| chr2 | 89319782 | G | C | 94 | 950 | 68 | 416 | 0.031692 | 0.115214287 | MIR4436A; | — | | intergenic | — | haematopoietic_ |
| | | | | | | | | | | LOC107985911 | | | | | and_lymphoid |
| | | | | | | | | | | | | | | | tissue |
| chr2 | 89319825 | T | C, | 114 | 1101 | 50 | 274 | 0.022449 | 0.105605322 | MIR4436A; | — | | intergenic | — | haematopoietic_ |
| | | | G, | | | | | | | LOC107985911 | | | | | and_lymphoid |
| | | | A | | | | | | | | | | | | tissue |
| chr2 | 89319827 | C | T, | 98 | 982 | 49 | 289 | 0.043838 | 0.105953318 | MIR4436A; | — | | intergenic | — | haematopoietic_ |
| | | | A, | | | | | | | LOC107985911 | | | | | and_lymphoid |
| | | | G | | | | | | | | | | | | tissue |
| chr2 | 89319865 | T | C, | 111 | 1057 | 42 | 216 | 0.020662 | 0.104831878 | MIR4436A; | — | | intergenic | — | haematopoietic_ |
| | | | G, | | | | | | | LOC107985911 | | | | | and_lymphoid |
| | | | A | | | | | | | | | | | | tissue |
| chr22 | 22712918 | G | T, | 42 | 529 | 44 | 238 | 0.004123 | 0.201328037 | GGTLC2; | — | | intergenic | — | large intestine |
| | | | A, | | | | | | | MIR650 | | | | | |
| | | | C | | | | | | | | | | | | |
| chr22 | 22712933 | C | G | 60 | 739 | 37 | 193 | 0.002672 | 0.174361754 | GGTLC2; | — | | intergenic | — | haematopoietic_ |
| | | | | | | | | | | MIR650 | | | | | and_lymphoid |
| | | | | | | | | | | | | | | | tissue |
| chr22 | 22712970 | A | G, | 71 | 785 | 21 | 84 | 0.003833 | 0.131598039 | GGTLC2; | — | | intergenic | — | haematopoietic_ |
| | | | T, | | | | | | | MIR650 | | | | | and_lymphoid |
| | | | C | | | | | | | | | | | | tissue |
| chr22 | 22712980 | G | C, | 59 | 695 | 37 | 219 | 0.024431 | 0.14581054 | GGTLC2; | — | | intergenic | — | haematopoietic_ |
| | | | A | | | | | | | MIR650 | | | | | and_lymphoid |
| | | | | | | | | | | | | | | | tissue |
| chr22 | 22712982 | A | G, | 42 | 596 | 52 | 307 | 0.001375 | 0.213213638 | GGTLC2; | — | | intergenic | — | haematopoietic_ |
| | | | C | | | | | | | MIR650 | | | | | and_lymphoid |
| | | | | | | | | | | | | | | | tissue |
| chr22 | 22712988 | A | C, | 52 | 611 | 39 | 225 | 0.02053 | 0.159432673 | GGTLC2; | — | | intergenic | — | haematopoietic_ |
| | | | G | | | | | | | MIR650 | | | | | _and_lymphoid |
| | | | | | | | | | | | | | | | tissue |
| chr22 | 22712997 | A | C, | 47 | 577 | 46 | 302 | 0.041735 | 0.151051415 | GGTLC2; | — | | intergenic | — | haematopoietic_ |
| | | | G, | | | | | | | MIR650 | | | | | and_lymphoid |
| | | | T | | | | | | | | | | | | tissue |
| chr22 | 22713005 | G | A, | 59 | 697 | 26 | 145 | 0.032566 | 0.133673327 | GGTLC2; | — | | intergenic | — | haematopoietic_ |
| | | | C, | | | | | | | MIR650 | | | | | and_lymphoid |
| | | | T | | | | | | | | | | | | tissue |
| chr22 | 22713022 | A | G, | 67 | 781 | 30 | 161 | 0.014607 | 0.138365399 | GGTLC2; | — | | intergenic | — | haematopoietic_ |
| | | | T | | | | | | | MIR650 | | | | | and_lymphoid |
| | | | | | | | | | | | | | | | tissue |
| chr22 | 22713038 | T | C. | 59 | 727 | 35 | 200 | 0.011145 | 0.156590695 | GGTLC2; | — | | intergenic | — | haematopoietic_ |
| | | | G | | | | | | | MIR650 | | | | | and_lymphoid |
| | | | | | | | | | | | | | | | tissue |
| chr22 | 22713044 | G | A | 53 | 553 | 43 | 280 | 0.034847 | 0.147809486 | GGTLC2; | — | | intergenic | — | haematopoietic_ |
| | | | | | | | | | | MIR650 | | | | | and_lymphoid |
| | | | | | | | | | | | | | | | tissue |
| chr22 | 22713060 | A | G, | 54 | 690 | 36 | 191 | 0.002731 | 0.183200908 | GGTLC2; | — | | intergenic | — | haematopoietic_ |
| | | | C, | | | | | | | MIR650 | | | | | and_lymphoid |
| | | | T | | | | | | | | | | | | tissue |
| chr22 | 22713061 | G | A, | 69 | 746 | 22 | 99 | 0.014946 | 0.124598478 | GGTLC2; | — | | intergenic | — | haematopoietic_ |
| | | | C, | | | | | | | MIR650 | | | | | and_lymphoid |
| | | | T | | | | | | | | | | | | tissue |
| chr22 | 22713064 | A | T, | 58 | 648 | 38 | 228 | 0.04829 | 0.135559361 | GGTLC2; | — | | intergenic | — | large intestine |
| | | | G, | | | | | | | MIR650 | | | | | |
| | | | C | | | | | | | | | | | | |
| chr22 | 22713166 | A | T, | 45 | 587 | 46 | 277 | 0.007861 | 0.184892654 | GGTLC2; | — | | intergenic | — | cervix |
| | | | G, | | | | | | | MIR650 | | | | | |
| | | | C | | | | | | | | | | | | |
| chr22 | 22713180 | G | C | 45 | 624 | 52 | 328 | 0.004234 | 0.191544659 | GGTLC2; | — | | intergenic | — | haematopoietic_ |
| | | | | | | | | | | MIR650 | | | | | and_lymphoid |
| | | | | | | | | | | | | | | | tissue |
| chr22 | 22895517 | C | T | 72 | 798 | 125 | 859 | 0.023839 | 0.116111007 | IGLL5 | NM_001256296 | c.C243T | exonic | synonymous_ | ovary |
| | | | | | | | | | | | | | | SNV | |
| chr22 | 28798829 | T | C | 81 | 320 | 85 | 139 | 8.86E−05 | 0.209215949 | XBP1 | — | | intronic | — | haematopoietic_ |
| | | | | | | | | | | | | | | | and_lymphoid |
| | | | | | | | | | | | | | | | tissue; lung |

TABLE 17
Expanded mutations in endothelial cells

| | | | | | | | | FDR | diff_ALT_ | | | | | | |
|---|---|---|---|---|---|---|---|---|---|---|---|---|---|---|---|
| | | | | REF_endothelial | REF_other | ALT_endothelial | ALT_other | adj | cellular | | | | | | |
| | | | | cell | cell | cell | cell | P- | prevalence_ | Gene | RefSeq_ | | Func_ | Mutation_ | |
| CHR | POS | REF | ALT | # | # | # | # | values | endothelial | Name | ID | Coding | type | type | Cosmic |
| chr10 | 1.7E+07 | C | T | 28 | 497 | 113 | 1019 | 0.0380461 | 0.129254851 | VIM | — | | UTR3 | — | lung;large_ |
| | | | | | | | | | | | | | | | intestine |
| chr11 | 6.6E+07 | C | T | 43 | 934 | 129 | 1477 | 0.0110995 | 0.137391124 | MALAT1; | — | | ncRNA_ | — | lung |
| | | | | | | | | | | TALAM1 | | | exonic | | |
| chr11 | 6.6E+07 | C | T | 18 | 559 | 158 | 1847 | 0.0032383 | 0.1300631 | MALAT1; | — | | ncRNA_ | — | central_nervous_ |
| | | | | | | | | | | TALAM1 | | | exonic | | system |
| chr15 | 4.5E+07 | C | T | 68 | 1437 | 93 | 976 | 0.0011808 | 0.173163995 | B2M | — | | UTR3 | — | lung;thyroid; |
| | | | | | | | | | | | | | | | large_intestine |
| chr16 | 1964501 | C | T | 67 | 1261 | 61 | 524 | 0.0011666 | 0.183005077 | RPS2 | NM_002952 | c.G125A | exonic | non- | lung |
| | | | | | | | | | | | | | | synonymous_ | |
| | | | | | | | | | | | | | | SNV | |
| chr17 | 6.4E+07 | G | A | 70 | 914 | 82 | 588 | 0.012879 | 0.147995655 | DDX5 | — | | UTR3 | — | haematopoietic_ |
| | | | | | | | | | | | | | | | and_lymphoid_ |
| | | | | | | | | | | | | | | | tissue |
| chr19 | 4.2E+07 | C | T | 62 | 1090 | 66 | 591 | 0.007725 | 0.164048557 | RPS19 | NM_001321485 | c.C268T | exonic | non- | lung |
| | | | | | | | | | | | | | | synonymous_ | |
| | | | | | | | | | | | | | | SNV | |
| chr4 | 8.8E+07 | C | T | 31 | 65 | 111 | 103 | 0.0367043 | 0.168594903 | SPARCL1 | — | | UTR5 | — | pancreas;large_ |
| | | | | | | | | | | | | | | | intestine |
| chr4 | 8.8E+07 | T | C | 32 | 61 | 107 | 89 | 0.03605 | 0.176450839 | SPARCL1 | — | | UTR5 | — | soft tissue |
| chr6 | 3E+07 | G | A | 72 | 1163 | 58 | 400 | 0.0003172 | 0.19023574 | HLA- | NM_001242758 | c.G203A | exonic | non- | thyroid |
| | | | | | | | | | | A | | | | synonymous_ | |
| | | | | | | | | | | | | | | SNV | |
| chr6 | 3E+07 | G | A | 67 | 1129 | 65 | 438 | 4.04E−05 | 0.212909246 | HLA- | NM_001242758 | c.G271A | exonic | non- | thyroid |
| | | | | | | | | | | A | | | | synonymous_ | |
| | | | | | | | | | | | | | | SNV | |
| chr6 | 3E+07 | G | C | 68 | 1117 | 62 | 444 | 0.0004052 | 0.192490021 | HLA- | NM_001242758 | c.G282C | exonic | non- | thyroid |
| | | | | | | | | | | A | | | | synonymous_ | |
| | | | | | | | | | | | | | | SNV | |
| chr6 | 3E+07 | T | C | 70 | 1080 | 66 | 495 | 0.0025336 | 0.171008403 | HLA- | NM_001242758 | c.T385C | exonic | non- | thyroid;urinary_ |
| | | | | | | | | | | A | | | | synonymous_ | tract |
| | | | | | | | | | | | | | | SNV | |
| chr6 | 3E+07 | G | A | 64 | 1019 | 72 | 558 | 0.0025336 | 0.175575366 | HLA- | NM_001242758 | c.G489A | exonic | synonymous_ | thyroid |
| | | | | | | | | | | A | | | | SNV | |
| chr6 | 3E+07 | C | T | 71 | 1115 | 65 | 470 | 0.0009615 | 0.181411208 | HLA- | NM_001242758 | c.C521T | exonic | non- | upper_aerodigestive_ |
| | | | | | | | | | | A | | | | synonymous_ | tract;skin |
| | | | | | | | | | | | | | | SNV | |
| chr6 | 3E+07 | T | C | 72 | 1117 | 64 | 465 | 0.0013231 | 0.176656503 | HLA- | NM_001242758 | c.T538C | exonic | synonymous_ | oesophagus;large_ |
| | | | | | | | | | | A | | | | SNV | intestine; |
| | | | | | | | | | | | | | | | haematopoietic_and_ |
| | | | | | | | | | | | | | | | lymphoid_tissue |
| chr6 | 3E+07 | T | G | 66 | 999 | 70 | 586 | 0.0229369 | 0.144989794 | HLA- | NM_001242758 | c.T539G | exonic | non- | soft_tissue;pancreas; |
| | | | | | | | | | | A | | | | synonymous_ | large_intestine |
| | | | | | | | | | | | | | | SNV | |
| chr6 | 3E+07 | C | T | 71 | 1096 | 66 | 493 | 0.0022885 | 0.171493801 | HLA- | NM_ | c.C545T | exonic | non- | breast;upper_ |
| | | | | | | | | | | A | 001242758 | | | synonymous_ | aerodigestive_tract |
| | | | | | | | | | | | | | | SNV | |
| chr6 | 3E+07 | A | C | 65 | 1101 | 69 | 487 | 1.00E−04 | 0.20825031 | HLA- | NM_ | c.A559C | exonic | non- | thyroid;upper_ |
| | | | | | | | | | | A | 001242758 | | | synonymous_ | aerodigestive_tract |
| | | | | | | | | | | | | | | SNV | |
| chr6 | 3E+07 | G | C | 71 | 1212 | 63 | 366 | 4.75E−07 | 0.23821009 | HLA- | NM_ | c.G570C | exonic | non- | thyroid;large_ |
| | | | | | | | | | | A | 001242758 | | | synonymous_ | intestine;kidney;biliary_ |
| | | | | | | | | | | | | | | SNV | tract;haematopoietic_ |
| | | | | | | | | | | | | | | | and_lymphoid_ |
| | | | | | | | | | | | | | | | tissue;skin |
| chr6 | 3E+07 | T | G, | 68 | 1129 | 65 | 422 | 2.22E−05 | 0.216639277 | HLA- | NM_ | c.T571C | exonic | non- | upper_aerodigestive_ |
| | | | A, | | | | | | | A | 001242758 | | | synonymous_ | tract |
| | | | C | | | | | | | | | | | SNV | |
| chr6 | 3E+07 | C | G | 70 | 1439 | 78 | 567 | 2.25E−07 | 0.244374983 | HLA- | — | | UTR3 | — | upper_aerodigestive_ |
| | | | | | | | | | | A | | | | | tract;haematopoietic_ |
| | | | | | | | | | | | | | | | and_lymphoid_ |
| | | | | | | | | | | | | | | | tissue |
| chr6 | 3E+07 | C | T | 74 | 1468 | 78 | 596 | 2.02E−06 | 0.224398205 | HLA- | — | | UTR3 | — | upper_aerodigestive_ |
| | | | | | | | | | | A | | | | | tract;lung |
| chr6 | 3.1E+07 | T | C | 52 | 1149 | 94 | 560 | 9.50E−11 | 0.316158612 | HLA- | — | | UTR3 | — | upper_aerodigestive_ |
| | | | | | | | | | | C | | | | | tract |
| chr6 | 3.1E+07 | C | G | 57 | 1028 | 71 | 426 | 4.75E−07 | 0.261702631 | HLA- | NM_001243042 | c.G270C | exonic | non- | kidney; lung |
| | | | | | | | | | | C | | | | synonymous_ | |
| | | | | | | | | | | | | | | SNV | |
| chr6 | 3.1E+07 | C | G | 78 | 1391 | 74 | 525 | 6.69E−06 | 0.212833755 | HLA- | NM_005514 | c.G540C | exonic | synonymous_ | upper_aerodigestive_ |
| | | | | | | | | | | B | | | | SNV | tract;haematopoietic_ |
| | | | | | | | | | | | | | | | and_lymphoid_ |
| | | | | | | | | | | | | | | | tissue |
| chr6 | 3.1E+07 | G | C | 80 | 1391 | 72 | 505 | 1.06E−05 | 0.207334 | HLA- | NM_005514 | c.C538G | exonic | non- | upper_aerodigestive_ |
| | | | | | | | | | | B | | | | synonymous_ | tract |
| | | | | | | | | | | | | | | SNV | |
| chr6 | 3.1E+07 | C | T | 76 | 1356 | 75 | 563 | 3.00E−05 | 0.203306772 | HLA- | NM_005514 | c.G412A | exonic | non- | soft_tissue;urinary_ |
| | | | | | | | | | | B | | | | synonymous_ | tract; thyroid; biliary_ |
| | | | | | | | | | | | | | | SNV | tract |
| chr6 | 3.1E+07 | A | C | 73 | 1334 | 72 | 518 | 8.69E−06 | 0.2168541 | HLA- | NM_005514 | c.T97G | exonic | non- | upper_aerodigestive_ |
| | | | | | | | | | | B | | | | synonymous_ | tract;central_ |
| | | | | | | | | | | | | | | SNV | nervous_system |
| chr6 | 3.2E+07 | A | T | 22 | 424 | 31 | 120 | 2.64E−06 | 0.364317425 | ATP6V1G2- | — | | ncRNA_ | — | upper_aerodigestive_ |
| | | | | | | | | | | DDX39B | | | intronic | | tract;thyroid;skin; |
| | | | | | | | | | | | | | | | haematopoietic_ |
| | | | | | | | | | | | | | | | and |
| | | | | | | | | | | | | | | | lymphoid_tissue |
| chr8 | 6562969 | C | G | 27 | 86 | 113 | 109 | 0.0002761 | 0.248168498 | ANGPT2 | — | | UTR5 | — | haematopoietic_and_ |
| | | | | | | | | | | | | | | | lymphoid_tissue; |
| | | | | | | | | | | | | | | | lung |
| chr8 | 6563013 | A | G | 22 | 89 | 117 | 92 | 3.39E−07 | 0.333439326 | ANGPT2 | — | | UTR5 | — | haematopoietic_and_ |
| | | | | | | | | | | | | | | | lymphoid_tissue; |
| | | | | | | | | | | | | | | | lung |
| chr8 | 6563053 | A | G | 29 | 86 | 111 | 106 | 0.0005323 | 0.24077381 | ANGPT2 | — | | UTR5 | — | large_intestine |
| chr9 | 1.4E+08 | G | A | 0 | 27 | 166 | 106 | 8.00E−07 | 0.203007519 | EGFL7 | — | | UTR3 | — | liver |
| chrM | 10685 | G | A | 44 | 751 | 63 | 525 | 0.0126112 | 0.17734304 | MIR12136; | — | | intergenic | — | large_intestine; |
| | | | | | | | | | | NONE | | | | | breast;haematopoietic_ |
| | | | | | | | | | | | | | | | and_lymphoid_tissue; |
| | | | | | | | | | | | | | | | stomach |
| chrM | 14159 | C | T, | 54 | 721 | 56 | 373 | 0.0150223 | 0.168140269 | MIR12136; | — | | intergenic | — | large_intestine; |
| | | | A | | | | | | | NONE | | | | | breast;haematopoietic_ |
| | | | | | | | | | | | | | | | and_lymphoid_tissue; |
| | | | | | | | | | | | | | | | stomach |
| chrM | 14846 | G | A | 76 | 1180 | 74 | 603 | 0.0054693 | 0.155139278 | MIR12136; | — | | intergenic | — | large_intestine; |
| | | | | | | | | | | NONE | | | | | breast;haematopoietic_ |
| | | | | | | | | | | | | | | | and_lymphoid_tissue; |
| | | | | | | | | | | | | | | | stomach |
| chrM | 15041 | G | A | 74 | 1210 | 82 | 646 | 0.0007761 | 0.177580681 | MIR12136; | — | | intergenic | — | large_intestine; |
| | | | | | | | | | | NONE | | | | | breast;haematopoietic_ |
| | | | | | | | | | | | | | | | and_lymphoid_tissue; |
| | | | | | | | | | | | | | | | stomach |
| chrM | 15437 | G | A | 81 | 1346 | 81 | 663 | 0.0009352 | 0.169985067 | MIR12136; | — | | intergenic | — | large_intestine; |
| | | | | | | | | | | NONE | | | | | breast;haematopoietic_ |
| | | | | | | | | | | | | | | | and_lymphoid_tissue; |
| | | | | | | | | | | | | | | | stomach |
| chrM | 1792 | G | A | 49 | 1108 | 127 | 1463 | 0.0033645 | 0.152551625 | NONE; | — | | intergenic | — | large_intestine; |
| | | | | | | | | | | MIR12136 | | | | | breast;haematopoietic_ |
| | | | | | | | | | | | | | | | and_lymphoid_tissue; |
| | | | | | | | | | | | | | | | stomach |
| chrM | 1905 | C | T, | 36 | 869 | 141 | 1698 | 0.0079399 | 0.135137633 | NONE; | — | | intergenic | — | large_intestine; |
| | | | G, | | | | | | | MIR12136 | | | | | breast;haematopoietic_ |
| | | | A | | | | | | | | | | | | and_lymphoid_tissue; |
| | | | | | | | | | | | | | | | stomach |
| chrM | 1948 | C | T | 32 | 777 | 145 | 1801 | 0.0188776 | 0.120605471 | NONE; | — | | intergenic | — | large_intestine; |
| | | | | | | | | | | MIR12136 | | | | | breast;haematopoietic_ |
| | | | | | | | | | | | | | | | and_lymphoid_tissue; |
| | | | | | | | | | | | | | | | stomach |
| chrM | 3021 | C | T | 64 | 1166 | 107 | 1187 | 0.0458633 | 0.121268606 | NONE; | — | | intergenic | — | large_intestine; |
| | | | | | | | | | | MIR12136 | | | | | breast;haematopoietic_ |
| | | | | | | | | | | | | | | | and_lymphoid_tissue; |
| | | | | | | | | | | | | | | | stomach |
| chrM | 3954 | C | T | 63 | 1269 | 92 | 1023 | 0.012032 | 0.147213309 | NONE; | — | | intergenic | — | large_intestine; |
| | | | | | | | | | | MIR12136 | | | | | breast;haematopoietic_ |
| | | | | | | | | | | | | | | | and_lymphoid_tissue; |
| | | | | | | | | | | | | | | | stomach |
| chrM | 5980 | C | T | 74 | 1380 | 84 | 742 | 0.0004052 | 0.181975447 | NONE; | — | | intergenic | — | large_intestine; |
| | | | | | | | | | | MIR12136 | | | | | breast;haematopoietic_ |
| | | | | | | | | | | | | | | | and_lymphoid_tissue; |
| | | | | | | | | | | | | | | | stomach |
| chrM | 6173 | C | T | 44 | 1016 | 116 | 1142 | 0.0001818 | 0.195806302 | NONE; | — | | intergenic | — | large_intestine; |
| | | | | | | | | | | MIR12136 | | | | | breast;haematopoietic_ |
| | | | | | | | | | | | | | | | and_lymphoid_tissue; |
| | | | | | | | | | | | | | | | stomach |
| chrM | 6957 | G | A | 35 | 906 | 135 | 1459 | 0.0003725 | 0.177204328 | MIR12136 | — | | downstream | — | large_intestine; |
| | | | | | | | | | | | | | | | breast;haematopoietic_ |
| | | | | | | | | | | | | | | | and_lymphoid_tissue; |
| | | | | | | | | | | | | | | | stomach |
| chrM | 7215 | C | T | 49 | 1030 | 125 | 1452 | 0.0160527 | 0.133378718 | MIR12136 | — | | downstream | — | large_intestine; |
| | | | | | | | | | | | | | | | breast;haematopoietic_ |
| | | | | | | | | | | | | | | | and_lymphoid_tissue; |
| | | | | | | | | | | | | | | | stomach |
| chrM | 7235 | C | T | 44 | 1026 | 130 | 1459 | 0.0019012 | 0.1600037 | MIR12136 | — | | downstream | — | large_intestine; |
| | | | | | | | | | | | | | | | breast;haematopoietic_ |
| | | | | | | | | | | | | | | | and_lymphoid_tissue; |
| | | | | | | | | | | | | | | | stomach |
| chrM | 7792 | C | T | 50 | 1194 | 124 | 1354 | 0.0003433 | 0.181246504 | MIR12136 | — | | upstream | — | large_intestine; |
| | | | | | | | | | | | | | | | breast;haematopoietic_ |
| | | | | | | | | | | | | | | | and_lymphoid_tissue; |
| | | | | | | | | | | | | | | | stomach |
| chrM | 8019 | C | T | 23 | 598 | 151 | 1964 | 0.0456478 | 0.101227489 | MIR12136 | — | | upstream | — | large_intestine; |
| | | | | | | | | | | | | | | | breast;haematopoietic_ |
| | | | | | | | | | | | | | | | and_lymphoid_tissue; |
| | | | | | | | | | | | | | | | stomach |
| chrM | 8140 | C | T, | 37 | 1054 | 137 | 1512 | 3.38E−05 | 0.198112362 | MIR12136 | — | | upstream | — | large_intestine; |
| | | | A | | | | | | | | | | | | breast;haematopoietic_ |
| | | | | | | | | | | | | | | | and_lymphoid_tissue; |
| | | | | | | | | | | | | | | | stomach |
| chrM | 8372 | C | T | 69 | 1290 | 88 | 853 | 0.0031903 | 0.162469423 | MIR12136 | — | | upstream | — | large_intestine; |
| | | | | | | | | | | | | | | | breast;haematopoietic_ |
| | | | | | | | | | | | | | | | and_lymphoid_tissue; |
| | | | | | | | | | | | | | | | stomach |
| chrM | 8841 | C | T | 61 | 1144 | 107 | 1200 | 0.0389821 | 0.124959369 | MIR12136; | — | | intergenic | — | large_intestine; |
| | | | | | | | | | | NONE | | | | | breast;haematopoietic_ |
| | | | | | | | | | | | | | | | and_lymphoid_tissue; |
| | | | | | | | | | | | | | | | stomach |
| chrM | 9777 | G | A | 54 | 1117 | 121 | 1419 | 0.0186474 | 0.131885985 | MIR12136; | — | | intergenic | — | large_intestine; |
| | | | | | | | | | | NONE | | | | | breast;haematopoietic_ |
| | | | | | | | | | | | | | | | and_lymphoid_tissue; |
| | | | | | | | | | | | | | | | stomach |
| chrX | 2.4E+07 | G | T | 56 | 822 | 96 | 460 | 6.03E−08 | 0.272764595 | SAT1 | — | | UTR5 | — | haematopoietic_and_ |
| | | | | | | | | | | | | | | | lymphoid_tissue; |
| | | | | | | | | | | | | | | | lung |
| chrX | 2.4E+07 | C | T | 31 | 324 | 71 | 282 | 0.0012703 | 0.230731897 | SAT1 | — | | intronic | — | lung |

TABLE 18
Expanded mutations in follicular B cells

| | | | | REF_follicular | | ALT_follicular | | | diff_ALT_cellular | | | | | | |
|---|---|---|---|---|---|---|---|---|---|---|---|---|---|---|---|
| | | | | B | REF_other | B | ALT_other | | prevalence_ | | | | | | |
| | | | | cell | cell | cell | cell | FDR adj | follicular | Gene | RefSeq_ | | | | |
| CHR | POS | REF | ALT | numbers | numbers | numbers | numbers | P-values | B vs | Name | ID | Coding | Func_type | Mutation_type | Cosmic |
| chr11 | 65499858 | A | G | 34 | 2024 | 150 | 632 | 1.98E−50 | 0.577265584 | MALAT1; | — | | ncRNA_ | — | lung |
| | | | | | | | | | | TALAM1 | | | exonic | | |
| chr22 | 22720669 | T | C | 19 | 313 | 95 | 110 | 1.06E−25 | 0.573286052 | GGTLC2; | — | | intergenic | — | haematopoietic_ |
| | | | | | | | | | | MIR650 | | | | | and_lymphoid_ |
| | | | | | | | | | | | | | | | tissue |
| chr22 | 22720862 | G | A, | 12 | 289 | 109 | 140 | 9.51E−26 | 0.57448612 | GGTLC2; | — | | intergenic | — | haematopoietic_ |
| | | | C | | | | | | | MIR650 | | | | | and_lymphoid_ |
| | | | | | | | | | | | | | | | tissue |
| chr22 | 22720863 | G | A, | 11 | 275 | 111 | 147 | 9.71E−25 | 0.561494833 | GGTLC2; | — | | intergenic | — | haematopoietic_ |
| | | | C, | | | | | | | MIR650 | | | | | and_lymphoid_ |
| | | | T | | | | | | | | | | | | tissue |
| chr22 | 22720950 | A | G, | 14 | 276 | 106 | 151 | 7.64E−22 | 0.529703357 | GGTLC2; | — | | intergenic | — | haematopoietic_ |
| | | | C, | | | | | | | MIR650 | | | | | and_lymphoid_ |
| | | | T | | | | | | | | | | | | tissue |
| chr22 | 22720978 | A | G | 9 | 188 | 113 | 261 | 3.58E−10 | 0.344937749 | GGTLC2; | — | | intergenic | — | haematopoietic_ |
| | | | | | | | | | | MIR650 | | | | | and_lymphoid_ |
| | | | | | | | | | | | | | | | tissue |
| chr22 | 22721004 | A | C, | 9 | 249 | 112 | 173 | 8.59E−21 | 0.515667228 | GGTLC2; | — | | intergenic | — | haematopoietic_ |
| | | | T, | | | | | | | MIR650 | | | | | and_lymphoid_ |
| | | | G | | | | | | | | | | | | tissue |
| chr22 | 22721048 | C | G, | 11 | 311 | 111 | 140 | 1.05E−28 | 0.59941478 | GGTLC2; | — | | intergenic | — | haematopoietic_ |
| | | | T | | | | | | | MIR650 | | | | | and_lymphoid_ |
| | | | | | | | | | | | | | | | tissue |
| chr22 | 22721060 | C | G, | 10 | 275 | 112 | 160 | 5.99E−24 | 0.550216695 | GGTLC2; | — | | intergenic | — | haematopoietic_ |
| | | | T | | | | | | | MIR650 | | | | | and_lymphoid_ |
| | | | | | | | | | | | | | | | tissue |
| chr22 | 22721063 | A | G, | 24 | 286 | 96 | 143 | 3.69E−17 | 0.466666667 | GGTLC2; | — | | intergenic | — | haematopoietic_ |
| | | | T | | | | | | | MIR650 | | | | | and_lymphoid_ |
| | | | | | | | | | | | | | | | tissue |
| chr22 | 22721066 | A | G, | 14 | 287 | 108 | 135 | 3.02E−25 | 0.565340688 | GGTLC2; | — | | intergenic | — | haematopoietic_ |
| | | | C | | | | | | | MIR650 | | | | | and_lymphoid_ |
| | | | | | | | | | | | | | | | tissue |
| chr22 | 22721096 | T | C | 14 | 298 | 109 | 152 | 2.93E−24 | 0.548401084 | GGTLC2; | — | | intergenic | — | haematopoietic_ |
| | | | | | | | | | | MIR650 | | | | | and_lymphoid_ |
| | | | | | | | | | | | | | | | tissue |
| chr22 | 22721123 | C | T | 10 | 217 | 111 | 227 | 2.30E−13 | 0.406094111 | GGTLC2; | — | | intergenic | — | haematopoietic_ |
| | | | | | | | | | | MIR650 | | | | | and_lymphoid_ |
| | | | | | | | | | | | | | | | tissue |
| chr22 | 22721127 | A | C, | 12 | 248 | 112 | 150 | 9.94E−22 | 0.526341384 | GGTLC2; | — | | intergenic | — | haematopoietic_ |
| | | | G, | | | | | | | MIR650 | | | | | and_lymphoid_ |
| | | | T | | | | | | | | | | | | tissue |
| chr22 | 22721129 | C | G, | 8 | 234 | 113 | 171 | 2.25E−20 | 0.511662075 | GGTLC2; | — | | intergenic | — | haematopoietic_ |
| | | | T | | | | | | | MIR650 | | | | | and_lymphoid_ |
| | | | | | | | | | | | | | | | tissue |
| chr22 | 22899624 | A | C, | 32 | 721 | 126 | 731 | 4.83E−10 | 0.294024828 | IGLL5; | — | | intergenic | — | ovary |
| | | | T, | | | | | | | RSPH14 | | | | | |
| | | | G | | | | | | | | | | | | |
| chr22 | 22906484 | C | T | 58 | 923 | 108 | 698 | 8.19E−06 | 0.220004014 | IGLL5; | — | | intergenic | — | large_intestine |
| | | | | | | | | | | RSPH14 | | | | | |
| chr6 | 31533435 | A | T | 8 | 438 | 15 | 136 | 0.00089 | 0.415240115 | ATP6V1G2- | — | | ncRNA_ | — | upper_ |
| | | | | | | | | | | DDX39B | | | intronic | | aerodigestive_ |
| | | | | | | | | | | | | | | | tract; |
| | | | | | | | | | | | | | | | thyroid; skin; |
| | | | | | | | | | | | | | | | haematopoietic_ |
| | | | | | | | | | | | | | | | and_lymphoid |
| | | | | | | | | | | | | | | | tissue |
| chr6 | 32444789 | T | A | 27 | 450 | 48 | 266 | 0.00052 | 0.26849162 | HLA-DRA | — | | UTR3 | — | upper_ |
| | | | | | | | | | | | | | | | aerodigestive_ |
| | | | | | | | | | | | | | | | tract |
| chr6 | 32444815 | G | A | 27 | 420 | 51 | 303 | 0.00317 | 0.234759017 | HLA-DRA | — | | UTR3 | — | upper_ |
| | | | | | | | | | | | | | | | aerodigestive_ |
| | | | | | | | | | | | | | | | tract |
| chr6 | 32445032 | T | A | 31 | 452 | 34 | 199 | 0.0111 | 0.217393359 | HLA-DRA | — | | UTR3 | — | upper_ |
| | | | | | | | | | | | | | | | aerodigestive_ |
| | | | | | | | | | | | | | | | tract |
| chr6 | 32578889 | G | A | 46 | 753 | 48 | 357 | 0.00663 | 0.189016676 | HLA-DRB1 | — | | UTR3 | — | haematopoietic_ |
| | | | | | | | | | | | | | | | and_ |
| | | | | | | | | | | | | | | | lymphoid_tissue; |
| | | | | | | | | | | | | | | | liver;large_ |
| | | | | | | | | | | | | | | | intestine |
| chr6 | 32578907 | A | G | 45 | 760 | 47 | 350 | 0.00483 | 0.19555425 | HLA-DRB1 | — | | UTR3 | — | haematopoietic_ |
| | | | | | | | | | | | | | | | and_ |
| | | | | | | | | | | | | | | | lymphoid_tissue; |
| | | | | | | | | | | | | | | | liver;large_ |
| | | | | | | | | | | | | | | | intestine |
| chr6 | 32578920 | G | A | 43 | 710 | 51 | 388 | 0.00804 | 0.189183428 | HLA-DRB1 | — | | UTR3 | — | haematopoietic_ |
| | | | | | | | | | | | | | | | and_ |
| | | | | | | | | | | | | | | | lymphoid_tissue; |
| | | | | | | | | | | | | | | | liver;large_ |
| | | | | | | | | | | | | | | | intestine |
| chr6 | 32578921 | G | A | 43 | 743 | 51 | 366 | 0.00169 | 0.21252614 | HLA-DRB1 | — | | UTR3 | — | haematopoietic_ |
| | | | | | | | | | | | | | | | and_ |
| | | | | | | | | | | | | | | | lymphoid_tissue; |
| | | | | | | | | | | | | | | | liver;large_ |
| | | | | | | | | | | | | | | | intestine |
| chr6 | 32579023 | A | G | 47 | 740 | 43 | 324 | 0.01684 | 0.1732665 | HLA-DRB1 | — | | UTR3 | — | haematopoietic_ |
| | | | | | | | | | | | | | | | and_ |
| | | | | | | | | | | | | | | | lymphoid_tissue; |
| | | | | | | | | | | | | | | | liver;large_ |
| | | | | | | | | | | | | | | | intestine |
| chr6 | 32579069 | C | T | 37 | 664 | 52 | 393 | 0.0032 | 0.212462662 | HLA-DRB1 | — | | UTR3 | — | haematopoietic_ |
| | | | | | | | | | | | | | | | and_ |
| | | | | | | | | | | | | | | | lymphoid_tissue; |
| | | | | | | | | | | | | | | | liver;large_ |
| | | | | | | | | | | | | | | | intestine |
| chr6 | 32584162 | G | T, | 38 | 665 | 36 | 300 | 0.03585 | 0.175605657 | HLA-DRB1 | NM_002124 | c.C317G | exonic | non- | haematopoietic_ |
| | | | C | | | | | | | | | | | synonymous_ | and_ |
| | | | | | | | | | | | | | | SNV | lymphoid_tissue; |
| | | | | | | | | | | | | | | | liver;large_ |
| | | | | | | | | | | | | | | | intestine |
| chr6 | 32589702 | G | A | 44 | 699 | 32 | 241 | 0.03585 | 0.164669653 | HLA-DRB1 | NM_002124 | c.C41T | exonic | non- | haematopoietic_ |
| | | | | | | | | | | | | | | synonymous_ | and_ |
| | | | | | | | | | | | | | | SNV | lymphoid_tissue; |
| | | | | | | | | | | | | | | | liver;large_ |
| | | | | | | | | | | | | | | | intestine |

TABLE 19
Expanded mutations in T & NK cells

| | | | | | | | | | diff_ALT_ | | | | | | |
|---|---|---|---|---|---|---|---|---|---|---|---|---|---|---|---|
| | | | | | | | | | cellular | | | | | | |
| | | | | | | | ALT | FDR | prevalence_ | | | | | | |
| | | | | REF_T&NK | REF_other | ALT_T&NK | other | adj | T&NK | | | | | | |
| | | | | cell | cell | cell | cell | P- | vs | Gene | RefSeq_ | | Func_ | Mutation_ | |
| CHR | POS | REF | ALT | numbers | numbers | numbers | numbers | values | others | Name | ID | Coding | type | type | Cosmic |
| chr14 | 1.06E+08 | C | G, | 24 | 245 | 43 | 157 | 0.001602 | 0.251243781 | ADAM6; | — | | intergenic | — | ovary |
| | | | A, | | | | | | | LINC00226 | | | | | |
| | | | T | | | | | | | | | | | | |
| chr14 | 1.06E+08 | G | A | 35 | 275 | 39 | 120 | 0.002481 | 0.223229559 | ADAM6; | — | | intergenic | — | ovary |
| | | | C | | | | | | | LINC00226 | | | | | |
| chr14 | 1.06E+08 | G | A | 37 | 279 | 37 | 131 | 0.023736 | 0.180487805 | ADAM6; | — | | intergenic | — | ovary |
| | | | | | | | | | | LINC00226 | | | | | |
| chr17 | 6774861 | A | C | 17 | 168 | 30 | 118 | 0.034784 | 0.22571046 | XAF1 | — | | UTR3 | — | stomach |
| chr6 | 29942886 | G | A | 146 | 1089 | 119 | 339 | 1.01E−10 | 0.211661646 | HLA-A | NM_001242758 | c.G203A | exonic | non- | thyroid |
| | | | | | | | | | | | | | | synonymous_ | |
| | | | | | | | | | | | | | | SNV | |
| chr6 | 29942954 | G | A | 126 | 1070 | 135 | 368 | 5.54E−15 | 0.261330392 | HLA-A | NM_001242758 | c.G271A | exonic | non- | thyroid |
| | | | | | | | | | | | | | | synonymous_ | |
| | | | | | | | | | | | | | | SNV | |
| chr6 | 29942965 | G | C | 128 | 1057 | 134 | 372 | 8.50E−14 | 0.251128478 | HLA-A | NM_001242758 | c.G282C | exonic | non- | thyroid |
| | | | | | | | | | | | | | | synonymous_ | |
| | | | | | | | | | | | | | | SNV | |
| chr6 | 29943309 | T | C | 126 | 1024 | 137 | 424 | 4.32E−11 | 0.228094868 | HLA-A | NM_001242758 | c.T385C | exonic | non- | thyroid;urinary_tract |
| | | | | | | | | | | | | | | synonymous_ | |
| | | | | | | | | | | | | | | SNV | |
| chr6 | 29943413 | G | A | 124 | 959 | 145 | 485 | 1.26E−08 | 0.203160881 | HLA-A | NM_001242758 | c.G489A | exonic | synonymous_ | thyroid |
| | | | | | | | | | | | | | | SNV | |
| chr6 | 29943445 | C | T | 131 | 1055 | 140 | 395 | 3.47E−13 | 0.244191373 | HLA-A | NM_001242758 | c.C521T | exonic | non- | upper_aerodigestive_ |
| | | | | | | | | | | | | | | synonymous_ | tract;skin |
| | | | | | | | | | | | | | | SNV | |
| chr6 | 29943462 | T | C | 136 | 1053 | 131 | 398 | 1.67E−10 | 0.216343113 | HLA-A | NM_001242758 | c.T538C | exonic | synonymous_ | oesophagus;large_ |
| | | | | | | | | | | | | | | SNV | intestine; |
| | | | | | | | | | | | | | | | haematopoietic_and_ |
| | | | | | | | | | | | | | | | lymphoid_tissue |
| chr6 | 29943463 | T | G | 124 | 941 | 145 | 511 | 2.53E−07 | 0.187105083 | HLA-A | NM_001242758 | c.T539G | exonic | non- | soft_tissue;pancreas;large_ |
| | | | | | | | | | | | | | | synonymous_ | intestine |
| | | | | | | | | | | | | | | SNV | |
| chr6 | 29943469 | C | T | 127 | 1040 | 144 | 415 | 3.81E−13 | 0.246141946 | HLA-A | NM_001242758 | c.C545T | exonic | non- | breast;upper_ |
| | | | | | | | | | | | | | | synonymous_ | aerodigestive_tract |
| | | | | | | | | | | | | | | SNV | |
| chr6 | 29943483 | A | C | 135 | 1031 | 130 | 426 | 1.26E−08 | 0.198184432 | HLA-A | NM_001242758 | c.A559C | exonic | non- | thyroid;upper_ |
| | | | | | | | | | | | | | | synonymous_ | aerodigestive_tract |
| | | | | | | | | | | | | | | SNV | |
| chr6 | 29943494 | G | C | 148 | 1135 | 113 | 316 | 1.84E−11 | 0.215169351 | HLA-A | NM_001242758 | c.G570C | exonic | non- | thyroid;large_intestine; |
| | | | | | | | | | | | | | | synonymous_ | kidney;biliary_tract; |
| | | | | | | | | | | | | | | SNV | haematopoietic_and_ |
| | | | | | | | | | | | | | | | lymphoid_tissue;skin |
| chr6 | 29943495 | T | G, | 138 | 1059 | 119 | 368 | 1.83E−09 | 0.205151347 | HLA-A | NM_001242758 | c.T571C | exonic | non- | upper_aerodigestive_tract |
| | | | A, | | | | | | | | | | | synonymous_ | |
| | | | C | | | | | | | | | | | SNV | |
| chr6 | 29945597 | C | G | 150 | 1359 | 169 | 476 | 1.51E−19 | 0.270380019 | HLA-A | — | | UTR3 | — | upper_aerodigestive_ |
| | | | | | | | | | | | | | | | tract; |
| | | | | | | | | | | | | | | | haematopoietic_and_ |
| | | | | | | | | | | | | | | | lymphoid_tissue |
| chr6 | 29945653 | C | T | 157 | 1385 | 174 | 500 | 1.23E−18 | 0.260427769 | HLA-A | — | | UTR3 | — | upper_aerodigestive_ |
| | | | | | | | | | | | | | | | tract;lung |
| chr6 | 31269077 | T | C | 114 | 1087 | 180 | 474 | 3.70E−21 | 0.308593393 | HLA-C | — | | UTR3 | — | upper_aerodigestive_tract |
| chr6 | 31271672 | C | G | 121 | 964 | 145 | 352 | 2.49E−16 | 0.277635578 | HLA-C | NM_001243042 | c.G270C | exonic | non- | kidney;lung |
| | | | | | | | | | | | | | | synonymous_ | |
| | | | | | | | | | | | | | | SNV | |
| chr6 | 31356246 | C | G | 177 | 1292 | 153 | 446 | 3.95E−12 | 0.207019563 | HLA-B | NM_005514 | c.G540C | exonic | synonymous_ | upper_aerodigestive_ |
| | | | | | | | | | | | | | | SNV | tract; |
| | | | | | | | | | | | | | | | haematopoietic_and_ |
| | | | | | | | | | | | | | | | lymphoid_tissue |
| chr6 | 31356248 | G | C | 179 | 1292 | 151 | 426 | 1.31E−12 | 0.20961301 | HLA-B | NM_005514 | c.C538G | exonic | non- | upper_aerodigestive_tract |
| | | | | | | | | | | | | | | synonymous_ | |
| | | | | | | | | | | | | | | SNV | |
| chr6 | 31356374 | C | T | 172 | 1260 | 161 | 477 | 4.81E−12 | 0.208872085 | HLA-B | NM_005514 | c.G412A | exonic | non- | soft_tissue;urinary_ |
| | | | | | | | | | | | | | | synonymous_ | tract;thyroid; |
| | | | | | | | | | | | | | | SNV | biliary_tract |
| chr6 | 31356934 | A | C | 173 | 1234 | 154 | 436 | 3.90E−12 | 0.209870168 | HLA-B | NM_005514 | c.T97G | exonic | non- | upper_aerodigestive_ |
| | | | | | | | | | | | | | | synonymous_ | tract;central |
| | | | | | | | | | | | | | | SNV | nervous_system |
| chr6 | 31533435 | A | T | 27 | 419 | 31 | 120 | 7.24E−06 | 0.31184825 | ATP6V1G2- | — | | ncRNA_ | — | upper_aerodigestive_ |
| | | | | | | | | | | DDX39B | | | intronic | | tract;thyroid;skin; |
| | | | | | | | | | | | | | | | haematopoietic_ |
| | | | | | | | | | | | | | | | and_lymphoid tissue |
| chr6 | 32578849 | C | G, | 4 | 110 | 62 | 439 | 0.0495 | 0.139758238 | HLA- | — | | UTR3 | — | haematopoietic_and_ |
| | | | A, | | | | | | | DRB1 | | | | | lymphoid_tissue;liver; |
| | | | T | | | | | | | | | | | | large_intestine |

TABLE 20
Expanded mutations in plasma B cells

| | | | | | | | | | diff_ALT_ | | | | | | |
|---|---|---|---|---|---|---|---|---|---|---|---|---|---|---|---|
| | | | | REF_ | REF_ | ALT_ | ALT_ | | cellular | | | | | | |
| | | | | plasma | other | plasma | other | FDR | prevalence_ | | | | | | |
| | | | | B cell | cell | B cell | cell | adj | plasma B | | | | | | |
| | | | | num- | num- | num- | num- | P- | vs | Gene | RefSeq_ | | Func_ | Mutation_ | |
| CHR | POS | REF | ALT | bers | bers | bers | bers | values | others | Name | ID | Coding | type | type | Cosmic |
| chrX | 4.8E+ | T | C | 14 | 446 | 29 | 126 | 1.97E− | 0.454138884 | TIMP1 | NM_ | c.T372C | exonic | synonymous_ | lung |
| | 07 | | | | | | | 07 | | | 003254 | | | SNV | |

The foregoing description of illustrative embodiments of the invention has been presented for purposes of illustration and of description. It is not intended to be exhaustive or to limit the invention to the precise form disclosed, and modifications and variations are possible in light of the above teachings or may be acquired from practice of the invention. The embodiments were chosen and described in order to explain the principles of the invention and as practical applications of the invention to enable one skilled in the art to utilize the invention in various embodiments and with various modifications as suited to the particular use contemplated. It is intended that the scope of the invention be defined by the claims appended hereto and their equivalents.

What is claimed is:

1. A method of performing single cell nanopore sequencing of genotype-phenotype simultaneously (scNanoGPS), the method comprising:
(1) obtaining barcoded full-length cDNAs sequencing information of single cells;
(2) scanning the barcoded full-length cDNAs sequencing information to acquire barcoded information;
(3) curating errors in the barcode information to produce curated barcoded information;
(4) producing at least one BAM file based on the curated barcoded information; and
(5) calculating multi-omics information of the single cells based on the at least one BAM file.

2. The method according to claim 1, wherein
the barcoded full-length cDNAs sequencing information is obtained via long-read single cell nanopore sequencing technology.

3. The method according to claim 1 further comprising
producing a FASTQ file based on the barcoded information acquired in step (2).

4. The method according to claim 3 further comprising
refining the barcoded information acquired in step (2) before step (3) to produce refined barcoded information independently through an algorithm iCARLO.

5. The method according to claim 4 further comprising
obtaining Unique Molecular Identifiers (UMIs) from the refined barcoded information; and
curating at least one error in the Unique Molecular Identifiers (UMIs) in step (3) for transcriptome analysis.

6. The method according to claim 4, wherein
obtaining transcripts from the refined barcoded information; and
curating at least one error in the transcripts in step (3) for the transcriptome analysis.

7. The method according to claim 4, wherein
the single cells multi-omics information includes at least one of gene expression matrix, isoforms profile and mutations profile.

8. The method according to claim 7, wherein the single cells gene expression matrix is generated via:
(a) calculating a UMI counts of genes in the single cells using the at least one BAM files; wherein each of the at least one BAM files is individually mapped;
(b) selecting consensus reads that mapped to mature mRNA references to detect transcriptional isoforms of the single cells; and
(c) calculating single cell mutation profiles from the consensus reads of the single cells.

9. The method according to claim 1, wherein
the method obviates using short read sequencing information of the single cells or barcode whitelist as guidance for processing the barcoded full-length cDNAs sequencing information.

10. The method according to claim 2, wherein the single cells include approximately 3000-6000 cells per run of the long-read single cell nanopore sequencing.

11. A non-transitory computer readable medium storing a program causing a computer to execute a process of performing single cell nanopore sequencing of genotype-phenotype simultaneously (scNanoGPS), the process comprising:
(1) obtaining barcoded full-length cDNAs sequencing information of single cells
(2) scanning the barcoded full-length cDNAs sequencing information to acquire barcoded information;
(3) curating errors in the barcode information to produce curated barcoded information;
(4) producing at least one BAM file based on the curated barcoded information; and
(5) calculating multi-omics information of the single cells based on the at least one BAM file.

12. The non-transitory computer readable medium according to claim 11, wherein
the barcoded full-length cDNAs sequencing information is obtained via long-read single cell nanopore sequencing technology.

13. The non-transitory computer readable medium according to claim 11, wherein the process further comprises
producing a FASTQ file based on the acquired barcoded information acquired in step (2).

14. The non-transitory computer readable medium according to claim 13, wherein the process further comprises
refining the barcoded information acquired in step (2) before step (3) to produce refined barcoded information.

15. The non-transitory computer readable medium according to claim 14, wherein the process further comprises
obtaining Unique Molecular Identifiers (UMIs) from the refined barcoded information; and
curating at least one error in the Unique Molecular Identifiers (UMIs) in step (3) for transcriptome analysis.

16. The non-transitory computer readable medium according to claim 14, wherein the process further comprises
obtaining transcripts from the refined barcoded information; and
curating at least one error in the transcripts in step (3) for the transcriptome analysis.

17. The non-transitory computer readable medium according to claim 14, wherein
the single cells multi-omics information includes at least one of gene expression matrix, isoforms profile, mutations and fusions profile.

18. The non-transitory computer readable medium according to claim 17, wherein the single cell gene expression matrix is generated via
(a) calculating a UMI counts of genes in the single cells using the at least one BAM files; wherein each of the at least one BAM files is individually mapped;
(b) selecting consensus reads that mapped to mature mRNA references to detect transcriptional isoforms of the single cells; and
(c) calculating single cell mutation profiles from the consensus reads of the single cells.

19. The non-transitory computer readable medium according to claim 11, wherein
the method obviates using guidance of short read sequencing information of the single cells for processing the barcoded full-length cDNAs sequencing information.

20. The non-transitory computer readable medium according to claim 12, wherein the single cells include 3000 cells per run of the long-read single cell nanopore sequencing.


SEQUENCE LISTING (10 sequences)

SEQ ID NO: 1 - DNA, 16 bp, unidentified
  source 1..16: mol_type = unassigned DNA
cctgctccattaggcc

SEQ ID NO: 2 - DNA, 16 bp, unidentified
  source 1..16: mol_type = other DNA
gagcttagttcgctca

SEQ ID NO: 3 - DNA, 16 bp, unidentified
  source 1..16: mol_type = other DNA
ttgatgtcattgtcct

SEQ ID NO: 4 - DNA, 16 bp, unidentified
  source 1..16: mol_type = other DNA
agcggatagcagctat

SEQ ID NO: 5 - DNA, 16 bp, unidentified
  source 1..16: mol_type = other DNA
gagcggtcaggctgtt

SEQ ID NO: 6 - DNA, 16 bp, unidentified
  source 1..16: mol_type = other DNA
gataacgagctgtaac

SEQ ID NO: 7 - DNA, 16 bp, unidentified
  source 1..16: mol_type = other DNA
gccatgatcaccaata

SEQ ID NO: 8 - DNA, 16 bp, unidentified
  source 1..16: mol_type = other DNA
cgcttaacaggtcctg

SEQ ID NO: 9 - DNA, 16 bp, unidentified
  source 1..16: mol_type = other DNA
gcaacagcatgtcagc

SEQ ID NO: 10 - DNA, 16 bp, unidentified
  source 1..16: mol_type = other DNA
gtatgtggtattgagt